(19) United States
(12) Patent Application Publication
(10) Pub. No.: US 2006/0234287 A1
(43) Pub. Date: Oct. 19, 2006

(54) BREAST CANCER PROGRESSION SIGNATURES

(75) Inventors: Mark G. Erlander, Encinitas, CA (US); Xia-Jun Ma, San Diego, CA (US); Dennis C. Sgroi, Winchester, MA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AviaraDx, Inc., Carlsbad, CA (US)

(21) Appl. No.: 11/426,572

(22) Filed: Jun. 26, 2006

Related U.S. Application Data
(63) Continuation of application No. 10/028,018, filed on Dec. 21, 2001, now abandoned.

(51) Int. Cl. C12Q 1/68

(52) U.S. Cl. 435/6

(57) ABSTRACT

Methods and compositions for the identification of breast cancer progression signatures are provided. The signature profiles are identified based upon multiple sampling of reference breast tissue samples from independent cases of breast cancer and provide a reliable set of molecular criteria for identification of cells as being in one or more particular stages of breast cancer.

### FIELD OF THE INVENTION

The invention relates to the identification and use of gene expression profiles, or patterns, involved in breast cancer progression. The gene expression profiles, whether embodied in nucleic acid expression, protein expression, or other expression formats, are used in the study and/or diagnosis of cells and tissue during breast cancer progression as well as for the study and/or determination of prognosis of a patient. When used for diagnosis or prognosis, the profiles are used to predict the status and/or phenotype of cells and tissues relative to breast cancer and the treatment thereof.

### BACKGROUND OF THE INVENTION

Breast cancer is by far the most common cancer among women. Each year, more than 180,000 and 1 million women in the U.S. and worldwide, respectively, are diagnosed with breast cancer. Breast cancer is the leading cause of death for women between ages 50-55, and is the most common non-preventable malignancy in women in the Western Hemisphere. An estimated 2,167,000 women in the United States are currently living with the disease (National Cancer Institute, Surveillance Epidemiology and End Results (NCI SEER) program, Cancer Statistics Review (CSR), www-seer.ims.nci.nih.gov/Publications/CSR1973 (1998)). Based on cancer rates from 1995 through 1997, a report from the National Cancer Institute (NCI) estimates that about 1 in 8 women in the United States (approximately 12.8 percent) will develop breast cancer during her lifetime (NCI's Surveillance, Epidemiology, and End Results Program (SEER) publication SEER Cancer Statistics Review 1973-1997). Breast cancer is the second most common form of cancer, after skin cancer, among women in the United States. An estimated 250,100 new cases of breast cancer are expected to be diagnosed in the United States in 2001. Of these, 192,200 new cases of more advanced (invasive) breast cancer are expected to occur among women (an increase of 5% over last year), 46,400 new cases of early stage (in situ) breast cancer are expected to occur among women (up 9% from last year), and about 1,500 new cases of breast cancer are expected to be diagnosed in men (Cancer Facts & Figures 2001 American Cancer Society). An estimated 40,600 deaths (40,300 women, 400 men) from breast cancer are expected in 2001. Breast cancer ranks second only to lung cancer among causes of cancer deaths in women. Nearly 86% of women who are diagnosed with breast cancer are likely to still be alive five years later, though 24% of them will die of breast cancer after 10 years, and nearly half (47%) will die of breast cancer after 20 years.

Every woman is at risk for breast cancer. Over 70 percent of breast cancers occur in women who have no identifiable risk factors other than age (U.S. General Accounting Office. Breast Cancer, 1971-1991: Prevention, Treatment and Research. GAO/PEMD-92-12; 1991). Only 5 to 10% of breast cancers are linked to a family history of breast cancer (Henderson IC, Breast Cancer. In: Murphy G P, Lawrence W L, Lenhard R E (eds). Clinical Oncology. Atlanta, Ga.: American Cancer Society; 1995:198-219).

Each breast has 15 to 20 sections called lobes. Within each lobe are many smaller lobules. Lobules end in dozens of tiny bulbs that can produce milk. The lobes, lobules, and bulbs are all linked by thin tubes called ducts. These ducts lead to the nipple in the center of a dark area of skin called the areola. Fat surrounds the lobules and ducts. There are no muscles in the breast, but muscles lie under each breast and cover the ribs. Each breast also contains blood vessels and lymph vessels. The lymph vessels carry colorless fluid called lymph, and lead to the lymph nodes. Clusters of lymph nodes are found near the breast in the axilla (under the arm), above the collarbone, and in the chest.

Breast tumors can be either benign or malignant. Benign tumors are not cancerous, they do not spread to other parts of the body, and are not a threat to life. They can usually be removed, and in most cases, do not come back. Malignant tumors are cancerous, and can invade and damage nearby tissues and organs. Malignant tumor cells may metastisize, entering the bloodstream or lymphatic system. When breast cancer cells metastisize outside the breast, they are often found in the lymph nodes under the arm (axillary lymph nodes). If the cancer has reached these nodes, it means that cancer cells may have spread to other lymph nodes or other organs, such as bones, liver, or lungs.

Major and intensive research has been focussed on early detection, treatment and prevention. This has included an emphasis on determining the presence of precancerous or cancerous ductal epithelial cells. These cells are analyzed, for example, for cell morphology, for protein markers, for nucleic acid markers, for chromosomal abnormalities, for biochemical markers, and for other characteristic changes that would signal the presence of cancerous or precancerous cells. This has led to various molecular alterations that have been reported in breast cancer, few of which have been well characterized in human clinical breast specimens. Molecular alterations include presence/absence of estrogen and progesterone steroid receptors, HER-2 expression/amplification (Mark H F, et al. HER-2/neu gene amplification in stages I-IV breast cancer detected by fluorescent in situ hybridization. Genet Med; 1(3):98-103 1999), Ki-67 (an antigen that is present in all stages of the cell cycle except G0 and used as a marker for tumor cell proliferation, and prognostic markers (including oncogenes, tumor suppressor genes, and angiogenesis markers) like p53, p27, Cathepsin D, pS2, multi-drug resistance (MDR) gene, and CD31.

Examination of cells by a trained pathologist has also been used to establish whether ductal epithelial cells are normal (i.e. not precancerous or cancerous or having another noncancerous abnormality), precancerous (i.e. comprising hyperplasia, atypical ductal hyperplasia (ADH)) or cancerous (comprising ductal carcinoma in situ, or DCIS, which includes low grade ductal carcinoma in situ, or LG-DCIS, and high grade ductal carcinoma in situ, or HG-DCIS) or invasive (ductal) carcinoma (IDC). Pathologists may also identify the occurrence of lobular carcinoma in situ (LCIS) or invasive lobular carcinoma (ILC). Breast cancer progression may be viewed as the occurrence of abnormal cells, such as those of ADH, DCIS, IDC, LCIS, and/or ILC, among normal cells.

It remains unclear whether normal cells become hyperplastic (such as ADH) and then progressing on to become malignant (DCIS, IDC, LCIS, and/or ILC) or whether normal cells are able to directly become malignant without transitioning through a hyperplastic stage. It has been observed, however, that the presence of ADH indicates a higher likelihood of developing a malignancy. This has resulting in treatment of patients with ADH to begin treatment with an antineoplastic/antitumor agent such as tamoxifen. This is in contrast to the treatment of patients with malignant breast cancer which usually includes surgical removal.

The rational development of preventive, diagnostic and therapeutic strategies for women at risk for breast cancer would be aided by a molecular map of the tumorigenesis process. Relatively little is known of the molecular events that mediate the transition of normal breast cells to the various stages of breast cancer progression. In particular, there is a significant paucity of information regarding the genetic changes that are associated with the earliest stages of human breast cancer, which include the transition of normal breast cells to atypical hyperplastic and/or pre-invasive malignant cells (carcinoma in situ).

Molecular means of identifying the differences between normal, non-cancerous cells and cancerous cells (in general) have also been the focus of intense study. The use of cDNA libraries to analyze differences in gene expression patterns in normal versus tumorigenic cells has been described (U.S. Pat. No. 4,981,783). DeRisi et al. (1996) describe the analysis of gene expression patterns between two cell lines: UACC-903, which is a tumorigenic human melanoma cell line, and UACC-903(+6), which is a chromosome 6 suppressed non-tumorigenic form of UACC-903. Labeled cDNA probes made from mRNA from these cell lines were applied to DNA microarrays containing 870 different cDNAs and controls. Genes that were preferentially expressed in one of the two cell lines were identified.

Golub et al. (1999) describe the use of gene expression monitoring as means to cancer class discovery and class prediction between acute myeloid leukemia (AML) and acute lymphoblastic leukemia (ALL). Their approach to class predictors used a neighborhood analysis followed by cross-validation of the validity of the predictors by withholding one sample and building a predictor based only on the remaining samples. This predictor is then used to predict the class of the withheld sample. They also used cluster analysis to identify new classes (or subtypes) within the AML and ALL.

Gene expression patterns in human breast cancers have been described by Perou et al. (1999), who studied gene expression between cultured human mammary epithelia cells (HMEC) and breast tissue samples by use of microarrays comprising about 5000 genes. They used a clustering algorithm to identify patterns of expression in HMEC and tissue samples. Perou et al. (2000) describe the use of clustered gene expression profiles to classify subtypes of human breast tumors. Hedenfalk et al. describe gene expression profiles in BRCA1 mutation positive, BRCA2 mutation positive, and sporadic tumors. Sgroi et al. also analyzed gene expression of normal and breast cancer cells from a single patient. Using gene expression patterns to distinguish breast tumor subclasses and predict clinical implications is described by Sorlie et al. and West et al.

All of the above described approaches, however, utilize heterogeneous populations of cells found in culture or in a biopsy to obtain information on gene expression patterns. The use of such populations may result in the inclusion or exclusion of multiple genes from the patterns. For this and the lack of statistical robustness reasons, the gene expression patterns observed by the above described approaches provide little confidence that the differences in gene expression may be meaningfully associated with the stages of breast cancer.

### SUMMARY OF THE INVENTION

The present invention relates to the identification and use of gene expression patterns (or profiles or “signatures”) which are correlated with (and thus able to discriminate between) cells in various stages of breast cancer. Broadly defined, these stages are non-malignant versus malignant, but may also be viewed as normal versus atypical (optionally including reactive and pre-neoplastic) versus cancerous. Another definition of the stages is normal versus precancerous (e.g. atypical ductal hyperplasia (ADH) or atypical lobular hyperplasia (ALH)) versus cancerous (e.g. carcinoma in situ such as DCIS and/or LCIS) versus invasive (e.g. carcinomas such as IDC and/or ILC). DCIS may be further viewed as low grade versus high grade or grade I through grade III.

The gene expression patterns comprise one or more than one gene capable of discriminating between various stages of breast cancer with significant accuracy. The gene(s) are identified as correlated with various stages of breast cancer such that the levels of their expression are relevant to a determination of the stage of breast cancer of a cell. Thus in one aspect, the invention provides a method to determine the stage of breast cancer of a subject afflicted with, or suspected of having, breast cancer by assaying a cell containing sample from said subject for expression of one or more than one gene disclosed herein as correlated with one or more stages of breast cancer.

Gene expression patterns of the invention are identified by analysis of gene expression in multiple samples of each stage to be studied. The overall gene expression profile of each sample is obtained by analyzing the expressed or unexpressed state of genes in each stage relative to each other (one gene to another across all genes). This overall profile is then analyzed to identify genes that are positively, or negatively, correlated, with a stage of breast cancer relative to other genes. An expression profile of a subset of human genes may then be identified by the methods of the present invention as correlated with breast cancer. The use of multiple samples increases the confidence which which a gene may be believed to be correlated with a particular stage. Without sufficient confidence, it remains unpredictable whether a particular gene is actually correlated with a stage of breast cancer and also unpredictable whether a particular gene may be successfully used to identify the stage of an unknown breast cancer cell sample.

A profile of genes that are highly correlated with one stage relative to another may be used to assay an sample from a subject afflicted with, or suspected of having, breast cancer to identify the stage of breast cancer to which the sample belongs. Such an assay may be used as part of a method to determine the therapeutic treatment for said subject based upon the stage(s) of breast cancer identified.

The correlated genes may be used singly with significant accuracy or in combination to increase the ability to accurately discriminate between various stages of breast cancer. The present invention thus provides means for correlating a molecular expression phenotype with a physiological (cellular) stage or state. This correlation provides a way to molecularly diagnose and/or monitor a cell's status in comparison to different cancerous versus non-cancerous phenotypes as disclosed herein. Additional uses of the correlated gene(s) are in the classification of cells and tissues; determination of diagnosis and/or prognosis; and determination and/or alteration of therapy.

The ability to discriminate is conferred by the identification of expression of the individual genes as relevant and not by the form of the assay used to determine the actual level of expression. An assay may utilize any identifying feature of an identified individual gene as disclosed herein as long as the assay reflects, quantitatively or qualitatively, expression of the gene. Identifying features include, but are not limited to, unique nucleic acid sequences used to encode (DNA), or express (RNA), said gene or epitopes specific to, or activities of, a protein encoded by said gene. All that is required is the identity of the gene(s) necessary to discriminate between stages of breast cancer and an appropriate cell containing sample for use in an expression assay.

In one aspect, the invention provides for the identification of the gene expression patterns by analyzing global, or near global, gene expression from single cells or homogenous cell populations which have been dissected away from, or otherwise isolated or purified from, contaminating cells beyond that possible by a simple biopsy. Because the expression of numerous genes fluctuate between cells from different patients as well as between cells from the same patient sample, multiple individual gene expression patterns are used as reference data to generate models which in turn permit the identification of individual gene(s) that are most highly correlated with particular breast cancer stages and/or have the best the ability to discriminate cells of one stage from another.

In another aspect, the invention provides physical and methodological means for detecting the expression of gene(s) identified by the models generated by individual expression patterns. These means may be directed to assaying one or more aspect of the DNA template(s) underlying the expression of the gene(s), of the RNA used as an intermediate to express the gene(s), or of the proteinaceous product expressed by the gene(s).

In a further aspect, the gene(s) identified by a model as capable of discriminating between breast cancer stages may be used to identify the cellular state of an unknown sample of cell(s) from the breast. Preferably, the sample is isolated via non-invasive means. The expression of said gene(s) in said unknown sample may be determined and compared to the expression of said gene(s) in reference data of gene expression patterns from the various stages of breast cancer. Optionally, the comparison to reference samples may be by comparison to the model(s) constructed based on the reference samples.

One advantage provided by the present invention is that contaminating, non-breast cells (such as infiltrating lymphocytes or other immune system cells) are not present to possibly affect the genes identified or the subsequent analysis of gene expression to identify the status of suspected breast cancer cells. Such contamination is present where a biopsy is used to generate gene expression profiles.

While the present invention has been described mainly in the context of human breast cancer, it may be practiced in the context of breast cancer of any animal known to be potentially afflicted by breast cancer. Preferred animals for the application of the present invention are mammals, particularly those important to agricultural applications (such as, but not limited to, cattle, sheep, horses, and other “farm animals”) and for human companionship (such as, but not limited to, dogs and cats).

### BRIEF DESCRIPTION OF THE FIGURES

FIG. 1 is a schematic representing a data matrix of a pair-wise comparison between Grade I and Grade III DCIS among 16 samples (across the top) and a large number of genes identified by “CloneID”) along the left hand side.

FIG. 2 is a table showing the actual weight data corresponding to Example II, where the data from ten genes (by CloneID number vertically) are compared to DCIS and ADH samples (across the top). Some data in the table has been vertically presented to permit the table to be displayed on a single sheet. The use of “−” with data in the table reflects genes that are more highly expressed in ADH relative to DCIS. The absence of “−”reflects genes that are more highly expressed in DCIS relative to ADH.

FIG. 3 is a table showing the actual weight data corresponding to Example VII, where the data from over 300 genes (by CloneID number vertically) are compared to DCIS and ADH samples (across the top). Some data in the table has been vertically presented solely for display purposes. The use of “−” with data in the table reflects genes that are more highly expressed in ADH relative to DCIS. The absence of “−” reflects genes that are more highly expressed in DCIS relative to ADH.

FIG. 4 is a table showing the actual weight data corresponding to Example VIII, where the data from over 300 genes (by CloneID number vertically) are compared to samples (across the top) from two grades of DCIS. The use of “−” with data in the table reflects genes that are more highly expressed in grade I relative to grade III. The absence of “−” reflects genes that are more highly expressed in grade III relative to grade I.

### DETAILED DESCRIPTION OF THE SPECIFIC EMBODIMENTS

Definitions of Terms as used Herein:

A gene expression “pattern” or “profile” or “signature” refers to the relative expression of a gene between two or more stages of breast cancer which is correlated with being able to distinguish between said stages.

A “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “stage” or “stages” (or equivalents thereof) of breast cancer refer to a physiologic state of a breast cell as defined by known cytological or histological (including immunohistology, histochemistry, and immunohistochemistry) procedures and are readily known to skilled in the art. Non-limiting examples include normal versus abnormal, non-cancerous versus cancerous, the different stages described herein (e.g. hyperplastic, carcinoma, and invasive), and grades within different stages (e.g. grades I, II, or III or the equivalents thereof within cancerous stages).

The terms “correlate” or “correlation” or equivalents thereof refer to an association between expression of one or more genes and a physiologic state of a breast cell to the exclusion of one or more other stages and/or identified by use of the methods as described herein. A gene may be expressed at higher or lower levels and still be correlated with one or more breast cancer stages.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence.

By corresponding is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17). Methods for amplifying mRNA are generally known in the art, and include reverse transcription PCR (RT-PCR) and those described in U.S. Patent Application (number to be assigned) entitled “Nucleic Acid Amplification” filed on Oct. 25, 2001 as attorney docket number 485772002900 as well as U.S. Provisional Patent Application 60/298,847 (filed Jun. 15, 2001) and 60/257,801 (filed Dec. 22, 2000), all of which are hereby incorporated by reference in their entireties as if fully set forth. Alternatively, RNA may be directly labeled as the corresponding cDNA by methods known in the art.

A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². Preferably, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of primers in the array is known, the identities of a sample polynucleotides can be determined based on their binding to a particular position in the microarray.

Because the invention relies upon the identification of genes that are over- or under-expressed, one embodiment of the invention involves determining expression by hybridization of mRNA, or an amplified or cloned version thereof, of a sample cell to a polynucleotide that is unique to a particular gene sequence. Preferred polynucleotides of this type contain at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Even more preferred are polynucleotides of at least about 50, at least about 100, and at least about 150 basepairs of a gene sequence that is not found in other gene sequences. The term “about” as used in the preceding sentence refers to an increase or decrease of 10% from the stated numerical value.

Alternatively, and in another embodiment of the invention, gene expression may be determined by analysis of expressed protein in a cell sample of interest by use of one or more antibodies specific for one or more epitopes of individual gene products (proteins) in said cell sample. Such antibodies are preferably labeled to permit their easy detection after binding to the gene product.

The term “label” refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

As used herein, a “breast tissue sample” or “breast cell sample” refers to a sample of breast tissue or fluid isolated from an individual suspected of being afflicted with, or at risk of developing, breast cancer. Such samples are primary isolates (in contrast to cultured cells) and may be collected by any non-invasive means, including, but not limited to, ductal lavage, fine needle aspiration, needle biopsy, the devices and methods described in U.S. Pat. No. 6,328,709, or any other suitable means recognized in the art. Alternatively, the “sample” may be collected by an invasive method, including, but not limited to, surgical biopsy.

“Expression” and “gene expression” include transcription and/or translation of nucleic acid material.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion or insertion. Single nucleotide polymorphism (SNP) is also a sequence mutation as used herein. Because the present invention is based on the relative level of gene expression, mutations in non-coding regions of genes as disclosed herein may also be assayed in the practice of the invention.

“Detection” includes any means of detecting, including direct and indirect detection of gene expression and changes therein. For example, “detectably less” products may be observed directly or indirectly, and the term indicates any reduction (including the absence of detectable signal). Similarly, “detectably more” product means any increase, whether observed directly or indirectly.

Unless defined otherwise all technical and scientific terms used herein have the same meaning as commonly understood to one of ordinary skill in the art to which this invention belongs.

### Specific Embodiments

The present invention relates to the identification and use of gene expression patterns (or profiles or “signatures”) which discriminate between (or are correlated with) cells in various stages of breast cancer. Such patterns may be determined by the methods of the invention by use of a number of reference cell or tissue samples, such as those reviewed by a pathologist of ordinary skill in the pathology of breast cancer, which reflect various stages of breast cancer. Because the overall gene expression profile differs from person to person, cancer to cancer, and cancer cell to cancer cell, correlations between certain cell states and genes expressed or underexpressed may be made as disclosed herein to identify genes that are capable of discriminating between different breast cancer states.

The present invention may be practiced with any number of genes believed, or likely to be, differentially expressed in breast cancer cells. In Example I below, approximately 12,000 genes were used to identify hundreds of genes capable of discriminating between various stages of breast cancer as shown in Examples 2-9. The identification may be made by using expression profiles of various homogenous normal and breast cancer cell populations, which were isolated by microdissection, such as, but not limited to, laser capture microdissection (LCM) of 100-1000 cells. Each gene of the expression profile may be assigned weights based on its ability to discriminate between two or more stages of breast cancer (see Example I). The magnitude of each assigned weight indicates the extent of difference in expression between the two groups and is an approximation of the ability of expression of the gene to discriminate between the two groups (and thus stages). The magnitude of each assigned weight also approximates the extent of correlation between expression of individual gene(s) and particular breast cancer stages.

It should be noted that merely high levels of expression in cells from a particular stage or stages does not necessarily mean that a gene will be identified as having a high absolute weight value.

Genes with top ranking weights (in absolute terms) may be used to generate models of gene expressions that would maximally discriminate between the two groups. Alternatively, genes with top ranking weights (in absolute terms) may be used in combination with genes with lower weights without signficant loss of ability to discriminate between groups. Such models may be generated by any appropriate means recognized in the art, including, but not limited to, cluster analysis, supported vector machines, neural networks or other algorithm known in the art. The models are capable of predicting the classification of a unknown sample based upon the expression of the genes used for discrimination in the models. “Leave one out” cross-validation may be used to test the performance of various models and to help identify weights (genes) that are uninformative or detrimental to the predictive ability of the models. Cross-validation may also be used to identify genes that enhance the predictive ability of the models.

The gene(s) identified as correlated with particular breast cancer stages by the above models provide the ability to focus gene expression analysis to only those genes that contribute to the ability to identify a cell as being in a particular stage of breast cancer relative to another stage or stages. The expression of other genes in a breast cancer cell would be relatively unable to provide information concerning, and thus assist in the discrimination of, different stages of breast cancer. For example, the alpha subunit of human topoisomerase II (identified by CloneID 825470) has been found to be useful in discriminations between normal and atypical cells (ADH and DCIS and IDC and LCIS), between normal and ADH cells compared to DCIS and IDC cells, between normal and DCIS cells, between ADH and DCIS cells, between grade I and grade III DCIS cells, and between grade I and grade III IDC cells but not between normal and ADH cells (see Examples II to IX below). Thus expression of this topoisomerase II subunit would be utilized in models to discriminate between the above listed stages but not for discerning normal from ADH cells. This type of analysis is readily incorporated into algorithms used to generate models with reference gene expression data.

As will be appreciated by those skilled in the art, the models are highly useful with even a small set of reference gene expression data and can become increasingly accurate with the inclusion of more reference data although the incremental increase in accuracy will likely diminish with each additional datum. The preparation of additional reference gene expression data using genes identified and disclosed herein for discriminating between different stages of breast cancer is routine and may be readily performed by the skilled artisan to permit the generation of models as described above to predict the status of an unknown sample based upon the expression levels of those genes.

To determine the expression levels of genes in the practice of the present invention, any method known in the art may be utilized. In one preferred embodiment of the invention, expression based on detection of RNA which hybridizes to the genes identified and disclosed herein is used. This is readily performed by any RNA detection or amplification+detection method known or recognized as equivalent in the art such as, but not limited to, reverse transcription-PCR, the methods disclosed in U.S. Patent Application (number to be assigned) entitled “Nucleic Acid Amplification” filed on Oct. 25, 2001 as attorney docket number 485772002900 as well as U.S. Provisional Patent Applications 60/298,847 (filed Jun. 15, 2001) and 60/257,801 (filed Dec. 22, 2000), and methods to detect the presence, or absence, of RNA stabilizing or destabilizing sequences.

Alternatively, expression based on detection of DNA status may be used. Detection of the DNA of an indentified gene as methylated or deleted may be used for genes that have decreased expression in correlation with a particular breast cancer stage. This may be readily performed by PCR based methods known in the art. Conversely, detection of the DNA of an indentified gene as amplified may be used for genes that have increased expression in correlation with a particular breast cancer stage. This may be readily performed by PCR based, fluorescent in situ hybridization (FISH) and chromosome in situ hybridization (CISH) methods known in the art.

Expression based on detection of a presence, increase, or decrease in protein levels or activity may also be used. Detection may be performed by any immunohistochemistry (IHC) based, blood based (especially for secreted proteins), antibody (including autoantibodies against the protein) based, ex foliate cell (from the cancer) based, mass spectroscopy based, and image (including used of labeled ligand) based method known in the art and recognized as appropriate for the detection of the protein. Antibody and image based methods are additionally useful for the localization of tumors after determination of cancer by use of cells obtained by a non-invasive procedure (such as ductal lavage or fine needle aspiration), where the source of the cancerous cells is not known. A labeled antibody or ligand may be used to localize the carcinoma(s) within a patient.

A preferred embodiment using a nucleic acid based assay to determine expression is by immobilization of one or more of the genes identified herein on a solid support, including, but not limited to, a solid substrate as an array or to beads or bead based technology as known in the art. Alternatively, solution based expression assays known in the art may also be used. The immobilized gene(s) may be in the form of polynucleotides that are unique or otherwise specific to the gene(s) such that the polynucleotide would be capable of hybridizing to a DNA or RNA corresponding to the gene(s). These polynucleotides may be the full length of the gene(s) or be short sequences of the genes that are optionally minimally interrupted (such as by mismatches or inserted non-complementary basespairs) such that hybridization with a DNA or RNA corresponding to the gene(s) is not affected.

The immobilized gene(s) may be used to determine the state of nucleic acid samples prepared from sample breast cell(s) for which the pre-cancer or cancer status is not known or for confirmation of a status that is already assigned to the sample breast cell(s). Without limiting the invention, such a cell may be from a patient suspected of being afflicted with, or at risk of developing, breast cancer. The immobilized polynucleotide(s) need only be sufficient to specifically hybridize to the corresponding nucleic acid molecules derived from the sample. While even a single correlated gene sequence may to able to provide adequate accuracy in discriminating between two breast cancer cell stages, two or more, three or more, four or more, five or more, six or more, seven or more, eight or more, nine or more, ten or more, or eleven or more of the genes identified herein may be used as a subset capable of discriminating may be used in combination to increase the accuracy of the method. The invention specifically contemplates the selection of more than one, two or more, three or more, four or more, five or more, six or more, seven or more, eight or more, nine or more, ten or more, or eleven or more of the genes disclosed in the tables and figures herein for use as a subset in the identification of whether an unknown or suspicious breast cancer sample is normal or is in one or more stages of breast cancer. Optionally, the genes used will not include CloneID 809507, which is also known as GenBank accession number AA454563, described as an EST with high similarity to CD63 but of unknown function.

In embodiments where only one or a few genes are to be analyzed, the nucleic acid derived from the sample breast cancer cell(s) may be preferentially amplified by use of appropriate primers such that only the genes to be analyzed are amplified to reduce contaminating background signals from other genes expressed in the breast cell. Alternatively, and where multiple genes are to be analyzed or where very few cells (or one cell) is used, the nucleic acid from the sample may be globally amplified before hybridization to the immobilized polynucleotides. Of course RNA, or the cDNA counterpart thereof may be directly labeled and used, without amplification, by methods known in the art.

The above assay embodiments may be used in a number of different ways to identify or detect the breast cancer stage, if any, of a breast cancer cell sample from a patient. In many cases, this would reflect a secondary screen for the patient, who may have already undergone mammography or physical exam as a primary screen. If positive, the subsequent needle biopsy, ductal lavage, fine needle aspiration, or other analogous methods may provide the sample for use in the above assay embodiments. The present invention is particularly useful in combination with non-invasive protocols, such as ductal lavage or fine needle aspiration, to prepare a breast cell sample. The current analysis of ductal lavage samples is by cytological examination by a trained pathologist who classifies the samples in terms that are at least partly subjective: unsatisfactory (too few cells), benign (including fibrocystic change), atypical (or mild atypia), suspicious (or marked atypia), or malignant.

The present invention provides a more objective set of criteria, in the form of gene expression profiles of a discrete set of genes, to discriminate (or delineate) between meaningful stages (or classes) of breast cancer cells. In particularly preferred embodiments of the invention, the assays are used to discriminate between ADH and DCIS or otherwise malignant cells, which is a critical determination for decisions concerning subsequent treatment and therapy for the patient. Another particularly preferred determination is between the three grades (I, II, III) of carcinomas in situ as well as the discrimination between grade III carcinomas in situ and invasive carcinomas. Other pairwise comparisons that are provided by the invention include, but are not limited to, normal versus ADH, normal versus carcinoma in situ, normal versus invasive, normal versus cancerous (i.e. carcinoma present), ADH versus cancerous, and carcinoma in situ versus invasive. With the use of alternative algorithms, such as neural networks, comparisons that discriminate between multiple (more than pairwise) classes may also be performed. It is believed by the inventors that the present invention is the first example of objective, molecular criteria for making these discriminations.

In one embodiment of the invention, the isolation and analysis of a breast cancer cell sample may be performed as follows:
(1) Ductal lavage or other non-invasive procedure is performed on a patient to obtain a sample.
(2) Sample is prepared and coated onto a microscope slide. Note that ductal lavage results in clusters of cells that are cytologically examined as stated above.
(3) Pathologist or image analysis software scans the sample for the presence of atypical cells.
(4) If atypical cells are observed, those cells are harvested (e.g. by microdissection such as LCM).
(5) RNA is extracted from the harvested cells.
(6) RNA is purified, amplified, and labeled.
(7) Labeled nucleic acid is contacted with a microarray containing polynucleotides of the genes identified herein as correlated to discriminations between two or more stages of breast cancer under hybridization conditions, then processed and scanned to obtain a pattern of intensities of each spot (relative to a control for general gene expression in cells) which determine the level of expression of the gene(s) in the cells.
(8) The pattern of intensities is analyzed by comparison to the expression patterns of the genes in known samples of normal and breast cancer cells (relative to the same control).

A specific example of the above method would be performing ductal lavage following a primary screen, observing and collecting atypical cells for analysis. The comparison to known expression patterns, such as that made possible by a model generated by an algorithm (such as, but not limited to nearest neighbor type analysis, SVM, or neural networks) with reference gene expression data for the different breast cancer stages, identifies the cells as being most likely ADH.

Alternatively, the sample may permit the collection of both normal and atypical cells for analysis. The gene expression patterns for each of these two samples will be compared to each other as well as the model and the normal versus individual abnormal comparisons therein based upon the reference data set. This approach can be significantly more powerful that the atypical cells only approach because it utilizes significantly more information from the normal cells and the differences between normal and atypical cells (in both the sample and reference data sets) to determine the status of the atypical cells from the sample.

By appropriate selection of the genes used in the analysis, identification of the relative amounts of atypical cells may also be possible, although in most clinical settings, the identification of the highest grade of breast cancer with confidence makes identification of lower grades less important. Stated differently, the identification of invasive cancer determines the clinical situation regardless of the presence of carcinoma in situ or hyperplastic cells, or the identification of carcinoma in situ makes determines the clinical situation regardless of the presence of hyperplastic cells.

With use of the present invention, skilled physicians may prescribe treatments based on non-invasive samples that they would have prescribed for a patient which had previously received a diagnosis via a solid tissue biopsy.

The above discussion is also applicable where a palpable lesion is detected followed by fine needle aspiration or needle biopsy of cells from the breast. The cells are plated and reviewed by a pathologist or automated imaging system which selects cells for analysis as described above. This again provides a means of linking visual to molecular cytology and provides a less subjective means of identifying the physiological state of breast cancer cells without the need for invasive solid tissue biopsies.

The present invention may also be used, however, with solid tissue biopsies. For example, a solid biopsy may be collected and prepared for visualization followed by determination of expression of one or more genes identified herein to determine the stage of breast cancer, if any. One preferred means is by use of in situ hybridization with polynucleotide or protein identifying probe(s) for assaying expression of said gene(s).

In an alternative method, the solid tissue biopsy may be used to extract molecules followed by analysis for expression of one or more gene(s). This provides the possibility of leaving out the need for visualization and collection of only those cells suspected of being atypical. This method may of course be modified such that only cells suspected of being atypical are collected and used to extract molecules for analysis. This would require visualization and selection as an prerequisite to gene expression analysis.

In a further modification of the above, both normal cells and cells suspected of being atypical are collected and used to extract molecules for analysis of gene expression. The approach, benefits and results are as described above using non-invasive sampling.

In a further alternative to all of the above, the gene(s) identified herein may be used as part of a simple PCR or array based assay simply to determine the presence of atypical cells in a sample from a non-invasive sampling procedure. This is simple to perform and utilizes genes identified to be the best discriminators of normal versus abnormal cells without the need for any cytological examination. If no atypical cells are identified, no cytological examination is necessary. If atypical cells are identified, cytological examination follows, and a more comprehensive analysis, as described above, may follow.

The genes identified herein may be used to generate a model capable of predicting the breast cancer stage (if any) of an unknown breast cell sample based on the expression of the identified genes in the sample. Such a model may be generated by any of the algorithms described herein or otherwise known in the art as well as those recognized as equivalent in the art using gene(s) (and subsets thereof) disclosed herein for the identification of whether an unknown or suspicious breast cancer sample is normal or is in one or more stages of breast cancer. The model provides a means for comparing expression profiles of gene(s) of the subset from the sample against the profiles of reference data used to build the model. The model can compare the sample profile against each of the reference profiles or against model defining delineations made based upon the reference profiles. Additionally, relative values from the sample profile may be used in comparison with the model or reference profiles.

In a preferred embodiment of the invention, breast cell samples identified as normal and abnormal (atypical) from the same subject may be analyzed for their expression profiles of the genes used to generate the model. This provides an advantageous means of identifying the stage of the abnormal sample based on relative differences from the expression profile of the normal sample. These differences can then be used in comparison to differences between normal and individual abnormal reference data which was also used to generate the model.

The detection of gene expression from the samples may be by use of a single microarray able to assay gene expression from all pairwise comparisons disclosed herein for convenience and accuracy.

Other uses of the present invention include providing the ability to identify breast cancer cell samples as being those of a particular stage of cancer for further research or study. This provides a particular advantage in many contexts requiring the identification of breast cancer stage based on objective genetic or molecular criteria rather than cytological observation. It is of particular utility to distinguish different grades of a particular breast cancer stage for further study, research or characterization because no objective criteria for such delineation was previously available.

The materials for use in the methods of the present invention are ideally suited for preparation of kits produced in accordance with well known procedures. The invention thus provides kits comprising agents for the detection of expression of the disclosed genes for identifying breast cancer stage. Such kits optionally comprising the agent with an identifying description or label or instructions relating to their use in the methods of the present invention, is provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

The methods provided by the present invention may also be automated in whole or in part. All aspects of the present invention may also be practiced such that they consist essentially of a subset of the disclosed genes to the exclusion of material irrelevant to the identification of breast cancer stages in a cell containing sample.

Having now generally described the invention, the same will be more readily understood through reference to the following examples which are provided by way of illustration, and are not intended to be limiting of the present invention, unless specified.

### EXAMPLES

### Example I

### Materials and Methods

Clinical Specimens

Clinical biopsies from 30 patients were obtained from the Massachusetts General Hospital with Institutional Review Board approval. The tissue from one of the patients was not associated with breast cancer of any kind since it was from a breast reduction procedure. Pathological and histological information for the biopsies were also obtained. Three independent captures of about 1000 breast epithelial cells of one or more of the four different disease stages (normal, N; atypical ductal hyperplasia, ADH; ductal carcinoma in situ, DCIS; invasive ductal carcinoma, IDC) were procured from each biopsy using Laser Capture Microdissection (LCM, Arcturus Engineering). Three independent captures of LCIS (lobular carcinoma in situ) in one biopsy were also made. Total RNA was extracted from the captured (procured) cells and amplified with a T7-promoter based RNA amplification protocol. The human universal reference RNA (Stratagene, La Jolla), was similarly amplified and used as the reference channel in a two-color microarray hybridization.

Microarrays

To maximize coverage of breast cancer-related genes on the microarrays used, 11,435 cDNA clones from the IMAGE consortium (Research Genetics) were obtained. These clones were selected based on literature knowledge (such as, but not limited to, preferential expression in cancer versus normal cells) and after mining (such as, but not limited to, preferential expression in breast tissues) gene expression information in the expressed sequence tags (EST) databases and the Serial Analysis of Gene Expression (SAGE) data sets available from the National Center for Biotechnology Information (NCBI, http://www/ncbi.nlm.nih.gov).

Microarray Data Processing

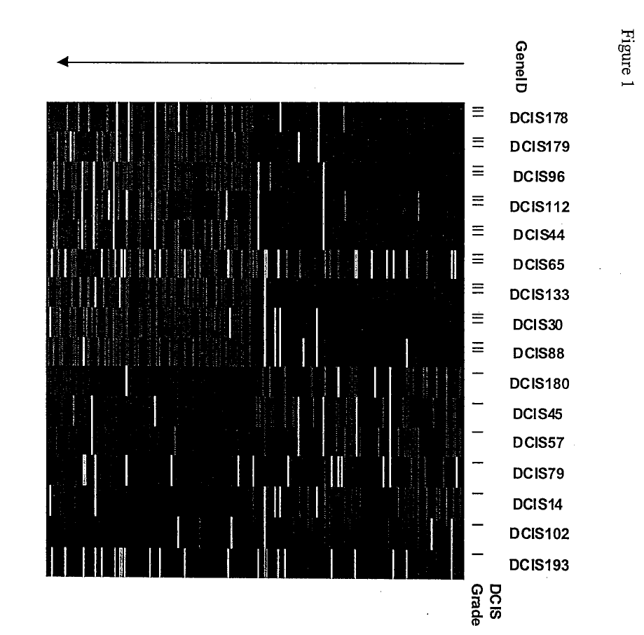

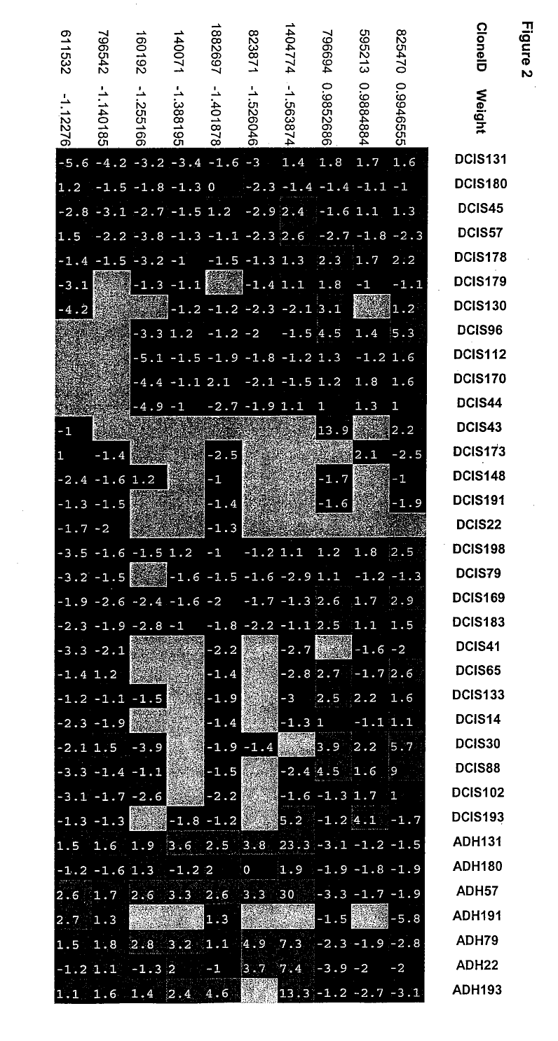

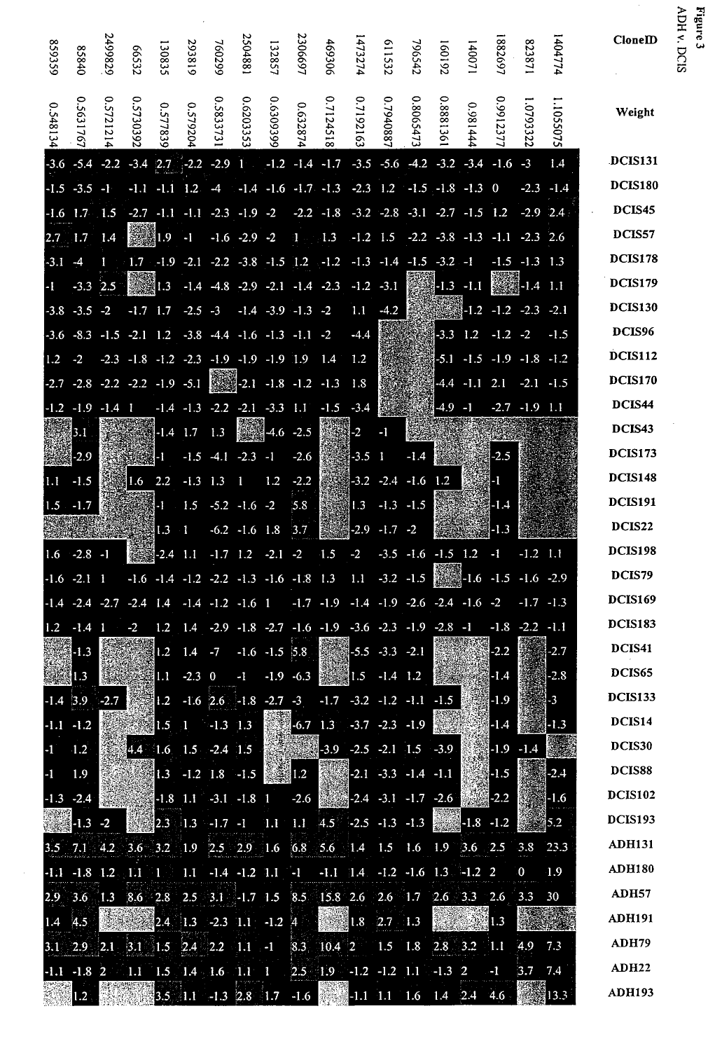

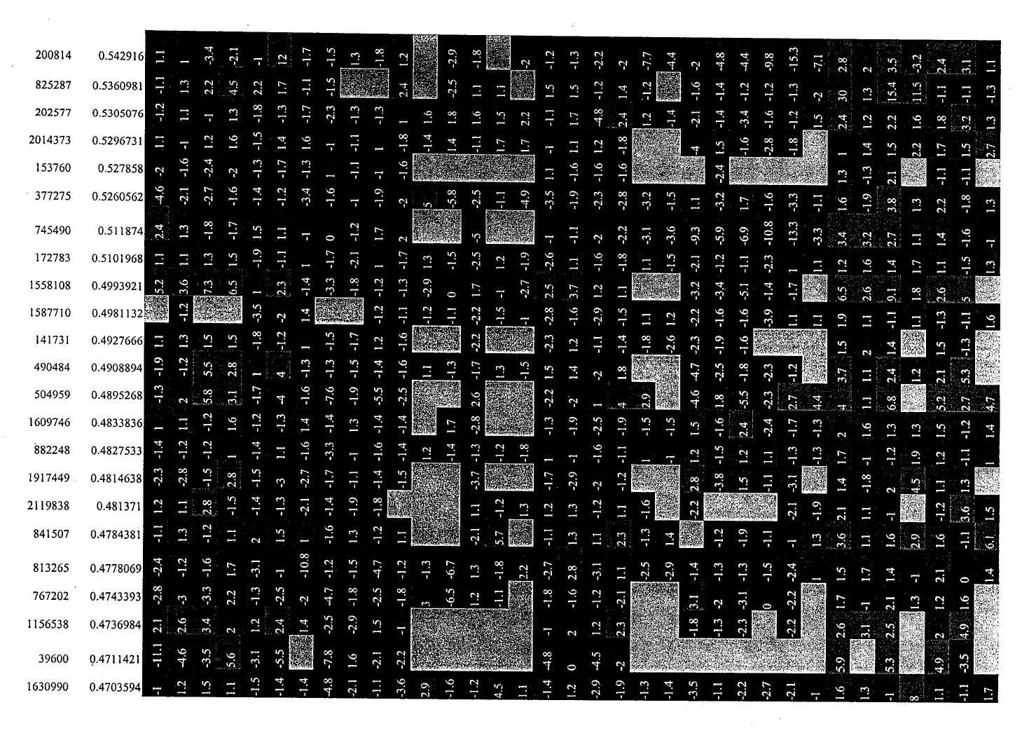

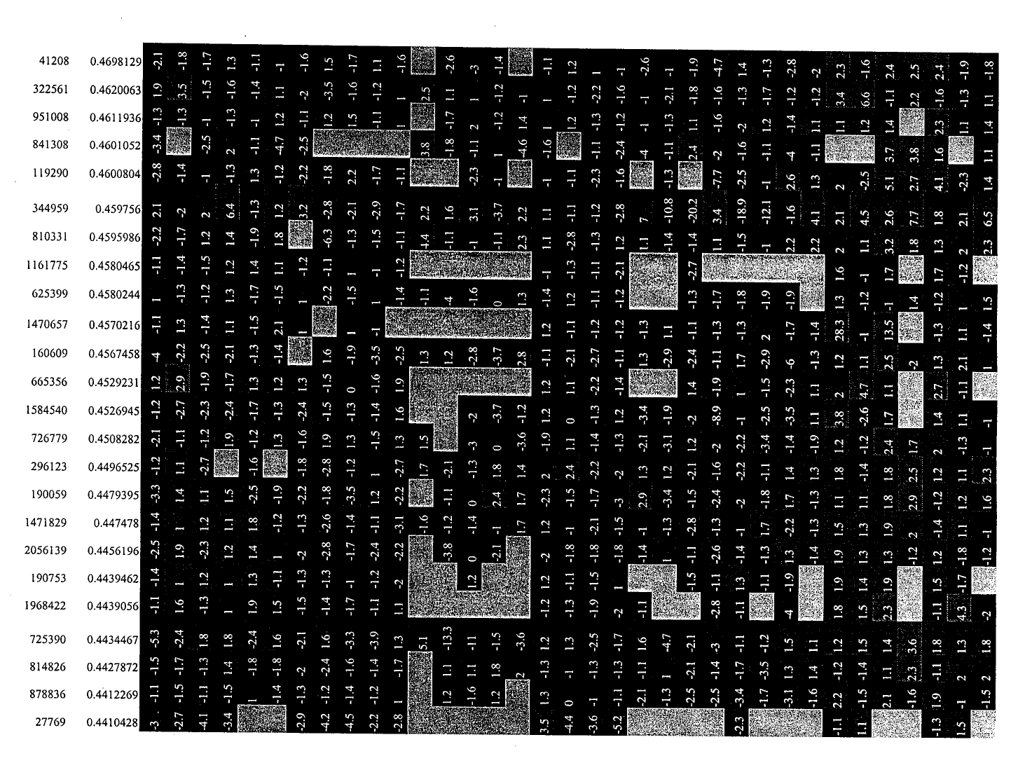

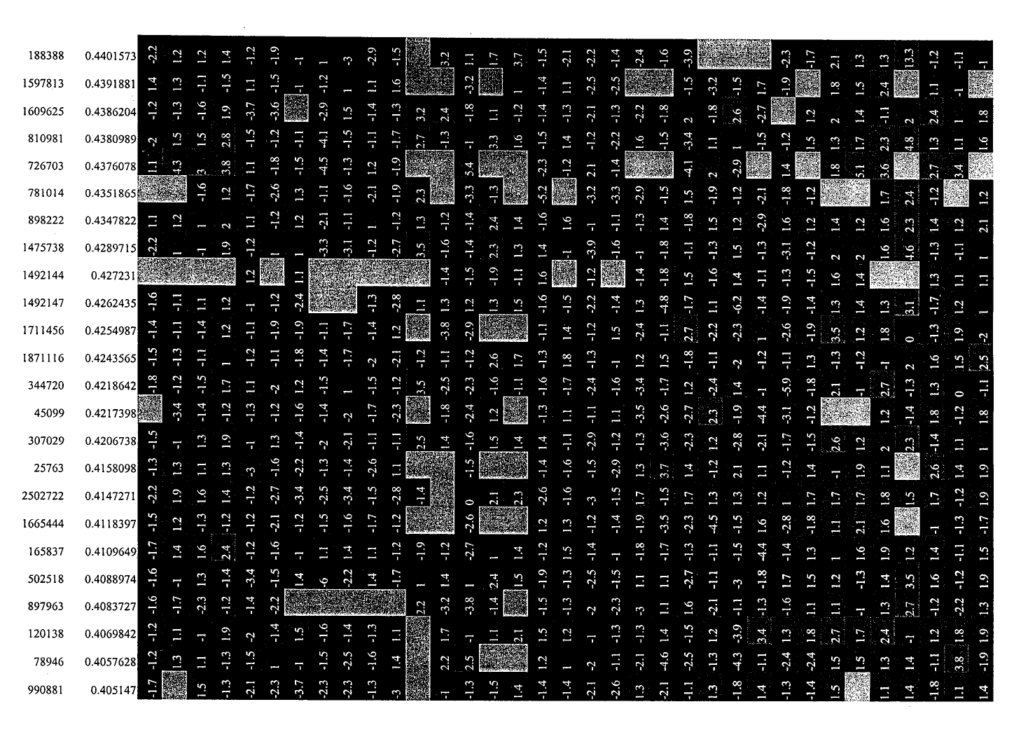

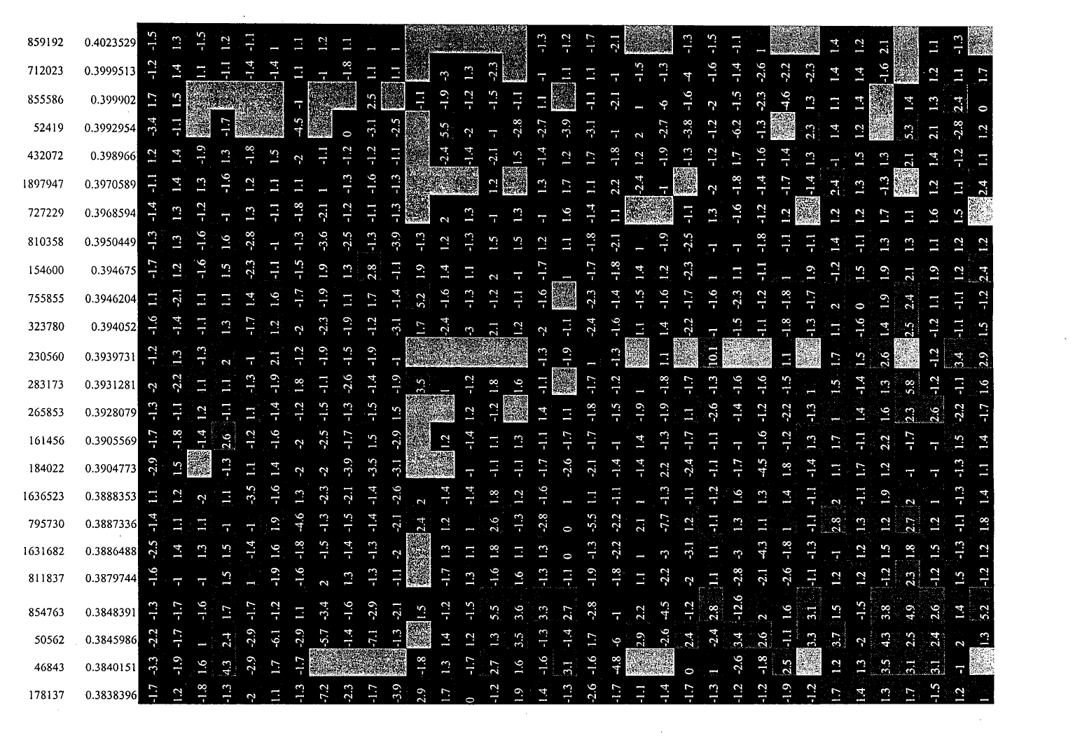

Microarray images were analyzed with ImaGene (BioDiscovery) to find and quantitate each spot on the microarray. Spots flagged by ImaGene as poor spots using standard criteria used with the software for the standardization of signals were excluded from further analysis. Raw Cy5 (sample channel) and Cy3 (reference channel) intensities and associated local background estimates for each spot were then examined. The signal/noise ratio, defined as the spot intensity over background intensity, was used as the second criteria for spot exclusion; spots with signal/noise ratio<3.0 in the reference channel or <1.5 in the sample channel were excluded from further analysis. Background-subtracted intensities across the chip were normalized to the 75th-percentile of the spot intensity distribution on the entire chip (alternative normalizations to the mean, median or other point may also be used as known in the art). Cy5/Cy3 ratios of each spot for each cellular state were averaged across each of six measurements (3 LCM captures×2 chips/capture=6 chips); outliers among the 6 data points were removed before taking the average. The resulting data were formatted as a data matrix (samples along the top horizontal axis and gene identity along the vertical axis) for data mining (see FIG. 1 with data).

Microarray Data Analysis

Before further analysis, each value in a row (gene) of the gene expression matrix was divided by the median value for the row, and the resulting matrix log-transformed. Normalized, median-centered, and log-transformed, gene expression data matrix was loaded into GeneMaths software (Applied-Maths, Belgium). Clustering and discriminant analysis were performed to identify sets of genes associated with different cellular states. For each pair-wise comparison between two breast cancer stages, samples are assigned to either the positive group or negative group, and genes were sorted by their discriminatory weights. The absolute value of the weight of a gene indicates the extent of difference in expression between the two groups; the positively signed genes are expressed higher in one group and the negatively singed genes are expressed higher in the other group.

The utility of the top-ranking genes as a diagnostic test was evaluated using the support vector machines (SVMs) algorithm (see Yeang, C. H., S. Ramaswamy, et al. (2001). “Molecular classification of multiple tumor types.” Bioinformatics 17 Suppl 1: S316-22; Xiong, M., X. Fang, et al. (2001). “Biomarker identification by feature wrappers.” Genome Res 11 (11): 1878-87 this one used linear discriminate analysis, logistic regression and svm; Furey, T. S., N. Cristianini, et al. (2000). “Support vector machine classification and validation of cancer tissue samples using microarray expression data.” Bioinformatics 16(10): 906-14; and Brown, M. P., W. N. Grundy, et al. (2000). “Knowledge-based analysis of microarray gene expression data by using support vector machines.” Proc Natl Acad Sci USA 97(1): 262-7, who state “SVMs are considered a supervised computer learning method because they exploit prior knowledge of gene function to identify unknown genes of similar function from expression data. SVMs avoid several problems associated with unsupervised clustering methods, such as hierarchical clustering and self-organizing maps.”) Other algorithms, such as, but not limited to, linear discriminate analysis, logistic regression, cluster analysis, K-th nearest neighbor, or neural nets.

The support vector machines algorithm finds the maximal margin hyperplane that separate the two groups under comparison. The method of leave-one-out cross-validation was used to test the performance of a given set of genes; one sample was taken in turn out of the training set and a model is built using the rest of the training set, which is in then applied to classify the left-out sample. The accuracy of the genes in the cross-validation procedure is the percentage of correct classifications over the total number of the training samples.

### Example II

### 10 Genes for Discriminating Between ADH and DCIS

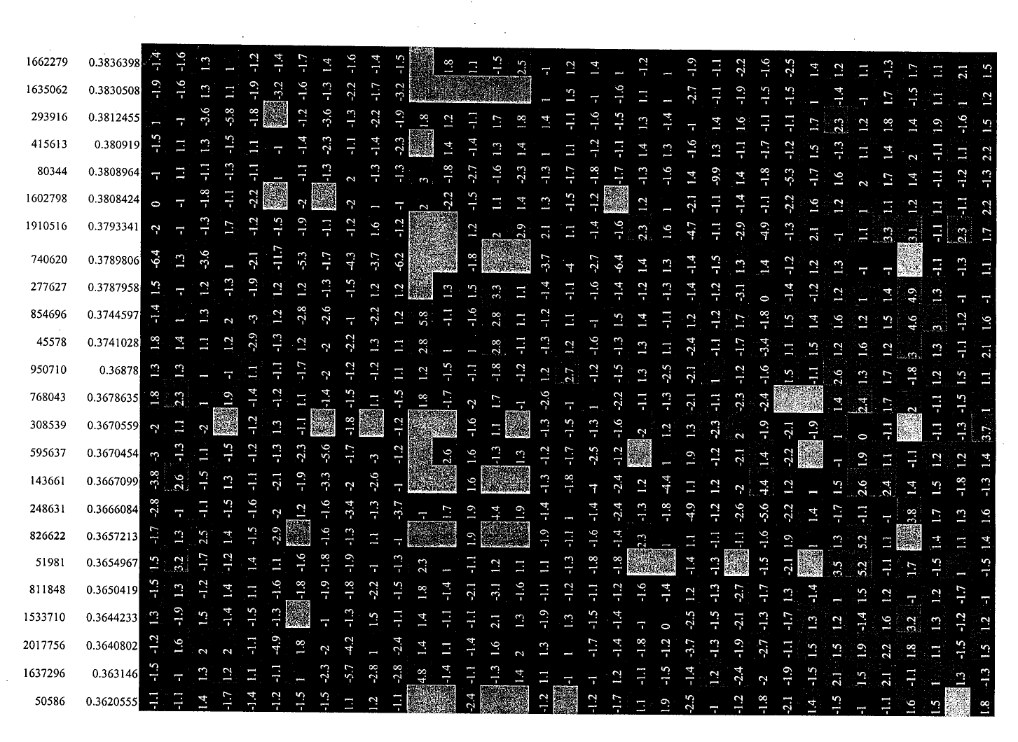

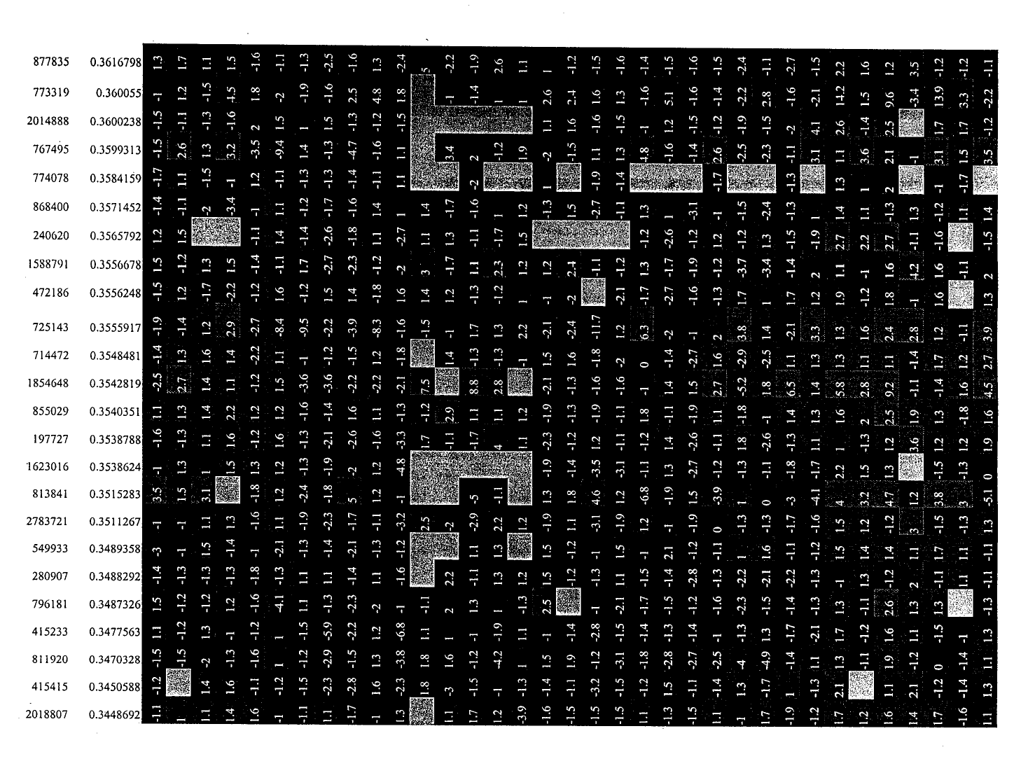

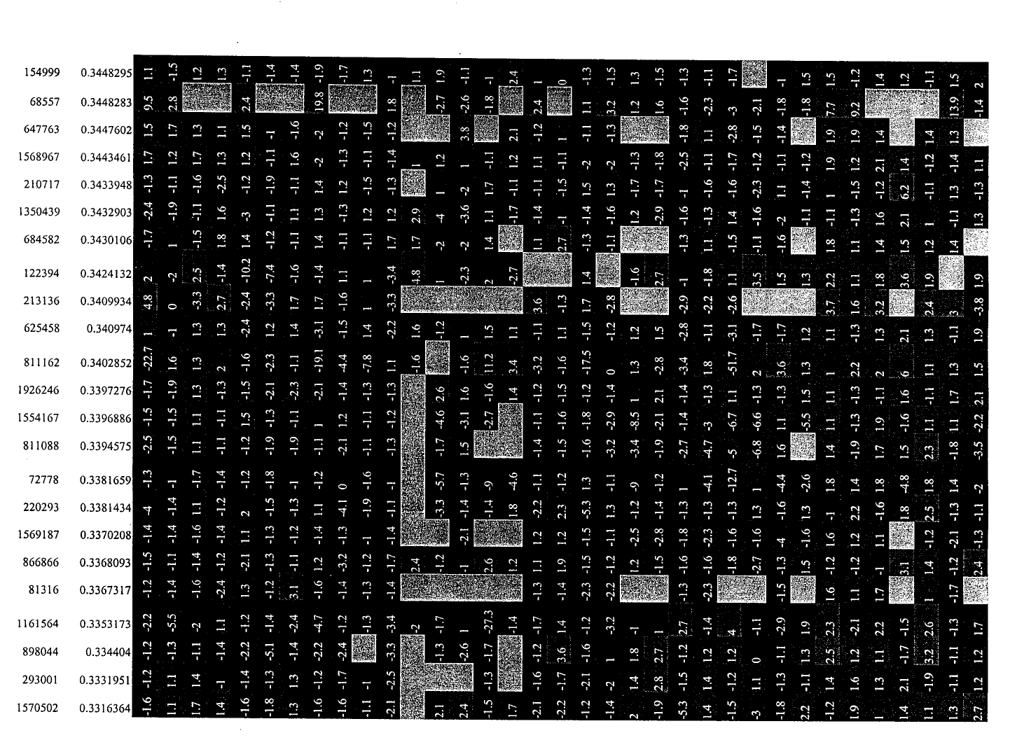

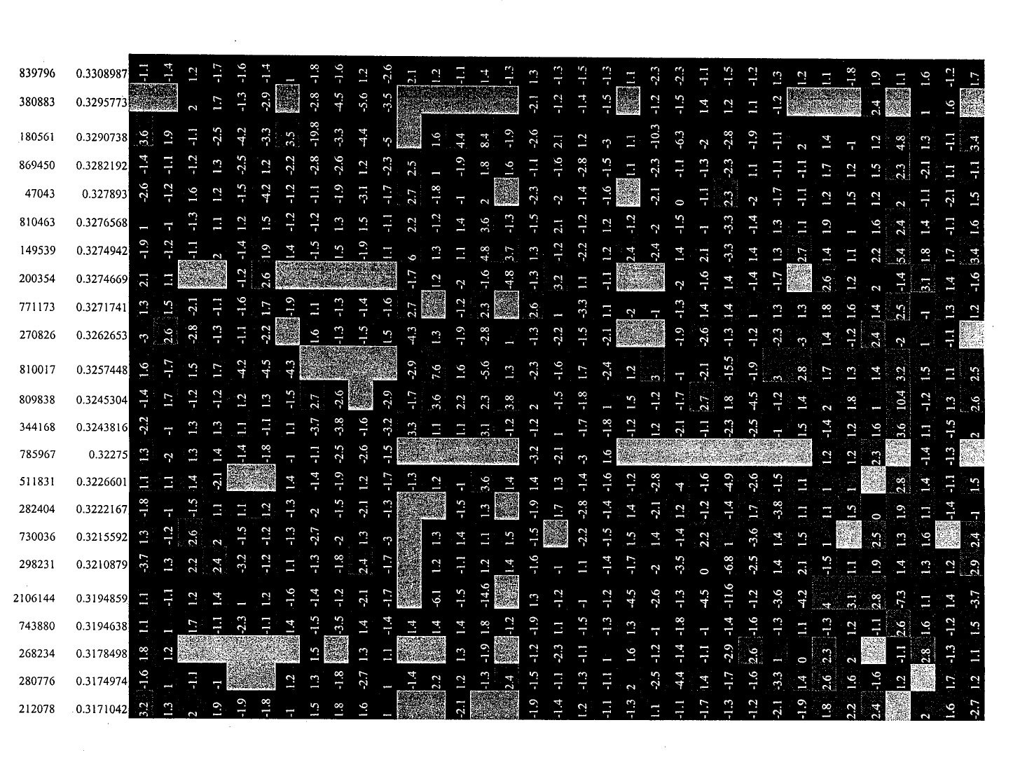

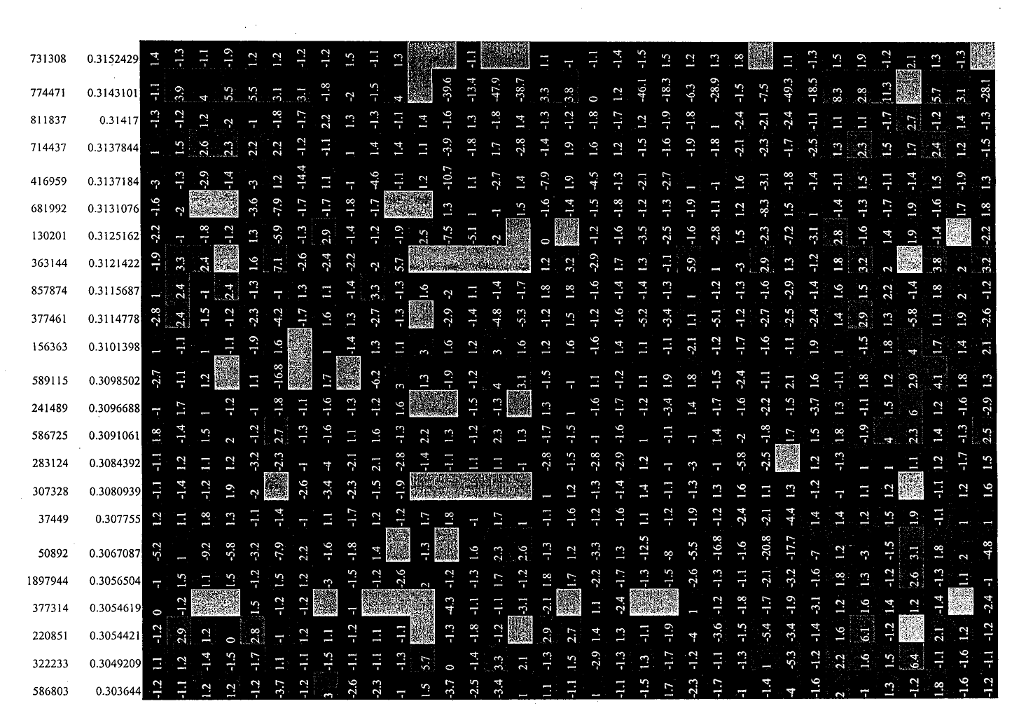

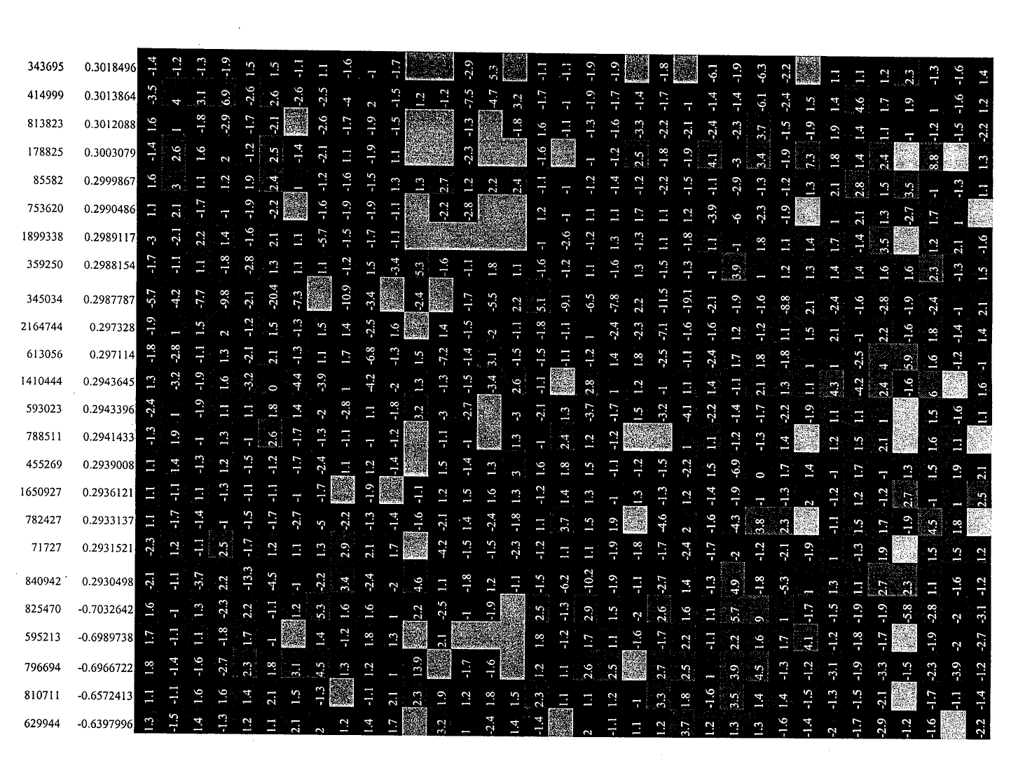

Based upon the methodology of Example I above, 10 genes identified as capable of discriminating between ADH and DCIS are listed in Table 1 below along with a brief description of the gene. CloneID as used in the context of the present invention refers to the IMAGE Consortium clone ID number of each gene, the sequences of which are hereby incorporated by reference in their entireties as they are available from the Consortium at http://image.llnl.gov/ as accessed on the filing date of the present application. Weight refers to the absolute value indicating the extent of difference in expression between ADH and DCIS where the positively signed values are expressed higher in ADH and the negatively signed values are expressed higher in DCIS; Chromosome Location refers to the human chromosome to which the gene has been assigned, and Description provides a brief identifier of what the gene encodes. The actual data corresponding to the assigned weights are shown in FIG. 2.

TABLE 1

| | | Chromosome | |
|---|---|---|---|
| GeneID | Weight | Location | Description |
| 825470 | 0.9946555 | 17q21-q22 | topoisomerase (DNA) II alpha |
| | | | (170 kD) |
| 595213 | 0.9884884 | 8 | hypothetical protein |
| 796694 | 0.9852686 | 17q25 | baculoviral IAP repeat- |
| | | | containing 5 (survivin) |
| 1404774 | −1.5638738 | 12p12.1-p11.2 | parathyroid hormone-like |
| | | | hormone |
| 823871 | −1.5260464 | 7 | SPARC-like 1 (mast9, hevin) |
| 1882697 | −1.401878 | 17q22-q23 | peanut (Drosophila)-like 2 |
| 140071 | −1.3881954 | 2 | frizzled-related protein |
| 160192 | −1.2551663 | 5 | ESTs, Weakly similar to |
| | | | 2004399A chromosomal |
| | | | protein [H. sapiens] |
| 796542 | −1.1401853 | 3q28 | ets variant gene 5 |
| | | | (ets-related molecule) |
| 611532 | −1.1227597 | 11p15.5 | troponin I, skeletal, fast |

### Examples III

### Genes for Discriminating Between Normal and Non-Normal (a Combination of ADH, DCIS, and IDC) Cells from Breast Tissue

As shown in Table 2 below, 850 genes were identified as being able to discriminate between normal and “abnormal” (defined in this instance as any sample that was not normal), which includes ADH, DCIS and IDC.

TABLE 2

| CloneID | Weight | Description |
|---|---|---|
| 1323448 | 1.5470535 | cysteine-rich protein 1 (intestinal) |
| 788654 | 1.4818381 | growth factor receptor-bound protein 2 |
| 35147 | 1.3764654 | “ESTs, Weakly similar to unnamed protein product |
| | | [H. sapiens]” |
| 745606 | 1.3470375 | hypothetical protein PP591 |
| 1500000 | 1.3204029 | “H2B histone family, member B” |
| 595037 | 1.3057353 | retinoic acid induced 3 |
| 565319 | 1.2369212 | Homo sapiens mRNA; cDNA DKFZp564B1264 (from clone |
| | | DKFZp564B1264) |
| 122077 | 1.2356703 | putative membrane protein |
| 1609836 | 1.2349106 | glutamate-ammonia ligase (glutamine synthase) |
| 1505038 | 1.2292506 | hypothetical protein FLJ20171 |
| 178805 | 1.2225746 | “Human DNA sequence from clone RP5-850E9 on |
| | | chromosome 20. Contains part of the gene for a novel |
| | | C2H2 type zinc finger protein similar to DrosophilaScratch |
| | | (Scrt), Slug and Xenopus Snail, a novel gene similar to |
| | | Drosophila CG6762, STSs, GSSs and five CpG |
| 812238 | 1.2132886 | hypothetical protein MGC4692 |
| 1492238 | 1.1991653 | HSPC003 protein |
| 366132 | 1.1911439 | “succinate dehydrogenase complex, subunit C, integral |
| | | membrane protein, 15 kD” |
| 796469 | 1.1850928 | HSPC150 protein similar to ubiquitin-conjugating enzyme |
| 488964 | 1.1545223 | “H2A histone family, member O” |
| 471568 | 1.1466811 | hematological and neurological expressed 1 |
| 1554549 | 1.1440029 | hydroxyacyl glutathione hydrolase |
| 283919 | 1.118257 | “H2A histone family, member L” |
| 1492463 | 1.087152 | “selenoprotein X, 1” |
| 199403 | 1.082569 | “lectin, galactoside-binding, soluble, 8 (galectin 8)” |
| 796723 | 1.0724133 | Homo sapiens clone CDABP0014 mRNA sequence |
| 138189 | 1.0692241 | Wolfram syndrome 1 (wolframin) |
| 1574058 | 1.0651351 | “1-acylglycerol-3-phosphate O-acyltransferase 2 |
| | | (lysophosphatidic acid acyltransferase, beta)” |
| 811774 | 1.0617336 | CGI-49 protein |
| 359887 | 1.052326 | translocase of inner mitochondrial membrane 17 (yeast) |
| | | homolog A |
| 1709791 | 1.0378948 | BAI1-associated protein 1 |
| 244801 | 1.0162794 | Rho guanine exchange factor (GEF) 11 |
| 1917941 | 0.9960315 | purine-rich element binding protein B |
| 1858892 | 0.9897362 | hypothetical protein MGC4825 |
| 288999 | 0.9896012 | small protein effector 1 of Cdc42 |
| 503215 | 0.9757636 | pilin-like transcription factor |
| 1911343 | 0.967716 | “RAB26, member RAS oncogene family” |
| 2029173 | 0.9672743 | “ESTs, Weakly similar to N-WASP [H. sapiens]” |
| 470061 | 0.9665514 | seven in absentia (Drosophila) homolog 2 |
| 2016908 | 0.9615102 | “ESTs, Weakly similar to CA13_HUMAN COLLAGEN |
| | | ALPHA 1(III) CHAIN PRECURSOR [H. sapiens]” |
| 1469425 | 0.9610542 | SRY (sex determining region Y)-box 22 |
| 814054 | 0.958989 | KIAA0040 gene product |
| 814528 | 0.9569471 | “Homo sapiens cDNA: FLJ22139 fis, clone HEP20959” |
| 68636 | 0.9562688 | hypothetical protein MGC2477 |
| 290841 | 0.9562201 | “H2B histone family, member A” |
| 236034 | 0.9534619 | “uncoupling protein 2 (mitochondrial, proton carrier)” |
| 782428 | 0.9524489 | KIAA0250 gene product |
| 131094 | 0.9518372 | “Homo sapiens cDNA: FLJ21587 fis, clone COL06946” |
| 488202 | 0.9501137 | “ESTs, Weakly similar to YZ28_HUMAN HYPOTHETICAL |
| | | PROTEIN ZAP128 [H. sapiens]” |
| 1435862 | 0.9500093 | “antigen identified by monoclonal antibodies 12E7, F21 and |
| | | O13” |
| 823598 | 0.9452282 | “proteasome (prosome, macropain) 26S subunit, non- |
| | | ATPase, 12” |
| 1492426 | 0.9397097 | chromosome 19 open reading frame 3 |
| 795185 | 0.9295627 | xenotropic and polytropic retrovirus receptor |
| 810558 | 0.9227709 | “proteasome (prosome, macropain) 26S subunit, ATPase, |
| | | 4” |
| 469686 | 0.9203022 | “Ric (Drosophila)-like, expressed in many tissues” |
| 280375 | 0.9155003 | PRO2000 protein |
| 729975 | 0.9149894 | meningioma expressed antigen 5 (hyaluronidase) |
| 810124 | 0.9124035 | “platelet-activating factor acetylhydrolase, isoform Ib, |
| | | gamma subunit (29 kD)” |
| 813281 | 0.9107661 | WW domain-containing protein 1 |
| 39884 | 0.9091807 | IMP (inosine monophosphate) dehydrogenase 1 |
| 76605 | 0.9028609 | nesca protein |
| 1636092 | 0.8997881 | hypothetical protein FLJ20657 |
| 741474 | 0.8925439 | glucose phosphate isomerase |
| 1605426 | 0.8909868 | hypothetical protein FLJ13352 |
| 788654 | 0.8886269 | |
| 868128 | 0.8873517 | JM4 protein |
| 308466 | 0.8803492 | GTP-binding protein Sara |
| 44292 | 0.8732235 | Homo sapiens mRNA; cDNA DKFZp434C107 (from clone |
| | | DKFZp434C107) |
| 826256 | 0.8700831 | transmembrane 7 superfamily member 1 (upregulated in |
| | | kidney) |
| 685516 | 0.8694652 | putative G protein-coupled receptor |
| 810711 | 0.869424 | stearoyl-CoA desaturase (delta-9-desaturase) |
| 753299 | 0.8679015 | hypothetical protein FLJ10504 |
| 731044 | 0.8657953 | glutaredoxin 2 |
| 824052 | 0.8650616 | chromosome 6 open reading frame 1 |
| 843195 | 0.86501 | phosphoserine phosphatase |
| 292770 | 0.8570995 | “Homo sapiens, clone IMAGE: 3627860, mRNA, partial cds” |
| 859761 | 0.8563964 | poliovirus receptor-related 2 (herpesvirus entry mediator B) |
| 347373 | 0.8555396 | “transcription elongation factor B (SIII), polypeptide 1 |
| | | (15 kD, elongin C)” |
| 1640821 | 0.8544081 | “ESTs, Weakly similar to I78885 serine/threonine-specific |
| | | protein kinase [H. sapiens]” |
| 704414 | 0.8520508 | small nuclear ribonucleoprotein polypeptides B and B1 |
| 810725 | 0.8510425 | “ATPase, H+ transporting, lysosomal (vacuolar proton |
| | | pump) 21 kD” |
| 782608 | 0.8507815 | mitochondrial ribosomal protein L9 |
| 2019223 | 0.849044 | mitochondrial ribosomal protein L17 |
| 2110511 | 0.8471736 | artemin |
| 1474955 | 0.8460414 | “TATA box binding protein (TBP)-associated factor, RNA |
| | | polymerase II, N, 68 kD (RNA-binding protein 56)” |
| 810612 | 0.842284 | S100 calcium-binding protein A11 (calgizzarin) |
| 744417 | 0.8409538 | carnitine acetyltransferase |
| 969877 | 0.8382113 | “synaptosomal-associated protein, 25 kD” |
| 272529 | 0.8371698 | phosphomannomutase 2 |
| 768570 | 0.8371438 | hypothetical protein FLJ11280 |
| 824879 | 0.8366695 | hypothetical protein MGC11275 |
| 2014034 | 0.8354357 | “methylene tetrahydrofolate dehydrogenase (NAD+ |
| | | dependent), methenyltetrahydrofolate cyclohydrolase” |
| 2054635 | 0.831532 | “proteasome (prosome, macropain) subunit, alpha type, 7” |
| 2052113 | 0.8307595 | hypothetical protein FLJ10903 |
| 742595 | 0.8291965 | cyclin-dependent kinase 5 |
| 2016648 | 0.8263671 | Homo sapiens mRNA; cDNA DKFZp434N1728 (from clone |
| | | DKFZp434N1728) |
| 1573251 | 0.823025 | peroxisomal long-chain acyl-coA thioesterase |
| 1869201 | 0.8202299 | hypothetical protein MGC2745 |
| 37708 | 0.8194577 | hypothetical protein MGC3101 |
| 241348 | 0.8182422 | prenylcysteine lyase |
| 810063 | 0.8168926 | “growth factor, erv1 (S. cerevisiae)-like (augmenter of liver |
| | | regeneration)” |
| 731308 | 0.8166873 | citrate synthase |
| 209066 | 0.8120797 | |
| 590774 | 0.8100386 | mitogen-activated protein kinase 13 |
| 1435003 | 0.8098225 | tumor suppressing subtransferable candidate 1 |
| 502774 | 0.8091404 | hypothetical protein FLJ20623 |
| 488505 | 0.808712 | accessory proteins BAP31/BAP29 |
| 109863 | 0.8066906 | epithelial membrane protein 2 |
| 813419 | 0.8043966 | “hydroxyacyl-Coenzyme A dehydrogenase, type II” |
| 1845169 | 0.8033348 | “RAB35, member RAS oncogene family” |
| 742707 | 0.7955239 | “ESTs, Weakly similar to MUC2_HUMAN MUCIN 2 |
| | | PRECURSOR [H. sapiens]” |
| 594500 | 0.7935958 | EST |
| 1456701 | 0.7934389 | B-cell CLL/lymphoma 9 |
| 271472 | 0.7901735 | C3HC4-like zinc finger protein |
| 1473922 | 0.7892502 | “actin related protein 2/3 complex, subunit 3 (21 kD)” |
| 51773 | 0.7858986 | hypothetical protein MGC3077 |
| 898032 | 0.7852733 | KIAA0097 gene product |
| 340558 | 0.7791736 | “actin related protein 2/3 complex, subunit 5 (16 kD)” |
| 773922 | 0.7767604 | KIAA0005 gene product |
| 768064 | 0.7743382 | “cytochrome P450, subfamily I (aromatic compound- |
| | | inducible), polypeptide 1” |
| 625923 | 0.7725056 | phosphoenolpyruvate carboxykinase 2 (mitochondrial) |
| 150003 | 0.77183 | hypothetical protein FLJ13187 |
| 741977 | 0.7715372 | “B-factor, properdin” |
| 365738 | 0.7709456 | ESTs |
| 814350 | 0.7701748 | insulin-degrading enzyme |
| 2108077 | 0.7700354 | CGI-112 protein |
| 209066 | 0.7693907 | serine/threonine kinase 15 |
| 564492 | 0.7672548 | mitochondrial carrier homolog 2 |
| 366353 | 0.7664569 | DKFZP564C186 protein |
| 813751 | 0.7659116 | “sialyltransferase 4C (beta-galactosidase alpha-2,3- |
| | | sialytransferase)” |
| 781097 | 0.7633668 | reticulon 3 |
| 233349 | 0.762952 | hypothetical protein FLJ10761 |
| 825327 | 0.7622414 | “Homo sapiens cDNA FLJ14105 fis, clone |
| | | MAMMA1001202” |
| 248649 | 0.761865 | hypothetical protein FLJ13910 |
| 589232 | 0.7583348 | hypothetical protein FLJ11506 |
| 564847 | 0.757516 | ESTs |
| 754628 | 0.7573763 | ESTs |
| 1500162 | 0.7571399 | ESTs |
| 1738208 | 0.7565056 | “a disintegrin-like and metalloprotease (reprolysin type) |
| | | with thrombospondin type 1 motif, 4” |
| 842994 | 0.7563101 | cathepsin Z |
| 42408 | 0.7559848 | hypothetical protein MGC4604 |
| 470124 | 0.7539249 | RAD1 (S. pombe) homolog |
| 809357 | 0.7535411 | Bernardinelli-Seip congenital lipodystrophy 2 (seipin) |
| 624667 | 0.7524325 | CGI-92 protein |
| 49351 | 0.75115 | SEX gene |
| 46248 | 0.7510436 | ADP-ribosyltransferase (NAD+; poly (ADP-ribose) |
| | | polymerase) |
| 686552 | 0.7506994 | golgi phosphoprotein 1 |
| 1631735 | 0.7505843 | “Homo sapiens, clone IMAGE: 3604336, mRNA, partial cds” |
| 1536006 | 0.7499887 | ESTs |
| 83363 | 0.748646 | protein-L-isoaspartate (D-aspartate) O-methyltransferase |
| 713782 | 0.7484589 | a disintegrin and metalloproteinase domain 15 (metargidin) |
| 727078 | 0.7472345 | “Homo sapiens cDNA: FLJ23602 fis, clone LNG15735” |
| 503889 | 0.7454098 | hypothetical protein FLJ10656 |
| 284261 | 0.7445419 | uncharacterized hematopoietic stem/progenitor cells |
| | | protein MDS030 |
| 1473289 | 0.7441483 | protective protein for beta-galactosidase (galactosialidosis) |
| 49273 | 0.742123 | “solute carrier family 27 (fatty acid transporter), member 4” |
| 785701 | 0.7413837 | “RAB31, member RAS oncogene family” |
| 41569 | 0.7412357 | hypothetical protein FLJ12650 |
| 810402 | 0.7407907 | hypothetical protein |
| 1456348 | 0.7397574 | N-acetylneuraminic acid phosphate synthase; sialic acid |
| | | synthase |
| 256619 | 0.7395032 | hydroxysteroid (17-beta) dehydrogenase 7 |
| 743589 | 0.7372744 | “ESTs, Weakly similar to T2D3_HUMAN TRANSCRIPTION |
| | | INITIATION FACTOR TFIID 135 KDA SUBUNIT |
| | | [H. sapiens]” |
| 1409509 | 0.7370811 | “troponin T1, skeletal, slow” |
| 122241 | 0.7352191 | “proteasome (prosome, macropain) subunit, beta type, 2” |
| 470099 | 0.7343404 | HT002 protein; hypertension-related calcium-regulated |
| | | gene |
| 725454 | 0.7335223 | CDC28 protein kinase 2 |
| 796694 | 0.7317383 | baculoviral IAP repeat-containing 5 (survivin) |
| 186768 | 0.7298378 | “Homo sapiens, clone IMAGE: 3604869, mRNA” |
| 813629 | 0.7288266 | YME1 (S. cerevisiae)-like 1 |
| 842980 | 0.7284256 | developmentally regulated GTP-binding protein 1 |
| 769921 | 0.726646 | ubiquitin carrier protein E2-C |
| 2322367 | 0.7249854 | reticulon 4 |
| 951233 | 0.7240615 | “proteasome (prosome, macropain) subunit, beta type, 3” |
| 842825 | 0.7235007 | G1 to S phase transition 1 |
| 782608 | 0.7230962 | |
| 2043167 | 0.7224903 | BCL2-associated athanogene 3 |
| 827171 | 0.7211896 | ESTs |
| 120271 | 0.7208543 | hypothetical protein MGC4692 |
| 1518890 | 0.7206844 | “metallothionein-like 5, testis-specific (tesmin)” |
| 60565 | 0.720547 | lethal giant larvae (Drosophila) homolog 2 |
| 2050827 | 0.7156518 | “proteasome (prosome, macropain) 26S subunit, ATPase, |
| | | 5” |
| 1635681 | 0.7152705 | “NADH dehydrogenase (ubiquinone) 1 alpha subcomplex, |
| | | 2 (8 kD, B8)” |
| 731023 | 0.7151176 | WD repeat domain 5 |
| 1518402 | 0.7150061 | KIAA1361 protein |
| 752631 | 0.7139862 | “fibroblast growth factor receptor 3 (achondroplasia, |
| | | thanatophoric dwarfism)” |
| 785616 | 0.7131593 | “signal sequence receptor, alpha (translocon-associated |
| | | protein alpha)” |
| 686172 | 0.7131363 | KIAA0008 gene product |
| 40173 | 0.7124913 | KIAA0807 protein |
| 123441 | 0.7124698 | ribosomal protein L7a |
| 123614 | 0.7117834 | hypothetical protein MGC4675 |
| 811024 | 0.7117211 | bone marrow stromal cell antigen 2 |
| 2302099 | 0.7117055 | sialidase 3 (membrane sialidase) |
| 756442 | 0.7115925 | P450 (cytochrome) oxidoreductase |
| 811585 | 0.7087617 | huntingtin (Huntington disease) |
| 279970 | 0.7040135 | adenosine A2a receptor |
| 1517171 | 0.7036866 | “interleukin 2 receptor, alpha” |
| 838366 | 0.702617 | 3-hydroxymethyl-3-methylglutaryl-Coenzyme A lyase |
| | | (hydroxymethylglutaricaciduria) |
| 809944 | 0.7021108 | KIAA0310 gene product |
| 1393018 | 0.701717 | “general transcription factor IIIC, polypeptide 1 (alpha |
| | | subunit, 220 kD)” |
| 725978 | 0.7010756 | “ESTs, Moderately similar to ALU7_HUMAN ALU |
| | | SUBFAMILY SQ SEQUENCE CONTAMINATION |
| | | WARNING ENTRY [H. sapiens]” |
| 325606 | 0.7008219 | hypothetical protein MGC14353 |
| 795256 | 0.6974866 | NPD007 protein |
| 365358 | 0.696963 | pM5 protein |
| 67765 | 0.6967324 | carboxypeptidase M |
| 358267 | 0.6962436 | “EST, Moderately similar to AF119917 63 PRO2831 |
| | | [H. sapiens]” |
| 149355 | 0.6943923 | translocating chain-associating membrane protein |
| 212542 | 0.6938079 | “Homo sapiens cDNA FLJ12900 fis, clone |
| | | NT2RP2004321” |
| 79520 | 0.6919579 | “RAB2, member RAS oncogene family” |
| 739126 | 0.6881663 | tissue specific transplantation antigen P35B |
| 202514 | 0.6872212 | DNA (cytosine-5-)-methyltransferase 3 alpha |
| 1884404 | 0.6864546 | KIAA0285 gene product |
| 814378 | 0.6857148 | “serine protease inhibitor, Kunitz type, 2” |
| 2018337 | 0.6828744 | “glucosidase, beta; acid (includes glucosylceramidase)” |
| 701751 | 0.6824676 | cut (Drosophila)-like 1 (CCAAT displacement protein) |
| 2018084 | 0.6822251 | Ste-20 related kinase |
| 781019 | 0.6819736 | paraoxonase 2 |
| 2244196 | 0.6807351 | B-cell receptor-associated protein BAP29 |
| 124447 | 0.679866 | KIAA1184 protein |
| 789012 | 0.6794432 | fibulin 2 |
| 490778 | 0.6788304 | low molecular mass ubiquinone-binding protein (9.5 kD) |
| 2011515 | 0.6774452 | DKFZP586B0923 protein |
| 80764 | 0.6765616 | hypothetical protein |
| 841679 | 0.6762195 | calcium and integrin binding protein (DNA-dependent |
| | | protein kinase interacting protein) |
| 108425 | 0.6761526 | |
| 878406 | 0.6750777 | metaxin 1 |
| 487733 | 0.6745496 | |
| 625693 | 0.6744622 | hypothetical protein MGC10911 |
| 1325816 | 0.6742078 | polymerase (RNA) II (DNA directed) polypeptide L (7.6 kD) |
| 278531 | 0.6737637 | cytochrome c oxidase subunit VIc |
| 744374 | 0.6732551 | putative ankyrin-repeat containing protein |
| 203003 | 0.6724027 | “non-metastatic cells 4, protein expressed in” |
| 564981 | 0.6712134 | ESTs |
| 812994 | 0.671102 | “retinoid X receptor, alpha” |
| 172517 | 0.6701228 | hippocalcin-like 1 |
| 205049 | 0.6682666 | protein kinase H11; small stress protein-like protein HSP22 |
| 1734309 | 0.6672454 | sperm associated antigen 4 |
| 1631699 | 0.6665952 | valosin-containing protein |
| 2028949 | 0.6662774 | hypothetical protein PRO1855 |
| 530197 | 0.6659784 | “ESTs, Moderately similar to ALU8_HUMAN ALU |
| | | SUBFAMILY SX SEQUENCE CONTAMINATION |
| | | WARNING ENTRY [H. sapiens]” |
| 199645 | 0.6654138 | nicastrin |
| 704254 | 0.6653635 | a disintegrin and metalloproteinase domain 8 |
| 1592715 | 0.6637095 | “Homer, neuronal immediate early gene, 3” |
| 302031 | 0.6626629 | Ste20-related serine threonine kinase |
| 239568 | 0.661735 | annexin A9 |
| 770785 | 0.6613519 | “1,2-alpha-mannosidase IC” |
| 785795 | 0.660789 | hypothetical protein FLJ12910 |
| 810734 | 0.6603412 | “polymerase (DNA-directed), delta 4” |
| 1696757 | 0.6591801 | hypothetical protein KIAA1165 |
| 811761 | 0.6587469 | Nijmegen breakage syndrome 1 (nibrin) |
| 150314 | 0.6584621 | lysophospholipase I |
| 489351 | 0.65846 | hypothetical protein DKFZp566J2046 |
| 593431 | 0.6578268 | “ESTs, Moderately similar to CEGT_HUMAN CERAMIDE |
| | | GLUCOSYLTRANSFERASE [H. sapiens]” |
| 37554 | 0.6574292 | hypothetical protein FLJ22353 |
| 126851 | 0.6559601 | hypothetical protein FLJ11160 |
| 265103 | 0.6548137 | Homo sapiens mRNA; cDNA DKFZp547M123 (from clone |
| | | DKFZp547M123) |
| 51083 | 0.6544472 | “catenin (cadherin-associated protein), delta 2 (neural |
| | | plakophilin-related arm-repeat protein)” |
| 431505 | 0.6534315 | “ESTs, Highly A31026 probable membrane |
| | | receptor protein [H. sapiens]” |
| 1420370 | 0.6531171 | biliverdin reductase B (flavin reductase (NADPH)) |
| 1476053 | 0.6522237 | RAD51 (S. cerevisiae) homolog (E coli RecA homolog) |
| 882484 | 0.6521026 | “chaperonin containing TCP1, subunit 7 (eta)” |
| 51532 | 0.6504769 | ADP-ribosylation factor-like 6 interacting protein |
| 280249 | 0.6503028 | Kruppel-like factor 7 (ubiquitous) |
| 138788 | 0.6497728 | prolactin receptor |
| 1492468 | 0.647846 | DEME-6 protein |
| 221295 | 0.6477514 | “inhibitor of DNA binding 2, dominant negative helix-loop- |
| | | helix protein” |
| 897806 | 0.6470379 | “hypoxia-inducible factor 1, alpha subunit (basic helix-loop- |
| | | helix transcription factor)” |
| 358936 | 0.6441198 | “ESTs, Weakly similar to T2D3_HUMAN TRANSCRIPTION |
| | | INITIATION FACTOR TFIID 135 KDA SUBUNIT |
| | | [H. sapiens]” |
| 840865 | 0.644011 | “myristoylated alanine-rich protein kinase C substrate |
| | | (MARCKS, 80K-L)” |
| 788334 | 0.6427785 | mitochondrial ribosomal protein L23 |
| 366067 | 0.6416356 | cerebellar degeneration-related protein (62 kD) |
| 825585 | 0.6413235 | tubulin-specific chaperone e |
| 1460110 | 0.6397172 | “proteasome (prosome, macropain) subunit, beta type, 5” |
| 344091 | 0.6381862 | ESTs |
| 196189 | 0.6338517 | cytochrome b-5 |
| 2248488 | 0.6337488 | ems1 sequence (mammary tumor and squamous cell |
| | | carcinoma-associated (p80/85 src substrate) |
| 509823 | 0.6334191 | carcinoembryonic antigen-related cell adhesion molecule 6 |
| | | (non-specific cross reacting antigen) |
| 144880 | 0.6332999 | hypothetical protein from EUROIMAGE 1759349 |
| 755599 | 0.6330221 | interferon induced transmembrane protein 1 (9-27) |
| 207288 | 0.632639 | insulin induced gene 1 |
| 1639531 | 0.6315732 | “RAB27A, member RAS oncogene family” |
| 144740 | 0.630134 | similar to phosphatidylcholine transfer protein 2 |
| 814306 | 0.630078 | tumor protein D52 |
| 301122 | 0.6299499 | extracellular matrix protein 1 |
| 124298 | 0.6294358 | microsomal glutathione S-transferase 3 |
| 1733262 | 0.6293971 | BLu protein |
| 376875 | 0.6286902 | flavin containing monooxygenase 1 |
| 773188 | 0.6270494 | “nuclear receptor subfamily 1, group D, member 2” |
| 756666 | 0.6269882 | “protein phosphatase 1, catalytic subunit, alpha isoform” |
| 810156 | 0.6266313 | deoxythymidylate kinase (thymidylate kinase) |
| 2015517 | 0.6259564 | hypothetical protein FLJ22237 |
| 770845 | 0.6253839 | hexokinase 1 |
| 813707 | 0.6252347 | regulator of G-protein signalling 16 |
| 611443 | 0.6244565 | myoglobin |
| 124331 | 0.624455 | “cleavage and polyadenylation specific factor 5, 25 kD |
| | | subunit” |
| 855800 | 0.6235647 | prolyl endopeptidase |
| 625234 | 0.62188 | KDEL (Lys-Asp-Glu-Leu) endoplasmic reticulum protein |
| | | retention receptor 3 |
| 753378 | 0.6218232 | hypothetical protein FLJ22649 similar to signal peptidase |
| | | SPC22/23 |
| 2055807 | 0.6216372 | protein kinase domains containing protein similar to |
| | | phosphoprotein C8FW |
| 840878 | 0.6216124 | seladin-1 |
| 1700436 | 0.6215741 | ESTs |
| 261472 | 0.6215341 | putative nuclear protein ORF1-FL49 |
| 121251 | 0.6209436 | hypothetical protein MGC5576 |
| 140289 | 0.6205892 | hypothetical protein |
| 2018821 | 0.6205344 | ATPase inhibitor precursor |
| 770766 | 0.6193342 | DKFZP564C1940 protein |
| 767798 | 0.6192232 | “ATX1 (antioxidant protein 1, yeast) homolog 1” |
| 845363 | 0.617901 | “non-metastatic cells 1, protein (NM23A) expressed in” |
| 120749 | 0.6172281 | “ESTs, Moderately similar to KIAA1215 protein [H. sapiens]” |
| 358162 | 0.616765 | protein predicted by clone 23627 |
| 781342 | 0.6159353 | hypothetical protein MGC11115 |
| 767761 | 0.6142782 | DKFZP434B168 protein |
| 191904 | 0.6141265 | “BUB3 (budding uninhibited by benzimidazoles 3, yeast) |
| | | homolog” |
| 726699 | 0.6131491 | “ESTs, Weakly similar to AAB47496 NG5 [H. sapiens]” |
| 2326057 | 0.61263 | MLN51 protein |
| 784140 | 0.6102245 | WD repeat domain 15 |
| 770355 | 0.6099761 | “lanosterol synthase (2,3-oxidosqualene-lanosterol |
| | | cyclase)” |
| 81336 | 0.6099072 | uteroglobin |
| 757328 | 0.6097389 | hypothetical protein FLJ22678 |
| 773674 | 0.6095732 | oncogene TC21 |
| 289978 | 0.6090234 | ubiquitin-like 4 |
| 66535 | 0.6088919 | ornithine decarboxylase antizyme 2 |
| 826363 | 0.6087183 | lysophospholipase II |
| 1758590 | 0.6074571 | “fatty-acid-Coenzyme A ligase, long-chain 3” |
| 1553306 | 0.6066787 | “proteasome (prosome, macropain) 26S subunit, non- |
| | | ATPase, 11” |
| 785766 | 0.6058332 | hypothetical protein |
| 725841 | 0.6051304 | KIAA0662 gene product |
| 2020898 | 0.6044163 | “procollagen-lysine, 2-oxoglutarate 5-dioxygenase 3” |
| 504308 | 0.6041122 | hypothetical protein FLJ10540 |
| 510575 | 0.6040713 | hypothetical protein FLJ22087 |
| 49630 | 0.6027878 | “calcium channel, voltage-dependent, L type, alpha 1D |
| | | subunit” |
| 142586 | 0.602037 | MCT-1 protein |
| 725284 | 0.6014099 | “phosphorylase kinase, gamma 2 (testis)” |
| 429799 | 0.6012713 | hypothetical protein FLJ21939 similar to 5-azacytidine |
| | | induced gene 2 |
| 1474424 | 0.6006702 | “Homo sapiens cDNA FLJ12758 fis, clone |
| | | NT2RP2001328” |
| 784105 | 0.6003082 | ESTs |
| 2018527 | 0.599584 | dolichyl-phosphate mannosyltransferase polypeptide 3 |
| 855563 | 0.5987092 | v-erb-b2 avian erythroblastic leukemia viral oncogene |
| | | homolog 3 |
| 823574 | 0.5978052 | endosulfine alpha |
| 323693 | 0.5975785 | “adaptor-related protein complex 1, sigma 1 subunit” |
| 824962 | 0.595868 | “karyopherin alpha 2 (RAG cohort 1, importin alpha 1)” |
| 773426 | 0.5941568 | KIAA0391 gene product |
| 772925 | 0.5940171 | HSPCO34 protein |
| 1656062 | 0.593408 | coagulation factor XII (Hageman factor) |
| 825740 | 0.5931142 | DKFZp434J1813 protein |
| 376516 | 0.5906248 | cell division cycle 4-like |
| 292213 | 0.5883092 | “guanine nucleotide binding protein (G protein), beta |
| | | polypeptide 2” |
| 1898619 | 0.5880791 | hypothetical protein MGC15737 |
| 414992 | 0.587733 | K562 cell-derived leucine-zipper-like protein 1 |
| 1573946 | 0.5875624 | programmed cell death 9 |
| 739109 | 0.5874334 | “adaptor-related protein complex 2, sigma 1 subunit” |
| 430235 | 0.585372 | “H2B histone family, member Q” |
| 2572170 | 0.5852444 | “ESTs, Weakly similar to T26581 hypothetical protein |
| | | Y32B12A.3 - Caenorhabditis elegans [C. elegans]” |
| 487733-2 | 0.5844776 | “NADH dehydrogenase (ubiquinone) 1 beta subcomplex, 2 |
| | | (8 kD, AGGG)” |
| 1698036 | 0.5843382 | ubiquitin-conjugating enzyme E2 variant 1 |
| 127646 | 0.5838788 | |
| 346696 | 0.5838269 | TEA domain family member 4 |
| 74738 | 0.5835427 | “Homo sapiens, clone IMAGE: 3535294, mRNA, partial cds” |
| 124781 | 0.5832065 | squalene epoxidase |
| 1883028 | 0.5828007 | Homo sapiens mRNA; cDNA DKFZp434J1912 (from clone |
| | | DKFZp434J1912) |
| 183200 | 0.5801312 | fumarylacetoacetate hydrolase (fumarylacetoacetase) |
| 51741 | 0.5795151 | GTP-binding protein |
| 590759 | 0.5791873 | sterol-C4-methyl oxidase-like |
| 755301 | 0.5790795 | “protein kinase C, delta” |
| 843054 | 0.5776102 | KIAA1533 protein |
| 595213 | 0.5775511 | hypothetical protein |
| 752643 | 0.5774681 | group XII secreted phospholipase A2 |
| 141852 | 0.5762886 | “purinergic receptor P2Y, G-protein coupled, 2” |
| 813631 | 0.5754418 | seven transmembrane protein TM7SF3 |
| 1601947 | 0.5744444 | cytochrome c oxidase subunit VIIa polypeptide 2 (liver) |
| 51657 | 0.5736969 | hypothetical protein ET |
| 814209 | 0.5736866 | ESTs |
| 72050 | 0.5722845 | “chloride channel, nucleotide-sensitive, 1A” |
| 1635665 | 0.5714089 | “Homo sapiens, RIKEN cDNA 2010100O12 gene, clone |
| | | MGC: 14813 IMAGE: 4133274, mRNA, complete cds” |
| 66406 | 0.5706229 | hypothetical protein DKFZp762E1312 |
| 530310 | 0.5686743 | KIAA0143 protein |
| 41356 | 0.5683316 | “protein phosphatase 2, regulatory subunit B (B56), alpha |
| | | isoform” |
| 1844765 | 0.5670398 | Homo sapiens mRNA; cDNA DKFZp564O1763 (from clone |
| | | DKFZp564O1763) |
| 489823 | 0.5666593 | “COX17 (yeast) homolog, cytochrome c oxidase assembly |
| | | protein” |
| 1517749 | 0.5665848 | ESTs |
| 813410 | 0.5664086 | polymerase (RNA) II (DNA directed) polypeptide K (7.0 kD) |
| 1416782 | 0.5657017 | “creatine kinase, brain” |
| 1422338 | 0.5652946 | ribonucleotide reductase M2 polypeptide |
| 250313 | 0.5649247 | ESTs |
| 235986 | 0.5649127 | “wingless-type MMTV integration site family, member 11” |
| 66317 | 0.5648355 | “H1 histone family, member 2” |
| 279720 | 0.5645072 | “ESTs, Moderately similar to A47582 B-cell growth factor |
| | | precursor [H. sapiens]” |
| 884498 | 0.5640535 | uncharacterized hypothalamus protein HT012 |
| 503851 | 0.564048 | nuclear receptor co-repressor/HDAC3 complex subunit |
| 823930 | 0.5633774 | “actin related protein 2/3 complex, subunit 1A (41 kD)” |
| 120271 | 0.5626611 | hypothetical protein MGC4692 |
| 782513 | 0.5625811 | “interferon, alpha-inducible protein (clone IFI-6-16)” |
| 246800 | 0.5625675 | hypothetical protein FLJ10803 |
| 2309073 | 0.5617024 | frizzled (Drosophila) homolog 5 |
| 784150 | 0.5598483 | “RAB31, member RAS oncogene family” |
| 139835 | 0.5590106 | UDP-glucose dehydrogenase |
| 1641894 | 0.5587185 | EST |
| 796757 | 0.5585876 | “adaptor-related protein complex 3, sigma 1 subunit” |
| 813616 | 0.5574238 | FK506-binding protein like |
| 2549634 | 0.5572052 | activator of S phase kinase |
| 741769 | 0.5565863 | “polymerase (DNA directed), beta” |
| 488642 | 0.5560728 | “ESTs, Weakly similar to I38022 hypothetical protein |
| | | [H. sapiens]” |
| 795498 | 0.5557245 | putative transmembrane protein |
| 1492780 | 0.5555908 | “Homo sapiens cDNA FLJ14459 fis, clone |
| | | HEMBB1002409” |
| 241043 | 0.5545302 | “Human clone 137308 mRNA, partial cds” |
| 295986 | 0.5544422 | emopamil-binding protein (sterol isomerase) |
| 839682 | 0.5542571 | ubiquitin-conjugating enzyme E2N (homologous to yeast |
| | | UBC13) |
| 1581941 | 0.5537015 | hypothetical protein FLJ14540 |
| 809466 | 0.5532437 | DNA segment on chromosome 19 (unique) 1177 |
| | | expressed sequence |
| 1947381 | 0.5524634 | hypothetical protein FLJ22329 |
| 825470 | 0.5523318 | topoisomerase (DNA) II alpha (170 kD) |
| 1848977 | 0.5519913 | glycerol kinase |
| 700792 | 0.5493223 | cyclin-dependent kinase inhibitor 3 (CDK2-associated dual |
| | | specificity phosphatase) |
| 825386 | 0.5487104 | “ATP synthase, H+ transporting, mitochondrial F1F0, |
| | | subunit d” |
| 826194 | 0.5486839 | synaptotagmin-like 2 |
| 299815 | 0.5485843 | hypothetical protein DC42 |
| 1753497 | 0.5485231 | ovo (Drosophila) homolog-like 1 |
| 744944 | 0.5482368 | myosin VI |
| 1499828 | 0.5479012 | “fucosyltransferase 1 (galactoside 2-alpha-L- |
| | | fucosyltransferase, Bombay phenotype included)” |
| 415102 | 0.5465851 | cell division cycle 25C |
| 813387 | 0.5460577 | diaphorase (NADH/NADPH) (cytochrome b-5 reductase) |
| 786265 | 0.5443881 | KIAA0750 gene product |
| 1591264 | 0.5440944 | transaldolase 1 |
| 756595 | 0.5433619 | “S100 calcium-binding protein A10 (annexin II ligand, |
| | | calpactin I, light polypeptide (p11))” |
| 855749 | 0.5432466 | triosephosphate isomerase 1 |
| 470092 | 0.5429717 | like-glycosyltransferase |
| 244764 | 0.5424555 | B7 homolog 3 |
| 1637282 | 0.542121 | hexokinase 2 |
| 417801 | 0.5414761 | mitochondrial ribosomal protein L27 |
| 826077 | 0.5405591 | pyruvate dehydrogenase (lipoamide) beta |
| 757489 | 0.5386267 | “tubulin, alpha 2” |
| 327635 | 0.5368033 | adenylate kinase 1 |
| 343731 | 0.5365018 | |
| 786067 | 0.5361882 | cell division cycle 25B |
| 2017415 | 0.5353408 | centromere protein A (17 kD) |
| 292996 | 0.5341429 | “tyrosine 3-monooxygenase/tryptophan 5-monooxygenase |
| | | activation protein, eta polypeptide” |
| 815781 | 0.5340254 | heat shock 105 kD |
| 346942 | 0.5326646 | “phosphatidylinositol glycan, class Q” |
| 823694 | 0.5321203 | “Homo sapiens chromosome 19, BAC CIT-HSPC_204F22 |
| | | (BC228680), complete sequence; contains bacterial |
| | | insertion element” |
| 139354 | 0.5318709 | hypothetical protein |
| 221499 | 0.5317861 | KIAA0508 protein |
| 263727 | 0.5313082 | “DNA segment, single copy probe LNS-CAI/LNS-CAII |
| | | (deleted in polyposis” |
| 489594 | 0.5308806 | hypothetical protein FLJ11565 |
| 824524 | 0.5296138 | UDP-galactose transporter related |
| 951216 | 0.5291832 | “NADH dehydrogenase (ubiquinone) 1 beta subcomplex, |
| | | 10 (22 kD, PDSW)” |
| 841260 | 0.5285587 | hypothetical protein |
| 283739 | 0.5276555 | “Homo sapiens cDNA FLJ14028 fis, clone |
| | | HEMBA1003838” |
| 49117 | 0.5274035 | KIAA0215 gene product |
| 1762111 | 0.5272967 | natriuretic peptide receptor C/guanylate cyclase C |
| | | (atrionatriuretic peptide receptor C) |
| 358609 | 0.5266332 | NADH dehydrogenase (ubiquinone) flavoprotein 3 (10 kD) |
| 884425 | 0.5265114 | “chaperonin containing TCP1, subunit 5 (epsilon)” |
| 795401 | 0.5264278 | diacylglycerol O-acyltransferase (mouse) homolog |
| 811907 | 0.5246416 | hypothetical protein FLJ22056 |
| 1631747 | 0.5245873 | male-enhanced antigen |
| 365060 | 0.5244569 | “RAB11A, member RAS oncogene family” |
| 743220 | 0.5242028 | hypothetical protein FLJ12517 |
| 705064 | 0.5238148 | “transforming, acidic coiled-coil containing protein 3” |
| 768452 | 0.5237494 | “Homo sapiens EST from clone 491476, full insert” |
| 743977 | 0.5237158 | Homo sapiens mRNA for TL132 |
| 1568825 | 0.5205165 | Arg/Abl-interacting protein ArgBP2 |
| 772898 | 0.520392 | ribosomal protein S15a |
| 366834 | 0.5191005 | envoplakin |
| 1616253 | 0.5190257 | breast carcinoma amplified sequence 1 |
| 322617 | 0.5188645 | v-ral simian leukemia viral oncogene homolog B (ras |
| | | related; GTP binding protein) |
| 825296 | 0.5178447 | low density lipoprotein receptor defect C complementing |
| 491524 | 0.5175752 | mitochondrial ribosomal protein L13 |
| 143426 | 0.5173212 | “ras homolog gene family, member B” |
| 73009 | 0.516678 | “ESTs, Weakly similar to A43932 mucin 2 precursor, |
| | | intestinal [H. sapiens]” |
| 293569 | 0.5162069 | chromosome 1 open reading frame 21 |
| 296702 | 0.5162041 | “deiodinase, iodothyronine, type I” |
| 773286 | 0.5158979 | “solute carrier family 9 (sodium/hydrogen exchanger), |
| | | isoform 3 regulatory factor 1” |
| 726658 | 0.5157542 | “non-metastatic cells 3, protein expressed in” |
| 753897 | 0.5153412 | autocrine motility factor receptor |
| 293727 | 0.5153298 | hypothetical protein MGC861 |
| 810947 | 0.5151346 | “LIS1-interacting protein NUDE1, rat homolog” |
| 491465 | 0.5150527 | hypothetical protein FLJ10035 |
| 814899 | 0.5142481 | BCL2/adenovirus E1B 19 kD-interacting protein 3-like |
| 266500 | 0.5135558 | ESTs |
| 1159963 | 0.5133656 | interferon regulatory factor 7 |
| 785707 | 0.5133005 | protein regulator of cytokinesis 1 |
| 346134 | 0.5132068 | calcium-regulated heat-stable protein (24 kD) |
| 486110 | 0.5127183 | profilin 2 |
| 2017403 | 0.5117711 | regulator of G-protein signalling 3 |
| 491527 | 0.5116183 | “Homo sapiens, Similar to CG7083 gene product, clone |
| | | MGC: 10534 IMAGE: 3957147, mRNA, complete cds” |
| 769600 | 0.5106262 | uracil-DNA glycosylase 2 |
| 1558233 | 0.5101529 | ESTs |
| 649084 | 0.5099122 | carbonic anhydrase XI |
| 825822 | 0.5097808 | hypothetical protein |
| 1461477 | 0.509331 | Homo sapiens mRNA; cDNA DKFZp586I0324 (from clone |
| | | DKFZp586I0324) |
| 327506 | 0.5092192 | Homo sapiens mRNA full length insert cDNA clone |
| | | EUROIMAGE 327506 |
| 769942 | 0.509086 | kinesin-like 4 |
| 505289 | 0.5080495 | “angiotensin II, type I receptor-associated protein” |
| 726439 | 0.5080059 | CGI-143 protein |
| 79726 | 0.5078677 | “ESTs, Highly similar to T46395 hypothetical protein |
| | | DKFZp434I1120.1 [H. sapiens]” |
| 810762 | 0.5072899 | SNARE protein |
| 785793 | 0.5072717 | “capping protein (actin filament) muscle Z-line, alpha 1” |
| 81599 | 0.5067033 | ubiquitin specific protease 14 (tRNA-guanine |
| | | transglycosylase) |
| 294397 | 0.5066683 | DKFZP586A0522 protein |
| 1947647 | 0.5064941 | CGI-147 protein |
| 795805 | 0.5064099 | KIAA0332 protein |
| 810959 | 0.505619 | Rho GDP dissociation inhibitor (GDI) alpha |
| 2322223 | 0.5047814 | small nuclear ribonucleoprotein polypeptide A |
| 472103 | 0.503987 | “soc-2 (suppressor of clear, C. elegans) homolog” |
| 810609 | 0.5036493 | hypothetical protein PP1226 |
| 897813 | 0.5034689 | polyadenylate binding protein-interacting protein 1 |
| 1500542 | 0.5032679 | regulator of G-protein signalling 11 |
| 839746 | 0.5031787 | “Homo sapiens, Similar to RIKEN cDNA 5830420C20 |
| | | gene, clone IMAGE: 3633379, mRNA, partial cds” |
| 343607 | 0.5029133 | AD-015 protein |
| 649977 | 0.5002695 | Homo sapiens clone CDABP0014 mRNA sequence |
| 823940 | 0.4997308 | “transducer of ERBB2, 1” |
| 2250839 | 0.4994848 | androgen receptor (dihydrotestosterone receptor; testicular |
| | | feminization; spinal and bulbar muscular atrophy; Kennedy |
| | | disease) |
| 731080 | 0.4979864 | hypothetical protein FLJ12661 |
| 753400 | 0.496254 | CGI-204 protein |
| 869375 | 0.4958886 | “isocitrate dehydrogenase 2 (NADP+), mitochondrial” |
| 154610 | 0.4948571 | dynactin 4 |
| 130835 | 0.4946783 | “Homo sapiens, Similar to clone FLB3816, clone |
| | | IMAGE: 3454380, mRNA” |
| 859228 | 0.4942316 | “isocitrate dehydrogenase 1 (NADP+), soluble” |
| 41698 | 0.4938629 | progesterone binding protein |
| 30170 | 0.4936252 | “caspase 3, apoptosis-related cysteine protease” |
| 266218 | 0.4934791 | hypothetical protein FLJ11350 |
| 509588 | 0.49319 | “TATA box binding protein (TBP)-associated factor, RNA |
| | | polymerase II, J, 20 kD” |
| 1916575 | 0.4923447 | BCL2-interacting killer (apoptosis-inducing) |
| 744994 | 0.4916322 | hypothetical protein FLJ12242 |
| 487444 | 0.4887966 | “cyclic AMP phosphoprotein, 19 kD” |
| 503866 | 0.4884193 | sperm autoantigenic protein 17 |
| 814353 | 0.4883684 | phorbol-12-myristate-13-acetate-induced protein 1 |
| 815501 | 0.4879053 | hypothetical protein MGC2721 |
| 746163 | 0.4863317 | “ESTs, Weakly similar to ALU1_HUMAN ALU SUBFAMILY |
| | | J SEQUENCE CONTAMINATION WARNING ENTRY |
| | | [H. sapiens]” |
| 347726 | 0.4837701 | homeo box D8 |
| 897770 | 0.4836347 | |
| 1854539 | 0.4824023 | GAP-associated tyrosine phosphoprotein p62 (Sam68) |
| 41123 | 0.481642 | “Homo sapiens, Similar to RIKEN cDNA 2210021G21 |
| | | gene, clone MGC: 14859 IMAGE: 3621871, mRNA, |
| | | complete cds” |
| 856447 | 0.4816395 | “interferon, gamma-inducible protein 30” |
| 1568561 | 0.4808662 | BCL2-like 1 |
| 744047 | 0.4802334 | polo (Drosophia)-like kinase |
| 290101 | 0.4800598 | ESTs |
| 455275 | 0.4788778 | hypothetical protein FLJ23469 |
| 2069602 | 0.4786416 | melanocortin 1 receptor (alpha melanocyte stimulating |
| | | hormone receptor) |
| 592801 | 0.4785497 | “serine palmitoyltransferase, long chain base subunit 2” |
| 741891 | 0.4775456 | “RAB2, member RAS oncogene family-like” |
| 1434948 | 0.4771454 | HIV TAT specific factor 1 |
| 430614 | 0.476288 | “2,3-bisphosphoglycerate mutase” |
| 454896 | 0.4760701 | “DnaJ (Hsp40) homolog, subfamily A, member 2” |
| 268946 | 0.4751692 | WD40 protein Ciao1 |
| 1572710 | 0.4750848 | hypothetical protein FLJ21213 |
| 626318 | 0.4746692 | ubinuclein 1 |
| 377384 | 0.4746085 | “nuclear receptor subfamily 2, group F, member 2” |
| 1632252 | 0.474559 | “complement component 1, q subcomponent, alpha |
| | | polypeptide” |
| 489444 | 0.4741367 | hypothetical protein FLJ20211 |
| 345787 | 0.4734963 | “highly expressed in cancer, rich in leucine heptad repeats” |
| 767487 | 0.4729158 | “ariadne (Drosophila) homolog, ubiquitin-conjugating |
| | | enzyme E2-binding protein, 1” |
| empty-31 | 0.4726918 | blank |
| 135640 | 0.4708685 | syntaxin 3A |
| 754653 | 0.4707927 | “cadherin, EGF LAG seven-pass G-type receptor 3, |
| | | flamingo (Drosophila) homolog” |
| 1557637 | 0.4705061 | ESTs |
| 788641 | 0.4703576 | “adaptor-related protein complex 1, sigma 2 subunit” |
| 810567 | 0.4702908 | “Homo sapiens, clone MGC: 3182 IMAGE: 3356293, mRNA, |
| | | complete cds” |
| 366039 | 0.4701924 | KIAA0892 protein |
| 781047 | 0.4699376 | budding uninhibited by benzimidazoles 1 (yeast homolog) |
| 782688 | 0.4694868 | “dynein, axonemal, light intermediate polypeptide” |
| 210862 | 0.4692392 | “acyl-Coenzyme A oxidase 1, palmitoyl” |
| 510794 | 0.4691467 | c-myc binding protein |
| 121436 | 0.4691298 | “Homo sapiens, clone MGC: 4677 IMAGE: 3532809, mRNA, |
| | | complete cds” |
| 325515 | 0.4689353 | hypothetical protein FLJ10980 |
| 965223 | 0.4687291 | “thymidine kinase 1, soluble” |
| 1469148 | 0.467634 | FGFR1 oncogene partner |
| 149539 | 0.4671676 | KIAA1700 |
| 2306987 | 0.4670155 | secreted and transmembrane 1 |
| 122147 | 0.4652151 | |
| 2063982 | 0.4651856 | “potassium channel, subfamily K, member 6 (TWIK-2)” |
| 769890 | 0.4647115 | nucleoside phosphorylase |
| 1642496 | 0.4645092 | hypothetical protein MGC11266 |
| 428582 | 0.4642013 | hypothetical protein FLJ20296 |
| 66599 | 0.4639123 | N-acetyltransferase 1 (arylamine N-acetyltransferase) |
| 1565455 | 0.4634802 | “nuclear receptor subfamily 2, group C, member 2” |
| 810939 | 0.4634658 | hypothetial protein FLJ22169 |
| 359723 | 0.4630712 | mitochondrial elongation factor G |
| 1027283 | 0.4628012 | ESTs |
| 1636844 | 0.4627748 | ring finger protein 14 |
| 469151 | 0.4622121 | “eukaryotic translation initiation factor 2, subunit 2 (beta, |
| | | 38 kD)” |
| 813675 | 0.4614894 | “Human D9 splice variant B mRNA, complete cds” |
| 489657 | 0.4609514 | tryptophan rich basic protein |
| 469383 | 0.4607375 | chromosome 8 open reading frame 1 |
| 40042 | 0.4604322 | hypothetical protein FLJ10747 |
| 726637 | 0.45959 | t-complex-associated-testis-expressed 1-like |
| 811142 | 0.4595393 | “phosphoinositide-3-kinase, regulatory subunit, polypeptide |
| | | 2 (p85 beta)” |
| 179212 | 0.4594831 | “ESTs, Moderately similar to T12539 hypothetical protein |
| | | DKFZp434J154.1 [H. sapiens]” |
| 292936 | 0.4593712 | hypothetical protein FLJ10468 |
| 2046679 | 0.4579596 | ESTs |
| 810497 | 0.4572069 | “ESTs, Weakly similar to A35363 synapsin I splice form a |
| | | [H. sapiens]” |
| 1585492 | 0.4571478 | ESTs |
| 782689 | 0.4570254 | “solute carrier family 6 (neurotransmitter transporter, |
| | | creatine), member 8” |
| 774446 | 0.4567242 | adrenomedullin |
| 366156 | 0.4563893 | “Homo sapiens cDNA FLJ14028 fis, clone |
| | | HEMBA1003838” |
| 725649 | 0.4559473 | “nuclear factor of activated T-cells, cytoplasmic, |
| | | calcineurin-dependent 4” |
| 76362 | 0.4553121 | “spectrin, alpha, non-erythrocytic 1 (alpha-fodrin)” |
| 754046 | 0.4550413 | DNA segment on chromosome X (unique) 9879 expressed |
| | | sequence |
| 345538 | 0.4550133 | cathepsin L |
| 782439 | 0.454474 | “ATP synthase, H+ transporting, mitochondrial F0 complex, |
| | | subunit e” |
| 1537001 | 0.4539586 | ESTs |
| 345423 | 0.4538841 | DKFZP564M112 protein |
| 1702742 | 0.453453 | “solute carrier family 7 (cationic amino acid transporter, y+ |
| | | system), member 5” |
| 825659 | 0.4515758 | N-myc downstream regulated |
| 1652310 | 0.4512689 | “Homo sapiens, clone MGC: 19613 IMAGE: 3833049, |
| | | mRNA, complete cds” |
| 1523225 | 0.4511104 | oncostatin M receptor |
| 40773 | 0.4510289 | “guanine nucleotide binding protein (G protein), alpha z |
| | | polypeptide” |
| 35626 | 0.4509825 | “Homo sapiens cDNA FLJ14201 fis, clone |
| | | NT2RP3002955” |
| 358217 | 0.4507923 | glypican 4 |
| 214996 | 0.45033 | ESTs |
| 177827 | 0.4500254 | synaptotagmin VII |
| 1075635 | 0.4495618 | MLSN1- and TRP-related |
| 810039 | 0.4491986 | defender against cell death 1 |
| 772220 | 0.4475721 | for protein disulfide isomerase-related |
| 70606 | 0.4475592 | ESTs |
| 1692195 | 0.4471181 | smg GDS-ASSOCIATED PROTEIN |
| 841621 | 0.4466003 | “ESTs, Weakly similar to JE0350 Anterior gradient-2 |
| | | [H. sapiens]” |
| 855707 | 0.4456433 | ESTs |
| 1455394 | 0.4455695 | cytochrome c |
| 85804 | 0.44449 | hypothetical protein FLJ21918 |
| 809512 | 0.4442565 | hypothetical protein FLJ10767 |
| 753457 | 0.4435792 | NADH dehydrogenase (ubiquinone) Fe—S protein 1 (75 kD) |
| | | (NADH-coenzyme Q reductase) |
| 767289 | 0.4432685 | hypothetical protein FLJ10055 |
| 565235 | 0.4428854 | spermine synthase |
| 611467 | 0.4428543 | NADH dehydrogenase (ubiquinone) Fe—S protein 6 (13 kD) |
| | | (NADH-coenzyme Q reductase) |
| 812105 | 0.4424976 | ALL1-fused gene from chromosome 1q |
| 1558642 | 0.4424721 | hypothetical protein MGC2771 |
| 814989 | 0.4419384 | “protein phosphatase 1G (formerly 2C), magnesium- |
| | | dependent, gamma isoform” |
| 1899312 | 0.4418248 | ESTs |
| 1486533 | 0.441804 | keratin 16 (focal non-epidermolytic palmoplantar |
| | | keratoderma) |
| 1565079 | 0.4415966 | B-cell linker |
| 783681 | 0.4412503 | upstream regulatory element binding protein 1 |
| 1422723 | 0.4411931 | interferon-induced protein 35 |
| 785368 | 0.4411742 | PDZ-binding kinase; T-cell originated protein kinase |
| 1517595 | 0.4409591 | KIAA0175 gene product |
| 713862 | 0.4400704 | ubiquitin-protein isopeptide ligase (E3) |
| 83358 | 0.4397161 | ESTs |
| 81203 | 0.4391758 | paraoxonase 3 |
| 74070 | 0.4390933 | endosulfine alpha |
| 2306221 | 0.4385881 | “wingless-type MMTV integration site family, member 10B” |
| 140635 | 0.4384907 | ESTs |
| 150118 | 0.4383146 | hypothetical protein DKFZp434F054 |
| 214068 | 0.4383007 | GATA-binding protein 3 |
| 1434905 | 0.4382073 | homeo box B7 |
| 450301 | 0.4380657 | mutL (E. coli) homolog 3 |
| 2027952 | 0.437854 | hypothetical protein FLJ20297 |
| 745394 | 0.4374406 | “Homo sapiens cDNA: FLJ23249 fis, clone COL04196” |
| 824108 | 0.4369933 | SCAN domain-containing 1 |
| 795543 | 0.4364791 | thioredoxin peroxidase (antioxidant enzyme) |
| 1592837 | 0.4355927 | interferon induced transmembrane protein 2 (1-8D) |
| 77533 | 0.435582 | “inositol polyphosphate-5-phosphatase, 40 kD” |
| 1583198 | 0.4350835 | “ESTs, Weakly similar to S65824 reverse transcriptase |
| | | homolog [H. sapiens]” |
| 84295 | 0.4349213 | interleukin 1 receptor antagonist |
| 770992 | 0.4346858 | |
| 837864 | 0.4345283 | progestin induced protein |
| 1909574 | 0.4342055 | mitochondrial ribosomal protein S11 |
| 128695 | 0.4340119 | “ESTs, Weakly similar to I38344 titin, cardiac muscle |
| | | [H. sapiens]” |
| 742581 | 0.433453 | “Homo sapiens cDNA FLJ10366 fis, clone |
| | | NT2RM2001420” |
| 52103 | 0.432756 | hypothetical protein FLJ23045 |
| 770675 | 0.4319407 | “Homo sapiens cDNA: FLJ21323 fis, clone COL02374” |
| 756373 | 0.4310628 | Rho guanine exchange factor (GEF) 16 |
| 43977 | 0.4309655 | KIAA0182 protein |
| 2322079 | 0.4309341 | |
| 25440 | 0.4304195 | “staufen (Drosophila, RNA-binding protein) homolog 2” |
| 207794 | 0.4297389 | “nuclear factor (erythroid-derived 2), 45 kD” |
| 746190 | 0.4291743 | hypothetical protein DKFZp761B1514 |
| 74566 | 0.4279059 | “exportin 1 (CRM1, yeast, homolog)” |
| 76196 | 0.4278385 | hypothetical protein FLJ20062 |
| 826173 | 0.4276784 | profilin 1 |
| 1587863 | 0.4270229 | acetyl-Coenzyme A acyltransferase 1 (peroxisomal 3- |
| | | oxoacyl-Coenzyme A thiolase) |
| 2017721 | 0.4268089 | “ESTs, Weakly similar to 2109260A B cell growth factor |
| | | [H. sapiens]” |
| 809464 | 0.426456 | “fibroblast growth factor receptor 2 (bacteria-expressed |
| | | kinase, keratinocyte growth factor receptor, craniofacial |
| | | dysostosis 1, Crouzon syndrome, Pfeiffer syndrome, |
| | | Jackson-Weiss syndrome)” |
| 41826 | 0.4264531 | ESTs |
| 1687976 | 0.4257058 | “endoplasmic reticulum chaperone SIL1, homolog of yeast” |
| 416436 | 0.4252319 | mitochondrial ribosomal protein L24 |
| 145491 | 0.4250102 | protocadherin 1 (cadherin-like 1) |
| 810316 | 0.4245985 | very long-chain acyl-CoA synthetase; lipidosin |
| 629944 | 0.4242364 | myosin VB |
| 1637302 | 0.424164 | DNAJ domain-containing |
| 754625 | 0.4236648 | “ATPase, Class II, type 9A” |
| 298417 | 0.4235025 | trefoil factor 3 (intestinal) |
| 47853 | 0.4231314 | “aldehyde dehydrogenase 4 family, member A1” |
| 809727 | 0.4224266 | unc-51 (C. elegans)-like kinase 1 |
| 774502 | 0.4222849 | “protein tyrosine phosphatase, non-receptor type 12” |
| 785840 | 0.4219115 | “SEC24 (S. cerevisiae) related gene family, member D” |
| 823907 | 0.4218216 | hypothetical protein FLJ10511 |
| 1679942 | 0.4215618 | KIAA1053 protein |
| 823909 | 0.4215098 | |
| 1600239 | 0.421459 | HSPC037 protein |
| 85195 | 0.4207753 | “growth arrest and DNA-damage-inducible, gamma” |
| 1640282 | 0.4205899 | KIAA1240 protein |
| 782503 | 0.4205398 | fatty acid desaturase 1 |
| 266312 | 0.4201146 | “ATPase, Cu++ transporting, beta polypeptide (Wilson |
| | | disease)” |
| 884511 | 0.4199518 | cytochrome c oxidase subunit VIIb |
| 2051697 | 0.4197703 | “inhibitor of kappa light polypeptide gene enhancer in B- |
| | | cells, kinase beta” |
| 724888 | 0.4196322 | “cytochrome P450, subfamily IVB, polypeptide 1” |
| 809507 | 0.4189998 | hypothetical protein FLJ20568 |
| 151449 | 0.4189856 | “protein tyrosine phosphatase, non-receptor type 21” |
| 743860 | 0.4189656 | “hypothetical protein, clone 2746033” |
| 345833 | 0.4187433 | heterogeneous nuclear ribonucleoprotein A/B |
| 259950 | 0.4187278 | hypothetical protein FLJ14991 |
| 283751 | 0.4179154 | cortistatin |
| 810875 | 0.4176896 | “solute carrier family 26, member 6” |
| 26883 | 0.4176151 | “protein kinase (cAMP-dependent, catalytic) inhibitor beta” |
| 38356 | 0.4173012 | “cytochrome P450, subfamily 46 (cholesterol 24- |
| | | hydroxylase)” |
| 35191 | 0.4165847 | stromal cell-derived factor 2 |
| 75859 | −2.4150177 | N-myc downstream-regulated gene 2 |
| 1569187 | −1.9090486 | heparan sulfate (glucosamine) 3-O-sulfotransferase 4 |
| 345670 | −1.889471 | “ESTs, Moderately similar to I59348 CCAAT binding |
| | | transcription factor CBF subunit C - rat [R. norvegicus]” |
| 760299 | −1.8654555 | dickkopf (Xenopus laevis) homolog 3 |
| 753071 | −1.8417236 | “Homo sapiens cDNA: FLJ22528 fis, clone HRC12825” |
| 344720 | −1.7951928 | glycophorin C (Gerbich blood group) |
| 810002 | −1.7256692 | “Homo sapiens, clone MGC: 19762 IMAGE: 3636045, |
| | | mRNA, complete cds” |
| 200814 | −1.7140538 | “membrane metallo-endopeptidase (neutral endopeptidase, |
| | | enkephalinase, CALLA, CD10)” |
| 131839 | −1.7087069 | folate receptor 1 (adult) |
| 300632 | −1.6969092 | hypothetical protein FLJ21044 similar to Rbig1 |
| 811920 | −1.6924158 | “interleukin 11 receptor, alpha” |
| 486683 | −1.6799604 | Homo sapiens mRNA; cDNA DKFZp564J0323 (from clone |
| | | DKFZp564J0323) |
| 1558675 | −1.6577507 | SRY (sex determining region Y)-box 10 |
| 840266 | −1.6530726 | “Homo sapiens cDNA: FLJ22667 fis, clone HSI08385” |
| 285377 | −1.6514168 | pellino (Drosophila) homolog 2 |
| 1469377 | −1.6473741 | lipoma HMGIC fusion partner-like 2 |
| 45099 | −1.6325981 | regucalcin (senescence marker protein-30) |
| 1161564 | −1.5917197 | desmuslin |
| 298122 | −1.5678888 | frizzled (Drosophila) homolog 7 |
| 796542 | −1.5553964 | ets variant gene 5 (ets-related molecule) |
| 289760 | −1.5534789 | “ESTs, Highly similar to T00391 hypothetical protein |
| | | KIAA0612 [H. sapiens]” |
| 22917 | −1.5156635 | Homo sapiens mRNA; cDNA DKFZp761M0111 (from clone |
| | | DKFZp761M0111) |
| 838478 | −1.5059801 | neurocalcin delta |
| 788234 | −1.4824055 | “inhibitor of DNA binding 4, dominant negative helix-loop- |
| | | helix protein” |
| 377461 | −1.4787671 | “caveolin 1, caveolae protein, 22 kD” |
| 67741 | −1.4786722 | PP2135 protein |
| 839736 | −1.4745405 | “crystallin, alpha B” |
| 712139 | −1.4736508 | ADP-ribosylation factor-like 7 |
| 160192 | −1.4695435 | “ESTs, Weakly similar to 2004399A chromosomal protein |
| | | [H. sapiens]” |
| 742685 | −1.4440371 | disabled (Drosophila) homolog 2 (mitogen-responsive |
| | | phosphoprotein) |
| 1556433 | −1.4191955 | GRO3 oncogene |
| 1554167 | −1.4126653 | hypothetical protein FLJ14529 |
| 877621 | −1.3987023 | nGAP-like protein |
| 1882697 | −1.3873266 | peanut (Drosophila)-like 2 |
| 72778 | −1.3873177 | “caspase 7, apoptosis-related cysteine protease” |
| 666879 | −1.3624072 | annexin A8 |
| 69002 | −1.3581187 | PPAR(gamma) angiopoietin related protein |
| 811848 | −1.3549757 | hypothetical protein |
| 490023 | −1.3516302 | hypothetical protein MGC2648 |
| 781017 | −1.3232108 | early growth response 2 (Krox-20 (Drosophila) homolog) |
| 767202 | −1.3217103 | latent transforming growth factor beta binding protein 2 |
| 291478 | −1.3155899 | runt-related transcription factor 3 |
| 757191 | −1.2902603 | ESTs |
| 130201 | −1.2885369 | intercellular adhesion molecule 2 |
| 1635320 | −1.2878152 | “amiloride-sensitive cation channel 2, neuronal” |
| 140574 | −1.2754214 | “small inducible cytokine subfamily D (Cys-X3-Cys), |
| | | member 1 (fractalkine, neurotactin)” |
| 416676 | −1.2751042 | pellino (Drosophila) homolog 1 |
| 814798 | −1.2748689 | “aldehyde dehydrogenase 1 family, member A3” |
| 611532 | −1.273871 | “troponin I, skeletal, fast” |
| 2056139 | −1.2697411 | LIM domain protein |
| 377275 | −1.2665702 | ataxia-telangiectasia group D-associated protein |
| 303109 | −1.2557454 | purinergic receptor (family A group 5) |
| 813265 | −1.2549431 | Homo sapiens mRNA; cDNA DKFZp564H1916 (from clone |
| | | DKFZp564H1916) |
| 878836 | −1.2507242 | “secretory granule, neuroendocrine protein 1 (7B2 protein)” |
| 1473471 | −1.2409979 | KIAA0194 protein |
| 529843 | −1.2405917 | “ESTs, Moderately similar to JC5238 galactosylceramide- |
| | | like protein, GCP [H. sapiens]” |
| 505864 | −1.2393277 | RalGDS-like gene |
| 781014 | −1.2359239 | suppression of tumorigenicity 5 |
| 470393 | −1.2354879 | “matrix metalloproteinase 7 (matrilysin, uterine)” |
| 290378 | −1.2346086 | podocalyxin-like |
| 196435 | −1.2282733 | ESTs |
| 71087 | −1.2181178 | “v-maf musculoaponeurotic fibrosarcoma (avian) oncogene |
| | | family, protein F” |
| 34093 | −1.2156885 | EST |
| 76182 | −1.2102812 | hypothetical protein DKFZp761F241 |
| 884462 | −1.2078038 | Down syndrome critical region gene 1 |
| 277571 | −1.2030738 | KIAA1706 protein |
| 841308 | −1.200964 | “myosin, light polypeptide kinase” |
| 162308 | −1.2006293 | ESTs |
| 52419 | −1.2004086 | Friedreich ataxia region gene X123 |
| 488404 | −1.1981268 | Homo sapiens clone TUA8 Cri-du-chat region mRNA |
| 712401 | −1.193113 | “phosphoinositide-3-kinase, catalytic, delta polypeptide” |
| 289428 | −1.1923798 | “neurotrophic tyrosine kinase, receptor, type 2” |
| 814443 | −1.1905978 | hypothetical protein MGC3232 |
| 153760 | −1.1793765 | EphB1 |
| 1917449 | −1.1785893 | “serum amyloid A4, constitutive” |
| 1493218 | −1.164886 | hypothetical protein FLJ22297 |
| 843070 | −1.1647509 | nucleoporin 88 kD |
| 753038 | −1.1629978 | kinesin family member C3 |
| 811088 | −1.1625002 | ephrin-B3 |
| 2106144 | −1.1619925 | regulated in glioma |
| 796181 | −1.1584587 | growth arrest-specific 6 |
| 1257131 | −1.1566479 | ESTs |
| 1473274 | −1.1557062 | “myosin regulatory light chain 2, smooth muscle isoform” |
| 208718 | −1.1498532 | annexin A1 |
| 811837 | −1.1468554 | eukaryotic translation elongation factor 1 alpha 1 |
| 768571 | −1.1461886 | SRY (sex determining region Y)-box 8 |
| 1474900 | −1.1347123 | keratin 15 |
| 756708 | −1.1346161 | “potassium intermediate/small conductance calcium- |
| | | activated channel, subfamily N, member 4” |
| 1555924 | −1.1342985 | CSR1 protein |
| 811837 | −1.1269309 | |
| 1878409 | −1.1144369 | catechol-O-methyltransferase |
| 786069 | −1.1143906 | beta-site APP-cleaving enzyme |
| 815737 | −1.1116223 | “ATP synthase, H+ transporting, mitochondrial F1 complex, |
| | | alpha subunit, isoform 1, cardiac muscle” |
| 132857 | −1.0989746 | Homo sapiens mRNA; cDNA DKFZp586N1323 (from clone |
| | | DKFZp586N1323) |
| 1871116 | −1.0975491 | Homo sapiens mRNA; cDNA DKFZp434C1714 (from clone |
| | | DKFZp434C1714); partial cds |
| 133236 | −1.0945253 | “RNA binding motif protein, X chromosome” |
| 283023 | −1.0825166 | chemokine (C—X3—C) receptor 1 |
| 2016775 | −1.0761731 | “G protein-coupled receptor, family C, group 5, member B” |
| 897963 | −1.0751021 | phosphatidic acid phosphatase type 2A |
| 814316 | −1.0654718 | ribosomal protein L13 |
| 47043 | −1.0559861 | tensin |
| 625399 | −1.0523138 | hypothetical protein similar to beta-transducin family |
| 416959 | −1.051868 | nuclear factor I/B |
| 366100 | −1.0467604 | matrilin 2 |
| 753162 | −1.0416438 | KIAA0603 gene product |
| 491403 | −1.0411657 | “tumor necrosis factor receptor superfamily, member 1B” |
| 813256 | −1.0407652 | “ATP-binding cassette, sub-family B (MDR/TAP), member |
| | | 1” |
| 740620 | −1.0395569 | tropomyosin 2 (beta) |
| 1577736 | −1.035115 | epidermal growth factor (beta-urogastrone) |
| 70245 | −1.0348655 | Homo sapiens mRNA full length insert cDNA clone |
| | | EUROIMAGE 50374 |
| 1626996 | −1.0328113 | c-fos induced growth factor (vascular endothelial growth |
| | | factor D) |
| 360254 | −1.0325653 | “cysteine-rich, angiogenic inducer 61” |
| 80344 | −1.0323882 | interleukin 7 receptor |
| 530958 | −1.0316423 | smoothened (Drosophila) homolog |
| 34150 | −1.0302576 | ESTs |
| 809784 | −1.030253 | “kallikrein 6 (neurosin, zyme)” |
| 990881 | −1.0289558 | |
| 416434 | −1.0284229 | SCN Circadian Oscillatory Protein (SCOP) |
| 162308 | −1.023071 | |
| 796475 | −1.0217348 | four and a half LIM domains 3 |
| 32489 | −1.0196444 | hypothetical protein DKFZp566A1524 |
| 2504881 | −1.0194516 | signal transducer and activator of transcription 5A |
| 188335 | −1.0161259 | “egf-like module containing, mucin-like, hormone receptor- |
| | | like sequence 2” |
| 593023 | −1.0135883 | “dystrobrevin, beta” |
| 139660 | −1.0134677 | ESTs |
| 772913 | −1.0122192 | calreticulin |
| 270826 | −1.0112851 | “Homo sapiens cDNA FLJ13329 fis, clone |
| | | OVARC1001795” |
| 1864302 | −1.0099902 | E74-like factor 5 (ets domain transcription factor) |
| 1587710 | −1.0092611 | period (Drosophila) homolog 1 |
| 1584540 | −1.0077508 | Homo sapiens mRNA; cDNA DKFZp586M2022 (from clone |
| | | DKFZp586M2022) |
| 788136 | −1.0073983 | “phosphodiesterase 4B, cAMP-specific (dunce |
| | | (Drosophila)-homolog phosphodiesterase E4)” |
| 40027 | −1.0057005 | ESTs |
| 1856063 | −1.0036618 | tweety (Drosophila) homolog 1 |
| 209537 | −0.9988461 | zinc finger protein 221 |
| 628955 | −0.997687 | forkhead box O1A (rhabdomyosarcoma) |
| 2095066 | −0.9972552 | “alcohol dehydrogenase 1C (class I), gamma polypeptide” |
| 1636166 | −0.9969091 | KIAA0668 protein |
| 882248 | −0.9965858 | transgelin |
| 415816 | −0.9954308 | ESTs |
| 897731 | −0.9927981 | latrophilin |
| 868396 | −0.9916319 | “tyrosine 3-monooxygenase/tryptophan 5-monooxygenase |
| | | activation protein, eta polypeptide” |
| 343760 | −0.9907562 | SH3 domain binding glutamic acid-rich protein like 2 |
| 1572298 | −0.9906095 | “CD3Z antigen, zeta polypeptide (TiT3 complex)” |
| 1474337 | −0.986117 | “phosphorylase, glycogen; brain” |
| 1609665 | −0.9851713 | BarH-like homeobox 2 |
| 2243051 | −0.9835274 | kallikrein 8 (neuropsin/ovasin) |
| 27544 | −0.9834143 | prominin (mouse)-like 1 |
| 281190 | −0.9830533 | ESTs |
| 161456 | −0.9820939 | serum amyloid A1 |
| 505243 | −0.980381 | “inositol 1,4,5-triphosphate receptor, type 2” |

### Example IV

### Genes for Discriminating Between Normal and ADH

As shown in Table 3 below, 600 genes were identified as being able to discriminate between normal and ADH.

TABLE 3

| CloneID | Weight | Description |
|---|---|---|
| 1323448 | 1.3237504 | cysteine-rich protein 1 (intestinal) |
| 594500 | 1.158305 | EST |
| 824052 | 1.034408 | chromosome 6 open reading frame 1 |
| 149539 | 0.9878366 | KIAA1700 |
| 788654 | 0.9585887 | growth factor receptor-bound protein 2 |
| 1517749 | 0.947675 | ESTs |
| 1492238 | 0.9155471 | HSPC003 protein |
| 366353 | 0.9154768 | DKFZP564C186 protein |
| 1609836 | 0.898716 | glutamate-ammonia ligase (glutamine synthase) |
| 138189 | 0.8950816 | Wolfram syndrome 1 (wolframin) |
| 344959 | 0.8945717 | gene for serine/threonine protein kinase |
| 1435862 | 0.8934643 | antigen identified by monoclonal antibodies 12E7, F21 and O13 |
| 2014373 | 0.8820247 | HNK-1 sulfotransferase |
| 23776 | 0.879215 | quinoid dihydropteridine reductase |
| 293569 | 0.8708985 | chromosome 1 open reading frame 21 |
| 130835 | 0.8659688 | Homo sapiens, Similar to clone FLB3816, clone |
| | | IMAGE: 3454380, mRNA |
| 236034 | 0.8541082 | uncoupling protein 2 (mitochondrial, proton carrier) |
| 488025 | 0.8525751 | Kruppel-associated box protein |
| 178805 | 0.8524069 | Human DNA sequence from clone RP5-850E9 on chromosome |
| | | 20. Contains part of the gene for a novel C2H2 type zinc finger |
| | | protein similar to Drosophila Scratch (Scrt), Slug and Xenopus |
| | | Snail, a novel gene similar to Drosophila CG6762, STSs, GSSs |
| | | and five CpG |
| 745606 | 0.8514931 | hypothetical protein PP591 |
| 1492463 | 0.8378401 | selenoprotein X, 1 |
| 530197 | 0.8206765 | ESTs, Moderately similar to ALU8_HUMAN ALU SUBFAMILY |
| | | SX SEQUENCE CONTAMINATION WARNING ENTRY |
| | | [H. sapiens] |
| 898222 | 0.8092364 | Homo sapiens clone 24418 mRNA sequence |
| 782608 | 0.8041049 | mitochondrial ribosomal protein L9 |
| 450301 | 0.8030667 | mutL (E. coli) homolog 3 |
| 196189 | 0.7992668 | cytochrome b-5 |
| 363144 | 0.7924672 | transcription factor AP-2 beta (activating enhancer-binding |
| | | protein 2 beta) |
| 68636 | 0.7904354 | hypothetical protein MGC2477 |
| 795185 | 0.7765346 | xenotropic and polytropic retrovirus receptor |
| 255754 | 0.7720883 | LUNX protein; PLUNC (palate lung and nasal epithelium clone); |
| | | tracheal epithelium enriched protein |
| 767798 | 0.7682106 | ATX1 (antioxidant protein 1, yeast) homolog 1 |
| 1558233 | 0.7654043 | ESTs |
| 769600 | 0.763743 | uracil-DNA glycosylase 2 |
| 1156538 | 0.7512287 | potassium inwardly-rectifying channel, subfamily J, member 11 |
| 1587863 | 0.7507482 | acetyl-Coenzyme A acyltransferase 1 (peroxisomal 3-oxoacyl- |
| | | Coenzyme A thiolase) |
| 1709791 | 0.740832 | BAI1-associated protein 1 |
| 741891 | 0.7360507 | RAB2, member RAS oncogene family-like |
| 788654 | 0.7336359 | |
| 811774 | 0.7223819 | CGI-49 protein |
| 1738208 | 0.7166687 | a disintegrin-like and metalloprotease (reprolysin type) with |
| | | thrombospondin type 1 motif, 4 |
| 120138 | 0.7096046 | J domain containing protein 1 |
| 2248488 | 0.7033736 | ems1 sequence (mammary tumor and squamous cell |
| | | carcinoma-associated (p80/85 src substrate) |
| 265103 | 0.702883 | Homo sapiens mRNA; cDNA DKFZp547M123 (from clone |
| | | DKFZp547M123) |
| 2054122 | 0.698129 | solute carrier family 11 (proton-coupled divalent metal ion |
| | | transporters), member 3 |
| 701751 | 0.6961443 | cut (Drosophila)-like 1 (CCAAT displacement protein) |
| 811582 | 0.690137 | golgi phosphoprotein 2 |
| 266312 | 0.6856592 | ATPase, Cu++ transporting, beta polypeptide (Wilson disease) |
| 35147 | 0.6842172 | ESTs, Weakly similar to unnamed protein product [H. sapiens] |
| 1325816 | 0.6829962 | polymerase (RNA) II (DNA directed) polypeptide L (7.6 kD) |
| 358217 | 0.6801945 | glypican 4 |
| 233349 | 0.6764709 | hypothetical protein FLJ10761 |
| 741977 | 0.6758385 | B-factor, properdin |
| 145132 | 0.6725186 | mannose-P-dolichol utilization defect 1 |
| 33267 | 0.6679827 | |
| 279720 | 0.6607182 | ESTs, Moderately similar to A47582 B-cell growth factor |
| | | precursor [H. sapiens] |
| 565319 | 0.6603668 | Homo sapiens mRNA; cDNA DKFZp564B1264 (from clone |
| | | DKFZp564B1264) |
| 771173 | 0.6594128 | mitochondrial ribosomal protein S21 |
| 271472 | 0.6582514 | C3HC4-like zinc finger protein |
| 768570 | 0.6581316 | hypothetical protein FLJ11280 |
| 744417 | 0.6563238 | carnitine acetyltransferase |
| 826256 | 0.6554059 | transmembrane 7 superfamily member 1 (upregulated in kidney) |
| 2250839 | 0.654954 | androgen receptor (dihydrotestosterone receptor; testicular |
| | | feminization; spinal and bulbar muscular atrophy; Kennedy |
| | | disease) |
| 179212 | 0.6499845 | ESTs, Moderately similar to T12539 hypothetical protein |
| | | DKFZp434J154.1 [H. sapiens] |
| 785701 | 0.6496359 | RAB31, member RAS oncogene family |
| 186768 | 0.6495706 | Homo sapiens, clone IMAGE: 3604869, mRNA |
| 503215 | 0.6457259 | pilin-like transcription factor |
| 37554 | 0.6440792 | hypothetical protein FLJ22353 |
| 2710524 | 0.6399293 | nuclear receptor coactivator 3 |
| 284261 | 0.6398576 | uncharacterized hematopoietic stem/progenitor cells protein |
| | | MDS030 |
| 741769 | 0.6390692 | polymerase (DNA directed), beta |
| 743146 | 0.6388187 | hypothetical protein FLJ23403 |
| 814528 | 0.6329458 | Homo sapiens cDNA: FLJ22139 fis, clone HEP20959 |
| 742595 | 0.6324947 | cyclin-dependent kinase 5 |
| 810063 | 0.630827 | growth factor, erv1 (S. cerevisiae)-like (augmenter of liver |
| | | regeneration) |
| 590310 | 0.6299822 | Homo sapiens mRNA; cDNA DKFZp434E2321 (from clone |
| | | DKFZp434E2321); partial cds |
| 841679 | 0.6283163 | calcium and integrin binding protein (DNA-dependent protein |
| | | kinase interacting protein) |
| 131094 | 0.6268883 | Homo sapiens cDNA: FLJ21587 fis, clone COL06946 |
| 788334 | 0.6264069 | mitochondrial ribosomal protein L23 |
| 1574058 | 0.6261947 | 1-acylglycerol-3-phosphate O-acyltransferase 2 |
| | | (lysophosphatidic acid acyltransferase, beta) |
| 731308 | 0.6259858 | citrate synthase |
| 826194 | 0.6205287 | synaptotagmin-like 2 |
| 2110511 | 0.6203153 | artemin |
| 811907 | 0.6180993 | hypothetical protein FLJ22056 |
| 814350 | 0.6173648 | insulin-degrading enzyme |
| 611443 | 0.6112713 | myoglobin |
| 782608 | 0.6105794 | |
| 22778 | 0.6100322 | sulfortranferase family 4A, member 1 |
| 128695 | 0.608873 | ESTs, Weakly similar to I38344 titin, cardiac muscle [H. sapiens] |
| 47853 | 0.6077928 | aldehyde dehydrogenase 4 family, member A1 |
| 814209 | 0.6076904 | ESTs |
| 590774 | 0.6071935 | mitogen-activated protein kinase 13 |
| 969877 | 0.6064355 | synaptosomal-associated protein, 25 kD |
| 1469425 | 0.6055267 | SRY (sex determining region Y)-box 22 |
| 301122 | 0.6041028 | extracellular matrix protein 1 |
| 199403 | 0.6027193 | lectin, galactoside-binding, soluble, 8 (galectin 8) |
| 813845 | 0.6018909 | RNA, U transporter 1 |
| 810331 | 0.6014164 | quiescin Q6 |
| 770766 | 0.600234 | DKFZP564C1940 protein |
| 279970 | 0.5992634 | adenosine A2a receptor |
| 490778 | 0.5957445 | low molecular mass ubiquinone-binding protein (9.5 kD) |
| 288999 | 0.5955672 | small protein effector 1 of Cdc42 |
| 504959 | 0.5953946 | Homo sapiens mRNA; cDNA DKFZp586G0321 (from clone |
| | | DKFZp586G0321) |
| 42408 | 0.5930231 | hypothetical protein MGC4604 |
| 263727 | 0.5926363 | DNA segment, single copy probe LNS-CAI/LNS-CAII (deleted in |
| | | polyposis |
| 854763 | 0.5918867 | Homo sapiens cDNA FLJ11341 fis, clone PLACE1010786 |
| 729975 | 0.5908615 | meningioma expressed antigen 5 (hyaluronidase) |
| 1733262 | 0.5891897 | BLu protein |
| 730313 | 0.5887579 | ESTs, Weakly similar to A43932 mucin 2 precursor, intestinal |
| | | [H. sapiens] |
| 1500000 | 0.5871787 | H2B histone family, member B |
| 595037 | 0.5870709 | retinoic acid induced 3 |
| 1635681 | 0.5859113 | NADH dehydrogenase (ubiquinone) 1 alpha subcomplex, 2 |
| | | (8 kD, B8) |
| 2325804 | 0.5840656 | 95 kDa retinoblastoma protein binding protein |
| 2014856 | 0.5837779 | major histocompatibility complex, class I-like sequence |
| 40173 | 0.5771304 | KIAA0807 protein |
| 796723 | 0.576028 | Homo sapiens clone CDABP0014 mRNA sequence |
| 239568 | 0.5750382 | annexin A9 |
| 2018527 | 0.5732092 | dolichyl-phosphate mannosyltransferase polypeptide 3 |
| 1649374 | 0.5731982 | homogentisate 1,2-dioxygenase (homogentisate oxidase) |
| 809357 | 0.571929 | Bernardinelli-Seip congenital lipodystrophy 2 (seipin) |
| 366132 | 0.5711651 | succinate dehydrogenase complex, subunit C, integral |
| | | membrane protein, 15 kD |
| 83358 | 0.5709886 | ESTs |
| 1762111 | 0.5703701 | natriuretic peptide receptor C/guanylate cyclase C |
| | | (atrionatriuretic peptide receptor C) |
| 244801 | 0.5693836 | Rho guanine exchange factor (GEF) 11 |
| 127646 | 0.5663395 | |
| 39884 | 0.5659552 | IMP (inosine monophosphate) dehydrogenase 1 |
| 202577 | 0.5629317 | histamine N-methyltransferase |
| 588262 | 0.5614929 | Homo sapiens, Similar to RIKEN cDNA 2600001A11 gene, |
| | | clone MGC: 9907 IMAGE: 3870073, mRNA, complete cds |
| 813419 | 0.5602324 | hydroxyacyl-Coenzyme A dehydrogenase, type II |
| 1911343 | 0.559091 | RAB26, member RAS oncogene family |
| 859761 | 0.5582503 | poliovirus receptor-related 2 (herpesvirus entry mediator B) |
| 342181 | 0.557997 | B-cell CLL/lymphoma 2 |
| 1558108 | 0.5577768 | ATP-binding cassette, sub-family C (CFTR/MRP), member 8 |
| 2017403 | 0.5568495 | regulator of G-protein signalling 3 |
| 122394 | 0.5554851 | guanine nucleotide binding protein (G protein), alpha 15 (Gq |
| | | class) |
| 824879 | 0.5548666 | hypothetical protein MGC11275 |
| 1708055 | 0.554593 | glioblastoma overexpressed |
| 1456701 | 0.5545861 | B-cell CLL/lymphoma 9 |
| 186301 | 0.5537115 | Homo sapiens cDNA FLJ12924 fis, clone NT2RP2004709 |
| 126851 | 0.5535041 | hypothetical protein FLJ11160 |
| 826622 | 0.5532463 | KIAA0430 gene product |
| 151449 | 0.5503739 | protein tyrosine phosphatase, non-receptor type 21 |
| 826668 | 0.550158 | KIAA0274 gene product |
| 76605 | 0.5482232 | nesca protein |
| 786072 | 0.5450231 | myosin IC |
| 2244196 | 0.5432772 | B-cell receptor-associated protein BAP29 |
| 704414 | 0.5431136 | small nuclear ribonucleoprotein polypeptides B and B1 |
| 739578 | 0.5402953 | GPI-anchored metastasis-associated protein homolog |
| 1404774 | 0.5399447 | parathyroid hormone-like hormone |
| 66599 | 0.5399379 | N-acetyltransferase 1 (arylamine N-acetyltransferase) |
| 359250 | 0.5382989 | carbonic anhydrase IV |
| 488202 | 0.5378254 | ESTs, Weakly similar to YZ28_HUMAN HYPOTHETICAL |
| | | PROTEIN ZAP128 [H. sapiens] |
| 235986 | 0.5371486 | wingless-type MMTV integration site family, member 11 |
| 214068 | 0.5357344 | GATA-binding protein 3 |
| 1500542 | 0.5341753 | regulator of G-protein signalling 11 |
| 470092 | 0.5319336 | like-glycosyltransferase |
| 589115 | 0.531314 | matrix metalloproteinase 1 (interstitial collagenase) |
| 810734 | 0.5308572 | polymerase (DNA-directed), delta 4 |
| 685516 | 0.5304998 | putative G protein-coupled receptor |
| 171912 | 0.5297738 | Homo sapiens cDNA FLJ10960 fis, clone PLACE1000564 |
| 1858892 | 0.5295858 | hypothetical protein MGC4825 |
| 414992 | 0.5293379 | K562 cell-derived leucine-zipper-like protein 1 |
| 37708 | 0.5283276 | hypothetical protein MGC3101 |
| 713782 | 0.5273402 | a disintegrin and metalloproteinase domain 15 (metargidin) |
| 471568 | 0.5255851 | hematological and neurological expressed 1 |
| 812238 | 0.5237287 | hypothetical protein MGC4692 |
| 1631735 | 0.5228778 | Homo sapiens, clone IMAGE: 3604336, mRNA, partial cds |
| 74070 | 0.5225238 | endosulfine alpha |
| 782497 | 0.5218113 | Homo sapiens, clone IMAGE: 3010666, mRNA, partial cds |
| 502774 | 0.5204735 | hypothetical protein FLJ20623 |
| 1517171 | 0.5204242 | interleukin 2 receptor, alpha |
| 50562 | 0.5195335 | chromosome 8 open reading frame 4 |
| 60565 | 0.5192032 | lethal giant larvae (Drosophila) homolog 2 |
| 1641894 | 0.5187462 | EST |
| 51083 | 0.5170561 | catenin (cadherin-associated protein), delta 2 (neural |
| | | plakophilin-related arm-repeat protein) |
| 296702 | 0.5164562 | deiodinase, iodothyronine, type I |
| 2018337 | 0.5163293 | glucosidase, beta; acid (includes glucosylceramidase) |
| 1605426 | 0.5150013 | hypothetical protein FLJ13352 |
| 1474149 | 0.5138729 | poliovirus receptor-related 1 (herpesvirus entry mediator C; |
| | | nectin) |
| 26294 | 0.5137265 | RNB6 |
| 810017 | 0.5135433 | plasminogen activator, urokinase receptor |
| 825822 | 0.5134094 | hypothetical protein |
| 810725 | 0.51329 | ATPase, H+ transporting, lysosomal (vacuolar proton pump) |
| | | 21 kD |
| 781019 | 0.5132896 | paraoxonase 2 |
| 726658 | 0.5132765 | non-metastatic cells 3, protein expressed in |
| 1916575 | 0.5127478 | BCL2-interacting killer (apoptosis-inducing) |
| 1469148 | 0.5125351 | FGFR1 oncogene partner |
| 726703 | 0.5118991 | Homo sapiens clone 23736 mRNA sequence |
| 725978 | 0.5108817 | ESTs, Moderately similar to ALU7_HUMAN ALU SUBFAMILY |
| | | SQ SEQUENCE CONTAMINATION WARNING ENTRY |
| | | [H. sapiens] |
| 1573946 | 0.509585 | programmed cell death 9 |
| 51741 | 0.5094912 | GTP-binding protein |
| 156363 | 0.5086284 | hypothetical protein FLJ12934 |
| 177827 | 0.5085582 | synaptotagmin VII |
| 294397 | 0.505999 | DKFZP586A0522 protein |
| 826077 | 0.503983 | pyruvate dehydrogenase (lipoamide) beta |
| 949938 | 0.5011384 | cystatin C (amyloid angiopathy and cerebral hemorrhage) |
| 1957136 | 0.5006732 | phenylethanolamine N-methyltransferase |
| 2018084 | 0.4999242 | Ste-20 related kinase |
| 784140 | 0.4984039 | WD repeat domain 15 |
| 810981 | 0.498146 | hypothetical protein FLJ20699 |
| 627248 | 0.4972176 | SBBI31 protein |
| 2302099 | 0.4967769 | sialidase 3 (membrane sialidase) |
| 302031 | 0.49677 | Ste20-related serine/threonine kinase |
| 183440 | 0.4967506 | arylsulfatase A |
| 1609748 | 0.4955206 | hypothetical protein MGC10882 |
| 183200 | 0.4955056 | fumarylacetoacetate hydrolase (fumarylacetoacetase) |
| 2306697 | 0.4953741 | neuromedin B |
| 292770 | 0.4944242 | Homo sapiens, clone IMAGE: 3627860, mRNA, partial cds |
| 856447 | 0.494183 | interferon, gamma-inducible protein 30 |
| 855563 | 0.4941473 | v-erb-b2 avian erythroblastic leukemia viral oncogene homolog 3 |
| 839081 | 0.4939384 | homolog of yeast long chain polyunsaturated fatty acid |
| | | elongation enzyme 2 |
| 49273 | 0.4937997 | solute carrier family 27 (fatty acid transporter), member 4 |
| 289505 | 0.4933344 | ESTs, Moderately similar to S65657 alpha-1C-adrenergic |
| | | receptor splice form 2 [H. sapiens] |
| 782689 | 0.4930071 | solute carrier family 6 (neurotransmitter transporter, creatine), |
| | | member 8 |
| 767495 | 0.4922026 | GLI-Kruppel family member GLI3 (Greig cephalopolysyndactyly |
| | | syndrome) |
| 782537 | 0.490998 | Homo sapiens cDNA: FLJ22562 fis, clone HSI01814 |
| 855029 | 0.4904632 | Ac-like transposable element |
| 705274 | 0.4904221 | diacylglycerol kinase, delta (130 kD) |
| 202514 | 0.4902228 | DNA (cytosine-5-)-methyltransferase 3 alpha |
| 725649 | 0.4878638 | nuclear factor of activated T-cells, cytoplasmic, calcineurin- |
| | | dependent 4 |
| 1884404 | 0.4878341 | KIAA0285 gene product |
| 825296 | 0.4858551 | low density lipoprotein receptor defect C complementing |
| 839580 | 0.4826315 | ESTs |
| 289857 | 0.4823282 | phenylethanolamine N-methyltransferase |
| 491465 | 0.4821169 | hypothetical protein FLJ10035 |
| 360778 | 0.4820033 | |
| 261472 | 0.4814013 | putative nuclear protein ORF1-FL49 |
| 840768 | 0.4812559 | ATPase, H+ transporting, lysosomal (vacuolar proton pump) |
| | | 16 kD |
| 123614 | 0.4800042 | hypothetical protein MGC4675 |
| 1603583 | 0.4785029 | SH3 domain binding glutamic acid-rich protein like |
| 782688 | 0.4780253 | dynein, axonemal, light intermediate polypeptide |
| 784150 | 0.4763599 | RAB31, member RAS oncogene family |
| 770869 | 0.4762758 | hypothetical protein MGC2592 |
| 455269 | 0.4761389 | |
| 365358 | 0.4756564 | pM5 protein |
| 795256 | 0.4753832 | NPD007 protein |
| 756666 | 0.474441 | protein phosphatase 1, catalytic subunit, alpha isoform |
| 767706 | 0.4731085 | ESTs |
| 488505 | 0.4727394 | accessory proteins BAP31/BAP29 |
| 344073 | 0.4726399 | ESTs, Weakly similar to K1CI_HUMAN KERATIN, TYPE I |
| | | CYTOSKELETAL 9 [H. sapiens] |
| 1558151 | 0.4723511 | basic leucine zipper transcription factor, ATF-like |
| 823615 | 0.471739 | Homo sapiens cDNA: FLJ21245 fis, clone COL01184 |
| 49351 | 0.4697521 | SEX gene |
| 1541711 | 0.4694413 | Homo sapiens cDNA: FLJ21513 fis, clone COL05778 |
| 868652 | 0.4682053 | complement component 4B |
| 430318 | 0.4681908 | parvalbumin |
| 143426 | 0.4679928 | ras homolog gene family, member B |
| 951125 | 0.4675003 | peroxisomal D3,D2-enoyl-CoA isomerase |
| 66535 | 0.466705 | ornithine decarboxylase antizyme 2 |
| 813281 | 0.4659334 | WW domain-containing protein 1 |
| 726699 | 0.4658249 | ESTs, Weakly similar to AAB47496 NG5 [H. sapiens] |
| 1492468 | 0.4643107 | DEME-6 protein |
| 1492426 | 0.4635224 | chromosome 19 open reading frame 3 |
| 489106 | 0.4628623 | hypothetical protein FLJ11210 |
| 488422 | 0.4626549 | high-mobility group 20B |
| 278430 | 0.4612914 | kinesin family member 5C |
| 1518890 | 0.4610867 | metallothionein-like 5, testis-specific (tesmin) |
| 2017756 | 0.4608709 | homolog of yeast MOG1 |
| 813631 | 0.4607567 | seven transmembrane protein TM7SF3 |
| 1557637 | 0.4588694 | ESTs |
| 298417 | 0.4587501 | trefoil factor 3 (intestinal) |
| 810402 | 0.4583168 | hypothetical protein |
| 767761 | 0.4565666 | DKFZP434B168 protein |
| 1473289 | 0.4540707 | protective protein for beta-galactosidase (galactosialidosis) |
| 773142 | 0.4530754 | hypothetical protein MGC2827 |
| 1652310 | 0.4525789 | Homo sapiens, clone MGC: 19613 IMAGE: 3833049, mRNA, |
| | | complete cds |
| 812143 | 0.4519186 | fibronectin leucine rich transmembrane protein 3 |
| 2326057 | 0.4506179 | MLN51 protein |
| 1075635 | 0.4502629 | MLSN1- and TRP-related |
| 825327 | 0.4497029 | Homo sapiens cDNA FLJ14105 fis, clone MAMMA1001202 |
| 809838 | 0.4466933 | |
| 786545 | 0.4453714 | protein phosphatase 1, regulatory (inhibitor) subunit 3B |
| 183556 | 0.4452493 | gap junction protein, alpha 4, 37 kD (connexin 37) |
| 503889 | 0.4433034 | hypothetical protein FLJ10656 |
| 725321 | 0.44288 | estrogen receptor 1 |
| 731459 | 0.4427978 | Homo sapiens cDNA: FLJ22296 fis, clone HRC04468 |
| 752631 | 0.4426961 | fibroblast growth factor receptor 3 (achondroplasia, |
| | | thanatophoric dwarfism) |
| 1558642 | 0.4426424 | hypothetical protein MGC2771 |
| 120271 | 0.4425879 | hypothetical protein MGC4692 |
| 487932 | 0.4419815 | synaptotagmin-like 2 |
| 1592715 | 0.4417898 | Homer, neuronal immediate early gene, 3 |
| 308466 | 0.4417728 | GTP-binding protein Sara |
| 840882 | 0.441569 | nucleotide binding protein |
| 823727 | 0.4406546 | Homo sapiens, clone IMAGE: 2905978, mRNA, partial cds |
| 1636092 | 0.4404649 | hypothetical protein FLJ20657 |
| 1505038 | 0.43988 | hypothetical protein FLJ20171 |
| 490965 | 0.4389746 | ESTs |
| 346696 | 0.437234 | TEA domain family member 4 |
| 345032 | 0.4360992 | ESTs |
| 487733 | 0.4356439 | |
| 810612 | 0.4352602 | S100 calcium-binding protein A11 (calgizzarin) |
| 122077 | 0.434495 | putative membrane protein |
| 417801 | 0.4325892 | mitochondrial ribosomal protein L27 |
| 878406 | 0.4311455 | metaxin 1 |
| 1947381 | 0.4311314 | hypothetical protein FLJ22329 |
| 232789 | 0.4292025 | polymerase (RNA) II (DNA directed) polypeptide J (13.3 kD) |
| 809944 | 0.4291762 | KIAA0310 gene product |
| 2019101 | 0.4289318 | angiotensinogen (serine (or cysteine) proteinase inhibitor, clade |
| | | A (alpha-1 antiproteinase, antitrypsin), member 8) |
| 1656062 | 0.4288838 | coagulation factor XII (Hageman factor) |
| 1390584 | 0.4284863 | mitochondrial intermediate peptidase |
| 1693357 | 0.4274493 | endothelin 2 |
| 75859 | −1.6337267 | N-myc downstream-regulated gene 2 |
| 1558675 | −1.4515145 | SRY (sex determining region Y)-box 10 |
| 753071 | −1.4473398 | Homo sapiens cDNA: FLJ22528 fis, clone HRC12825 |
| 810002 | −1.2720931 | Homo sapiens, clone MGC: 19762 IMAGE: 3636045, mRNA, |
| | | complete cds |
| 712139 | −1.2371074 | ADP-ribosylation factor-like 7 |
| 815737 | −1.1857965 | ATP synthase, H+ transporting, mitochondrial F1 complex, alpha |
| | | subunit, isoform 1, cardiac muscle |
| 503671 | −1.1528584 | Homo sapiens cDNA FLJ14368 fis, clone HEMBA1001122 |
| 345670 | −1.135654 | ESTs, Moderately similar to I59348 CCAAT binding transcription |
| | | factor CBF subunit C - rat [R. norvegicus] |
| 753038 | −1.1168529 | kinesin family member C3 |
| 741139 | −1.1025542 | eyes absent (Drosophila) homolog 2 |
| 839736 | −1.1019142 | crystallin, alpha B |
| 2013178 | −1.0884449 | death associated transcription factor 1 |
| 1577736 | −1.076549 | epidermal growth factor (beta-urogastrone) |
| 788234 | −1.0610946 | inhibitor of DNA binding 4, dominant negative helix-loop-helix |
| | | protein |
| 150897 | −1.0221539 | UDP-GlcNAc:betaGal beta-1,3-N-acetylglucosaminyltransferase 3 |
| 757873 | −1.0188169 | cyclin-dependent kinase 5, regulatory subunit 1 (p35) |
| 291478 | −1.0093382 | runt-related transcription factor 3 |
| 486683 | −1.0075654 | Homo sapiens mRNA; cDNA DKFZp564J0323 (from clone |
| | | DKFZp564J0323) |
| 290378 | −1.0043647 | podocalyxin-like |
| 772913 | −0.9823487 | calreticulin |
| 544639 | −0.9767278 | ESTs |
| 814798 | −0.9611553 | aldehyde dehydrogenase 1 family, member A3 |
| 131839 | −0.959265 | folate receptor 1 (adult) |
| 840266 | −0.9425178 | Homo sapiens cDNA: FLJ22667 fis, clone HSI08385 |
| 1555924 | −0.9421074 | CSR1 protein |
| 756708 | −0.9396531 | potassium intermediate/small conductance calcium-activated |
| | | channel, subfamily N, member 4 |
| 838478 | −0.934433 | neurocalcin delta |
| 742562 | −0.9269494 | chromosome 16 open reading frame 5 |
| 1469377 | −0.9086679 | lipoma HMGIC fusion partner-like 2 |
| 884462 | −0.9065675 | Down syndrome critical region gene 1 |
| 796542 | −0.9052403 | ets variant gene 5 (ets-related molecule) |
| 32493 | −0.9043423 | integrin, alpha 6 |
| 284592 | −0.9019931 | PRO1659 protein |
| 788136 | −0.8998648 | phosphodiesterase 4B, cAMP-specific (dunce (Drosophila)- |
| | | homolog phosphodiesterase E4) |
| 1161564 | −0.8865787 | desmuslin |
| 1635320 | −0.8845852 | amiloride-sensitive cation channel 2, neuronal |
| 344720 | −0.8822302 | glycophorin C (Gerbich blood group) |
| 1534700 | −0.8804685 | KIAA0830 protein |
| 193913 | −0.8724313 | v-yes-1 Yamaguchi sarcoma viral related oncogene homolog |
| 71087 | −0.870641 | v-maf musculoaponeurotic fibrosarcoma (avian) oncogene |
| | | family, protein F |
| 300632 | −0.8586688 | hypothetical protein FLJ21044 similar to Rbig1 |
| 416676 | −0.8568853 | pellino (Drosophila) homolog 1 |
| 69002 | −0.8528453 | PPAR(gamma) angiopoietin related protein |
| 742685 | −0.852598 | disabled (Drosophila) homolog 2 (mitogen-responsive |
| | | phosphoprotein) |
| 140574 | −0.8498606 | small inducible cytokine subfamily D (Cys-X3-Cys), member 1 |
| | | (fractalkine, neurotactin) |
| 23831 | −0.8493217 | aldolase C, fructose-bisphosphate |
| 470393 | −0.848482 | matrix metalloproteinase 7 (matrilysin, uterine) |
| 1474337 | −0.844539 | phosphorylase, glycogen; brain |
| 753301 | −0.8393651 | carcinoembryonic antigen-related cell adhesion molecule 1 |
| | | (biliary glycoprotein) |
| 51078 | −0.8348835 | ribosomal protein L44 |
| 3172883 | −0.8293392 | ESTs, Weakly similar to 1709359A dopamine D4 receptor |
| | | [H. sapiens] |
| 877621 | −0.8289253 | nGAP-like protein |
| 811920 | −0.8286379 | interleukin 11 receptor, alpha |
| 156211 | −0.8269263 | ATPase, H+ transporting, lysosomal (vacuolar proton pump), |
| | | beta polypeptide, 56/58 kD, isoform 1 (Renal tubular acidosis |
| | | with deafness) |
| 760299 | −0.8251086 | dickkopf (Xenopus laevis) homolog 3 |
| 285377 | −0.8214706 | pellino (Drosophila) homolog 2 |
| 345034 | −0.8161772 | small inducible cytokine subfamily B (Cys-X-Cys), member 14 |
| | | (BRAK) |
| 298122 | −0.8158372 | frizzled (Drosophila) homolog 7 |
| 842896 | −0.8123068 | hypothetical protein DKFZp762L0311 |
| 843070 | −0.8071984 | nucleoporin 88 kD |
| 1257131 | −0.8069059 | ESTs |
| 796539 | −0.803124 | KRAB-associated protein 1 |
| 139660 | −0.79833 | ESTs |
| 666879 | −0.7950485 | annexin A8 |
| 200814 | −0.7920988 | membrane metallo-endopeptidase (neutral endopeptidase, |
| | | enkephalinase, CALLA, CD10) |
| 2016775 | −0.7914834 | G protein-coupled receptor, family C, group 5, member B |
| 1946448 | −0.790849 | caveolin 2 |
| 1473471 | −0.7906678 | KIAA0194 protein |
| 1556433 | −0.7877792 | GRO3 oncogene |
| 1636156 | −0.7827089 | hypothetical protein FLJ21709 |
| 40299 | −0.78007 | growth differentiation factor 10 |
| 1636166 | −0.7788972 | KIAA0668 protein |
| 1609665 | −0.7765472 | BarH-like homeobox 2 |
| 250797 | −0.7739669 | hypothetical protein FLJ20038 |
| 220395 | −0.7687195 | hypothetical protein FLJ23293 similar to ARL-6 interacting |
| | | protein-2 |
| 208718 | −0.7660278 | annexin A1 |
| 41869 | −0.7646494 | hypothetical protein FLJ11017 |
| 490023 | −0.7621736 | hypothetical protein MGC2648 |
| 1607229 | −0.7562938 | tumor protein D52-like 1 |
| 133236 | −0.7561129 | RNA binding motif protein, X chromosome |
| 785733 | −0.7554682 | hypothetical protein FLJ12892 |
| 32489 | −0.7521767 | hypothetical protein DKFZp566A1524 |
| 70201 | −0.7513314 | mitochondrial solute carrier |
| 611481 | −0.7507264 | HMG-box transcription factor TCF-3 |
| 1554167 | −0.7503289 | hypothetical protein FLJ14529 |
| 768571 | −0.7476813 | SRY (sex determining region Y)-box 8 |
| 783698 | −0.7411614 | lipin 1 |
| 341759 | −0.7393558 | lung type-I cell membrane-associated glycoprotein |
| 665384 | −0.7388404 | KIAA1609 protein |
| 142259 | −0.7380151 | tumor necrosis factor alpha-inducible cellular protein containing |
| | | leucine zipper domains; Huntingtin interacting protein L; |
| | | transcrption factor IIIA-interacting protein |
| 810057 | −0.7363941 | vasoactive intestinal peptide receptor 1 |
| 27544 | −0.7342174 | prominin (mouse)-like 1 |
| 739193 | −0.7338774 | cellular retinoic acid-binding protein 1 |
| 813256 | −0.7334866 | ATP-binding cassette, sub-family B (MDR/TAP), member 1 |
| 293964 | −0.7275302 | butyrophilin, subfamily 3, member A1 |
| 272706 | −0.7274769 | alpha2,3-sialyltransferase |
| 209537 | −0.7245778 | zinc finger protein 221 |
| 67741 | −0.7229251 | PP2135 protein |
| 823714 | −0.7215045 | nuclear receptor co-repressor/HDAC3 complex subunit |
| 471196 | −0.7192752 | integral membrane protein 3 |
| 813265 | −0.7192289 | Homo sapiens mRNA; cDNA DKFZp564H1916 (from clone |
| | | DKFZp564H1916) |
| 42681 | −0.718704 | NY-REN-25 antigen |
| 1601845 | −0.7178673 | Ca2+-promoted Ras inactivator |
| 725152 | −0.7173869 | hypothetical protein DKFZp762A227 |
| 22917 | −0.7173328 | Homo sapiens mRNA; cDNA DKFZp761M0111 (from clone |
| | | DKFZp761M0111) |
| 703541 | −0.7118124 | KIAA1858 protein |
| 529843 | −0.7117107 | ESTs, Moderately similar to JC5238 galactosylceramide-like |
| | | protein, GCP [H. sapiens] |
| 377461 | −0.7093974 | caveolin 1, caveolae protein, 22 kD |
| 1569187 | −0.7091259 | heparan sulfate (glucosamine) 3-O-sulfotransferase 4 |
| 1456118 | −0.7074166 | proteasome (prosome, macropain) subunit, beta type, 9 (large |
| | | multifunctional protease 2) |
| 289760 | −0.7058375 | ESTs, Highly similar to T00391 hypothetical protein KIAA0612 |
| | | [H. sapiens] |
| 341774 | −0.7044768 | major histocompatibility complex, class I, A |
| 75078 | −0.7028415 | ESTs |
| 505864 | −0.7020337 | RalGDS-like gene |
| 415191 | −0.700303 | KIAA0161 gene product |
| 188335 | −0.7002579 | egf-like module containing, mucin-like, hormone receptor-like |
| | | sequence 2 |
| 132711 | −0.6985643 | Kruppel-like factor 5 (intestinal) |
| 1864302 | −0.6943806 | E74-like factor 5 (ets domain transcription factor) |
| 344854 | −0.692296 | ankyrin repeat domain 3 |
| 454970 | −0.6853932 | DKFZP434G032 protein |
| 79629 | −0.6849525 | |
| 767068 | −0.6819032 | DKFZP586G1517 protein |
| 378813 | −0.679848 | secretory leukocyte protease inhibitor (antileukoproteinase) |
| 725076 | −0.6798005 | 5′-nucleotidase (purine), cytosolic type B |
| 491403 | −0.6760814 | tumor necrosis factor receptor superfamily, member 1B |
| 43764 | −0.6743049 | hypothetical protein FLJ14033 similar to hypoxia inducible factor |
| | | 3, alpha subunit |
| 725622 | −0.6731681 | ESTs |
| 431231 | −0.6692022 | EGF-containing fibulin-like extracellular matrix protein 2 |
| 488956 | −0.6682156 | CUG triplet repeat, RNA-binding protein 2 |
| 72778 | −0.6614808 | caspase 7, apoptosis-related cysteine protease |
| 360254 | −0.6612871 | cysteine-rich, angiogenic inducer, 61 |
| 76182 | −0.6603322 | hypothetical protein DKFZp761F241 |
| 773487 | −0.6598647 | hypothetical protein MGC3032 |
| 162308 | −0.6580605 | ESTs |
| 283023 | −0.6577611 | chemokine (C-X3-C) receptor 1 |
| 196992 | −0.6573113 | aldo-keto reductase family 1, member C1 (dihydrodiol |
| | | dehydrogenase 1; 20-alpha (3-alpha)-hydroxysteroid |
| | | dehydrogenase) |
| 303109 | −0.6571713 | purinergic receptor (family A group 5) |
| 897731 | −0.6548064 | latrophilin |
| 289428 | −0.6542789 | neurotrophic tyrosine kinase, receptor, type 2 |
| 196435 | −0.653913 | ESTs |
| 504791 | −0.6537859 | glutathione S-transferase A4 |
| 812975 | −0.6527728 | KIAA0172 protein |
| 488404 | −0.6521658 | Homo sapiens clone TUA8 Cri-du-chat region mRNA |
| 345056 | −0.6490368 | KIAA1404 protein |
| 786069 | −0.6484842 | beta-site APP-cleaving enzyme |
| 48518 | −0.6455041 | ATP-binding cassette, sub-family A (ABC1), member 5 |
| 812048 | −0.6438062 | prion protein (p27-30) (Creutzfeld-Jakob disease, Gerstmann- |
| | | Strausler-Scheinker syndrome, fatal familial insomnia) |
| 868396 | −0.6426678 | tyrosine 3-monooxygenase/tryptophan 5-monooxygenase |
| | | activation protein, eta polypeptide |
| 70245 | −0.6422928 | Homo sapiens mRNA full length insert cDNA clone |
| | | EUROIMAGE 50374 |
| 40027 | −0.6418674 | ESTs |
| 840944 | −0.6399619 | early growth response 1 |
| 809784 | −0.6348991 | kallikrein 6 (neurosin, zyme) |
| 1881774 | −0.6332545 | KIAA1678 |
| 52419 | −0.6326767 | Friedreich ataxia region gene X123 |
| 120106 | −0.631375 | caspase 1, apoptosis-related cysteine protease (interleukin 1, |
| | | beta, convertase) |
| 725680 | −0.6295778 | transcription factor AP-2 gamma (activating enhancer-binding |
| | | protein 2 gamma) |
| 1493218 | −0.6292715 | hypothetical protein FLJ22297 |
| 2018423 | −0.6275903 | death-associated protein kinase 2 |
| 1486082 | −0.6241275 | heparin-binding growth factor binding protein |
| 868169 | −0.6224058 | lipoprotein lipase |
| 209137 | −0.6161588 | gamma-aminobutyric acid (GABA) A receptor, epsilon |
| 248886 | −0.6161134 | rab3 GTPase-activating protein, non-catalytic subunit (150 kD) |
| 73252 | −0.6115602 | 3-hydroxy-3-methylglutaryl-Coenzyme A synthase 1 (soluble) |
| 612274 | −0.6108452 | tubulin, alpha 1 (testis specific) |
| 346545 | −0.6098367 | laminin, beta 1 |
| 84464 | −0.6091345 | hypothetical protein FLJ12806 |
| 628955 | −0.6088522 | forkhead box O1A (rhabdomyosarcoma) |
| 490414 | −0.6078858 | chromosome 2 open reading frame 2 |
| 752837 | −0.6056177 | Homo sapiens mRNA for FLJ00074 protein, partial cds |
| 796904 | −0.604755 | pleiomorphic adenoma gene-like 1 |
| 418159 | −0.6038708 | synaptogyrin 1 |
| 323238 | −0.6032171 | GRO1 oncogene (melanoma growth stimulating activity, alpha) |
| 1570427 | −0.601608 | hypothetical protein MGC4309 |
| 1500815 | −0.6008126 | Homo sapiens cDNA: FLJ22130 fis, clone HEP19632 |
| 416959 | −0.5999728 | nuclear factor I/B |
| 782460 | −0.5988226 | Homo sapiens cDNA FLJ10500 fis, clone NT2RP2000369 |
| 824602 | −0.5965816 | interferon, gamma-inducible protein 16 |
| 878798 | −0.5956349 | beta-2-microglobulin |
| 32299 | −0.5945347 | inositol(myo)-1(or 4)-monophosphatase 2 |
| 277571 | −0.59335 | KIAA1706 protein |
| 703964 | −0.5918604 | inositol polyphosphate phosphatase-like 1 |
| 796498 | −0.5900328 | hypothetical protein FLJ14007 |
| 504927 | −0.5894078 | epithelial protein up-regulated in carcinoma, membrane |
| | | associated protein 17 |
| 813520 | −0.5889772 | EphB3 |
| 341763 | −0.5868818 | caspase 5, apoptosis-related cysteine protease |
| 1856063 | −0.5866417 | tweety (Drosophila) homolog 1 |
| 825356 | −0.5850287 | Homo sapiens cDNA FLJ11997 fis, clone HEMBB1001458 |
| 745011 | −0.5836864 | Homo sapiens mRNA for KIAA1750 protein, partial cds |
| 812256 | −0.5832398 | Homo sapiens cDNA: FLJ21693 fis, clone COL09609 |
| 2017960 | −0.5822336 | PP1201 protein |
| 713129 | −0.5822078 | granzyme A (granzyme 1, cytotoxic T-lymphocyte-associated |
| | | serine esterase 3) |
| 815142 | −0.5782827 | spastic ataxia of Charlevoix-Saguenay (sacsin) |
| 80727 | −0.5766546 | receptor tyrosine kinase-like orphan receptor 1 |
| 45099 | −0.5753698 | regucalcin (senescence marker protein-30) |
| 415816 | −0.5750184 | ESTs |
| 306798 | −0.5742664 | NGFI-A binding protein 1 (EGR1 binding protein 1) |
| 563634 | −0.5737239 | dendritic cell protein |
| 757191 | −0.5735888 | ESTs |
| 740620 | −0.5730915 | tropomyosin 2 (beta) |
| 1470048 | −0.5729078 | lymphocyte antigen 6 complex, locus E |
| 788518 | −0.5724497 | peroxisomal membrane protein 3 (35 kD, Zellweger syndrome) |
| 177665 | −0.5718286 | |
| 221846 | −0.5714352 | checkpoint suppressor 1 |
| 37671 | −0.5712258 | hypothetical protein FLJ21610 |
| 2072862 | −0.5694163 | v-akt murine thymoma viral oncogene homolog 2 |
| 132637 | −0.5694152 | grancalcin, EF-hand calcium-binding protein |
| 1474900 | −0.5693091 | keratin 15 |
| 34093 | −0.5692081 | EST |
| 1881689 | −0.568518 | hypothetical protein FLJ20281 |
| 1883327 | −0.5684295 | ESTs |
| 1902764 | −0.5683052 | 6-phosphofructo-2-kinase/fructose-2,6-biphosphatase 3 |
| 781017 | −0.5676944 | early growth response 2 (Krox-20 (Drosophila) homolog) |
| 34150 | −0.5673709 | ESTs |
| 137602 | −0.5667198 | Homo sapiens mRNA; cDNA DKFZp434G0972 (from clone |
| | | DKFZp434G0972) |
| 460126 | −0.5652362 | KIAA0819 protein |
| 470128 | −0.5652125 | myosin IE |
| 1880885 | −0.5638973 | ESTs |
| 811848 | −0.5637994 | hypothetical protein |
| 1417886 | −0.563564 | hypothetical protein FLJ23239 |
| 756847 | −0.5632582 | deformed epidermal autoregulatory factor 1 (Drosophila) |
| 505243 | −0.5616577 | inositol 1,4,5-triphosphate receptor, type 2 |
| 840677 | −0.5611985 | immunoglobulin kappa constant |
| 1911663 | −0.5610314 | ESTs |
| 66491 | −0.5579872 | plasmolipin |
| 1420842 | −0.5574834 | mucosa associated lymphoid tissue lymphoma translocation |
| | | gene 1 |
| 1626996 | −0.5573507 | c-fos induced growth factor (vascular endothelial growth factor |
| | | D) |
| 281190 | −0.5572026 | ESTs |
| 796475 | −0.5563457 | four and a half LIM domains 3 |
| 1870305 | −0.5558155 | heat shock 27 kD protein 2 |
| 767456 | −0.5555493 | hypothetical protein FLJ22167 |
| 292388 | −0.554297 | |
| 795178 | −0.5532479 | lactate dehydrogenase C |
| 809998 | −0.5519459 | amylase, alpha 2A; pancreatic |
| 1734754 | −0.5512835 | hypothetical protein B |
| 27769 | −0.5501268 | ESTs |
| 950355 | −0.550114 | ESTs, Weakly similar to S13495 pregnancy zone protein |
| | | [H. sapiens] |
| 144924 | −0.5498123 | chromosome 6 open reading frame 5 |
| 502436 | −0.5489864 | insulin receptor substrate 2 |
| 138672 | −0.5483104 | ESTs |
| 160192 | −0.5482009 | ESTs, Weakly similar to 2004399A chromosomal protein |
| | | [H. sapiens] |
| 768007 | −0.547374 | hypothetical protein |
| 130201 | −0.5468031 | intercellular adhesion molecule 2 |
| 1587847 | −0.5466442 | minichromosome maintenance deficient (mis5, S. pombe) 6 |
| 1558212 | −0.5458261 | ESTs |
| 1470278 | −0.5449844 | hypothetical protein FLJ21841 |
| 811088 | −0.5445374 | ephrin-B3 |
| 197056 | −0.5440514 | ESTs |
| 868630 | −0.5426831 | transforming growth factor beta-stimulated protein TSC-22 |
| 530958 | −0.5423532 | smoothened (Drosophila) homolog |
| 810097 | −0.5423072 | Homo sapiens cDNA: FLJ21721 fis, clone COLF0381 |
| 207735 | −0.5422739 | serine (or cysteine) proteinase inhibitor, clade A (alpha-1 |
| | | antiproteinase, antitrypsin), member 1 |
| 79254 | −0.541673 | MHC class I region ORF |
| 85634 | −0.5410684 | complement component 1, s subcomponent |
| 531319 | −0.5404527 | serine/threonine kinase 12 |
| 754028 | −0.5396001 | KIAA0469 gene product |
| 470148 | −0.5393193 | ESTs |
| 35828 | −0.5391332 | diphtheria toxin receptor (heparin-binding epidermal growth |
| | | factor-like growth factor) |
| 970590 | −0.5383645 | Homo sapiens mRNA; cDNA DKFZp434A115 (from clone |
| | | DKFZp434A115) |
| 753162 | −0.5377767 | KIAA0603 gene product |
| 241066 | −0.5369493 | Homo sapiens cDNA: FLJ21028 fis, clone CAE07155 |
| 1909935 | −0.5368632 | ESTs |
| 153760 | −0.5361176 | EphB1 |
| 247616 | −0.5358709 | lipoma HMGIC fusion partner |
| 1031640 | −0.5354869 | ESTs |
| 24958 | −0.5349519 | Homo sapiens mRNA; cDNA DKFZp434C2016 (from clone |
| | | DKFZp434C2016) |
| 1686766 | −0.5342051 | Rag D protein |
| 2009491 | −0.5334821 | cyclin-E binding protein 1 |
| 611532 | −0.5320107 | troponin I, skeletal, fast |
| 811149 | −0.5319742 | chromosome 9 open reading frame 3 |
| 844703 | −0.5309356 | Sam68-like phosphotyrosine protein, T-STAR |
| 70749 | −0.5295091 | Homo sapiens cDNA: FLJ21874 fis, clone HEP02488 |
| 324927 | −0.5284875 | KIAA0375 gene product |
| 491519 | −0.5282605 | Homo sapiens clone 24775 mRNA sequence |
| 754040 | −0.5282144 | NS1-associated protein 1 |
| 767765 | −0.5282008 | GTP-binding protein overexpressed in skeletal muscle |
| 2028722 | −0.5277252 | osteoblast specific factor 2 (fasciclin I-like) |
| 509458 | −0.5266016 | hypothetical protein from clone 643 |
| 360518 | −0.5253745 | guanine nucleotide binding protein (G protein), gamma |
| | | transducing activity polypeptide 1 |
| 40021 | −0.5240809 | hairless protein (putative single zinc finger transcription factor |
| | | protein, responsible for autosomal recessive universal |
| | | congenital alopecia, HR gene) |
| 1901310 | −0.5229967 | KIAA1209 protein |
| 33949 | −0.5222135 | phosphoribosyl pyrophosphate synthetase-associated protein 1 |
| 814316 | −0.521831 | ribosomal protein L13 |
| 488130 | −0.5201845 | Homo sapiens cDNA FLJ20767 fis, clone COL06986 |
| 462939 | −0.5200097 | ESTs |
| 770848 | −0.5188717 | ESTs, Weakly similar to ALU1_HUMAN ALU SUBFAMILY J |
| | | SEQUENCE CONTAMINATION WARNING ENTRY [H. sapiens] |
| 245296 | −0.5184238 | RAD52 (S. cerevisiae) homolog |
| 753034 | −0.5182019 | nuclear factor I/X (CCAAT-binding transcription factor) |
| 712829 | −0.5181236 | LIM domain only 2 (rhombotin-like 1) |
| 1577920 | −0.5161377 | ESTs |
| 814526 | −0.5156162 | seb4D |
| 773301 | −0.5147295 | cadherin 3, type 1, P-cadherin (placental) |
| 77972 | −0.5144916 | complement component 3 |
| 1704155 | −0.5133015 | hypothetical protein SBBI48 |
| 1572298 | −0.5126426 | CD3Z antigen, zeta polypeptide (TiT3 complex) |
| 755689 | −0.5122586 | retinoic acid receptor, gamma |
| 1876217 | −0.5108977 | DnaJ (Hsp40) homolog, subfamily A, member 2 |
| 712401 | −0.5107426 | phosphoinositide-3-kinase, catalytic, delta polypeptide |
| 713839 | −0.5088558 | transcription factor AP-4 (activating enhancer-binding protein 4) |
| 180803 | −0.5083354 | inositol polyphosphate-1-phosphatase |
| 814443 | −0.5075293 | hypothetical protein MGC3232 |

### Example V

### Genes for Discriminating Between Normal and DCIS

As shown in Table 4 below, 1300 genes were identified as being able to discriminate between normal and DCIS.

TABLE 4

| CloneID | Weight | Description |
|---|---|---|
| 1323448 | 1.397391 | cysteine-rich protein 1 (intestinal) |
| 788654 | 1.2615322 | growth factor receptor-bound protein 2 |
| 1505038 | 1.1904802 | hypothetical protein FLJ20171 |
| 745606 | 1.1825585 | hypothetical protein PP591 |
| 565319 | 1.0969429 | Homo sapiens mRNA; cDNA DKFZp564B1264 (from clone |
| | | DKFZp564B1264) |
| 35147 | 1.0886084 | ESTs, Weakly similar to unnamed protein product [H. sapiens] |
| 178805 | 1.0274897 | Human DNA sequence from clone RP5-850E9 on chromosome |
| | | 20. Contains part of the gene for a novel C2H2 type zinc finger |
| | | protein similar to Drosophila Scratch (Scrt), Slug and Xenopus |
| | | Snail, a novel gene similar to Drosophila CG6762, STSs, GSSs |
| | | and five CpG |
| 796469 | 1.0269115 | HSPC150 protein similar to ubiquitin-conjugating enzyme |
| 1609836 | 1.0164283 | glutamate-ammonia ligase (glutamine synthase) |
| 366132 | 1.013995 | succinate dehydrogenase complex, subunit C, integral |
| | | membrane protein, 15 kD |
| 1500000 | 1.0118809 | H2B histone family, member B |
| 812238 | 1.0043787 | hypothetical protein MGC4692 |
| 595037 | 0.990461 | retinoic acid induced 3 |
| 1554549 | 0.9764206 | hydroxyacyl glutathione hydrolase |
| 488964 | 0.9569176 | H2A histone family, member O |
| 138189 | 0.9386802 | Wolfram syndrome 1 (wolframin) |
| 283919 | 0.937383 | H2A histone family, member L |
| 823598 | 0.9153521 | proteasome (prosome, macropain) 26S subunit, non-ATPase, |
| | | 12 |
| 810711 | 0.9128832 | stearoyl-CoA desaturase (delta-9-desaturase) |
| 1911343 | 0.9116745 | RAB26, member RAS oncogene family |
| 503215 | 0.9108688 | pilin-like transcription factor |
| 811774 | 0.9073627 | CGI-49 protein |
| 122077 | 0.9021901 | putative membrane protein |
| 1492238 | 0.9019233 | HSPC003 protein |
| 469686 | 0.8919992 | Ric (Drosophila)-like, expressed in many tissues |
| 685516 | 0.8762873 | putative G protein-coupled receptor |
| 359887 | 0.8692353 | translocase of inner mitochondrial membrane 17 (yeast) |
| | | homolog A |
| 131094 | 0.8675719 | Homo sapiens cDNA: FLJ21587 fis, clone COL06946 |
| 1492463 | 0.8634521 | selenoprotein X, 1 |
| 843195 | 0.8603524 | phosphoserine phosphatase |
| 796723 | 0.8573155 | Homo sapiens clone CDABP0014 mRNA sequence |
| 488202 | 0.8463961 | ESTs, Weakly similar to YZ28_HUMAN HYPOTHETICAL |
| | | PROTEIN ZAP128 [H. sapiens] |
| 1492426 | 0.8445255 | chromosome 19 open reading frame 3 |
| 280375 | 0.8392836 | PRO2000 protein |
| 290841 | 0.8346933 | H2B histone family, member A |
| 1917941 | 0.8340565 | purine-rich element binding protein B |
| 1469425 | 0.8296141 | SRY (sex determining region Y)-box 22 |
| 2016908 | 0.8282806 | ESTs, Weakly similar to CA13_HUMAN COLLAGEN ALPHA |
| | | 1(III) CHAIN PRECURSOR [H. sapiens] |
| 471568 | 0.8264546 | hematological and neurological expressed 1 |
| 150003 | 0.8251408 | hypothetical protein FLJ13187 |
| 2029173 | 0.8213721 | ESTs, Weakly similar to N-WASP [H. sapiens] |
| 731044 | 0.8169506 | glutaredoxin 2 |
| 244801 | 0.8066357 | Rho guanine exchange factor (GEF) 11 |
| 2054635 | 0.7914755 | proteasome (prosome, macropain) subunit, alpha type, 7 |
| 1605426 | 0.787298 | hypothetical protein FLJ13352 |
| 1709791 | 0.7837549 | BAI1-associated protein 1 |
| 470061 | 0.77977 | seven in absentia (Drosophila) homolog 2 |
| 1640821 | 0.7790286 | ESTs, Weakly similar to I78885 serine/threonine-specific |
| | | protein kinase [H. sapiens] |
| 199403 | 0.7754748 | lectin, galactoside-binding, soluble, 8 (galectin 8) |
| 741474 | 0.7732047 | glucose phosphate isomerase |
| 1435862 | 0.7697423 | antigen identified by monoclonal antibodies 12E7, F21 and O13 |
| 239568 | 0.767277 | annexin A9 |
| 841679 | 0.7666686 | calcium and integrin binding protein (DNA-dependent protein |
| | | kinase interacting protein) |
| 729975 | 0.7649717 | meningioma expressed antigen 5 (hyaluronidase) |
| 2016648 | 0.7633048 | Homo sapiens mRNA; cDNA DKFZp434N1728 (from clone |
| | | DKFZp434N1728) |
| 2052113 | 0.7627076 | hypothetical protein FLJ10903 |
| 347373 | 0.7563599 | transcription elongation factor B (SIII), polypeptide 1 (15 kD, |
| | | elongin C) |
| 814054 | 0.7539407 | KIAA0040 gene product |
| 741977 | 0.7538359 | B-factor, properdin |
| 272529 | 0.7486269 | phosphomannomutase 2 |
| 824879 | 0.7432221 | hypothetical protein MGC11275 |
| 122241 | 0.7394284 | proteasome (prosome, macropain) subunit, beta type, 2 |
| 744417 | 0.7356767 | carnitine acetyltransferase |
| 868128 | 0.7347171 | JM4 protein |
| 241348 | 0.7324115 | prenylcysteine lyase |
| 509823 | 0.7304507 | carcinoembryonic antigen-related cell adhesion molecule 6 |
| | | (non-specific cross reacting antigen) |
| 1636092 | 0.7270017 | hypothetical protein FLJ20657 |
| 42408 | 0.725109 | hypothetical protein MGC4604 |
| 686552 | 0.7231319 | golgi phosphoprotein 1 |
| 898032 | 0.7216 | KIAA0097 gene product |
| 754628 | 0.7195265 | ESTs |
| 826256 | 0.7190206 | transmembrane 7 superfamily member 1 (upregulated in |
| | | kidney) |
| 2043167 | 0.7167431 | BCL2-associated athanogene 3 |
| 827171 | 0.7154964 | ESTs |
| 502774 | 0.7148695 | hypothetical protein FLJ20623 |
| 781097 | 0.7126945 | reticulon 3 |
| 810725 | 0.7115051 | ATPase, H+ transporting, lysosomal (vacuolar proton pump) |
| | | 21 kD |
| 768570 | 0.7102463 | hypothetical protein FLJ11280 |
| 813281 | 0.7092607 | WW domain-containing protein 1 |
| 39884 | 0.7075527 | IMP (inosine monophosphate) dehydrogenase 1 |
| 969877 | 0.707469 | synaptosomal-associated protein, 25 kD |
| 704254 | 0.7061648 | a disintegrin and metalloproteinase domain 8 |
| 236034 | 0.7039329 | uncoupling protein 2 (mitochondrial, proton carrier) |
| 782428 | 0.701077 | KIAA0250 gene product |
| 489351 | 0.7003616 | hypothetical protein DKFZp566J2046 |
| 37708 | 0.6986007 | hypothetical protein MGC3101 |
| 271472 | 0.6978431 | C3HC4-like zinc finger protein |
| 782608 | 0.6978285 | mitochondrial ribosomal protein L9 |
| 704414 | 0.6940045 | small nuclear ribonucleoprotein polypeptides B and B1 |
| 288999 | 0.6938843 | small protein effector 1 of Cdc42 |
| 51773 | 0.6925666 | hypothetical protein MGC3077 |
| 124447 | 0.6918546 | KIAA1184 protein |
| 470099 | 0.6915952 | HT002 protein; hypertension-related calcium-regulated gene |
| 429799 | 0.6915155 | hypothetical protein FLJ21939 similar to 5-azacytidine induced |
| | | gene 2 |
| 1858892 | 0.6904662 | hypothetical protein MGC4825 |
| 796694 | 0.6893307 | baculoviral IAP repeat-containing 5 (survivin) |
| 284261 | 0.6890531 | uncharacterized hematopoietic stem/progenitor cells protein |
| | | MDS030 |
| 814528 | 0.6878182 | Homo sapiens cDNA: FLJ22139 fis, clone HEP20959 |
| 788654 | 0.6866898 | |
| 68636 | 0.6838604 | hypothetical protein MGC2477 |
| 308466 | 0.6800103 | GTP-binding protein Sara |
| 1574058 | 0.6796357 | 1-acylglycerol-3-phosphate O-acyltransferase 2 |
| | | (lysophosphatidic acid acyltransferase, beta) |
| 79520 | 0.6769129 | RAB2, member RAS oncogene family |
| 1473289 | 0.674396 | protective protein for beta-galactosidase (galactosialidosis) |
| 365738 | 0.6740773 | ESTs |
| 768064 | 0.673441 | cytochrome P450, subfamily I (aromatic compound-inducible), |
| | | polypeptide 1 |
| 773922 | 0.6730903 | KIAA0005 gene product |
| 1869201 | 0.6727191 | hypothetical protein MGC2745 |
| 686172 | 0.6724684 | KIAA0008 gene product |
| 810558 | 0.6698194 | proteasome (prosome, macropain) 26S subunit, ATPase, 4 |
| 753299 | 0.6694573 | hypothetical protein FLJ10504 |
| 725454 | 0.6669973 | CDC28 protein kinase 2 |
| 470124 | 0.6641768 | RAD1 (S. pombe) homolog |
| 2014034 | 0.6620303 | methylene tetrahydrofolate dehydrogenase (NAD+ dependent), |
| | | methenyltetrahydrofolate cyclohydrolase |
| 209066 | 0.6611038 | |
| 859761 | 0.6573769 | poliovirus receptor-related 2 (herpesvirus entry mediator B) |
| 589232 | 0.6551447 | hypothetical protein FLJ11506 |
| 340558 | 0.6550804 | actin related protein 2/3 complex, subunit 5 (16 kD) |
| 186768 | 0.6550741 | Homo sapiens, clone IMAGE: 3604869, mRNA |
| 813629 | 0.6549767 | YME1 (S. cerevisiae)-like 1 |
| 1492780 | 0.6544659 | Homo sapiens cDNA FLJ14459 fis, clone HEMBB1002409 |
| 809944 | 0.6530236 | KIAA0310 gene product |
| 83363 | 0.6526164 | protein-L-isoaspartate (D-aspartate) O-methyltransferase |
| 149355 | 0.6520333 | translocating chain-associating membrane protein |
| 752631 | 0.650399 | fibroblast growth factor receptor 3 (achondroplasia, |
| | | thanatophoric dwarfism) |
| 785616 | 0.6484744 | signal sequence receptor, alpha (translocon-associated protein |
| | | alpha) |
| 76605 | 0.6484522 | nesca protein |
| 742707 | 0.6476434 | ESTs, Weakly similar to MUC2_HUMAN MUCIN 2 |
| | | PRECURSOR [H. sapiens] |
| 325606 | 0.647638 | hypothetical protein MGC14353 |
| 769921 | 0.6464245 | ubiquitin carrier protein E2-C |
| 1435003 | 0.6457486 | tumor suppressing subtransferable candidate 1 |
| 1473922 | 0.6450042 | actin related protein 2/3 complex, subunit 3 (21 kD) |
| 1456348 | 0.6417276 | N-acetylneuraminic acid phosphate synthase; sialic acid |
| | | synthase |
| 1500162 | 0.6416738 | ESTs |
| 1573251 | 0.6410435 | peroxisomal long-chain acyl-coA thioesterase |
| 46248 | 0.640944 | ADP-ribosyltransferase (NAD+; poly (ADP-ribose) polymerase) |
| 49351 | 0.6386132 | SEX gene |
| 824052 | 0.6375787 | chromosome 6 open reading frame 1 |
| 1420370 | 0.6344209 | biliverdin reductase B (flavin reductase (NADPH)) |
| 842994 | 0.6343858 | cathepsin Z |
| 810612 | 0.6341098 | S100 calcium-binding protein A11 (calgizzarin) |
| 123614 | 0.633308 | hypothetical protein MGC4675 |
| 124781 | 0.6324418 | squalene epoxidase |
| 814378 | 0.6317382 | serine protease inhibitor, Kunitz type, 2 |
| 2108077 | 0.6312583 | CGI-112 protein |
| 289978 | 0.6308861 | ubiquitin-like 4 |
| 67765 | 0.6300886 | carboxypeptidase M |
| 742595 | 0.6265846 | cyclin-dependent kinase 5 |
| 727078 | 0.6263882 | Homo sapiens cDNA: FLJ23602 fis, clone LNG15735 |
| 487733 | 0.6262778 | |
| 366067 | 0.625758 | cerebellar degeneration-related protein (62 kD) |
| 292770 | 0.6231261 | Homo sapiens, clone IMAGE: 3627860, mRNA, partial cds |
| 366353 | 0.6203012 | DKFZP564C186 protein |
| 810063 | 0.620086 | growth factor, erv1 (S. cerevisiae)-like (augmenter of liver |
| | | regeneration) |
| 810124 | 0.6162712 | platelet-activating factor acetylhydrolase, isoform Ib, gamma |
| | | subunit (29 kD) |
| 713782 | 0.6149877 | a disintegrin and metalloproteinase domain 15 (metargidin) |
| 782608 | 0.6137782 | |
| 530197 | 0.6122721 | ESTs, Moderately similar to ALU8_HUMAN ALU SUBFAMILY |
| | | SX SEQUENCE CONTAMINATION WARNING ENTRY |
| | | [H. sapiens] |
| 44292 | 0.6122043 | Homo sapiens mRNA; cDNA DKFZp434C107 (from clone |
| | | DKFZp434C107) |
| 2018084 | 0.6119069 | Ste-20 related kinase |
| 789012 | 0.6104002 | fibulin 2 |
| 256619 | 0.6102239 | hydroxysteroid (17-beta) dehydrogenase 7 |
| 825327 | 0.6094796 | Homo sapiens cDNA FLJ14105 fis, clone MAMMA1001202 |
| 842980 | 0.6081528 | developmentally regulated GTP-binding protein 1 |
| 811761 | 0.6045809 | Nijmegen breakage syndrome 1 (nibrin) |
| 1601947 | 0.6045149 | cytochrome c oxidase subunit VIIa polypeptide 2 (liver) |
| 1474955 | 0.6045103 | TATA box binding protein (TBP)-associated factor, RNA |
| | | polymerase II, N, 68 kD (RNA-binding protein 56) |
| 1758590 | 0.6040962 | fatty-acid-Coenzyme A ligase, long-chain 3 |
| 1456701 | 0.6038789 | B-cell CLL/lymphoma 9 |
| 207288 | 0.603346 | insulin induced gene 1 |
| 1631699 | 0.6027911 | valosin-containing protein |
| 1635681 | 0.6022549 | NADH dehydrogenase (ubiquinone) 1 alpha subcomplex, 2 |
| | | (8 kD, B8) |
| 768452 | 0.6018854 | Homo sapiens EST from clone 491476, full insert |
| 897806 | 0.6015789 | hypoxia-inducible factor 1, alpha subunit (basic helix-loop-helix |
| | | transcription factor) |
| 108425 | 0.6008772 | |
| 770785 | 0.600311 | 1,2-alpha-mannosidase IC |
| 2110511 | 0.5987863 | artemin |
| 785795 | 0.598504 | hypothetical protein FLJ12910 |
| 120271 | 0.5982507 | hypothetical protein MGC4692 |
| 248649 | 0.5975539 | hypothetical protein FLJ13910 |
| 2244196 | 0.5972111 | B-cell receptor-associated protein BAP29 |
| 66406 | 0.5966701 | hypothetical protein DKFZp762E1312 |
| 1537001 | 0.59528 | ESTs |
| 2028949 | 0.5946445 | hypothetical protein PRO1855 |
| 40173 | 0.5940582 | KIAA0807 protein |
| 490778 | 0.5932928 | low molecular mass ubiquinone-binding protein (9.5 kD) |
| 503889 | 0.5930018 | hypothetical protein FLJ10656 |
| 781019 | 0.5918462 | paraoxonase 2 |
| 196189 | 0.5916252 | cytochrome b-5 |
| 1734309 | 0.5894492 | sperm associated antigen 4 |
| 814350 | 0.588877 | insulin-degrading enzyme |
| 2018821 | 0.5887114 | ATPase inhibitor precursor |
| 365358 | 0.5883524 | pM5 protein |
| 430235 | 0.5874504 | H2B histone family, member Q |
| 1422338 | 0.5874097 | ribonucleotide reductase M2 polypeptide |
| 770845 | 0.5870877 | hexokinase 1 |
| 172517 | 0.5869599 | hippocalcin-like 1 |
| 1884404 | 0.5849803 | KIAA0285 gene product |
| 625923 | 0.5839066 | phosphoenolpyruvate carboxykinase 2 (mitochondrial) |
| 2322367 | 0.5832711 | reticulon 4 |
| 624667 | 0.5828923 | CGI-92 protein |
| 826363 | 0.5823873 | lysophospholipase II |
| 859228 | 0.5817344 | isocitrate dehydrogenase 1 (NADP+), soluble |
| 2302099 | 0.5813788 | sialidase 3 (membrane sialidase) |
| 209066 | 0.5794211 | serine/threonine kinase 15 |
| 1844765 | 0.57783 | Homo sapiens mRNA; cDNA DKFZp564O1763 (from clone |
| | | DKFZp564O1763) |
| 191904 | 0.5770884 | BUB3 (budding uninhibited by benzimidazoles 3, yeast) |
| | | homolog |
| 951233 | 0.5767874 | proteasome (prosome, macropain) subunit, beta type, 3 |
| 809357 | 0.5767382 | Bernardinelli-Seip congenital lipodystrophy 2 (seipin) |
| 825740 | 0.5762093 | DKFZp434J1813 protein |
| 51532 | 0.5758765 | ADP-ribosylation factor-like 6 interacting protein |
| 203003 | 0.5743416 | non-metastatic cells 4, protein expressed in |
| 2015517 | 0.5742448 | hypothetical protein FLJ22237 |
| 205049 | 0.5734444 | protein kinase H11; small stress protein-like protein HSP22 |
| 752643 | 0.5732323 | group XII secreted phospholipase A2 |
| 753400 | 0.5732322 | CGI-204 protein |
| 564847 | 0.573202 | ESTs |
| 785766 | 0.5731938 | hypothetical protein |
| 825585 | 0.5722829 | tubulin-specific chaperone e |
| 37554 | 0.5706008 | hypothetical protein FLJ22353 |
| 144880 | 0.5704416 | hypothetical protein from EUROIMAGE 1759349 |
| 49273 | 0.5691181 | solute carrier family 27 (fatty acid transporter), member 4 |
| 743589 | 0.568516 | ESTs, Weakly similar to T2D3_HUMAN TRANSCRIPTION |
| | | INITIATION FACTOR TFIID 135 KDA SUBUNIT [H. sapiens] |
| 1616253 | 0.567837 | breast carcinoma amplified sequence 1 |
| 1581941 | 0.5676045 | hypothetical protein FLJ14540 |
| 431505 | 0.5670119 | ESTs, Highly similar to A31026 probable membrane receptor |
| | | protein [H. sapiens] |
| 2019223 | 0.5663073 | mitochondrial ribosomal protein L17 |
| 840878 | 0.5648009 | seladin-1 |
| 811024 | 0.5646461 | bone marrow stromal cell antigen 2 |
| 564492 | 0.5626905 | mitochondrial carrier homolog 2 |
| 725841 | 0.561197 | KIAA0662 gene product |
| 701751 | 0.561139 | cut (Drosophila)-like 1 (CCAAT displacement protein) |
| 767798 | 0.560181 | ATX1 (antioxidant protein 1, yeast) homolog 1 |
| 109863 | 0.559767 | epithelial membrane protein 2 |
| 1536006 | 0.5590182 | ESTs |
| 1845169 | 0.5588842 | RAB35, member RAS oncogene family |
| 343607 | 0.5588222 | AD-015 protein |
| 212542 | 0.5587394 | Homo sapiens cDNA FLJ12900 fis, clone NT2RP2004321 |
| 1700436 | 0.5569949 | ESTs |
| 795256 | 0.5567908 | NPD007 protein |
| 2011515 | 0.5566484 | DKFZP586B0923 protein |
| 504308 | 0.5564295 | hypothetical protein FLJ10540 |
| 772925 | 0.5562164 | HSPCO34 protein |
| 469383 | 0.5558732 | chromosome 8 open reading frame 1 |
| 810402 | 0.5555749 | hypothetical protein |
| 1409509 | 0.554703 | troponin T1, skeletal, slow |
| 2050827 | 0.5544641 | proteasome (prosome, macropain) 26S subunit, ATPase, 5 |
| 770355 | 0.5518948 | lanosterol synthase (2,3-oxidosqualene-lanosterol cyclase) |
| 813410 | 0.5513568 | polymerase (RNA) II (DNA directed) polypeptide K (7.0 kD) |
| 80764 | 0.5510626 | hypothetical protein |
| 1631735 | 0.5502891 | Homo sapiens, clone IMAGE: 3604336, mRNA, partial cds |
| 625234 | 0.550211 | KDEL (Lys-Asp-Glu-Leu) endoplasmic reticulum protein |
| | | retention receptor 3 |
| 279970 | 0.5502099 | adenosine A2a receptor |
| 1518402 | 0.5497179 | KIAA1361 protein |
| 753378 | 0.548977 | hypothetical protein FLJ22649 similar to signal peptidase |
| | | SPC22/23 |
| 594500 | 0.5474088 | EST |
| 590774 | 0.5460342 | mitogen-activated protein kinase 13 |
| 2055807 | 0.5460123 | protein kinase domains containing protein similar to |
| | | phosphoprotein C8FW |
| 773188 | 0.5454398 | nuclear receptor subfamily 1, group D, member 2 |
| 126851 | 0.5454014 | hypothetical protein FLJ11160 |
| 244764 | 0.5450377 | B7 homolog 3 |
| 813419 | 0.5447006 | hydroxyacyl-Coenzyme A dehydrogenase, type II |
| 233349 | 0.5446353 | hypothetical protein FLJ10761 |
| 785701 | 0.5446211 | RAB31, member RAS oncogene family |
| 268946 | 0.5440003 | WD40 protein Ciao1 |
| 611443 | 0.5432426 | myoglobin |
| 510575 | 0.5430211 | hypothetical protein FLJ22087 |
| 491465 | 0.5420241 | hypothetical protein FLJ10035 |
| 1393018 | 0.5404243 | general transcription factor IIIC, polypeptide 1 (alpha subunit, |
| | | 220 kD) |
| 785840 | 0.5401209 | SEC24 (S. cerevisiae) related gene family, member D |
| 1460110 | 0.5392177 | proteasome (prosome, macropain) subunit, beta type, 5 |
| 731308 | 0.5371204 | citrate synthase |
| 301122 | 0.5368073 | extracellular matrix protein 1 |
| 66535 | 0.5363248 | ornithine decarboxylase antizyme 2 |
| 265103 | 0.5361177 | Homo sapiens mRNA; cDNA DKFZp547M123 (from clone |
| | | DKFZp547M123) |
| 788334 | 0.5347204 | mitochondrial ribosomal protein L23 |
| 813631 | 0.534683 | seven transmembrane protein TM7SF3 |
| 1553306 | 0.5344506 | proteasome (prosome, macropain) 26S subunit, non-ATPase, |
| | | 11 |
| 1461477 | 0.5343834 | Homo sapiens mRNA; cDNA DKFZp586I0324 (from clone |
| | | DKFZp586I0324) |
| 199645 | 0.5334951 | nicastrin |
| 884425 | 0.5327862 | chaperonin containing TCP1, subunit 5 (epsilon) |
| 1518890 | 0.5314577 | metallothionein-like 5, testis-specific (tesmin) |
| 756442 | 0.5313744 | P450 (cytochrome) oxidoreductase |
| 2326057 | 0.5310995 | MLN51 protein |
| 138788 | 0.5302936 | prolactin receptor |
| 1698036 | 0.5298397 | ubiquitin-conjugating enzyme E2 variant 1 |
| 811585 | 0.5288102 | huntingtin (Huntington disease) |
| 795185 | 0.5276861 | xenotropic and polytropic retrovirus receptor |
| 629944 | 0.5273447 | myosin VB |
| 784105 | 0.5259815 | ESTs |
| 1696757 | 0.5254296 | hypothetical protein KIAA1165 |
| 139835 | 0.5253386 | UDP-glucose dehydrogenase |
| 1738208 | 0.5240868 | a disintegrin-like and metalloprotease (reprolysin type) with |
| | | thrombospondin type 1 motif, 4 |
| 593431 | 0.5235073 | ESTs, Moderately similar to CEGT_HUMAN CERAMIDE |
| | | GLUCOSYLTRANSFERASE [H. sapiens] |
| 2309073 | 0.5226599 | frizzled (Drosophila) homolog 5 |
| 825470 | 0.5218731 | topoisomerase (DNA) II alpha (170 kD) |
| 839682 | 0.5208954 | ubiquitin-conjugating enzyme E2N (homologous to yeast |
| | | UBC13) |
| 1517171 | 0.5195255 | interleukin 2 receptor, alpha |
| 376516 | 0.5195127 | cell division cycle 4-like |
| 774446 | 0.5181672 | adrenomedullin |
| 1476053 | 0.5179015 | RAD51 (S. cerevisiae) homolog (E coli RecA homolog) |
| 489594 | 0.5178551 | hypothetical protein FLJ11565 |
| 1573946 | 0.5173691 | programmed cell death 9 |
| 503851 | 0.5170168 | nuclear receptor co-repressor/HDAC3 complex subunit |
| 882484 | 0.5165756 | chaperonin containing TCP1, subunit 7 (eta) |
| 51083 | 0.5162686 | catenin (cadherin-associated protein), delta 2 (neural |
| | | plakophilin-related arm-repeat protein) |
| 221295 | 0.5157877 | inhibitor of DNA binding 2, dominant negative helix-loop-helix |
| | | protein |
| 725978 | 0.5147267 | ESTs, Moderately similar to ALU7_HUMAN ALU SUBFAMILY |
| | | SQ SEQUENCE CONTAMINATION WARNING ENTRY |
| | | [H. sapiens] |
| 796757 | 0.5122331 | adaptor-related protein complex 3, sigma 1 subunit |
| 150314 | 0.5114988 | lysophospholipase I |
| 302031 | 0.5111067 | Ste20-related serine/threonine kinase |
| 293569 | 0.5103638 | chromosome 1 open reading frame 21 |
| 838366 | 0.5072623 | 3-hydroxymethyl-3-methylglutaryl-Coenzyme A lyase |
| | | (hydroxymethylglutaricaciduria) |
| 122147 | 0.5067128 | |
| 120749 | 0.5060444 | ESTs, Moderately similar to KIAA1215 protein [H. sapiens] |
| 855563 | 0.5059683 | v-erb-b2 avian erythroblastic leukemia viral oncogene homolog 3 |
| 487733 | 0.5057167 | NADH dehydrogenase (ubiquinone) 1 beta subcomplex, 2 |
| | | (8 kD, AGGG) |
| 625693 | 0.5055986 | hypothetical protein MGC10911 |
| 813751 | 0.5051993 | sialyltransferase 4C (beta-galactosidase alpha-2,3- |
| | | sialytransferase) |
| 842825 | 0.5047729 | G1 to S phase transition 1 |
| 323693 | 0.5047237 | adaptor-related protein complex 1, sigma 1 subunit |
| 202514 | 0.5045411 | DNA (cytosine-5-)-methyltransferase 3 alpha |
| 1517749 | 0.5045134 | ESTs |
| 124331 | 0.5025464 | cleavage and polyadenylation specific factor 5, 25 kD subunit |
| 1474424 | 0.5021308 | Homo sapiens cDNA FLJ12758 fis, clone NT2RP2001328 |
| 1848977 | 0.5015371 | glycerol kinase |
| 454896 | 0.5013449 | DnaJ (Hsp40) homolog, subfamily A, member 2 |
| 1702742 | 0.5011725 | solute carrier family 7 (cationic amino acid transporter, y+ |
| | | system), member 5 |
| 746163 | 0.5009715 | ESTs, Weakly similar to ALU1_HUMAN ALU SUBFAMILY J |
| | | SEQUENCE CONTAMINATION WARNING ENTRY |
| | | [H. sapiens] |
| 810734 | 0.4998276 | polymerase (DNA-directed), delta 4 |
| 725284 | 0.4992842 | phosphorylase kinase, gamma 2 (testis) |
| 52103 | 0.4988158 | hypothetical protein FLJ23045 |
| 739109 | 0.4986821 | adaptor-related protein complex 2, sigma 1 subunit |
| 488505 | 0.4981852 | accessory proteins BAP31/BAP29 |
| 365060 | 0.4976091 | RAB11A, member RAS oncogene family |
| 41569 | 0.4974126 | hypothetical protein FLJ12650 |
| 81336 | 0.4955353 | uteroglobin |
| 700792 | 0.4949149 | cyclin-dependent kinase inhibitor 3 (CDK2-associated dual |
| | | specificity phosphatase) |
| 1592715 | 0.4944665 | Homer, neuronal immediate early gene, 3 |
| 142586 | 0.4942961 | MCT-1 protein |
| 261472 | 0.4941066 | putative nuclear protein ORF1-FL49 |
| 246800 | 0.4927231 | hypothetical protein FLJ10803 |
| 124298 | 0.4922937 | microsomal glutathione S-transferase 3 |
| 1898619 | 0.4916939 | hypothetical protein MGC15737 |
| 739126 | 0.4905859 | tissue specific transplantation antigen P35B |
| 376875 | 0.490328 | flavin containing monooxygenase 1 |
| 358162 | 0.4901786 | protein predicted by clone 23627 |
| 590759 | 0.4895021 | sterol-C4-methyl oxidase-like |
| 1947647 | 0.4887766 | CGI-147 protein |
| 120271 | 0.4886584 | hypothetical protein MGC4692 |
| 488642 | 0.4881613 | ESTs, Weakly similar to I38022 hypothetical protein [H. sapiens] |
| 1492468 | 0.4878466 | DEME-6 protein |
| 345538 | 0.4875796 | cathepsin L |
| 344091 | 0.4871044 | ESTs |
| 123441 | 0.4862068 | ribosomal protein L7a |
| 202901 | 0.48608 | vav 2 oncogene |
| 757489 | 0.4859039 | tubulin, alpha 2 |
| 49117 | 0.4856399 | KIAA0215 gene product |
| 812994 | 0.4854155 | retinoid X receptor, alpha |
| 731023 | 0.4853217 | WD repeat domain 5 |
| 814899 | 0.4840969 | BCL2/adenovirus E1B 19 kD-interacting protein 3-like |
| 756666 | 0.4835569 | protein phosphatase 1, catalytic subunit, alpha isoform |
| 2018337 | 0.4833207 | glucosidase, beta; acid (includes glucosylceramidase) |
| 1568825 | 0.4829928 | Arg/Abl-interacting protein ArgBP2 |
| 869375 | 0.4827093 | isocitrate dehydrogenase 2 (NADP+), mitochondrial |
| 595213 | 0.4824381 | hypothetical protein |
| 744374 | 0.4824256 | putative ankyrin-repeat containing protein |
| 73009 | 0.4822587 | ESTs, Weakly similar to A43932 mucin 2 precursor, intestinal |
| | | [H. sapiens] |
| 70606 | 0.481356 | ESTs |
| 358456 | 0.4812268 | Sec61 gamma |
| 810762 | 0.4806851 | SNARE protein |
| 823930 | 0.4800704 | actin related protein 2/3 complex, subunit 1A (41 kD) |
| 2020898 | 0.4797032 | procollagen-lysine, 2-oxoglutarate 5-dioxygenase 3 |
| 564981 | 0.4782207 | ESTs |
| 1637282 | 0.4777562 | hexokinase 2 |
| 855749 | 0.476968 | triosephosphate isomerase 1 |
| 744944 | 0.4769116 | myosin VI |
| 263727 | 0.4767486 | DNA segment, single copy probe LNS-CAI/LNS-CAII (deleted |
| | | in polyposis |
| 470092 | 0.476018 | like-glycosyltransferase |
| 782513 | 0.4759571 | interferon, alpha-inducible protein (clone IFI-6-16) |
| 897813 | 0.4749348 | polyadenylate binding protein-interacting protein 1 |
| 141852 | 0.4748862 | purinergic receptor P2Y, G-protein coupled, 2 |
| 855800 | 0.4748367 | prolyl endopeptidase |
| 41356 | 0.4746307 | protein phosphatase 2, regulatory subunit B (B56), alpha |
| | | isoform |
| 753320 | 0.4744893 | hypothetical protein FLJ20533 |
| 784140 | 0.474314 | WD repeat domain 15 |
| 530310 | 0.4739401 | KIAA0143 protein |
| 813387 | 0.4727653 | diaphorase (NADH/NADPH) (cytochrome b-5 reductase) |
| 358267 | 0.4719017 | EST, Moderately similar to AF119917 63 PRO2831 [H. sapiens] |
| 2018527 | 0.4717524 | dolichyl-phosphate mannosyltransferase polypeptide 3 |
| 781342 | 0.4716127 | hypothetical protein MGC11115 |
| 785707 | 0.4708376 | protein regulator of cytokinesis 1 |
| 140635 | 0.4706227 | ESTs |
| 814306 | 0.4705205 | tumor protein D52 |
| 784150 | 0.4702303 | RAB31, member RAS oncogene family |
| 839746 | 0.4699443 | Homo sapiens, Similar to RIKEN cDNA 5830420C20 gene, |
| | | clone IMAGE: 3633379, mRNA, partial cds |
| 377384 | 0.4698441 | nuclear receptor subfamily 2, group F, member 2 |
| 183200 | 0.4697062 | fumarylacetoacetate hydrolase (fumarylacetoacetase) |
| 345423 | 0.4694596 | DKFZP564M112 protein |
| 204686 | 0.4687565 | FXYD domain-containing ion transport regulator 1 |
| | | (phospholemman) |
| 428582 | 0.4684705 | hypothetical protein FLJ20296 |
| 210862 | 0.4683996 | acyl-Coenzyme A oxidase 1, palmitoyl |
| 51741 | 0.4678807 | GTP-binding protein |
| 296702 | 0.4670993 | deiodinase, iodothyronine, type I |
| 840865 | 0.4666048 | myristoylated alanine-rich protein kinase C substrate |
| | | (MARCKS, 80K-L) |
| 278531 | 0.4663008 | cytochrome c oxidase subunit VIc |
| 60565 | 0.4659865 | lethal giant larvae (Drosophila) homolog 2 |
| 1639531 | 0.4658349 | RAB27A, member RAS oncogene family |
| 346942 | 0.4657601 | phosphatidylinositol glycan, class Q |
| 884498 | 0.4648731 | uncharacterized hypothalamus protein HT012 |
| 345787 | 0.4647372 | highly expressed in cancer, rich in leucine heptad repeats |
| 825296 | 0.4646099 | low density lipoprotein receptor defect C complementing |
| 2248488 | 0.4645687 | ems1 sequence (mammary tumor and squamous cell |
| | | carcinoma-associated (p80/85 src substrate) |
| 824962 | 0.4638758 | karyopherin alpha 2 (RAG cohort 1, importin alpha 1) |
| 843054 | 0.4637278 | KIAA1533 protein |
| 140289 | 0.4624364 | hypothetical protein |
| 1558233 | 0.4622449 | ESTs |
| 265592 | 0.4615543 | activated RNA polymerase II transcription cofactor 4 |
| 767761 | 0.4608709 | DKFZP434B168 protein |
| 770766 | 0.4603527 | DKFZP564C1940 protein |
| 814209 | 0.4602081 | ESTs |
| 773674 | 0.4601667 | oncogene TC21 |
| 703707 | 0.4599434 | aspartate beta-hydroxylase |
| 951216 | 0.4594172 | NADH dehydrogenase (ubiquinone) 1 beta subcomplex, 10 |
| | | (22 kD, PDSW) |
| 139354 | 0.4582974 | hypothetical protein |
| 79710 | 0.4579008 | KIAA0174 gene product |
| 823574 | 0.457224 | endosulfine alpha |
| 845363 | 0.4570873 | non-metastatic cells 1, protein (NM23A) expressed in |
| 283751 | 0.4567022 | cortistatin |
| 810156 | 0.4561539 | deoxythymidylate kinase (thymidylate kinase) |
| 1416782 | 0.4558415 | creatine kinase, brain |
| 810609 | 0.4551167 | hypothetical protein PP1226 |
| 770675 | 0.454729 | Homo sapiens cDNA: FLJ21323 fis, clone COL02374 |
| 626318 | 0.4529715 | ubinuclein 1 |
| 38356 | 0.4513099 | cytochrome P450, subfamily 46 (cholesterol 24-hydroxylase) |
| 705064 | 0.4497555 | transforming, acidic coiled-coil containing protein 3 |
| 127646 | 0.4493747 | |
| 299815 | 0.4482635 | hypothetical protein DC42 |
| 815026 | 0.4472842 | cleavage stimulation factor, 3′ pre-RNA, subunit 3, 77 kD |
| 489823 | 0.4468978 | COX17 (yeast) homolog, cytochrome c oxidase assembly |
| | | protein |
| 469151 | 0.4462659 | eukaryotic translation initiation factor 2, subunit 2 (beta, 38 kD) |
| 41826 | 0.4456463 | ESTs |
| 47853 | 0.445149 | aldehyde dehydrogenase 4 family, member A1 |
| 144740 | 0.4449229 | similar to phosphatidylcholine transfer protein 2 |
| 726658 | 0.4446657 | non-metastatic cells 3, protein expressed in |
| 325515 | 0.444486 | hypothetical protein FLJ10980 |
| 815781 | 0.4441245 | heat shock 105 kD |
| 746190 | 0.4430243 | hypothetical protein DKFZp761B1514 |
| 2016194 | 0.4417536 | glia maturation factor, beta |
| 1584287 | 0.4413331 | KIAA0241 protein |
| 147834 | 0.4412795 | zinc finger protein 217 |
| 154610 | 0.4410751 | dynactin 4 |
| 754653 | 0.4405873 | cadherin, EGF LAG seven-pass G-type receptor 3, flamingo |
| | | (Drosophila) homolog |
| 742581 | 0.440367 | Homo sapiens cDNA FLJ10366 fis, clone NT2RM2001420 |
| 2306987 | 0.4402912 | secreted and transmembrane 1 |
| 878406 | 0.4396935 | metaxin 1 |
| 430614 | 0.438809 | 2,3-bisphosphoglycerate mutase |
| 415102 | 0.4387892 | cell division cycle 25C |
| 358217 | 0.4385343 | glypican 4 |
| 1636669 | 0.4384707 | hypothetical protein FLJ21939 similar to 5-azacytidine induced |
| | | gene 2 |
| 83358 | 0.4380365 | ESTs |
| 1325816 | 0.4379227 | polymerase (RNA) II (DNA directed) polypeptide L (7.6 kD) |
| 824524 | 0.4375695 | UDP-galactose transporter related |
| 795805 | 0.4370764 | KIAA0332 protein |
| 743977 | 0.4368155 | Homo sapiens mRNA for TL132 |
| 785793 | 0.4367093 | capping protein (actin filament) muscle Z-line, alpha 1 |
| 266218 | 0.4362517 | hypothetical protein FLJ11350 |
| 769600 | 0.4362014 | uracil-DNA glycosylase 2 |
| 1656062 | 0.4355127 | coagulation factor XII (Hageman factor) |
| 2017415 | 0.43546 | centromere protein A (17 kD) |
| 772220 | 0.4352917 | for protein disulfide isomerase-related |
| 811907 | 0.4340842 | hypothetical protein FLJ22056 |
| 789376 | 0.4339171 | thioredoxin reductase 1 |
| 782503 | 0.4334388 | fatty acid desaturase 1 |
| 753897 | 0.4324066 | autocrine motility factor receptor |
| 767289 | 0.4323485 | hypothetical protein FLJ10055 |
| 1159963 | 0.4323117 | interferon regulatory factor 7 |
| 1652310 | 0.4321289 | Homo sapiens, clone MGC: 19613 IMAGE: 3833049, mRNA, |
| | | complete cds |
| 358936 | 0.4314082 | ESTs, Weakly similar to T2D3_HUMAN TRANSCRIPTION |
| | | INITIATION FACTOR TFIID 135 KDA SUBUNIT [H. sapiens] |
| 41698 | 0.4310061 | progesterone binding protein |
| 1635665 | 0.430087 | Homo sapiens, RIKEN cDNA 2010100O12 gene, clone |
| | | MGC: 14813 IMAGE: 4133274, mRNA, complete cds |
| 1558642 | 0.4298376 | hypothetical protein MGC2771 |
| 1733262 | 0.4289711 | BLu protein |
| 823694 | 0.4280476 | Homo sapiens chromosome 19, BAC CIT-HSPC_204F22 |
| | | (BC228680), complete sequence; contains bacterial insertion |
| | | element |
| 1641894 | 0.4277815 | EST |
| 2572170 | 0.4277651 | ESTs, Weakly similar to T26581 hypothetical protein |
| | | Y32B12A.3 - Caenorhabditis elegans [C. elegans] |
| 731080 | 0.4276128 | hypothetical protein FLJ12661 |
| 649084 | 0.4275738 | carbonic anhydrase XI |
| 795498 | 0.4270889 | putative transmembrane protein |
| 897770 | 0.4270685 | |
| 2250839 | 0.4266248 | androgen receptor (dihydrotestosterone receptor; testicular |
| | | feminization; spinal and bulbar muscular atrophy; Kennedy |
| | | disease) |
| 510794 | 0.4264564 | c-myc binding protein |
| 825822 | 0.4255725 | hypothetical protein |
| 825659 | 0.424655 | N-myc downstream regulated |
| 826077 | 0.4242762 | pyruvate dehydrogenase (lipoamide) beta |
| 293727 | 0.4242722 | hypothetical protein MGC861 |
| 503866 | 0.4230404 | sperm autoantigenic protein 17 |
| 491527 | 0.4219324 | Homo sapiens, Similar to CG7083 gene product, clone |
| | | MGC: 10534 IMAGE: 3957147, mRNA, complete cds |
| 292936 | 0.4217115 | hypothetical protein FLJ10468 |
| 823940 | 0.4214277 | transducer of ERBB2, 1 |
| 414992 | 0.420994 | K562 cell-derived leucine-zipper-like protein 1 |
| 346134 | 0.4209894 | calcium-regulated heat-stable protein (24 kD) |
| 51657 | 0.4208387 | hypothetical protein ET |
| 2009779 | 0.420743 | rabaptin-5 |
| 1523225 | 0.4195302 | oncostatin M receptor |
| 826194 | 0.4177687 | synaptotagmin-like 2 |
| 85804 | 0.4171445 | hypothetical protein FLJ21918 |
| 725223 | 0.4167519 | KIAA0077 protein |
| 79726 | 0.4162264 | ESTs, Highly similar to T46395 hypothetical protein |
| | | DKFZp434I1120.1 [H. sapiens] |
| 1762111 | 0.4160469 | natriuretic peptide receptor C/guanylate cyclase C |
| | | (atrionatriuretic peptide receptor C) |
| 509588 | 0.4157059 | TATA box binding protein (TBP)-associated factor, RNA |
| | | polymerase II, J, 20 kD |
| 327506 | 0.4152106 | Homo sapiens mRNA full length insert cDNA clone |
| | | EUROIMAGE 327506 |
| 744047 | 0.4151137 | polo (Drosophia)-like kinase |
| 221499 | 0.4149929 | KIAA0508 protein |
| 745394 | 0.4147627 | Homo sapiens cDNA: FLJ23249 fis, clone COL04196 |
| 149539 | 0.4138942 | KIAA1700 |
| 823907 | 0.4135955 | hypothetical protein FLJ10511 |
| 66317 | 0.4132405 | H1 histone family, member 2 |
| 295986 | 0.413087 | emopamil-binding protein (sterol isomerase) |
| 565235 | 0.4124667 | spermine synthase |
| 825386 | 0.4121622 | ATP synthase, H+ transporting, mitochondrial F1F0, subunit d |
| 250313 | 0.4121296 | ESTs |
| 72050 | 0.4114235 | chloride channel, nucleotide-sensitive, 1A |
| 417801 | 0.4112944 | mitochondrial ribosomal protein L27 |
| 841501 | 0.4111707 | KIAA0102 gene product |
| 813707 | 0.410787 | regulator of G-protein signalling 16 |
| 745083 | 0.4107735 | ubiquitin specific protease 18 |
| 244974 | 0.4105439 | hypothetical protein FLJ22875 |
| 26883 | 0.4103824 | protein kinase (cAMP-dependent, catalytic) inhibitor beta |
| 280249 | 0.4096162 | Kruppel-like factor 7 (ubiquitous) |
| 74738 | 0.409593 | Homo sapiens, clone IMAGE: 3535294, mRNA, partial cds |
| 1753497 | 0.409432 | ovo (Drosophila) homolog-like 1 |
| 204299 | 0.4073087 | replication protein A3 (14 kD) |
| 358609 | 0.4071867 | NADH dehydrogenase (ubiquinone) flavoprotein 3 (10 kD) |
| 290101 | 0.4071477 | ESTs |
| 2306221 | 0.4068933 | wingless-type MMTV integration site family, member 10B |
| 726439 | 0.4067151 | CGI-143 protein |
| 431805 | 0.405447 | granulin |
| 111362 | 0.4045961 | |
| 472103 | 0.4043572 | soc-2 (suppressor of clear, C. elegans) homolog |
| 788444 | 0.4042598 | KIAA0033 protein |
| 1632252 | 0.404193 | complement component 1, q subcomponent, alpha polypeptide |
| 150118 | 0.4039789 | hypothetical protein DKFZp434F054 |
| 594226 | 0.403676 | Homo sapiens cDNA FLJ14459 fis, clone HEMBB1002409 |
| 1499828 | 0.4030396 | fucosyltransferase 1 (galactoside 2-alpha-L-fucosyltransferase, |
| | | Bombay phenotype included) |
| 810939 | 0.40256 | hypothetical protein FLJ22169 |
| 841621 | 0.4020253 | ESTs, Weakly similar to JE0350 Anterior gradient-2 [H. sapiens] |
| 200144 | 0.4019827 | ring-box 1 |
| 486110 | 0.4017126 | profilin 2 |
| 292213 | 0.4014458 | guanine nucleotide binding protein (G protein), beta polypeptide 2 |
| 1631132 | 0.401216 | peptide transporter 3 |
| 669379 | 0.4011648 | Homo sapiens BAC clone RP11-505D17 from 7p22-p21 |
| 773685 | 0.4009493 | phosphodiesterase 4D interacting protein (myomegalin) |
| 810411 | 0.400096 | hypothetical protein FLJ13222 |
| 1583198 | 0.39981 | ESTs, Weakly similar to S65824 reverse transcriptase homolog |
| | | [H. sapiens] |
| 84295 | 0.3989864 | interleukin 1 receptor antagonist |
| 757328 | 0.3985331 | hypothetical protein FLJ22678 |
| 35626 | 0.39822 | Homo sapiens cDNA FLJ14201 fis, clone NT2RP3002955 |
| 1587863 | 0.3980812 | acetyl-Coenzyme A acyltransferase 1 (peroxisomal 3-oxoacyl- |
| | | Coenzyme A thiolase) |
| 713862 | 0.3965573 | ubiquitin-protein isopeptide ligase (E3) |
| 343731 | 0.3962504 | |
| 43977 | 0.3961848 | KIAA0182 protein |
| 78869 | 0.3954821 | cell membrane glycoprotein, 110000M(r) (surface antigen) |
| 756595 | 0.395121 | S100 calcium-binding protein A10 (annexin II ligand, calpactin I, |
| | | light polypeptide (p11)) |
| 701115 | 0.3936994 | hypothetical protein PRO2013 |
| 81599 | 0.3932769 | ubiquitin specific protease 14 (tRNA-guanine transglycosylase) |
| 1636844 | 0.3926235 | ring finger protein 14 |
| 703739 | 0.392298 | nuclear cap binding protein subunit 1, 80 kD |
| 279633 | 0.3907503 | ESTs, Highly similar to I38759 zinc finger/leucine zipper protein |
| | | [H. sapiens] |
| 122091 | 0.3903482 | casein kinase 2, alpha 1 polypeptide |
| 786067 | 0.390151 | cell division cycle 25B |
| 1909574 | 0.3900357 | mitochondrial ribosomal protein S11 |
| 263013 | 0.3894536 | procollagen-lysine, 2-oxoglutarate 5-dioxygenase (lysine |
| | | hydroxylase) 2 |
| 491001 | 0.3884144 | glyoxalase I |
| 455275 | 0.3869874 | hypothetical protein FLJ23469 |
| 85450 | 0.3868399 | acyl-Coenzyme A oxidase 2, branched chain |
| 773286 | 0.3861543 | solute carrier family 9 (sodium/hydrogen exchanger), isoform 3 |
| | | regulatory factor 1 |
| 366156 | 0.3861394 | Homo sapiens cDNA FLJ14028 fis, clone HEMBA1003838 |
| 49630 | 0.3858335 | calcium channel, voltage-dependent, L type, alpha 1D subunit |
| 121251 | 0.385792 | hypothetical protein MGC5576 |
| 2322223 | 0.3853156 | small nuclear ribonucleoprotein polypeptide A |
| 276915 | 0.3851081 | DNA (cytosine-5-)-methyltransferase 3 beta |
| 1591264 | 0.3849595 | transaldolase 1 |
| 1434948 | 0.3849593 | HIV TAT specific factor 1 |
| 489657 | 0.3847527 | tryptophan rich basic protein |
| 810947 | 0.3833137 | LIS1-interacting protein NUDE1, rat homolog |
| 298417 | 0.3833014 | trefoil factor 3 (intestinal) |
| 154707 | 0.3831976 | MpV17 transgene, murine homolog, glomerulosclerosis |
| 79032 | 0.3816428 | CGI-82 protein |
| 1916575 | 0.3814206 | BCL2-interacting killer (apoptosis-inducing) |
| 1585492 | 0.3812472 | ESTs |
| 1455394 | 0.3811391 | cytochrome c |
| 769942 | 0.3810371 | kinesin-like 4 |
| 429387 | 0.3810004 | chimerin (chimaerin) 2 |
| 768377 | 0.3809674 | activity-dependent neuroprotective protein |
| 214996 | 0.380548 | ESTs |
| 1557637 | 0.3805285 | ESTs |
| 1517595 | 0.3804132 | KIAA0175 gene product |
| 841260 | 0.3801713 | hypothetical protein |
| 491524 | 0.3785047 | mitochondrial ribosomal protein L13 |
| 30170 | 0.3782337 | caspase 3, apoptosis-related cysteine protease |
| 76196 | 0.3781541 | hypothetical protein FLJ20062 |
| 2017721 | 0.3781067 | ESTs, Weakly similar to 2109260A B cell growth factor |
| | | [H. sapiens] |
| 134918 | 0.3779855 | ESTs |
| 347726 | 0.377767 | homeo box D8 |
| 1469148 | 0.3776558 | FGFR1 oncogene partner |
| 1649374 | 0.3771383 | homogentisate 1,2-dioxygenase (homogentisate oxidase) |
| 241043 | 0.3770901 | Human clone 137308 mRNA, partial cds |
| 815835 | 0.3764754 | hypothetical protein R33729_1 |
| 143426 | 0.3764075 | ras homolog gene family, member B |
| 1412245 | 0.3762847 | carboxypeptidase A2 (pancreatic) |
| 786265 | 0.3760397 | KIAA0750 gene product |
| 266500 | 0.3758559 | ESTs |
| 755301 | 0.3749416 | protein kinase C, delta |
| 294397 | 0.3744208 | DKFZP586A0522 protein |
| 155806 | 0.3741516 | |
| 41123 | 0.3739475 | Homo sapiens, Similar to RIKEN cDNA 2210021G21 gene, |
| | | clone MGC: 14859 IMAGE: 3621871, mRNA, complete cds |
| 454339 | 0.3738929 | thiopurine S-methyltransferase |
| 810316 | 0.3732635 | very long-chain acyl-CoA synthetase; lipidosin |
| 812105 | 0.3731726 | ALL1-fused gene from chromosome 1q |
| 773426 | 0.3727398 | KIAA0391 gene product |
| 1572710 | 0.3725734 | hypothetical protein FLJ21213 |
| 772898 | 0.372064 | ribosomal protein S15a |
| 283739 | 0.3714571 | Homo sapiens cDNA FLJ14028 fis, clone HEMBA1003838 |
| 289505 | 0.3714361 | ESTs, Moderately similar to S65657 alpha-1C-adrenergic |
| | | receptor splice form 2 [H. sapiens] |
| 897177 | 0.3712716 | phosphoglycerate mutase 1 (brain) |
| 782547 | 0.3708058 | ESTs |
| 647866 | 0.3706822 | Homo sapiens cDNA FLJ13975 fis, clone Y79AA1001585 |
| 1679942 | 0.3706798 | KIAA1053 protein |
| 566443 | 0.3705134 | Homo sapiens cDNA FLJ12793 fis, clone NT2RP2002033 |
| 302996 | 0.3698028 | chloride intracellular channel 3 |
| 726699 | 0.3695061 | ESTs, Weakly similar to AAB47496 NG5 [H. sapiens] |
| 2094232 | 0.3693257 | chromosome 1 open reading frame 12 |
| 2549634 | 0.3692338 | activator of S phase kinase |
| 177827 | 0.3691459 | synaptotagmin VII |
| 852829 | 0.3691079 | karyopherin alpha 3 (importin alpha 4) |
| 207794 | 0.3679245 | nuclear factor (erythroid-derived 2), 45 kD |
| 823909 | 0.3675927 | |
| 743810 | 0.3667087 | hypothetical protein MGC2577 |
| 810039 | 0.3662086 | defender against cell death 1 |
| 53039 | 0.3659853 | carbohydrate (keratan sulfate Gal-6) sulfotransferase 1 |
| 809727 | 0.365892 | unc-51 (C. elegans)-like kinase 1 |
| 26171 | 0.3656147 | KIAA0856 protein |
| 1570663 | 0.3656056 | FK506-binding protein 4 (59 kD) |
| 179212 | 0.3649976 | ESTs, Moderately similar to T12539 hypothetical protein |
| | | DKFZp434J154.1 [H. sapiens] |
| 292996 | 0.36439 | tyrosine 3-monooxygenase/tryptophan 5-monooxygenase |
| | | activation protein, eta polypeptide |
| 259950 | 0.364359 | hypothetical protein FLJ14991 |
| 815794 | 0.3643266 | nucleobindin 2 |
| 965223 | 0.3642603 | thymidine kinase 1, soluble |
| 592801 | 0.3638731 | serine palmitoyltransferase, long chain base subunit 2 |
| 42831 | 0.3638165 | N-terminal kinase-like |
| 788641 | 0.3638059 | adaptor-related protein complex 1, sigma 2 subunit |
| 813845 | 0.3637519 | RNA, U transporter 1 |
| 52076 | 0.3633002 | neuroblastoma (nerve tissue) protein |
| 154493 | 0.3629466 | interferon-induced protein 75, 52 kD |
| 1519013 | 0.3628651 | Homo sapiens, clone IMAGE: 3537447, mRNA, partial cds |
| 1434905 | 0.3624537 | homeo box B7 |
| 825282 | 0.3624112 | DKFZP586L0724 protein |
| 322617 | 0.3623149 | v-ral simian leukemia viral oncogene homolog B (ras related; |
| | | GTP binding protein) |
| 814353 | 0.3622592 | phorbol-12-myristate-13-acetate-induced protein 1 |
| 2017917 | 0.360911 | Homo sapiens cDNA: FLJ23371 fis, clone HEP16068, highly |
| | | similar to HSTFIISH Homo sapiens mRNA for transcription |
| | | elongation factor TFIIS |
| 813616 | 0.3608471 | FK506-binding protein like |
| 1846982 | 0.3602851 | inhibin, beta C |
| 505289 | 0.3595549 | angiotensin II, type I receptor-associated protein |
| 1565079 | 0.358728 | B-cell linker |
| 795401 | 0.3573481 | diacylglycerol O-acyltransferase (mouse) homolog |
| 781047 | 0.357207 | budding uninhibited by benzimidazoles 1 (yeast homolog) |
| 346696 | 0.3570344 | TEA domain family member 4 |
| 809466 | 0.3568464 | DNA segment on chromosome 19 (unique) 1177 expressed |
| | | sequence |
| 743220 | 0.3566593 | hypothetical protein FLJ12517 |
| 131268 | 0.3564734 | growth factor receptor-bound protein 14 |
| 119133 | 0.3564449 | Homo sapiens mRNA; cDNA DKFZp434B231 (from clone |
| | | DKFZp434B231) |
| 242706 | 0.3562469 | Homo sapiens, Similar to RIKEN cDNA 5730494N06 gene, |
| | | clone MGC: 13349 IMAGE: 4249231, mRNA, complete cds |
| 66599 | 0.3561699 | N-acetyltransferase 1 (arylamine N-acetyltransferase) |
| 46716 | 0.3557556 | ESTs |
| 271899 | 0.355689 | Ser-Thr protein kinase related to the myotonic dystrophy protein |
| | | kinase |
| 25440 | 0.3554359 | staufen (Drosophila, RNA-binding protein) homolog 2 |
| 1899312 | 0.3551595 | ESTs |
| 783681 | 0.354881 | upstream regulatory element binding protein 1 |
| 131091 | 0.3548154 | Not56 (D. melanogaster)-like protein |
| 300099 | 0.3547857 | ESTs |
| 588436 | 0.3544892 | butyrate-induced transcript 1 |
| 2069602 | 0.3539588 | melanocortin 1 receptor (alpha melanocyte stimulating hormone |
| | | receptor) |
| 810959 | 0.3534647 | Rho GDP dissociation inhibitor (GDI) alpha |
| 649977 | 0.3534567 | Homo sapiens clone CDABP0014 mRNA sequence |
| 345069 | 0.3532976 | nuclear factor (erythroid-derived 2)-like 3 |
| 767487 | 0.3530592 | ariadne (Drosophila) homolog, ubiquitin-conjugating enzyme |
| | | E2-binding protein, 1 |
| 824943 | 0.3527414 | hypothetical protein |
| 744994 | 0.3526118 | hypothetical protein FLJ12242 |
| 773381 | 0.3525581 | N-ethylmaleimide-sensitive factor attachment protein, alpha |
| 1568561 | 0.3518952 | BCL2-like 1 |
| 2028916 | 0.3516982 | Homo sapiens mRNA for Hmob33 protein, 3′ untranslated |
| | | region |
| 753457 | 0.3515361 | NADH dehydrogenase (ubiquinone) Fe—S protein 1 (75 kD) |
| | | (NADH-coenzyme Q reductase) |
| 2161427 | 0.3509243 | progesterone receptor |
| 1947381 | 0.3509235 | hypothetical protein FLJ22329 |
| 82421 | 0.3506778 | zinc finger protein 6 (CMPX1) |
| 504461 | 0.3506753 | opsin 3 (encephalopsin) |
| 754625 | 0.3503938 | ATPase, Class II, type 9A |
| 795543 | 0.3500184 | thioredoxin peroxidase (antioxidant enzyme) |
| 1910316 | 0.3498986 | KIAA0535 gene product |
| 172785 | 0.3493683 | NAG-5 protein |
| 1910078 | 0.3491481 | ESTs, Weakly similar to YK54_YEAST HYPOTHETICAL 18.4 KD |
| | | PROTEIN IN SIS2-MTD1 INTERGENIC REGION |
| | | [S. cerevisiae] |
| 321354 | 0.3489273 | mitochondrial ribosomal protein L15 |
| 878815 | 0.3478601 | ADP-ribosylation factor 3 |
| 741769 | 0.3475255 | polymerase (DNA directed), beta |
| 487831 | 0.3464379 | Homo sapiens cDNA FLJ14059 fis, clone HEMBB1000573 |
| 754046 | 0.3464097 | DNA segment on chromosome X (unique) 9879 expressed |
| | | sequence |
| 344988 | 0.3464047 | |
| 530093 | 0.3463461 | myelin protein zero-like 1 |
| 823615 | 0.3462131 | Homo sapiens cDNA: FLJ21245 fis, clone COL01184 |
| 144881 | 0.3461767 | calumenin |
| 128695 | 0.3455381 | ESTs, Weakly similar to I38344 titin, cardiac muscle [H. sapiens] |
| 884511 | 0.3440809 | cytochrome c oxidase subunit VIIb |
| 128711 | 0.3437491 | anillin (Drosophila Scraps homolog), actin binding protein |
| 470035 | 0.3436575 | prokineticin 1 precursor |
| 1568126 | 0.3434836 | solute carrier family 37 (glycerol-3-phosphate transporter), |
| | | member 1 |
| 588911 | 0.3427563 | 2′,5′-oligoadenylate synthetase 1 (40-46 kD) |
| 35191 | 0.3427094 | stromal cell-derived factor 2 |
| 814989 | 0.3426862 | protein phosphatase 1G (formerly 2C), magnesium-dependent, |
| | | gamma isoform |
| 73638 | 0.3425644 | protein tyrosine phosphatase type IVA, member 2 |
| 1605178 | 0.3416196 | amino acid transporter system A1 |
| 214068 | 0.3414185 | GATA-binding protein 3 |
| 755599 | 0.3413351 | interferon induced transmembrane protein 1 (9-27) |
| 782439 | 0.3413092 | ATP synthase, H+ transporting, mitochondrial F0 complex, |
| | | subunit e |
| 595070 | 0.3408109 | stress-associated endoplasmic reticulum protein 1; ribosome |
| | | associated membrane protein 4 |
| 183440 | 0.3406314 | arylsulfatase A |
| 774502 | 0.3402891 | protein tyrosine phosphatase, non-receptor type 12 |
| 235986 | 0.338857 | wingless-type MMTV integration site family, member 11 |
| 289936 | 0.3382708 | solute carrier family 7, (cationic amino acid transporter, y+ |
| | | system) member 11 |
| 824108 | 0.3381616 | SCAN domain-containing 1 |
| 26617 | 0.3371923 | activated leucocyte cell adhesion molecule |
| 810497 | 0.3367408 | ESTs, Weakly similar to A35363 synapsin I splice form a |
| | | [H. sapiens] |
| 40042 | 0.3362557 | hypothetical protein FLJ10747 |
| 811142 | 0.3361932 | phosphoinositide-3-kinase, regulatory subunit, polypeptide 2 |
| | | (p85 beta) |
| 2046679 | 0.3358043 | ESTs |
| 487444 | 0.3357395 | cyclic AMP phosphoprotein, 19 kD |
| 78736 | 0.3354693 | Homo sapiens clone 24877 mRNA sequence |
| 1637302 | 0.3353374 | DNAJ domain-containing |
| 825606 | 0.335057 | kinesin-like 1 |
| 76362 | 0.3349202 | spectrin, alpha, non-erythrocytic 1 (alpha-fodrin) |
| 1883028 | 0.3345488 | Homo sapiens mRNA; cDNA DKFZp434J1912 (from clone |
| | | DKFZp434J1912) |
| 789383 | 0.3341667 | cAMP responsive element modulator |
| 302549 | 0.3340582 | basic transcription element binding protein 1 |
| 309288 | 0.334016 | replication factor C (activator 1) 4 (37 kD) |
| 712314 | 0.3338146 | myosin regulatory light chain interacting protein |
| 241677 | 0.3332521 | Homo sapiens, clone MGC: 18110 IMAGE: 4152745, mRNA, |
| | | complete cds |
| 898253 | 0.3331924 | reticulocalbin 2, EF-hand calcium binding domain |
| 770992 | 0.3331315 | |
| 81203 | 0.3328573 | paraoxonase 3 |
| 416436 | 0.3325571 | mitochondrial ribosomal protein L24 |
| 1660666 | 0.332029 | carbonic anhydrase VB, mitochondrial |
| 811582 | 0.3319191 | golgi phosphoprotein 2 |
| 2054122 | 0.3318413 | solute carrier family 11 (proton-coupled divalent metal ion |
| | | transporters), member 3 |
| 837864 | 0.3314764 | progestin induced protein |
| 1632247 | 0.3312131 | hypothetical protein FLJ23436 |
| 298143 | 0.3307258 | ESTs |
| 2027952 | 0.3307056 | hypothetical protein FLJ20297 |
| 1600281 | 0.3304323 | nucleolar protein 3 (apoptosis repressor with CARD domain) |
| 814270 | 0.3300091 | polymyositis/scleroderma autoantigen 1 (75 kD) |
| 415589 | 0.3296266 | Homo sapiens clone PP1498 unknown mRNA |
| 1912951 | 0.329484 | uterine-derived 14 kDa protein |
| 726637 | 0.3292392 | t-complex-associated-testis-expressed 1-like |
| 1474684 | 0.3291536 | ephrin-A1 |
| 1843843 | 0.3290269 | KIAA1304 protein |
| 266312 | 0.3289707 | ATPase, Cu++ transporting, beta polypeptide (Wilson disease) |
| 135640 | 0.3288537 | syntaxin 3A |
| 279720 | 0.3282308 | ESTs, Moderately similar to A47582 B-cell growth factor |
| | | precursor [H. sapiens] |
| 724888 | 0.3272422 | cytochrome P450, subfamily IVB, polypeptide 1 |
| 811808 | 0.326728 | nucleoside diphosphate kinase type 6 (inhibitor of p53-induced |
| | | apoptosis-alpha) |
| 131566 | 0.3261265 | hypothetical protein MGC11061 |
| 154466 | 0.3258377 | STIP1 homology and U-Box containing protein 1 |
| 1692195 | 0.3254467 | smg GDS-ASSOCIATED PROTEIN |
| 785368 | 0.3254362 | PDZ-binding kinase; T-cell originated protein kinase |
| 1637516 | 0.325364 | nuclear autoantigen |
| 1637829 | 0.3246232 | ESTs |
| 810567 | 0.3243984 | Homo sapiens, clone MGC: 3182 IMAGE: 3356293, mRNA, |
| | | complete cds |
| 1027283 | 0.3242547 | ESTs |
| 306806 | 0.324179 | Homo sapiens, Similar to hypothetical protein FLJ12838, clone |
| | | IMAGE: 4130879, mRNA, partial cds |
| 855406 | 0.3241172 | Homo sapiens clone TCCCIA00176 mRNA sequence |
| 429222 | 0.3239231 | CGI-107 protein |
| 1500542 | 0.3228485 | regulator of G-protein signalling 11 |
| 269606 | 0.3228482 | N-methylpurine-DNA glycosylase |
| 74566 | 0.3226444 | exportin 1 (CRM1, yeast, homolog) |
| 754192 | 0.3225824 | Homo sapiens mRNA; cDNA DKFZp586H0924 (from clone |
| | | DKFZp586H0924) |
| 180785 | 0.322529 | KIAA0726 gene product |
| 824479 | 0.3225263 | exonuclease NEF-sp |
| 842863 | 0.3225116 | N-myc downstream regulated |
| 1708055 | 0.3225042 | glioblastoma overexpressed |
| 1631194 | 0.3219004 | t-complex-associated-testis-expressed 1-like 1 |
| 366834 | 0.3217743 | envoplakin |
| 839580 | 0.3216628 | ESTs |
| 245485 | 0.3215041 | hypothetical protein MGC5178 |
| 815501 | 0.3214849 | hypothetical protein MGC2721 |
| 2017403 | 0.3211799 | regulator of G-protein signalling 3 |
| 281476 | 0.3210873 | aspartylglucosaminidase |
| 130835 | 0.3209345 | Homo sapiens, Similar to clone FLB3816, clone |
| | | IMAGE: 3454380, mRNA |
| 77533 | 0.3205713 | inositol polyphosphate-5-phosphatase, 40 kD |
| 42076 | 0.3194462 | TRK-fused gene |
| 810998 | 0.3179036 | DKFZP434C131 protein |
| 75859 | −1.8328232 | N-myc downstream-regulated gene 2 |
| 760299 | −1.7010462 | dickkopf (Xenopus laevis) homolog 3 |
| 344720 | −1.5196431 | glycophorin C (Gerbich blood group) |
| 345670 | −1.4445337 | ESTs, Moderately similar to I59348 CCAAT binding |
| | | transcription factor CBF subunit C - rat [R. norvegicus] |
| 200814 | −1.4325283 | membrane metallo-endopeptidase (neutral endopeptidase, |
| | | enkephalinase, CALLA, CD10) |
| 1882697 | −1.4318896 | peanut (Drosophila)-like 2 |
| 1161564 | −1.3891189 | desmuslin |
| 788234 | −1.3761312 | inhibitor of DNA binding 4, dominant negative helix-loop-helix |
| | | protein |
| 1558675 | −1.3749762 | SRY (sex determining region Y)-box 10 |
| 285377 | −1.3699153 | pellino (Drosophila) homolog 2 |
| 45099 | −1.3515907 | regucalcin (senescence marker protein-30) |
| 611532 | −1.3158379 | troponin I, skeletal, fast |
| 377275 | −1.3102234 | ataxia-telangiectasia group D-associated protein |
| 810002 | −1.3038896 | Homo sapiens, clone MGC: 19762 IMAGE: 3636045, mRNA, |
| | | complete cds |
| 1569187 | −1.2971252 | heparan sulfate (glucosamine) 3-O-sulfotransferase 4 |
| 377461 | −1.2798035 | caveolin 1, caveolae protein, 22 kD |
| 712139 | −1.2738321 | ADP-ribosylation factor-like 7 |
| 742685 | −1.272916 | disabled (Drosophila) homolog 2 (mitogen-responsive |
| | | phosphoprotein) |
| 811920 | −1.2690713 | interleukin 11 receptor, alpha |
| 796542 | −1.2625621 | ets variant gene 5 (ets-related molecule) |
| 486683 | −1.2614406 | Homo sapiens mRNA; cDNA DKFZp564J0323 (from clone |
| | | DKFZp564J0323) |
| 753071 | −1.2529315 | Homo sapiens cDNA: FLJ22528 fis, clone HRC12825 |
| 131839 | −1.2372478 | folate receptor 1 (adult) |
| 300632 | −1.2368481 | hypothetical protein FLJ21044 similar to Rbig1 |
| 839736 | −1.2271618 | crystallin, alpha B |
| 813265 | −1.2230435 | Homo sapiens mRNA; cDNA DKFZp564H1916 (from clone |
| | | DKFZp564H1916) |
| 1469377 | −1.2127781 | lipoma HMGIC fusion partner-like 2 |
| 490023 | −1.2114805 | hypothetical protein MGC2648 |
| 298122 | −1.1962478 | frizzled (Drosophila) homolog 7 |
| 72778 | −1.1752838 | caspase 7, apoptosis-related cysteine protease |
| 160192 | −1.1751869 | ESTs, Weakly similar to 2004399A chromosomal protein |
| | | [H. sapiens] |
| 22917 | −1.1590598 | Homo sapiens mRNA; cDNA DKFZp761M0111 (from clone |
| | | DKFZp761M0111) |
| 877621 | −1.1501074 | nGAP-like protein |
| 840266 | −1.1476896 | Homo sapiens cDNA: FLJ22667 fis, clone HSI08385 |
| 289760 | −1.1390686 | ESTs, Highly similar to T00391 hypothetical protein KIAA0612 |
| | | [H. sapiens] |
| 811848 | −1.1350073 | hypothetical protein |
| 1556433 | −1.1222978 | GRO3 oncogene |
| 838478 | −1.1175047 | neurocalcin delta |
| 1473274 | −1.1172693 | myosin regulatory light chain 2, smooth muscle isoform |
| 1917449 | −1.0894686 | serum amyloid A4, constitutive |
| 811088 | −1.0851546 | ephrin-B3 |
| 1554167 | −1.0818834 | hypothetical protein FLJ14529 |
| 67741 | −1.0705249 | PP2135 protein |
| 2106144 | −1.067765 | regulated in glioma |
| 2056139 | −1.0673271 | LIM domain protein |
| 767202 | −1.0654145 | latent transforming growth factor beta binding protein 2 |
| 345034 | −1.0600859 | small inducible cytokine subfamily B (Cys-X-Cys), member 14 |
| | | (BRAK) |
| 666879 | −1.0463245 | annexin A8 |
| 130201 | −1.0401997 | intercellular adhesion molecule 2 |
| 878836 | −1.037352 | secretory granule, neuroendocrine protein 1 (7B2 protein) |
| 52419 | −1.0368509 | Friedreich ataxia region gene X123 |
| 291478 | −1.0212528 | runt-related transcription factor 3 |
| 781014 | −1.0161379 | suppression of tumorigenicity 5 |
| 132857 | −1.0084069 | Homo sapiens mRNA; cDNA DKFZp586N1323 (from clone |
| | | DKFZp586N1323) |
| 529843 | −1.0036824 | ESTs, Moderately similar to JC5238 galactosylceramide-like |
| | | protein, GCP [H. sapiens] |
| 2504881 | −1.000863 | signal transducer and activator of transcription 5A |
| 69002 | −0.9960916 | PPAR(gamma) angiopoietin related protein |
| 841308 | −0.9953716 | myosin, light polypeptide kinase |
| 488404 | −0.9880176 | Homo sapiens clone TUA8 Cri-du-chat region mRNA |
| 277571 | −0.9841522 | KIAA1706 protein |
| 1871116 | −0.9821709 | Homo sapiens mRNA; cDNA DKFZp434C1714 (from clone |
| | | DKFZp434C1714); partial cds |
| 897963 | −0.9775616 | phosphatidic acid phosphatase type 2A |
| 1584540 | −0.9754693 | Homo sapiens mRNA; cDNA DKFZp586M2022 (from clone |
| | | DKFZp586M2022) |
| 196435 | −0.9665947 | ESTs |
| 140574 | −0.9652218 | small inducible cytokine subfamily D (Cys-X3-Cys), member 1 |
| | | (fractalkine, neurotactin) |
| 740620 | −0.96496 | tropomyosin 2 (beta) |
| 1473471 | −0.9623482 | KIAA0194 protein |
| 1635320 | −0.9586226 | amiloride-sensitive cation channel 2, neuronal |
| 416959 | −0.957248 | nuclear factor I/B |
| 814798 | −0.9559895 | aldehyde dehydrogenase 1 family, member A3 |
| 823871 | −0.9429443 | SPARC-like 1 (mast9, hevin) |
| 303109 | −0.9414602 | purinergic receptor (family A group 5) |
| 270826 | −0.9389651 | Homo sapiens cDNA FLJ13329 fis, clone OVARC1001795 |
| 360254 | −0.9286811 | cysteine-rich, angiogenic inducer, 61 |
| 66491 | −0.9280245 | plasmolipin |
| 814316 | −0.9276916 | ribosomal protein L13 |
| 153760 | −0.9227865 | EphB1 |
| 811149 | −0.9216006 | chromosome 9 open reading frame 3 |
| 786069 | −0.9203158 | beta-site APP-cleaving enzyme |
| 208718 | −0.9190244 | annexin A1 |
| 416676 | −0.9140494 | pellino (Drosophila) homolog 1 |
| 248631 | −0.9106505 | aminomethyltransferase (glycine cleavage system protein T) |
| 76182 | −0.9106484 | hypothetical protein DKFZp761F241 |
| 756708 | −0.910505 | potassium intermediate/small conductance calcium-activated |
| | | channel, subfamily N, member 4 |
| 712401 | −0.9068788 | phosphoinositide-3-kinase, catalytic, delta polypeptide |
| 625399 | −0.9066615 | hypothetical protein similar to beta-transducin family |
| 753038 | −0.893161 | kinesin family member C3 |
| 162308 | −0.8910356 | ESTs |
| 796181 | −0.8888496 | growth arrest-specific 6 |
| 133236 | −0.8888316 | RNA binding motif protein, X chromosome |
| 415816 | −0.8887425 | ESTs |
| 323780 | −0.8879854 | Homo sapiens cDNA FLJ11177 fis, clone PLACE1007402 |
| 1493218 | −0.884795 | hypothetical protein FLJ22297 |
| 1636166 | −0.8845211 | KIAA0668 protein |
| 1856063 | −0.8833091 | tweety (Drosophila) homolog 1 |
| 34093 | −0.8800259 | EST |
| 2028722 | −0.8800075 | osteoblast specific factor 2 (fasciclin I-like) |
| 811837 | −0.8764231 | eukaryotic translation elongation factor 1 alpha 1 |
| 628955 | −0.8761473 | forkhead box O1A (rhabdomyosarcoma) |
| 884462 | −0.8743987 | Down syndrome critical region gene 1 |
| 306798 | −0.8718483 | NGFI-A binding protein 1 (EGR1 binding protein 1) |
| 882248 | −0.8700731 | transgelin |
| 770935 | −0.8700104 | hypothetical protein FLJ13511 |
| 41208 | −0.865227 | bone morphogenetic protein 1 |
| 768571 | −0.8572018 | SRY (sex determining region Y)-box 8 |
| 781017 | −0.8526926 | early growth response 2 (Krox-20 (Drosophila) homolog) |
| 811837 | −0.848137 | |
| 815737 | −0.8444322 | ATP synthase, H+ transporting, mitochondrial F1 complex, |
| | | alpha subunit, isoform 1, cardiac muscle |
| 814826 | −0.8443265 | ESTs |
| 767164 | −0.842198 | ESTs, Weakly similar to MUC2_HUMAN MUCIN 2 |
| | | PRECURSOR [H. sapiens] |
| 71087 | −0.8416951 | v-maf musculoaponeurotic fibrosarcoma (avian) oncogene |
| | | family, protein F |
| 283023 | −0.8413949 | chemokine (C-X3-C) receptor 1 |
| 160609 | −0.8405669 | ESTs |
| 814443 | −0.8385965 | hypothetical protein MGC3232 |
| 142259 | −0.8376566 | tumor necrosis factor alpha-inducible cellular protein containing |
| | | leucine zipper domains; Huntingtin interacting protein L; |
| | | transcrption factor IIIA-interacting protein |
| 990881 | −0.8335353 | |
| 970590 | −0.8333968 | Homo sapiens mRNA; cDNA DKFZp434A115 (from clone |
| | | DKFZp434A115) |
| 470393 | −0.830751 | matrix metalloproteinase 7 (matrilysin, uterine) |
| 772913 | −0.8306579 | calreticulin |
| 741139 | −0.8299902 | eyes absent (Drosophila) homolog 2 |
| 27769 | −0.8298821 | ESTs |
| 544639 | −0.8275232 | ESTs |
| 1587710 | −0.8252065 | period (Drosophila) homolog 1 |
| 757191 | −0.8247438 | ESTs |
| 80344 | −0.8243418 | interleukin 7 receptor |
| 1635062 | −0.8243024 | DKFZP586A011 protein |
| 119290 | −0.8224756 | cortic al thymocyte receptor (X. laevis CTX) like |
| 161456 | −0.8215502 | serum amyloid A1 |
| 1909935 | −0.8169271 | ESTs |
| 1257131 | −0.8163443 | ESTs |
| 868396 | −0.808677 | tyrosine 3-monooxygenase/tryptophan 5-monooxygenase |
| | | activation protein, eta polypeptide |
| 1609665 | −0.8086161 | BarH-like homeobox 2 |
| 2016775 | −0.8063264 | G protein-coupled receptor, family C, group 5, member B |
| 796475 | −0.8041136 | four and a half LIM domains 3 |
| 1555924 | −0.8025163 | CSR1 protein |
| 416434 | −0.8006527 | SCN Circadian Oscillatory Protein (SCOP) |
| 322561 | −0.7970805 | ribosomal protein L31 |
| 132711 | −0.7962294 | Kruppel-like factor 5 (intestinal) |
| 70245 | −0.795375 | Homo sapiens mRNA full length insert cDNA clone |
| | | EUROIMAGE 50374 |
| 283124 | −0.7933772 | ESTs, Moderately similar to LONG-CHAIN FATTY ACID |
| | | TRANSPORT PROTEIN [M. musculus] |
| 281190 | −0.7927001 | ESTs |
| 250883 | −0.7925068 | ubiquitin-activating enzyme E1-like |
| 346545 | −0.7887371 | laminin, beta 1 |
| 1878409 | −0.7869766 | catechol-O-methyltransferase |
| 530958 | −0.7809384 | smoothened (Drosophila) homolog |
| 40027 | −0.7779473 | ESTs |
| 788136 | −0.7778375 | phosphodiesterase 4B, cAMP-specific (dunce (Drosophila)- |
| | | homolog phosphodiesterase E4) |
| 1626304 | −0.7769866 | spinal cord-derived growth factor-B |
| 1474337 | −0.7766266 | phosphorylase, glycogen; brain |
| 85840 | −0.7754435 | nicotinamide N-methyltransferase |
| 290378 | −0.7751825 | podocalyxin-like |
| 2243051 | −0.7736287 | kallikrein 8 (neuropsin/ovasin) |
| 813256 | −0.7731864 | ATP-binding cassette, sub-family B (MDR/TAP), member 1 |
| 47043 | −0.7729643 | tensin |
| 343695 | −0.7706809 | hypothetical protein FLJ10875 |
| 491403 | −0.7700599 | tumor necrosis factor receptor superfamily, member 1B |
| 767765 | −0.7691523 | GTP-binding protein overexpressed in skeletal muscle |
| 681992 | −0.7691299 | Homo sapiens cDNA FLJ13384 fis, clone PLACE1001062, |
| | | highly similar to Homo sapiens mRNA for lysine-ketoglutarate |
| | | reductase/saccharopine dehydrogenase |
| 505864 | −0.7677235 | RalGDS-like gene |
| 139660 | −0.7677153 | ESTs |
| 869450 | −0.7659922 | ribosomal protein L11 |
| 162308 | −0.7654139 | |
| 209537 | −0.763749 | zinc finger protein 221 |
| 753162 | −0.7617972 | KIAA0603 gene product |
| 810728 | −0.7607171 | hypothetical gene ZD52F10 |
| 366591 | −0.7595643 | T-cell lymphoma invasion and metastasis 2 |
| 488258 | −0.7574184 | collagen, type XVI, alpha 1 |
| 32489 | −0.7571259 | hypothetical protein DKFZp566A1524 |
| 1519147 | −0.7548183 | |
| 418159 | −0.7545888 | synaptogyrin 1 |
| 80727 | −0.7538606 | receptor tyrosine kinase-like orphan receptor 1 |
| 593023 | −0.7516846 | dystrobrevin, beta |
| 586803 | −0.7423407 | placental growth factor, vascular endothelial growth factor- |
| | | related protein |
| 188335 | −0.7399294 | egf-like module containing, mucin-like, hormone receptor-like |
| | | sequence 2 |
| 1161775 | −0.7380353 | villin 1 |
| 490668 | −0.7364434 | Homo sapiens mRNA; cDNA DKFZp586B211 (from clone |
| | | DKFZp586B211) |
| 1474900 | −0.736252 | keratin 15 |
| 378813 | −0.7346408 | secretory leukocyte protease inhibitor (antileukoproteinase) |
| 1630990 | −0.7312981 | ribosomal protein L29 |
| 280907 | −0.7307519 | Kruppel-type zinc finger protein |
| 742562 | −0.730047 | chromosome 16 open reading frame 5 |
| 73252 | −0.7283898 | 3-hydroxy-3-methylglutaryl-Coenzyme A synthase 1 (soluble) |
| 26566 | −0.7278186 | protein-O-mannosyltransferase 1 |
| 1471829 | −0.7275378 | uncharacterized hypothalamus protein HSMNP1 |
| 289428 | −0.7270494 | neurotrophic tyrosine kinase, receptor, type 2 |
| 898092 | −0.7267434 | connective tissue growth factor |
| 34150 | −0.7263513 | ESTs |
| 24958 | −0.7225195 | Homo sapiens mRNA; cDNA DKFZp434C2016 (from clone |
| | | DKFZp434C2016) |
| 725680 | −0.7197488 | transcription factor AP-2 gamma (activating enhancer-binding |
| | | protein 2 gamma) |
| 77972 | −0.7194713 | complement component 3 |
| 1686766 | −0.7192745 | Rag D protein |
| 471196 | −0.7192268 | integral membrane protein 3 |
| 431231 | −0.7182897 | EGF-containing fibulin-like extracellular matrix protein 2 |
| 1908834 | −0.7177051 | ubiquitin A-52 residue ribosomal protein fusion product 1 |
| 207735 | −0.7167416 | serine (or cysteine) proteinase inhibitor, clade A (alpha-1 |
| | | antiproteinase, antitrypsin), member 1 |
| 754157 | −0.7165332 | Homo sapiens mRNA; cDNA DKFZp434K2172 (from clone |
| | | DKFZp434K2172) |
| 35828 | −0.7163689 | diphtheria toxin receptor (heparin-binding epidermal growth |
| | | factor-like growth factor) |
| 1946448 | −0.7150027 | caveolin 2 |
| 470148 | −0.712917 | ESTs |
| 840944 | −0.7099353 | early growth response 1 |
| 809784 | −0.7095414 | kallikrein 6 (neurosin, zyme) |
| 877827 | −0.7093855 | ribosomal protein S27a |
| 32493 | −0.7093777 | integrin, alpha 6 |
| 813520 | −0.7092422 | EphB3 |
| 782501 | −0.7086066 | hypothetical protein PP1665 |
| 27544 | −0.708383 | prominin (mouse)-like 1 |
| 184022 | −0.7070266 | amyloid beta (A4) precursor protein-binding, family B, member |
| | | 1 (Fe65) |
| 323238 | −0.7042807 | GRO1 oncogene (melanoma growth stimulating activity, alpha) |
| 488956 | −0.70192 | CUG triplet repeat, RNA-binding protein 2 |
| 1926246 | −0.701769 | ESTs, Moderately similar to T46371 hypothetical protein |
| | | DKFZp434P1018.1 [H. sapiens] |
| 325365 | −0.7012106 | HIV-1 rev binding protein 2 |
| 221846 | −0.6982802 | checkpoint suppressor 1 |
| 1870305 | −0.6958213 | heat shock 27 kD protein 2 |
| 277044 | −0.6947855 | KIAA1183 protein |
| 503335 | −0.6944661 | hypothetical protein FLJ11196 |
| 757873 | −0.6940266 | cyclin-dependent kinase 5, regulatory subunit 1 (p35) |
| 2306682 | −0.6934667 | A kinase (PRKA) anchor protein (yotiao) 9 |
| 1864302 | −0.6932327 | E74-like factor 5 (ets domain transcription factor) |
| 843070 | −0.6925728 | nucleoporin 88 kD |
| 812975 | −0.6921755 | KIAA0172 protein |
| 46129 | −0.6919169 | ESTs, Highly similar to T17245 hypothetical protein |
| | | DKFZp586J0917.1 [H. sapiens] |
| 270917 | −0.6918383 | secreted frizzled-related protein 1 |
| 789382 | −0.6898974 | Notch (Drosophila) homolog 4 |
| 611481 | −0.6894079 | HMG-box transcription factor TCF-3 |
| 35300 | −0.688788 | KIAA0869 protein |
| 50892 | −0.6887497 | Homo sapiens, clone MGC: 9913 IMAGE: 3870821, mRNA, |
| | | complete cds |
| 366100 | −0.6885372 | matrilin 2 |
| 345056 | −0.6863529 | KIAA1404 protein |
| 2783721 | −0.6854649 | cholinergic receptor, nicotinic, beta polypeptide 1 (muscle) |
| 178137 | −0.6839022 | ribosomal protein L34 |
| 1626996 | −0.682449 | c-fos induced growth factor (vascular endothelial growth factor |
| | | D) |
| 188388 | −0.6824191 | integrin, alpha 10 |
| 133518 | −0.6817662 | microtubule-associated protein, RP/EB family, member 2 |
| 839796 | −0.6807912 | candidate tumor suppressor p33 ING1 homolog |
| 726582 | −0.6807781 | actin-related protein 3-beta |
| 50586 | −0.6794282 | KIAA1545 protein |
| 491763 | −0.679034 | interleukin 1, beta |
| 52096 | −0.6789228 | platelet-derived growth factor receptor, alpha polypeptide |
| 263716 | −0.6767498 | collagen, type VI, alpha 1 |
| 2095066 | −0.676021 | alcohol dehydrogenase 1C (class I), gamma polypeptide |
| 1475738 | −0.6753091 | ribosomal protein S25 |
| 41869 | −0.6747514 | hypothetical protein FLJ11017 |
| 1350439 | −0.6740833 | KIAA0015 gene product |
| 502689 | −0.6739476 | Homo sapiens clone HH409 unknown mRNA |
| 773373 | −0.6730112 | hypothetical protein MGC14258 |
| 308539 | −0.6726187 | Homo sapiens cDNA FLJ12777 fis, clone NT2RP2001720 |
| 343760 | −0.6724144 | SH3 domain binding glutamic acid-rich protein like 2 |
| 769959 | −0.6720196 | collagen, type IV, alpha 2 |
| 2252954 | −0.6687686 | enhancer of filamentation 1 (cas-like docking; Crk-associated |
| | | substrate related) |
| 70201 | −0.6675012 | mitochondrial solute carrier |
| 505243 | −0.6671218 | inositol 1,4,5-triphosphate receptor, type 2 |
| 897731 | −0.6670507 | latrophilin |
| 296880 | −0.6655878 | membrane protein, palmitoylated 1 (55 kD) |
| 1486082 | −0.6635259 | heparin-binding growth factor binding protein |
| 1470278 | −0.6633411 | hypothetical protein FLJ21841 |
| 177665 | −0.6624986 | |
| 725152 | −0.6591675 | hypothetical protein DKFZp762A227 |
| 247616 | −0.6585351 | lipoma HMGIC fusion partner |
| 752668 | −0.65723 | KIAA0440 protein |
| 837870 | −0.657186 | proline arginine-rich end leucine-rich repeat protein |
| 744918 | −0.6552361 | Homo sapiens mRNA; cDNA DKFZp761I0911 (from clone |
| | | DKFZp761I0911) |
| 39600 | −0.6540275 | adenylate kinase 5 |
| 795288 | −0.6528792 | ubiquitin specific protease 4 (proto-oncogene) |
| 1665444 | −0.651994 | tumor endothelial marker 1 precursor |
| 774078 | −0.6508021 | leiomodin 1 (smooth muscle) |
| 415233 | −0.6506593 | ribosomal protein L37a |
| 307029 | −0.6500872 | ribosomal protein L26 |
| 78946 | −0.648366 | Homo sapiens pyruvate dehydrogenase kinase 4 mRNA, 3′ |
| | | untranslated region, partial sequence |
| 785733 | −0.6476493 | hypothetical protein FLJ12892 |
| 415415 | −0.646492 | EST, Moderately similar to RL1X_HUMAN 60S RIBOSOMAL |
| | | PROTEIN L18A [H. sapiens] |
| 858167 | −0.6453859 | fatty-acid-Coenzyme A ligase, long-chain 4 |
| 823714 | −0.642903 | nuclear receptor co-repressor/HDAC3 complex subunit |
| 193913 | −0.6425581 | v-yes-1 Yamaguchi sarcoma viral related oncogene homolog |
| 220395 | −0.6418175 | hypothetical protein FLJ23293 similar to ARL-6 interacting |
| | | protein-2 |
| 840942 | −0.6383764 | major histocompatibility complex, class II, DP beta 1 |
| 725308 | −0.6371261 | ras inhibitor |
| 1492147 | −0.636656 | ribosomal protein S4, X-linked |
| 1466893 | −0.6364598 | ESTs |
| 75078 | −0.6362307 | ESTs |
| 796152 | −0.6356106 | Homo sapiens cDNA FLJ11685 fis, clone HEMBA1004934 |
| 190059 | −0.634814 | guanine nucleotide binding protein (G protein), gamma 7 |
| 840511 | −0.6341661 | vimentin |
| 81409 | −0.6338327 | GABA(A) receptor-associated protein like 1 |
| 745490 | −0.6329354 | hypothetical protein FLJ20607 |
| 530036 | −0.6324794 | hypothetical protein FLJ13078 |
| 755855 | −0.6298333 | solute carrier family 25 (mitochondrial carrier; adenine |
| | | nucleotide translocator), member 6 |
| 785538 | −0.6289951 | Homo sapiens mRNA; cDNA DKFZp434N2116 (from clone |
| | | DKFZp434N2116) |
| 2284803 | −0.6265431 | secretory carrier membrane protein 1 |
| 1623016 | −0.6263561 | EST |
| 154472 | −0.6253276 | fibroblast growth factor receptor 1 (fms-related tyrosine kinase |
| | | 2, Pfeiffer syndrome) |
| 774754 | −0.6243946 | catenin (cadherin-associated protein), beta 1 (88 kD) |
| 1572298 | −0.6228686 | CD3Z antigen, zeta polypeptide (TiT3 complex) |
| 2252417 | −0.6222381 | ribosomal protein S10 |
| 180803 | −0.621787 | inositol polyphosphate-1-phosphatase |
| 683059 | −0.6216483 | Cdc42 effector protein 3 |
| 1577736 | −0.6215965 | epidermal growth factor (beta-urogastrone) |
| 241489 | −0.6212938 | adrenergic, beta-2-, receptor, surface |
| 2316441 | −0.6206597 | ras homolog gene family, member C |
| 1712825 | −0.6204409 | ESTs |
| 52724 | −0.6199204 | hypothetical protein FLJ20241 |
| 150897 | −0.6188264 | UDP-GlcNAc:betaGal beta-1,3-N- |
| | | acetylglucosaminyltransferase 3 |
| 823851 | −0.6182341 | AE-binding protein 1 |
| 810057 | −0.6178096 | vasoactive intestinal peptide receptor 1 |
| 81316 | −0.6176265 | ESTs |
| 298134 | −0.6152731 | frizzled (Drosophila) homolog 1 |
| 2028238 | −0.6141182 | KIAA0300 protein |
| 1898758 | −0.6126872 | BCE-1 protein |
| 250797 | −0.6124667 | hypothetical protein FLJ20038 |
| 754040 | −0.610807 | NS1-associated protein 1 |
| 1584628 | −0.6102937 | alpha-actinin-2-associated LIM protein |
| 230910 | −0.6096107 | |
| 172783 | −0.6088873 | hypothetical protein FLJ10390 |
| 824799 | −0.6054867 | ART-4 protein |
| 785967 | −0.6049171 | erythrocyte membrane protein band 4.1-like 2 |
| 342349 | −0.603487 | mitogen-activated protein kinase kinase kinase 14 |
| 67067 | −0.6003961 | Homo sapiens mRNA; cDNA DKFZp564O222 (from clone |
| | | DKFZp564O222) |
| 1533710 | −0.5988282 | ortholog of mouse integral membrane glycoprotein LIG-1 |
| 768007 | −0.5983563 | hypothetical protein |
| 1707637 | −0.5964339 | ESTs |
| 1634832 | −0.5931096 | class I cytokine receptor |
| 1609625 | −0.5920312 | selectin P ligand |
| 1592530 | −0.5918818 | mammalian inositol hexakisphosphate kinase 2 |
| 272706 | −0.5914377 | alpha2,3-sialyltransferase |
| 897720 | −0.5904719 | trophinin |
| 1472735 | −0.5888169 | metallothionein 1E (functional) |
| 767641 | −0.5881449 | mitogen-activated protein kinase 8 interacting protein 2 |
| 1651906 | −0.5863631 | hypothetical protein FLJ23544 |
| 564801 | −0.5824549 | Homo sapiens cDNA: FLJ21409 fis, clone COL03924 |
| 248886 | −0.5823315 | rab3 GTPase-activating protein, non-catalytic subunit (150 kD) |
| 250654 | −0.5815576 | secreted protein, acidic, cysteine-rich (osteonectin) |
| 1968422 | −0.5802058 | Homo sapiens mRNA full length insert cDNA clone |
| | | EUROIMAGE 1968422 |
| 712829 | −0.5797871 | LIM domain only 2 (rhombotin-like 1) |
| 752837 | −0.5794612 | Homo sapiens mRNA for FLJ00074 protein, partial cds |
| 346902 | −0.5794362 | ESTs, Weakly similar to ALU2_HUMAN ALU SUBFAMILY SB |
| | | SEQUENCE CONTAMINATION WARNING ENTRY |
| | | [H. sapiens] |
| 782193 | −0.5788147 | thioredoxin |
| 358531 | −0.5784457 | v-jun avian sarcoma virus 17 oncogene homolog |
| 2271240 | −0.5782432 | hypothetical protein |
| 66532 | −0.5773248 | endothelin 3 |
| 120106 | −0.5770131 | caspase 1, apoptosis-related cysteine protease (interleukin 1, |
| | | beta, convertase) |
| 810552 | −0.5768989 | B-cell associated protein |
| 770848 | −0.5767154 | ESTs, Weakly similar to ALU1_HUMAN ALU SUBFAMILY J |
| | | SEQUENCE CONTAMINATION WARNING ENTRY |
| | | [H. sapiens] |
| 1032831 | −0.5764161 | gycosyltransferase |
| 460403 | −0.5739604 | laminin, gamma 2 (nicein (100 kD), kalinin (105 kD), BM600 |
| | | (100 kD), Herlitz junctional epidermolysis bullosa)) |
| 489755 | −0.5730453 | a disintegrin and metalloproteinase domain 12 (meltrin alpha) |
| 502753 | −0.5724623 | angiopoietin 2 |
| 68534 | −0.5724394 | Homo sapiens cDNA: FLJ22290 fis, clone HRC04405 |
| 854645 | −0.5723062 | CDC-like kinase 3 |
| 322024 | −0.5714933 | ESTs, Highly similar to T12495 hypothetical protein |
| | | DKFZp434H071.1 [H. sapiens] |
| 140071 | −0.5708303 | frizzled-related protein |
| 788518 | −0.5697316 | peroxisomal membrane protein 3 (35 kD, Zellweger syndrome) |
| 240752 | −0.56913 | hypothetical protein MGC14797 |
| 773487 | −0.5686324 | hypothetical protein MGC3032 |
| 1404774 | −0.5685596 | parathyroid hormone-like hormone |
| 814815 | −0.5675649 | plakophilin 4 |
| 462939 | −0.5662456 | ESTs |
| 240248 | −0.5660209 | transferrin |
| 208001 | −0.5640857 | CD59 antigen p18-20 (antigen identified by monoclonal |
| | | antibodies 16.3A5, EJ16, EJ30, EL32 and G344) |
| 1909433 | −0.5638242 | DKFZP727C091 protein |
| 1636360 | −0.5629882 | hypothetical protein FLJ14957 |
| 138242 | −0.5621088 | ESTs, Moderately similar to MAS2_HUMAN MANNAN- |
| | | BINDING LECTIN SERINE PROTEASE 2 PRECURSOR |
| | | [H. sapiens] |
| 703964 | −0.5620699 | inositol polyphosphate phosphatase-like 1 |
| 1522734 | −0.5618289 | ESTs |
| 843094 | −0.5617478 | ubiquitin-like 1 (sentrin) |
| 491519 | −0.561401 | Homo sapiens clone 24775 mRNA sequence |
| 377314 | −0.561312 | casein kinase 2, alpha prime polypeptide |
| 208969 | −0.561267 | EST |
| 788558 | −0.5600825 | KIAA1479 protein |
| 785530 | −0.5600048 | integrin, alpha 1 |
| 1473131 | −0.5594101 | transducin-like enhancer of split 2, homolog of Drosophila |
| | | E(sp1) |
| 812959 | −0.5591846 | KIAA1638 protein |
| 868169 | −0.5584677 | lipoprotein lipase |
| 322233 | −0.5578912 | ribosomal protein, large, P0 |
| 50519 | −0.5577761 | aryl hydrocarbon receptor nuclear translocator-like |
| 299720 | −0.5565137 | |
| 215000 | −0.555725 | vasoactive intestinal peptide receptor 1 |
| 51981 | −0.5554908 | ribosomal protein L7a |
| 703541 | −0.5534921 | KIAA1858 protein |
| 238907 | −0.5534598 | hypothetical protein, clone |
| | | Telethon(Italy_B41)_Strait02270_FL142 |
| 415613 | −0.5528761 | DHHC1 protein |
| 282404 | −0.5528532 | Homo sapiens mRNA for KIAA1671 protein, partial cds |
| 344588 | −0.55146 | kallikrein 5 |
| 746373 | −0.5513143 | RNA polymerase I transcription factor RRN3 |
| 491186 | −0.5507413 | Homo sapiens cDNA: FLJ23131 fis, clone LNG08502 |
| 75059 | −0.5504962 | poly(A)-binding protein, cytoplasmic 1-like |
| 345764 | −0.5504113 | special AT-rich sequence binding protein 1 (binds to nuclear |
| | | matrix/scaffold-associating DNA's) |
| 1577920 | −0.5496944 | ESTs |
| 288748 | −0.5493871 | Homo sapiens, clone MGC: 5352 IMAGE: 3048106, mRNA, |
| | | complete cds |
| 810097 | −0.5487174 | Homo sapiens cDNA: FLJ21721 fis, clone COLF0381 |
| 755881 | −0.5473964 | aquaporin 5 |
| 1568391 | −0.5467858 | plastin 3 (T isoform) |
| 290866 | −0.5467613 | v-raf-1 murine leukemia viral oncogene homolog 1 |
| 503671 | −0.5461871 | Homo sapiens cDNA FLJ14368 fis, clone HEMBA1001122 |
| 2388571 | −0.5459823 | A kinase (PRKA) anchor protein 8 |
| 739193 | −0.5457998 | cellular retinoic acid-binding protein 1 |
| 756372 | −0.5447652 | retinoic acid receptor responder (tazarotene induced) 2 |
| 53081 | −0.5446617 | ESTs |
| 486179 | −0.5443525 | Homo sapiens cDNA FLJ10205 fis, clone HEMBA1004954 |
| 84464 | −0.5426397 | hypothetical protein FLJ12806 |
| 2213824 | −0.5412089 | protease inhibitor 3, skin-derived (SKALP) |
| 1597813 | −0.5406089 | hypothetical protein PP1044 |
| 810358 | −0.5403705 | acyl-Coenzyme A dehydrogenase, very long chain |
| 341759 | −0.5403406 | lung type-I cell membrane-associated glycoprotein |
| 309449 | −0.5401954 | ribosomal protein S4, Y-linked |
| 42681 | −0.5388424 | NY-REN-25 antigen |
| 299720 | −0.5388238 | eukaryotic translation elongation factor 1 alpha 1 |
| 162775 | −0.5385174 | DEAD/H (Asp-Glu-Ala-Asp/His) box polypeptide 5 (RNA |
| | | helicase, 68 kD) |
| 490102 | −0.5383385 | TBP-associated factor 172 |
| 877835 | −0.5382125 | ribosomal protein L35 |
| 299720 | −0.5378801 | eukaryotic translation elongation factor 1 alpha 1 |
| 1534493 | −0.5376323 | ESTs |
| 595637 | −0.5373697 | Homo sapiens mRNA; cDNA DKFZp586N012 (from clone |
| | | DKFZp586N012) |
| 725622 | −0.5370309 | ESTs |
| 233679 | −0.5356625 | hypothetical protein FLJ22362 |
| 785933 | −0.5340762 | sushi-repeat-containing protein, X chromosome |
| 825356 | −0.533771 | Homo sapiens cDNA FLJ11997 fis, clone HEMBB1001458 |
| 767982 | −0.533231 | Homo sapiens, clone IMAGE: 4134852, mRNA, partial cds |
| 277627 | −0.5329107 | Human SH3 domain-containing protein SH3P18 mRNA, |
| | | complete cds |
| 460126 | −0.5320146 | KIAA0819 protein |
| 346688 | −0.5315035 | melanoma inhibitory activity |
| 712023 | −0.5302372 | AT-binding transcription factor 1 |
| 24729 | −0.5302207 | cholinergic receptor, muscarinic 1 |
| 1590021 | −0.5302114 | ets variant gene 6 (TEL oncogene) |
| 809998 | −0.5297152 | |
| 811779 | −0.5291718 | phosphoinositide-3-kinase, regulatory subunit, polypeptide 1 |
| | | (p85 alpha) |
| 488130 | −0.5289721 | Homo sapiens cDNA FLJ20767 fis, clone COL06986 |
| 299720 | −0.5284407 | |
| 159462 | −0.528299 | serum constituent protein |
| 1570427 | −0.5280955 | hypothetical protein MGC4309 |
| 2308346 | −0.5268833 | cyclin-dependent kinase 2 |
| 1881774 | −0.5265891 | KIAA1678 |
| 591055 | −0.5258289 | protein kinase C and casein kinase substrate in neurons 2 |
| 470128 | −0.5253542 | myosin IE |
| 687990 | −0.5253354 | Rac/Cdc42 guanine exchange factor (GEF) 6 |
| 753620 | −0.5248489 | insulin-like growth factor binding protein 6 |
| 80374 | −0.5236475 | pyruvate dehydrogenase (lipoamide) alpha 1 |
| 878421 | −0.5233873 | transgelin 2 |
| 343079 | −0.5224833 | Homo sapiens mRNA; cDNA DKFZp761P0114 (from clone |
| | | DKFZp761P0114) |
| 284592 | −0.5205573 | PRO1659 protein |
| 855061 | −0.5203046 | vascular endothelial growth factor B |
| 183704 | −0.5184644 | Homo sapiens, clone MGC: 13446 IMAGE: 4275731, mRNA, |
| | | complete cds |
| 155896 | −0.5182247 | ORF |
| 842939 | −0.5172329 | adenosine deaminase, RNA-specific, B1 (homolog of rat RED1) |
| 685185 | −0.5172158 | reticulon 2 |
| 753301 | −0.5171758 | carcinoembryonic antigen-related cell adhesion molecule 1 |
| | | (biliary glycoprotein) |
| 795178 | −0.5169102 | lactate dehydrogenase C |
| 1911663 | −0.5168962 | ESTs |
| 345023 | −0.5166203 | ESTs, Highly similar to T08701 hypothetical protein |
| | | DKFZp564N123.1 [H. sapiens] |
| 1631682 | −0.5162406 | peptidylprolyl isomerase E (cyclophilin E) |
| 868308 | −0.5157133 | ribosomal protein S23 |
| 809998 | −0.5144681 | amylase, alpha 2A; pancreatic |
| 771004 | −0.5144276 | KIAA1201 protein |
| 842896 | −0.5137089 | hypothetical protein DKFZp762L0311 |
| 714213 | −0.5129593 | tumor necrosis factor receptor superfamily, member 6 |
| 197056 | −0.5128128 | ESTs |
| 415191 | −0.512457 | KIAA0161 gene product |
| 2306752 | −0.5123647 | stathmin-like 2 |
| 469306 | −0.5113377 | gastrin-releasing peptide |
| 824602 | −0.5107682 | interferon, gamma-inducible protein 16 |
| 307687 | −0.5106552 | protease, serine, 16 (thymus) |
| 1637296 | −0.5105214 | ribosomal protein S24 |
| 1358393 | −0.5090372 | mitogen-activated protein kinase kinase 3 |
| 504940 | −0.508056 | |
| 713839 | −0.5080365 | transcription factor AP-4 (activating enhancer-binding protein 4) |
| 220293 | −0.5078291 | Homo sapiens cDNA: FLJ21800 fis, clone HEP00618 |
| 125342 | −0.5078237 | SUMO-1-specific protease |
| 1601845 | −0.5074894 | Ca2+-promoted Ras inactivator |
| 240620 | −0.5065007 | vascular Rab-GAP/TBC-containing |
| 43679 | −0.5063146 | ESTs |
| 825013 | −0.505654 | acidic protein rich in leucines |
| 2116188 | −0.5046972 | histone deacetylase 5 |
| 161998 | −0.5044691 | hypothetical protein FLJ23138 |
| 83999 | −0.5044612 | Human DNA sequence from clone 889N15 on chromosome |
| | | Xq22.1-22.3. Contains part of the gene for a novel protein |
| | | similar to X. laevis Cortical Thymocyte Marker CTX, the |
| | | possibly alternatively spliced gene for 26S Proteasome subunit |
| | | p28 (Ankyrin repeat prote |
| 593840 | −0.5033966 | DKFZP564K1964 protein |
| 51448 | −0.5031992 | activating transcription factor 3 |
| 383501 | −0.502319 | regulator of G-protein signalling 9 |
| 2018423 | −0.501705 | death-associated protein kinase 2 |
| 796984 | −0.5010588 | cytochrome b-245, beta polypeptide (chronic granulomatous |
| | | disease) |
| 1160723 | −0.5005952 | LIM domain kinase 2 |
| 414999 | −0.5002542 | ets variant gene 4 (E1A enhancer-binding protein, E1AF) |
| 770212 | −0.4981295 | chitinase 3-like 1 (cartilage glycoprotein-39) |
| 48518 | −0.4966991 | ATP-binding cassette, sub-family A (ABC1), member 5 |
| 85634 | −0.4961026 | complement component 1, s subcomponent |
| 341763 | −0.4953701 | caspase 5, apoptosis-related cysteine protease |
| 812161 | −0.4948424 | ESTs |
| 1417886 | −0.4941304 | hypothetical protein FLJ23239 |
| 1500815 | −0.4934665 | Homo sapiens cDNA: FLJ22130 fis, clone HEP19632 |
| 868400 | −0.4928479 | glutaminyl-tRNA synthetase |
| 809894 | −0.491862 | acetyl-CoA synthetase |
| 753745 | −0.4917192 | ESTs, Weakly similar to S57447 HPBRII-7 protein [H. sapiens] |
| 85394 | −0.4913773 | phosphatidic acid phosphatase type 2B |
| 134192 | −0.4908497 | ESTs |
| 813698 | −0.4898644 | sprouty (Drosophila) homolog 2 |
| 253884 | −0.4897811 | Human BAC clone GS1-99H8 |
| 131012 | −0.4897744 | hypothetical protein FLJ10633 |
| 855755 | −0.4897236 | fibrillarin |
| 1711456 | −0.4896574 | H factor (complement)-like 1 |
| 815142 | −0.4881454 | spastic ataxia of Charlevoix-Saguenay (sacsin) |
| 2413337 | −0.4879268 | sortilin-related receptor, L(DLR class) A repeats-containing |
| 327432 | −0.4870045 | semaphorin Y |
| 1897944 | −0.4855156 | ESTs, Weakly similar to T00366 hypothetical protein KIAA0669 |
| | | [H. sapiens] |

### Example VI

### Genes for Discriminating Between Normal and ADH (Non-Malignant) Versus DCIS and IDC (Malignant)

As shown in Table 5 below, 400 genes were identified as being able to discriminate between normal and ADH (non-malignant) versus DCIS and IDC (malignant).

TABLE 5

| CloneID | Weight | Description |
|---|---|---|
| 796469 | 1.4207633 | HSPC150 protein similar to ubiquitin-conjugating enzyme |
| 488964 | 1.3447179 | H2A histone family, member O |
| 1505038 | 1.3277637 | hypothetical protein FLJ20171 |
| 1500000 | 1.2926116 | H2B histone family, member B |
| 1554549 | 1.2787033 | hydroxyacyl glutathione hydrolase |
| 812238 | 1.2664748 | hypothetical protein MGC4692 |
| 35147 | 1.2581066 | ESTs, Weakly similar to unnamed protein product |
| | | [H. sapiens] |
| 122077 | 1.2576139 | putative membrane protein |
| 788654 | 1.2573483 | growth factor receptor-bound protein 2 |
| 595037 | 1.2486446 | retinoic acid induced 3 |
| 565319 | 1.2155833 | Homo sapiens mRNA; cDNA DKFZp564B1264 (from |
| | | clone DKFZp564B1264) |
| 283919 | 1.2112507 | H2A histone family, member L |
| 1917941 | 1.1872008 | purine-rich element binding protein B |
| 359887 | 1.1837896 | translocase of inner mitochondrial membrane 17 (yeast) |
| | | homolog A |
| 471568 | 1.1673113 | hematological and neurological expressed 1 |
| 290841 | 1.1670252 | H2B histone family, member A |
| 796694 | 1.1580364 | baculoviral IAP repeat-containing 5 (survivin) |
| 366132 | 1.1440486 | succinate dehydrogenase complex, subunit C, |
| | | integral membrane protein, 15 kD |
| 823598 | 1.1425746 | proteasome (prosome, macropain) 26S subunit, |
| | | non-ATPase, 12 |
| 1323448 | 1.1309009 | cysteine-rich protein 1 (intestinal) |
| 810711 | 1.1074523 | stearoyl-CoA desaturase (delta-9-desaturase) |
| 741474 | 1.0870449 | glucose phosphate isomerase |
| 745606 | 1.0663136 | hypothetical protein PP591 |
| 2054635 | 1.0613961 | proteasome (prosome, macropain) subunit, alpha |
| | | type, 7 |
| 178805 | 1.0513873 | Human DNA sequence from clone RP5-850E9 on |
| | | chromosome 20. Contains part of the gene for a |
| | | novel C2H2 type zinc finger protein similar to |
| | | Drosophila Scratch (Scrt), Slug and Xenopus |
| | | Snail, a novel gene similar to Drosophila |
| | | CG6762, STSs, GSSs and five CpG |
| 347373 | 1.0454939 | transcription elongation factor B (SIII), |
| | | polypeptide 1 (15 kD, elongin C) |
| 199403 | 1.0142329 | lectin, galactoside-binding, soluble, 8 (galectin 8) |
| 814054 | 1.0130821 | KIAA0040 gene product |
| 2029173 | 1.0094499 | ESTs, Weakly similar to N-WASP [H. sapiens] |
| 209066 | 1.0066096 | |
| 782428 | 1.0008279 | KIAA0250 gene product |
| 769921 | 0.9944462 | ubiquitin carrier protein E2-C |
| 470061 | 0.9920108 | seven in absentia (Drosophila) homolog 2 |
| 796723 | 0.9915885 | Homo sapiens clone CDABP0014 mRNA |
| | | sequence |
| 1609836 | 0.9886168 | glutamate-ammonia ligase (glutamine synthase) |
| 2322367 | 0.9859632 | reticulon 4 |
| 280375 | 0.9793036 | PRO2000 protein |
| 2016908 | 0.969649 | ESTs, Weakly similar to CA13_HUMAN |
| | | COLLAGEN ALPHA 1(III) CHAIN PRECURSOR |
| | | [H. sapiens] |
| 1858892 | 0.9669022 | hypothetical protein MGC4825 |
| 46248 | 0.9628117 | ADP-ribosyltransferase (NAD+; poly (ADP-ribose) |
| | | polymerase) |
| 2014034 | 0.9619198 | methylene tetrahydrofolate dehydrogenase |
| | | (NAD+ dependent), methenyltetrahydrofolate |
| | | cyclohydrolase |
| 898032 | 0.9547022 | KIAA0097 gene product |
| 725454 | 0.9476507 | CDC28 protein kinase 2 |
| 79520 | 0.9457391 | RAB2, member RAS oncogene family |
| 810558 | 0.943041 | proteasome (prosome, macropain) 26S subunit, |
| | | ATPase, 4 |
| 272529 | 0.9423688 | phosphomannomutase 2 |
| 122241 | 0.939204 | proteasome (prosome, macropain) subunit, beta |
| | | type, 2 |
| 469686 | 0.9381847 | Ric (Drosophila)-like, expressed in many tissues |
| 624667 | 0.9336899 | CGI-92 protein |
| 488202 | 0.932881 | ESTs, Weakly similar to YZ28_HUMAN |
| | | HYPOTHETICAL PROTEIN ZAP128 [H. sapiens] |
| 825470 | 0.9274271 | topoisomerase (DNA) II alpha (170 kD) |
| 1640821 | 0.920874 | ESTs, Weakly similar to I78885 serine/threonine- |
| | | specific protein kinase [H. sapiens] |
| 686552 | 0.9182272 | golgi phosphoprotein 1 |
| 2016648 | 0.917373 | Homo sapiens mRNA; cDNA DKFZp434N1728 |
| | | (from clone DKFZp434N1728) |
| 1911343 | 0.9110591 | RAB26, member RAS oncogene family |
| 781097 | 0.9100493 | reticulon 3 |
| 244801 | 0.908481 | Rho guanine exchange factor (GEF) 11 |
| 754628 | 0.9061145 | ESTs |
| 1574058 | 0.9058211 | 1-acylglycerol-3-phosphate O-acyltransferase 2 |
| | | (lysophosphatidic acid acyltransferase, beta) |
| 753299 | 0.9019921 | hypothetical protein FLJ10504 |
| 811774 | 0.9014801 | CGI-49 protein |
| 595213 | 0.8978322 | hypothetical protein |
| 868128 | 0.8970146 | JM4 protein |
| 810124 | 0.8891272 | platelet-activating factor acetylhydrolase, isoform |
| | | Ib, gamma subunit (29 kD) |
| 66406 | 0.8888639 | hypothetical protein DKFZp762E1312 |
| 1636092 | 0.8841788 | hypothetical protein FLJ20657 |
| 1869201 | 0.8825788 | hypothetical protein MGC2745 |
| 625923 | 0.8821749 | phosphoenolpyruvate carboxykinase 2 |
| | | (mitochondrial) |
| 1492238 | 0.8804306 | HSPC003 protein |
| 731044 | 0.8716644 | glutaredoxin 2 |
| 839682 | 0.8696528 | ubiquitin-conjugating enzyme E2N (homologous |
| | | to yeast UBC13) |
| 951233 | 0.8614727 | proteasome (prosome, macropain) subunit, beta |
| | | type, 3 |
| 843195 | 0.8606568 | phosphoserine phosphatase |
| 288999 | 0.8593924 | small protein effector 1 of Cdc42 |
| 51773 | 0.8588635 | hypothetical protein MGC3077 |
| 209066 | 0.8582298 | serine/threonine kinase 15 |
| 1474955 | 0.8573467 | TATA box binding protein (TBP)-associated |
| | | factor, RNA polymerase II, N, 68 kD (RNA-binding |
| | | protein 56) |
| 2043167 | 0.8551193 | BCL2-associated athanogene 3 |
| 742707 | 0.8515067 | ESTs, Weakly similar to MUC2_HUMAN MUCIN |
| | | 2 PRECURSOR [H. sapiens] |
| 743589 | 0.8514377 | ESTs, Weakly similar to T2D3_HUMAN |
| | | TRANSCRIPTION INITIATION FACTOR TFIID |
| | | 135 KDA SUBUNIT [H. sapiens] |
| 704414 | 0.8498631 | small nuclear ribonucleoprotein polypeptides B |
| | | and B1 |
| 2309073 | 0.8484971 | frizzled (Drosophila) homolog 5 |
| 2052113 | 0.8477245 | hypothetical protein FLJ10903 |
| 686172 | 0.846207 | KIAA0008 gene product |
| 150003 | 0.8447372 | hypothetical protein FLJ13187 |
| 705064 | 0.8401441 | transforming, acidic coiled-coil containing protein 3 |
| 1709791 | 0.8397779 | BAI1-associated protein 1 |
| 1469425 | 0.8391993 | SRY (sex determining region Y)-box 22 |
| 429799 | 0.8386406 | hypothetical protein FLJ21939 similar to 5- |
| | | azacytidine induced gene 2 |
| 729975 | 0.8385313 | meningioma expressed antigen 5 (hyaluronidase) |
| 1393018 | 0.8376676 | general transcription factor IIIC, polypeptide 1 |
| | | (alpha subunit, 220 kD) |
| 1492463 | 0.8360771 | selenoprotein X, 1 |
| 2028949 | 0.8358024 | hypothetical protein PRO1855 |
| 789012 | 0.8351735 | fibulin 2 |
| 470124 | 0.8347241 | RAD1 (S. pombe) homolog |
| 1409509 | 0.8339967 | troponin T1, skeletal, slow |
| 1605426 | 0.8317254 | hypothetical protein FLJ13352 |
| 44292 | 0.831599 | Homo sapiens mRNA; cDNA DKFZp434C107 |
| | | (from clone DKFZp434C107) |
| 1435003 | 0.8311727 | tumor suppressing subtransferable candidate 1 |
| 503215 | 0.8286483 | pilin-like transcription factor |
| 504308 | 0.8199799 | hypothetical protein FLJ10540 |
| 785707 | 0.8176557 | protein regulator of cytokinesis 1 |
| 1500162 | 0.8155519 | ESTs |
| 149355 | 0.8134342 | translocating chain-associating membrane |
| | | protein |
| 1845169 | 0.8131362 | RAB35, member RAS oncogene family |
| 869375 | 0.808792 | isocitrate dehydrogenase 2 (NADP+), |
| | | mitochondrial |
| 1492426 | 0.8032542 | chromosome 19 open reading frame 3 |
| 782513 | 0.8030042 | interferon, alpha-inducible protein (clone IFI-6-16) |
| 813281 | 0.8016742 | WW domain-containing protein 1 |
| 814378 | 0.8015236 | serine protease inhibitor, Kunitz type, 2 |
| 700792 | 0.8005506 | cyclin-dependent kinase inhibitor 3 (CDK2- |
| | | associated dual specificity phosphatase) |
| 340558 | 0.7990715 | actin related protein 2/3 complex, subunit 5 (16 kD) |
| 842994 | 0.7983201 | cathepsin Z |
| 138189 | 0.7953361 | Wolfram syndrome 1 (wolframin) |
| 289978 | 0.7931469 | ubiquitin-like 4 |
| 2019223 | 0.7893602 | mitochondrial ribosomal protein L17 |
| 2110511 | 0.7863117 | artemin |
| 814528 | 0.7845635 | Homo sapiens cDNA: FLJ22139 fis, clone |
| | | HEP20959 |
| 1573251 | 0.7816689 | peroxisomal long-chain acyl-coA thioesterase |
| 773922 | 0.7799164 | KIAA0005 gene product |
| 172517 | 0.7779159 | hippocalcin-like 1 |
| 564981 | 0.7745626 | ESTs |
| 1420370 | 0.7726877 | biliverdin reductase B (flavin reductase (NADPH)) |
| 308466 | 0.771216 | GTP-binding protein Sara |
| 199645 | 0.769591 | nicastrin |
| 1422338 | 0.7690604 | ribonucleotide reductase M2 polypeptide |
| 1474424 | 0.7689082 | Homo sapiens cDNA FLJ12758 fis, clone |
| | | NT2RP2001328 |
| 813751 | 0.7665427 | sialyltransferase 4C (beta-galactosidase alpha- |
| | | 2,3-sialytransferase) |
| 131094 | 0.7626373 | Homo sapiens cDNA: FLJ21587 fis, clone |
| | | COL06946 |
| 842980 | 0.762412 | developmentally regulated GTP-binding protein 1 |
| 212542 | 0.7585053 | Homo sapiens cDNA FLJ12900 fis, clone |
| | | NT2RP2004321 |
| 108425 | 0.7581954 | |
| 811761 | 0.7578696 | Nijmegen breakage syndrome 1 (nibrin) |
| 241348 | 0.757138 | prenylcysteine lyase |
| 810725 | 0.7561061 | ATPase, H+ transporting, lysosomal (vacuolar |
| | | proton pump) 21 kD |
| 1460110 | 0.7559869 | proteasome (prosome, macropain) subunit, beta |
| | | type, 5 |
| 120749 | 0.7545697 | ESTs, Moderately similar to KIAA1215 protein |
| | | [H. sapiens] |
| 827171 | 0.7543905 | ESTs |
| 39884 | 0.7526496 | IMP (inosine monophosphate) dehydrogenase 1 |
| 124298 | 0.7507816 | microsomal glutathione S-transferase 3 |
| 753378 | 0.7492212 | hypothetical protein FLJ22649 similar to signal |
| | | peptidase SPC22/23 |
| 210862 | 0.7483198 | acyl-Coenzyme A oxidase 1, palmitoyl |
| 785766 | 0.7476331 | hypothetical protein |
| 1553306 | 0.7471557 | proteasome (prosome, macropain) 26S subunit, |
| | | non-ATPase, 11 |
| 470099 | 0.7443747 | HT002 protein; hypertension-related calcium- |
| | | regulated gene |
| 83363 | 0.7435058 | protein-L-isoaspartate (D-aspartate) O- |
| | | methyltransferase |
| 489351 | 0.7422879 | hypothetical protein DKFZp566J2046 |
| 810612 | 0.7419597 | S100 calcium-binding protein A11 (calgizzarin) |
| 825585 | 0.741258 | tubulin-specific chaperone e |
| 1456348 | 0.7401571 | N-acetylneuraminic acid phosphate synthase; |
| | | sialic acid synthase |
| 1473922 | 0.7399948 | actin related protein 2/3 complex, subunit 3 (21 kD) |
| 150314 | 0.739762 | lysophospholipase I |
| 897806 | 0.7388301 | hypoxia-inducible factor 1, alpha subunit (basic |
| | | helix-loop-helix transcription factor) |
| 811585 | 0.738212 | huntingtin (Huntington disease) |
| 685516 | 0.7378926 | putative G protein-coupled receptor |
| 76605 | 0.7376829 | nesca protein |
| 1476053 | 0.7367106 | RAD51 (S. cerevisiae) homolog (E coli RecA |
| | | homolog) |
| 221295 | 0.7342275 | inhibitor of DNA binding 2, dominant negative |
| | | helix-loop-helix protein |
| 824879 | 0.7333071 | hypothetical protein MGC11275 |
| 768570 | 0.732628 | hypothetical protein FLJ11280 |
| 73009 | 0.731401 | ESTs, Weakly similar to A43932 mucin 2 |
| | | precursor, intestinal [H. sapiens] |
| 564492 | 0.7301895 | mitochondrial carrier homolog 2 |
| 2017415 | 0.7281714 | centromere protein A (17 kD) |
| 788654 | 0.7244749 | |
| 488505 | 0.7242619 | accessory proteins BAP31/BAP29 |
| 824962 | 0.7238234 | karyopherin alpha 2 (RAG cohort 1, importin |
| | | alpha 1) |
| 1616253 | 0.7231756 | breast carcinoma amplified sequence 1 |
| 1435862 | 0.723081 | antigen identified by monoclonal antibodies 12E7, |
| | | F21 and O13 |
| 786067 | 0.7228184 | cell division cycle 25B |
| 2050827 | 0.7211304 | proteasome (prosome, macropain) 26S subunit, |
| | | ATPase, 5 |
| 248649 | 0.7210707 | hypothetical protein FLJ13910 |
| 51532 | 0.7195798 | ADP-ribosylation factor-like 6 interacting protein |
| 727078 | 0.7184618 | Homo sapiens cDNA: FLJ23602 fis, clone |
| | | LNG15735 |
| 37708 | 0.7163849 | hypothetical protein MGC3101 |
| 430235 | 0.7162503 | H2B histone family, member Q |
| 897770 | 0.715754 | |
| 292936 | 0.7154295 | hypothetical protein FLJ10468 |
| 365738 | 0.7152855 | ESTs |
| 845363 | 0.714201 | non-metastatic cells 1, protein (NM23A) |
| | | expressed in |
| 809944 | 0.7139515 | KIAA0310 gene product |
| 1631699 | 0.7115561 | valosin-containing protein |
| 813629 | 0.7102574 | YME1 (S. cerevisiae)-like 1 |
| 813410 | 0.7093173 | polymerase (RNA) II (DNA directed) polypeptide |
| | | K (7.0 kD) |
| 826256 | 0.7092586 | transmembrane 7 superfamily member 1 |
| | | (upregulated in kidney) |
| 124331 | 0.7081481 | cleavage and polyadenylation specific factors 5, |
| | | 25 kD subunit |
| 770845 | 0.7068283 | hexokinase 1 |
| 67765 | 0.7064266 | carboxypeptidase M |
| 207288 | 0.7058528 | insulin induced gene 1 |
| 1639531 | 0.7033264 | RAB27A, member RAS oncogene family |
| 731023 | 0.7032815 | WD repeat domain 5 |
| 756442 | 0.7016064 | P450 (cytochrome) oxidoreductase |
| 358162 | 0.6999211 | protein predicted by clone 23627 |
| 782608 | 0.698569 | mitochondrial ribosomal protein L9 |
| 509823 | 0.6982362 | carcinoembryonic antigen-related cell adhesion |
| | | molecule 6 (non-specific cross reacting antigen) |
| 345787 | 0.6981453 | highly expressed in cancer, rich in leucine heptad |
| | | repeats |
| 810402 | 0.6978141 | hypothetical protein |
| 744417 | 0.6975761 | carnitine acetyltransferase |
| 814306 | 0.6963874 | tumor protein D52 |
| 41356 | 0.6961169 | protein phosphatase 2, regulatory subunit B |
| | | (B56), alpha isoform |
| 813419 | 0.6951349 | hydroxyacyl-Coenzyme A dehydrogenase, type II |
| 629944 | 0.6950339 | myosin VB |
| 327506 | 0.6943362 | Homo sapiens mRNA full length insert cDNA |
| | | clone EUROIMAGE 327506 |
| 768064 | 0.6936336 | cytochrome P450, subfamily I (aromatic |
| | | compound-inducible), polypeptide 1 |
| 770992 | 0.693225 | |
| 469383 | 0.6925975 | chromosome 8 open reading frame 1 |
| 1492780 | 0.6923749 | Homo sapiens cDNA FLJ14459 fis, clone |
| | | HEMBB1002409 |
| 41569 | 0.691145 | hypothetical protein FLJ12650 |
| 509588 | 0.6903363 | TATA box binding protein (TBP)-associated |
| | | factor, RNA polymerase II, J, 20 kD |
| 144880 | 0.6902434 | hypothetical protein from EUROIMAGE 1759349 |
| 789376 | 0.6883473 | thioredoxin reductase 1 |
| 268946 | 0.6881593 | WD40 protein Ciao1 |
| 810156 | 0.6879064 | deoxythymidylate kinase (thymidylate kinase) |
| 84295 | 0.6876167 | interleukin 1 receptor antagonist |
| 246800 | 0.6867481 | hypothetical protein FLJ10803 |
| 589232 | 0.6865999 | hypothetical protein FLJ11506 |
| 859761 | 0.6846134 | poliovirus receptor-related 2 (herpesvirus entry |
| | | mediator B) |
| 431505 | 0.6840493 | ESTs, Highly similar to A31026 probable |
| | | membrane receptor protein [H. sapiens] |
| 109863 | 0.683291 | epithelial membrane protein 2 |
| 770355 | 0.6829507 | lanosterol synthase (2,3-oxidosqualene- |
| | | lanosterol cyclase) |
| 344091 | 0.6818771 | ESTs |
| 813707 | 0.6814476 | regulator of G-protein signalling 16 |
| 124781 | 0.6809199 | squalene epoxidase |
| 502774 | 0.6807524 | hypothetical protein FLJ20623 |
| 825740 | 0.6805157 | DKFZp434J1813 protein |
| 1536006 | 0.6805004 | ESTs |
| 203003 | 0.6802818 | non-metastatic cells 4, protein expressed in |
| 785616 | 0.6800658 | signal sequence receptor, alpha (translocon- |
| | | associated protein alpha) |
| 785840 | 0.6800437 | SEC24 (S. cerevisiae) related gene family, |
| | | member D |
| 256619 | 0.6797698 | hydroxysteroid (17-beta) dehydrogenase 7 |
| 2108077 | 0.6796871 | CGI-112 protein |
| 742595 | 0.6784804 | cyclin-dependent kinase 5 |
| 1476065 | 0.6783519 | stathmin 1/oncoprotein 18 |
| 68636 | 0.6778817 | hypothetical protein MGC2477 |
| 564847 | 0.6770455 | ESTs |
| 744047 | 0.6748419 | polo (Drosophila)-like kinase |
| 897813 | 0.6741253 | polyadenylate binding protein-interacting protein 1 |
| 823930 | 0.6738266 | actin related protein 2/3 complex, subunit 1A (41 kD) |
| 236034 | 0.673506 | uncoupling protein 2 (mitochondrial, proton |
| | | carrier) |
| 743977 | 0.6729967 | Homo sapiens mRNA for TL132 |
| 293727 | 0.6728542 | hypothetical protein MGC861 |
| 590759 | 0.672734 | sterol-C4-methyl oxidase-like |
| 811024 | 0.6706275 | bone marrow stromal cell antigen 2 |
| 785795 | 0.6683919 | hypothetical protein FLJ12910 |
| 530310 | 0.6682042 | KIAA0143 protein |
| 1734309 | 0.666164 | sperm associated antigen 4 |
| 1898619 | 0.6651268 | hypothetical protein MGC15737 |
| 358267 | 0.6648183 | EST, Moderately similar to AF119917 63 |
| | | PRO2831 [H. sapiens] |
| 753400 | 0.6633015 | CGI-204 protein |
| 292770 | 0.6609782 | Homo sapiens, clone IMAGE: 3627860, mRNA, |
| | | partial cds |
| 884425 | 0.6608337 | chaperonin containing TCP1, subunit 5 (epsilon) |
| 1696757 | 0.6599512 | hypothetical protein KIAA1165 |
| 121251 | 0.6596925 | hypothetical protein MGC5576 |
| 770785 | 0.658751 | 1,2-alpha-mannosidase IC |
| 590774 | 0.657447 | mitogen-activated protein kinase 13 |
| 969877 | 0.6564571 | synaptosomal-associated protein, 25 kD |
| 415102 | 0.6562436 | cell division cycle 25C |
| 120271 | 0.6562017 | hypothetical protein MGC4692 |
| 713782 | 0.6552297 | a disintegrin and metalloproteinase domain 15 |
| | | (metargidin) |
| 49351 | 0.6551453 | SEX gene |
| 488642 | 0.6541915 | ESTs, Weakly similar to I38022 hypothetical |
| | | protein [H. sapiens] |
| 343607 | 0.6510034 | AD-015 protein |
| 2306987 | 0.6501071 | secreted and transmembrane 1 |
| 280249 | 0.64759 | Kruppel-like factor 7 (ubiquitous) |
| 752631 | 0.6473517 | fibroblast growth factor receptor 3 |
| | | (achondroplasia, thanatophoric dwarfism) |
| 489594 | 0.645574 | hypothetical protein FLJ11565 |
| 773188 | 0.6448143 | nuclear receptor subfamily 1, group D, member 2 |
| 2015517 | 0.6441737 | hypothetical protein FLJ22237 |
| 343731 | 0.6424907 | |
| 491524 | 0.6424448 | mitochondrial ribosomal protein L13 |
| 824524 | 0.6424419 | UDP-galactose transporter related |
| 593431 | 0.6422897 | ESTs, Moderately similar to CEGT_HUMAN |
| | | CERAMIDE GLUCOSYLTRANSFERASE |
| | | [H. sapiens] |
| 812994 | 0.6420817 | retinoid X receptor, alpha |
| 1631735 | 0.6404771 | Homo sapiens, clone IMAGE: 3604336, mRNA, |
| | | partial cds |
| 278531 | 0.6404612 | cytochrome c oxidase subunit VIc |
| 2302099 | 0.6386686 | sialidase 3 (membrane sialidase) |
| 454896 | 0.6384516 | DnaJ (Hsp40) homolog, subfamily A, member 2 |
| 1456701 | 0.6383709 | B-cell CLL/lymphoma 9 |
| 2055807 | 0.638184 | protein kinase domains containing protein similar |
| | | to phosphoprotein C8FW |
| 1518402 | 0.6378481 | KIAA1361 protein |
| 810762 | 0.6371461 | SNARE protein |
| 124447 | 0.6363079 | KIAA1184 protein |
| 49273 | 0.635859 | solute carrier family 27 (fatty acid transporter), |
| | | member 4 |
| 365060 | 0.6350631 | RAB11A, member RAS oncogene family |
| 1591264 | 0.6337293 | transaldolase 1 |
| 41698 | 0.6327738 | progesterone binding protein |
| 810316 | 0.6323393 | very long-chain acyl-CoA synthetase; lipidosin |
| 826363 | 0.6322533 | lysophospholipase II |
| 2011515 | 0.6319712 | DKFZP586B0923 protein |
| 770675 | 0.6315109 | Homo sapiens cDNA: FLJ21323 fis, clone |
| | | COL02374 |
| 1461477 | 0.6300096 | Homo sapiens mRNA; cDNA DKFZp586I0324 |
| | | (from clone DKFZp586I0324) |
| 366834 | 0.629867 | envoplakin |
| 1601947 | 0.6297475 | cytochrome c oxidase subunit VIIa polypeptide 2 |
| | | (liver) |
| 510575 | 0.6295917 | hypothetical protein FLJ22087 |
| 503851 | 0.6291771 | nuclear receptor co-repressor/HDAC3 complex |
| | | subunit |
| 810063 | 0.6286184 | growth factor, erv1 (S. cerevisiae)-like |
| | | (augmenter of liver regeneration) |
| 754653 | 0.6277098 | cadherin, EGF LAG seven-pass G-type receptor |
| | | 3, flamingo (I Drosophila) homolog |
| 1518890 | 0.6251884 | metallothionein-like 5, testis-specific (tesmin) |
| 784105 | 0.6244805 | ESTs |
| 205049 | 0.6231646 | protein kinase H11; small stress protein-like |
| | | protein HSP22 |
| 325606 | 0.6225147 | hypothetical protein MGC14353 |
| 760299 | −1.8441097 | dickkopf (Xenopus laevis) homolog 3 |
| 200814 | −1.8295958 | membrane metallo-endopeptidase (neutral |
| | | endopeptidase, enkephalinase, CALLA, CD10) |
| 1882697 | −1.715818 | peanut (Drosophila)-like 2 |
| 344720 | −1.6567437 | glycophorin C (Gerbich blood group) |
| 1161564 | −1.5877154 | desmuslin |
| 45099 | −1.566311 | regucalcin (senescence marker protein-30) |
| 75859 | −1.5303427 | N-myc downstream-regulated gene 2 |
| 811920 | −1.5255258 | interleukin 11 receptor, alpha |
| 1569187 | −1.4872982 | heparan sulfate (glucosamine) 3-O- |
| | | sulfotransferase 4 |
| 796542 | −1.4697418 | ets variant gene 5 (ets-related molecule) |
| 767202 | −1.4573536 | latent transforming growth factor beta binding |
| | | protein 2 |
| 285377 | −1.4496786 | pellino (Drosophila) homolog 2 |
| 300632 | −1.449587 | hypothetical protein FLJ21044 similar to Rbig1 |
| 160192 | −1.4231519 | ESTs, Weakly similar to 2004399A chromosomal |
| | | protein [H. sapiens ] |
| 611532 | −1.3689616 | troponin I, skeletal, fast |
| 813265 | −1.3632094 | Homo sapiens mRNA; cDNA DKFZp564H1916 |
| | | (from clone DKFZp564H1916) |
| 1469377 | −1.359583 | lipoma HMGIC fusion partner-like 2 |
| 810002 | −1.3553375 | Homo sapiens, clone MGC: 19762 |
| | | IMAGE: 3636045, mRNA, complete cds |
| 2056139 | −1.3506352 | LIM domain protein |
| 377275 | −1.3392122 | ataxia-telangiectasia group D-associated protein |
| 298122 | −1.3364021 | frizzled (Drosophila) homolog 7 |
| 72778 | −1.3299927 | caspase 7, apoptosis-related cysteine protease |
| 841308 | −1.3246996 | myosin, light polypeptide kinase |
| 377461 | −1.3214357 | caveolin 1, caveolae protein, 22 kD |
| 1473274 | −1.3200174 | myosin regulatory light chain 2, smooth muscle |
| | | isoform |
| 1554167 | −1.3141843 | hypothetical protein FLJ14529 |
| 131839 | −1.3048208 | folate receptor 1 (adult) |
| 67741 | −1.3014364 | PP2135 protein |
| 132857 | −1.280579 | Homo sapiens mRNA; cDNA DKFZp586N1323 |
| | | (from clone DKFZp586N1323) |
| 345670 | −1.2805684 | ESTs, Moderately similar to I59348 CCAAT |
| | | binding transcription factor CBF subunit C - rat |
| | | [R. norvegicus] |
| 781014 | −1.2659158 | suppression of tumorigenicity 5 |
| 289760 | −1.2610055 | ESTs, Highly similar to T00391 hypothetical |
| | | protein KIAA0612 [H. sapiens] |
| 742685 | −1.248683 | disabled (Drosophila) homolog 2 (mitogen- |
| | | responsive phosphoprotein) |
| 52419 | −1.2447753 | Friedreich ataxia region gene X123 |
| 838478 | −1.2349342 | neurocalcin delta |
| 839736 | −1.2337192 | crystallin, alpha B |
| 1558675 | −1.2313679 | SRY (sex determining region Y)-box 10 |
| 796181 | −1.2276581 | growth arrest-specific 6 |
| 878836 | −1.2200837 | secretory granule, neuroendocrine protein 1 (7B2 |
| | | protein) |
| 130201 | −1.2181641 | intercellular adhesion molecule 2 |
| 1917449 | −1.217371 | serum amyloid A4, constitutive |
| 22917 | −1.2164705 | Homo sapiens mRNA; cDNA DKFZp761M0111 |
| | | (from clone DKFZp761M0111) |
| 1871116 | −1.209237 | Homo sapiens mRNA; cDNA DKFZp434C1714 |
| | | (from clone DKFZp434C1714); partial cds |
| 823871 | −1.2090693 | SPARC-like 1 (mast9, hevin) |
| 811837 | −1.2030392 | |
| 811848 | −1.2011809 | hypothetical protein |
| 840266 | −1.200789 | Homo sapiens cDNA: FLJ22667 fis, clone |
| | | HSI08385 |
| 753071 | −1.2005994 | Homo sapiens cDNA: FLJ22528 fis, clone |
| | | HRC12825 |
| 2106144 | −1.1719133 | regulated in glioma |
| 2504881 | −1.1674204 | signal transducer and activator of transcription 5A |
| 811837 | −1.157587 | eukaryotic translation elongation factor 1 alpha 1 |
| 486683 | −1.1548164 | Homo sapiens mRNA; cDNA DKFZp564J0323 |
| | | (from clone DKFZp564J0323) |
| 712139 | −1.1491129 | ADP-ribosylation factor-like 7 |
| 196435 | −1.1475545 | ESTs |
| 877621 | −1.1422087 | nGAP-like protein |
| 811088 | −1.140941 | ephrin-B3 |
| 322561 | −1.1270333 | ribosomal protein L31 |
| 712401 | −1.1218827 | phosphoinositide-3-kinase, catalytic, delta |
| | | polypeptide |
| 788234 | −1.1061158 | inhibitor of DNA binding 4, dominant negative |
| | | helix-loop-helix protein |
| 140574 | −1.103064 | small inducible cytokine subfamily D (Cys-X3- |
| | | Cys), member 1 (fractalkine, neurotactin) |
| 490023 | −1.1020527 | hypothetical protein MGC2648 |
| 1584540 | −1.0933558 | Homo sapiens mRNA; cDNA DKFZp586M2022 |
| | | (from clone DKFZp586M2022) |
| 1556433 | −1.0887923 | GRO3 oncogene |
| 47043 | −1.0841886 | tensin |
| 303109 | −1.0807576 | purinergic receptor (family A group 5) |
| 343760 | −1.0803279 | SH3 domain binding glutamic acid-rich protein |
| | | like 2 |
| 248631 | −1.0784377 | aminomethyltransferase (glycine cleavage |
| | | system protein T) |
| 740620 | −1.071175 | tropomyosin 2 (beta) |
| 529843 | −1.0698218 | ESTs, Moderately similar to JC5238 |
| | | galactosylceramide-like protein, GCP [H. sapiens] |
| 990881 | −1.0640807 | |
| 80344 | −1.0602825 | interleukin 7 receptor |
| 625399 | −1.0594452 | hypothetical protein similar to beta-transducin |
| | | family |
| 416676 | −1.0568729 | pellino (Drosophila) homolog 1 |
| 416959 | −1.0566462 | nuclear factor I/B |
| 307029 | −1.0497874 | ribosomal protein L26 |
| 488404 | −1.0484323 | Homo sapiens clone TUA8 Cri-du-chat region |
| | | mRNA |
| 291478 | −1.0400846 | runt-related transcription factor 3 |
| 153760 | −1.0340645 | EphB1 |
| 119290 | −1.025797 | cortic al thymocyte receptor (X. laevis CTX) like |
| 814443 | −1.0250673 | hypothetical protein MGC3232 |
| 757191 | −1.0238476 | ESTs |
| 208718 | −1.0229324 | annexin A1 |
| 161456 | −1.0220494 | serum amyloid A1 |
| 1587710 | −1.0209983 | period (Drosophila) homolog 1 |
| 160609 | −1.0208819 | ESTs |
| 593023 | −1.0158099 | dystrobrevin, beta |
| 1878409 | −1.013872 | catechol-O-methyltransferase |
| 781017 | −1.0125987 | early growth response 2 (Krox-20 (Drosophila) |
| | | homolog) |
| 753162 | −1.0119485 | KIAA0603 gene product |
| 897963 | −1.0075423 | phosphatidic acid phosphatase type 2A |
| 505864 | −1.0033263 | RalGDS-like gene |
| 323780 | −1.0027215 | Homo sapiens cDNA FLJ11177 fis, clone |
| | | PLACE1007402 |
| 343695 | −1.002495 | hypothetical protein FLJ10875 |
| 277571 | −0.9977509 | KIAA1706 protein |
| 869450 | −0.9934083 | ribosomal protein L11 |
| 280907 | −0.9932806 | Kruppel-type zinc finger protein |
| 290378 | −0.9932643 | podocalyxin-like |
| 1635062 | −0.9905245 | DKFZP586A011 protein |
| 270826 | −0.9896888 | Homo sapiens cDNA FLJ13329 fis, clone |
| | | OVARC1001795 |
| 1257131 | −0.9869928 | ESTs |

### Example VII

### Genes for Discriminating Between ADH and DCIS

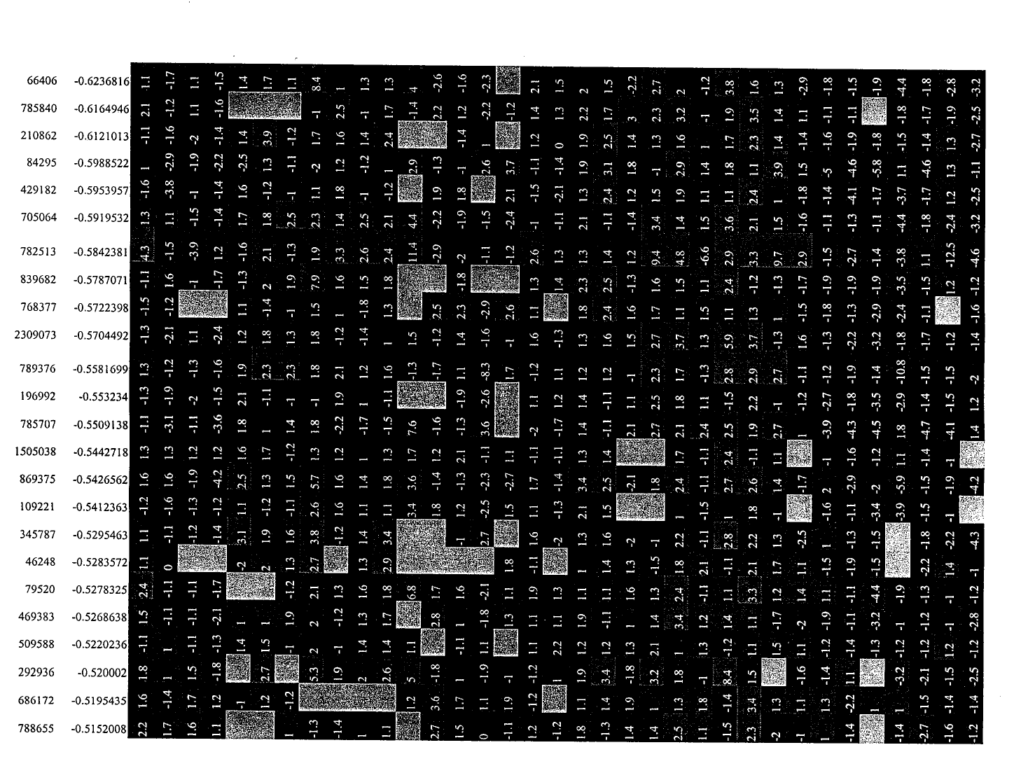

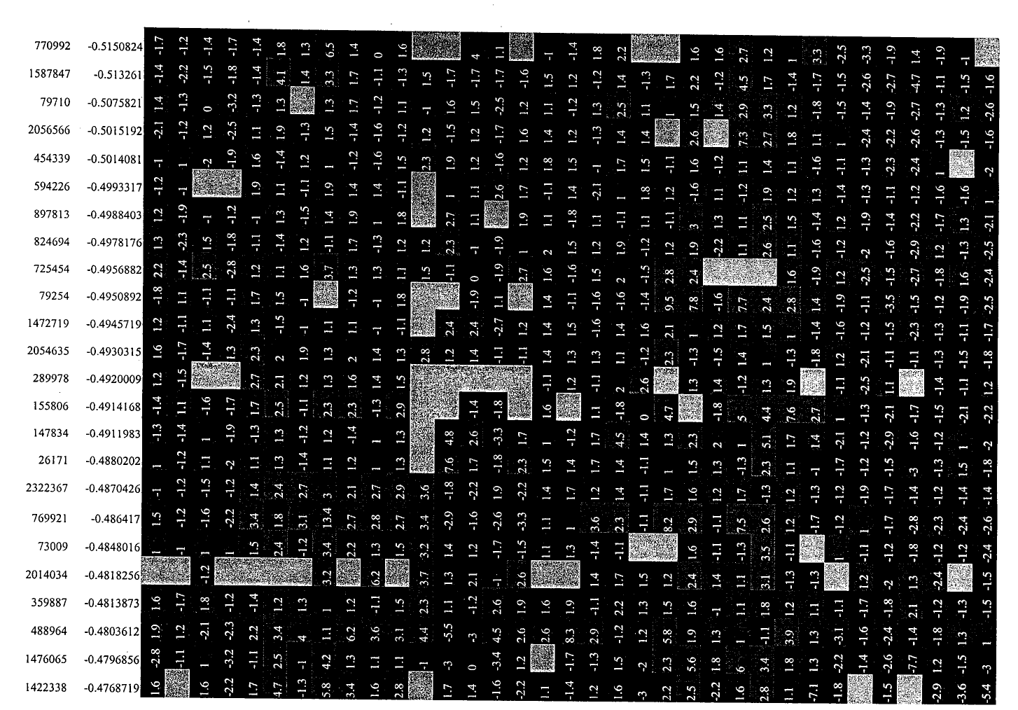

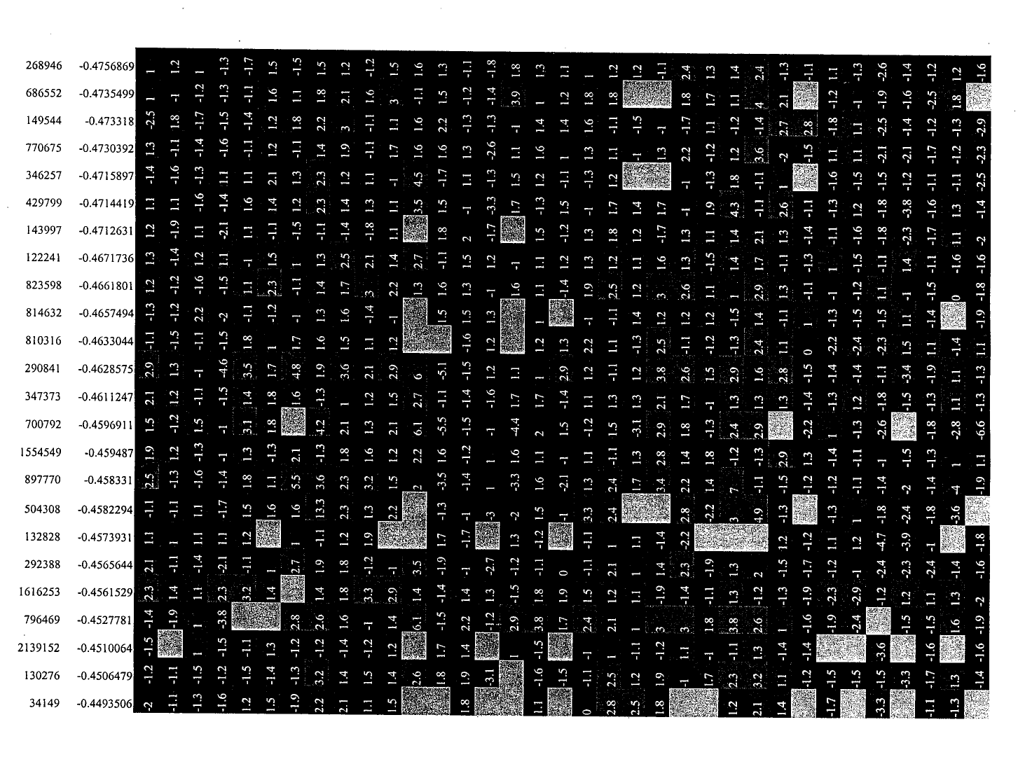

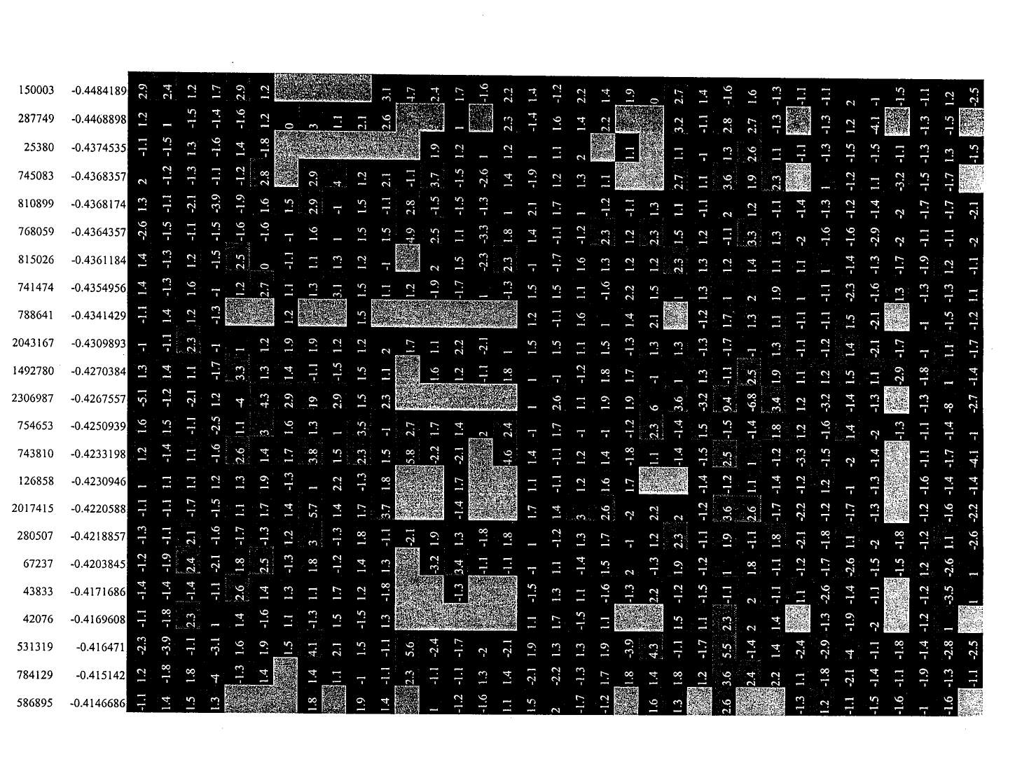

As shown in Table 6 below, 350 genes were identified as being able to discriminate between ADH and DCIS. The actual data is shown in FIG. 3.

TABLE 6

| CloneID | Weight | Description |
|---|---|---|
| 1404774 | −0.5685596 | parathyroid hormone-like hormone |
| 823871 | −0.9429443 | SPARC-like 1 (mast9, hevin) |
| 1882697 | −1.4318896 | peanut (Drosophila)-like 2 |
| 140071 | −0.5708303 | frizzled-related protein |
| 160192 | −1.1751869 | ESTs, Weakly similar to 2004399A chromosomal protein |
| | | [H. sapiens] |
| 796542 | −1.2625621 | ets variant gene 5 (ets-related molecule) |
| 611532 | −1.3158379 | troponin I, skeletal, fast |
| 1473274 | −1.1172693 | myosin regulatory light chain 2, smooth muscle isoform |
| 469306 | −0.5113377 | gastrin-releasing peptide |
| 2306697 | −0.4494025 | neuromedin B |
| 132857 | −1.0084069 | Homo sapiens mRNA; cDNA DKFZp586N1323 (from clone |
| | | DKFZp586N1323) |
| 2504881 | −1.000863 | signal transducer and activator of transcription 5A |
| 760299 | −1.7010462 | dickkopf (Xenopus laevis) homolog 3 |
| 293819 | −0.4051233 | oxidoreductase UCPA |
| 130835 | 0.3209345 | Homo sapiens, Similar to clone FLB3816, clone |
| | | IMAGE: 3454380, mRNA |
| 66532 | −0.5773248 | endothelin 3 |
| 2499829 | −0.3619416 | zinc finger protein 145 (Kruppel-like, expressed in |
| | | promyelocytic leukemia) |
| 85840 | −0.7754435 | nicotinamide N-methyltransferase |
| 859359 | −0.4669761 | quinone oxidoreductase homolog |
| 200814 | −1.4325283 | membrane metallo-endopeptidase (neutral endopeptidase, |
| | | enkephalinase, CALLA, CD10) |
| 825287 | −0.2783909 | tumor necrosis factor (ligand) superfamily, member 11 |
| 202577 | −0.2283149 | histamine N-methyltransferase |
| 2014373 | 0.0232775 | HNK-1 sulfotransferase |
| 153760 | −0.9227865 | EphB1 |
| 377275 | −1.3102234 | ataxia-telangiectasia group D-associated protein |
| 745490 | −0.6329354 | hypothetical protein FLJ20607 |
| 172783 | −0.6088873 | hypothetical protein FLJ10390 |
| 1558108 | 0.0044098 | ATP-binding cassette, sub-family C (CFTR/MRP), member 8 |
| 1587710 | −0.8252065 | period (Drosophila) homolog 1 |
| 141731 | −0.4531468 | |
| 490484 | −0.3388325 | ESTs |
| 504959 | −0.1620065 | Homo sapiens mRNA; cDNA DKFZp586G0321 (from clone |
| | | DKFZp586G0321) |
| 1609746 | −0.4792809 | vitelliform macular dystrophy (Best disease, bestrophin) |
| 882248 | −0.8700731 | transgelin |
| 1917449 | −1.0894686 | serum amyloid A4, constitutive |
| 2119838 | −0.3045374 | a disintegrin-like and metalloprotease (reprolysin type) with |
| | | thrombospondin type 1 motif, 8 |
| 841507 | 0.0247736 | surfactant, pulmonary-associated protein A2 |
| 813265 | −1.2230435 | Homo sapiens mRNA; cDNA DKFZp564H1916 (from clone |
| | | DKFZp564H1916) |
| 767202 | −1.0654145 | latent transforming growth factor beta binding protein 2 |
| 1156538 | 0.2252628 | potassium inwardly-rectifying channel, subfamily J, member |
| | | 11 |
| 39600 | −0.6540275 | adenylate kinase 5 |
| 1630990 | −0.7312981 | ribosomal protein L29 |
| 41208 | −0.865227 | bone morphogenetic protein 1 |
| 322561 | −0.7970805 | ribosomal protein L31 |
| 951008 | −0.2221875 | ESTs |
| 841308 | −0.9953716 | myosin, light polypeptide kinase |
| 119290 | −0.8224756 | cortic al thymocyte receptor (X. laevis CTX) like |
| 344959 | −0.0998837 | gene for serine/threonine protein kinase |
| 810331 | −0.1389612 | quiescin Q6 |
| 1161775 | −0.7380353 | villin 1 |
| 625399 | −0.9066615 | hypothetical protein similar to beta-transducin family |
| 1470657 | −0.3557985 | deiodinase, iodothyronine, type II |
| 160609 | −0.8405669 | ESTs |
| 665356 | −0.1575968 | tumor necrosis factor receptor superfamily, member 11b |
| | | (osteoprotegerin) |
| 1584540 | −0.9754693 | Homo sapiens mRNA; cDNA DKFZp586M2022 (from clone |
| | | DKFZp586M2022) |
| 726779 | −0.4459955 | calponin 1, basic, smooth muscle |
| 296123 | −0.2814132 | Homo sapiens PRO1851 mRNA, complete cds |
| 190059 | −0.634814 | guanine nucleotide binding protein (G protein), gamma 7 |
| 1471829 | −0.7275378 | uncharacterized hypothalamus protein HSMNP1 |
| 2056139 | −1.0673271 | LIM domain protein |
| 190753 | −0.4423669 | ESTs |
| 1968422 | −0.5802058 | Homo sapiens mRNA full length insert cDNA clone |
| | | EUROIMAGE 1968422 |
| 725390 | −0.4620278 | glutathione S-transferase pi |
| 814826 | −0.8443265 | ESTs |
| 878836 | −1.037352 | secretory granule, neuroendocrine protein 1 (7B2 protein) |
| 27769 | −0.8298821 | ESTs |
| 188388 | −0.6824191 | integrin, alpha 10 |
| 1597813 | −0.5406089 | hypothetical protein PP1044 |
| 1609625 | −0.5920312 | selectin P ligand |
| 810981 | −0.1805871 | hypothetical protein FLJ20699 |
| 726703 | −0.070084 | Homo sapiens clone 23736 mRNA sequence |
| 781014 | −1.0161379 | suppression of tumorigenicity 5 |
| 898222 | 0.1795078 | Homo sapiens clone 24418 mRNA sequence |
| 1475738 | −0.6753091 | ribosomal protein S25 |
| 1492144 | −0.2356446 | butyrophilin, subfamily 3, member A2 |
| 1492147 | −0.636656 | ribosomal protein S4, X-linked |
| 1711456 | −0.4896574 | H factor (complement)-like 1 |
| 1871116 | −0.9821709 | Homo sapiens mRNA; cDNA DKFZp434C1714 (from clone |
| | | DKFZp434C1714); partial cds |
| 344720 | −1.5196431 | glycophorin C (Gerbich blood group) |
| 45099 | −1.3515907 | regucalcin (senescence marker protein-30) |
| 307029 | −0.6500872 | ribosomal protein L26 |
| 25763 | −0.2725443 | ankylosis, progressive (mouse) homolog |
| 2502722 | −0.3315871 | loss of heterozygosity, 11, chromosomal region 2, gene A |
| 1665444 | −0.651994 | tumor endothelial marker 1 precursor |
| 165837 | −0.3443143 | translocase of inner mitochondrial membrane 22 (yeast) |
| | | homolog |
| 502518 | −0.4342228 | laminin, beta 2 (laminin S) |
| 897963 | −0.9775616 | phosphatidic acid phosphatase type 2A |
| 120138 | 0.1570107 | J domain containing protein 1 |
| 78946 | −0.648366 | Homo sapiens pyruvate dehydrogenase kinase 4 mRNA, 3′ |
| | | untranslated region, partial sequence |
| 990881 | −0.8335353 | |
| 859192 | −0.2265317 | vesicle-associated membrane protein 1 (synaptobrevin 1) |
| 712023 | −0.5302372 | AT-binding transcription factor 1 |
| 855586 | −0.3944697 | nuclear receptor subfamily 3, group C, member 1 |
| 52419 | −1.0368509 | Friedreich ataxia region gene X123 |
| 432072 | −0.4193898 | nuclear factor of activated T-cells, cytoplasmic, calcineurin- |
| | | dependent 1 |
| 1897947 | −0.163146 | surfactant, pulmonary-associated protein A2 |
| 727229 | −0.2668972 | mitogen-activated protein kinase kinase kinase 4 |
| 810358 | −0.5403705 | acyl-Coenzyme A dehydrogenase, very long chain |
| 154600 | −0.0683999 | phospholipase C, delta 1 |
| 755855 | −0.6298333 | solute carrier family 25 (mitochondrial carrier; adenine |
| | | nucleotide translocator), member 6 |
| 323780 | −0.8879854 | Homo sapiens cDNA FLJ11177 fis, clone PLACE1007402 |
| 230560 | −0.1803944 | ESTs |
| 283173 | −0.4293696 | EBP50-PDZ interactor of 64 kD |
| 265853 | −0.3519023 | Homo sapiens cDNA FLJ11298 fis, clone PLACE1009794 |
| 161456 | −0.8215502 | serum amyloid A1 |
| 184022 | −0.7070266 | amyloid beta (A4) precursor protein-binding, family B, |
| | | member 1 (Fe65) |
| 1636523 | −0.1496249 | glutathione S-transferase subunit 13 homolog |
| 795730 | −0.2892776 | signal transduction protein (SH3 containing) |
| 1631682 | −0.5162406 | peptidylprolyl isomerase E (cyclophilin E) |
| 811837 | −0.848137 | |
| 854763 | −0.0493902 | Homo sapiens cDNA FLJ11341 fis, clone PLACE1010786 |
| 50562 | −0.0943363 | chromosome 8 open reading frame 4 |
| 46843 | −0.1736687 | neuronal Shc adaptor homolog |
| 178137 | −0.6839022 | ribosomal protein L34 |
| 1662279 | −0.2290724 | plakophilin 1 (ectodermal dysplasia/skin fragility syndrome) |
| 1635062 | −0.8243024 | DKFZP586A011 protein |
| 293916 | −0.3671832 | FKBP-associated protein |
| 415613 | −0.5528761 | DHHC1 protein |
| 80344 | −0.8243418 | interleukin 7 receptor |
| 1602798 | −0.4300808 | choline kinase-like |
| 1910516 | −0.2845842 | ESTs |
| 740620 | −0.96496 | tropomyosin 2 (beta) |
| 277627 | −0.5329107 | Human SH3 domain-containing protein SH3P18 mRNA, |
| | | complete cds |
| 854696 | 0.008949 | siah binding protein 1; FBP interacting repressor; pyrimidine |
| | | tract binding splicing factor; Ro ribonucleoprotein-binding |
| | | protein 1 |
| 45578 | −0.1295485 | mitogen-activated protein kinase kinase 6 |
| 950710 | −0.346361 | propionyl Coenzyme A carboxylase, alpha polypeptide |
| 768043 | −0.4005598 | ECSIT |
| 308539 | −0.6726187 | Homo sapiens cDNA FLJ12777 fis, clone NT2RP2001720 |
| 595637 | −0.5373697 | Homo sapiens mRNA; cDNA DKFZp586N012 (from clone |
| | | DKFZp586N012) |
| 143661 | −0.4713778 | netrin 4 |
| 248631 | −0.9106505 | aminomethyltransferase (glycine cleavage system protein T) |
| 826622 | 0.1339287 | KIAA0430 gene product |
| 51981 | −0.5554908 | ribosomal protein L7a |
| 811848 | −1.1350073 | hypothetical protein |
| 1533710 | −0.5988282 | ortholog of mouse integral membrane glycoprotein LIG-1 |
| 2017756 | −0.2549438 | homolog of yeast MOG1 |
| 1637296 | −0.5105214 | ribosomal protein S24 |
| 50586 | −0.6794282 | KIAA1545 protein |
| 877835 | −0.5382125 | ribosomal protein L35 |
| 773319 | −0.285447 | ribosomal protein S6 kinase, 70 kD, polypeptide 1 |
| 2014888 | −0.2871529 | sushi-repeat protein |
| 767495 | −0.0366213 | GLI-Kruppel family member GLI3 (Greig |
| | | cephalopolysyndactyly syndrome) |
| 774078 | −0.6508021 | leiomodin 1 (smooth muscle) |
| 868400 | −0.4928479 | glutaminyl-tRNA synthetase |
| 240620 | −0.5065007 | vascular Rab-GAP/TBC-containing |
| 1588791 | −0.1606643 | O-6-methylguanine-DNA methyltransferase |
| 472186 | −0.2687871 | RAB32, member RAS oncogene family |
| 725143 | −0.3410957 | hypothetical protein FLJ22418 |
| 714472 | −0.3677387 | KIAA0397 gene product |
| 1854648 | 0.0089186 | hemopexin |
| 855029 | 0.076972 | Ac-like transposable element |
| 197727 | −0.3906866 | phosphatidylethanolamine N-methyltransferase |
| 1623016 | −0.6263561 | EST |
| 813841 | 0.0885625 | plasminogen activator, tissue |
| 2783721 | −0.6854649 | cholinergic receptor, nicotinic, beta polypeptide 1 (muscle) |
| 549933 | −0.3547881 | interleukin 8 |
| 280907 | −0.7307519 | Kruppel-type zinc finger protein |
| 796181 | −0.8888496 | growth arrest-specific 6 |
| 415233 | −0.6506593 | ribosomal protein L37a |
| 811920 | −1.2690713 | interleukin 11 receptor, alpha |
| 415415 | −0.646492 | EST, Moderately similar to RL1X_HUMAN 60S RIBOSOMAL |
| | | PROTEIN L18A [H. sapiens] |
| 2018807 | −0.2989936 | KIAA0468 gene product |
| 154999 | −0.3876757 | hypothetical protein FLJ21007 |
| 68557 | 0.0637586 | fatty acid binding protein 1, liver |
| 647763 | −0.1436566 | ESTs |
| 1568967 | −0.2938468 | ESTs |
| 210717 | −0.4522432 | syndecan 2 (heparan sulfate proteoglycan 1, cell surface- |
| | | associated, fibroglycan) |
| 1350439 | −0.6740833 | KIAA0015 gene product |
| 684582 | −0.0089016 | tryptophanyl tRNA synthetase 2 (mitochondrial) |
| 122394 | −0.049399 | guanine nucleotide binding protein (G protein), alpha 15 (Gq |
| | | class) |
| 213136 | −0.1139004 | BTG family, member 2 |
| 625458 | −0.2394796 | hypothetical protein MGC3234 |
| 811162 | −0.3821116 | fibromodulin |
| 1926246 | −0.701769 | ESTs, Moderately similar to T46371 hypothetical protein |
| | | DKFZp434P1018.1 [H. sapiens] |
| 1554167 | −1.0818834 | hypothetical protein FLJ14529 |
| 811088 | −1.0851546 | ephrin-B3 |
| 72778 | −1.1752838 | caspase 7, apoptosis-related cysteine protease |
| 220293 | −0.5078291 | Homo sapiens cDNA: FLJ21800 fis, clone HEP00618 |
| 1569187 | −1.2971252 | heparan sulfate (glucosamine) 3-O-sulfotransferase 4 |
| 866866 | −0.4801351 | Ras association (RalGDS/AF-6) domain family 1 |
| 81316 | −0.6176265 | ESTs |
| 1161564 | −1.3891189 | desmuslin |
| 898044 | −0.4691863 | metallocarboxypeptidase CPX-1 |
| 293001 | −0.4472585 | hypothetical protein DKFZp434E2318 |
| 1570502 | −0.3314609 | Homo sapiens cDNA FLJ12936 fis, clone NT2RP2005018 |
| 839796 | −0.6807912 | candidate tumor suppressor p33 ING1 homolog |
| 380883 | −0.4438655 | Homo sapiens cDNA FLJ10158 fis, clone HEMBA1003463 |
| 180561 | −0.3250633 | glutathione S-transferase M4 |
| 869450 | −0.7659922 | ribosomal protein L11 |
| 47043 | −0.7729643 | tensin |
| 810463 | −0.0829995 | DKFZP566O084 protein |
| 149539 | 0.4138942 | KIAA1700 |
| 200354 | −0.0613983 | thymidine kinase 2, mitochondrial |
| 771173 | 0.1757755 | mitochondrial ribosomal protein S21 |
| 270826 | −0.9389651 | Homo sapiens cDNA FLJ13329 fis, clone OVARC1001795 |
| 810017 | −0.1076821 | plasminogen activator, urokinase receptor |
| 809838 | 0.0956351 | |
| 344168 | −0.386185 | polymerase (DNA directed), lambda |
| 785967 | −0.6049171 | erythrocyte membrane protein band 4.1-like 2 |
| 511831 | −0.2912672 | hypothetical protein MGC12936 |
| 282404 | −0.5528532 | Homo sapiens mRNA for KIAA1671 protein, partial cds |
| 730036 | −0.079736 | Mad4 homolog |
| 298231 | −0.3550893 | gamma-aminobutyric acid (GABA) B receptor, 1 |
| 2106144 | −1.067765 | regulated in glioma |
| 743880 | −0.3262292 | KIAA0263 gene product |
| 268234 | −0.1535073 | Dmx-like 1 |
| 280776 | −0.2922639 | acyl-Coenzyme A dehydrogenase, C-2 to C-3 short chain |
| 212078 | −0.1831462 | integrin, alpha 1 |
| 731308 | 0.5371204 | citrate synthase |
| 774471 | −0.1753902 | laminin, beta 1 |
| 811837 | −0.8764231 | eukaryotic translation elongation factor 1 alpha 1 |
| 714437 | 0.0258281 | sema domain, immunoglobulin domain (Ig), transmembrane |
| | | domain (TM) and short cytoplasmic domain, (semaphorin) 4B |
| 416959 | −0.957248 | nuclear factor I/B |
| 681992 | −0.7691299 | Homo sapiens cDNA FLJ13384 fis, clone PLACE1001062, |
| | | highly similar to Homo sapiens mRNA for lysine- |
| | | ketoglutarate reductase/saccharopine dehydrogenase |
| 130201 | −1.0401997 | intercellular adhesion molecule 2 |
| 363144 | 0.1937891 | transcription factor AP-2 beta (activating enhancer-binding |
| | | protein 2 beta) |
| 857874 | −0.3703645 | transforming growth factor beta-activated kinase-binding |
| | | protein 1 |
| 377461 | −1.2798035 | caveolin 1, caveolae protein, 22 kD |
| 156363 | 0.2685238 | hypothetical protein FLJ12934 |
| 589115 | −0.0115547 | matrix metalloproteinase 1 (interstitial collagenase) |
| 241489 | −0.6212938 | adrenergic, beta-2-, receptor, surface |
| 586725 | 0.0683397 | protein phosphatase 2, regulatory subunit B (B56), beta |
| | | isoform |
| 283124 | −0.7933772 | ESTs, Moderately similar to LONG-CHAIN FATTY ACID |
| | | TRANSPORT PROTEIN [M. musculus] |
| 307328 | −0.4022704 | hypothetical protein FLJ10948 |
| 37449 | −0.3539978 | GAS2-related on chromosome 22 |
| 50892 | −0.6887497 | Homo sapiens, clone MGC: 9913 IMAGE: 3870821, mRNA, |
| | | complete cds |
| 1897944 | −0.4855156 | ESTs, Weakly similar to T00366 hypothetical protein |
| | | KIAA0669 [H. sapiens] |
| 377314 | −0.561312 | casein kinase 2, alpha prime polypeptide |
| 220851 | −0.0351418 | crystallin, alpha A |
| 322233 | −0.3025167 | |
| 586803 | −0.7423407 | placental growth factor, vascular endothelial growth factor- |
| | | related protein |
| 343695 | −0.7706809 | hypothetical protein FLJ10875 |
| 414999 | −0.5002542 | ets variant gene 4 (E1A enhancer-binding protein, E1AF) |
| 813823 | −0.4463267 | lumican |
| 178825 | −0.1525189 | neurogranin (protein kinase C substrate, RC3) |
| 85582 | −0.0716296 | target of myb1 (chicken) homolog-like 2 |
| 753620 | −0.5248489 | insulin-like growth factor binding protein 6 |
| 1899338 | −0.066168 | mannosidase, alpha, class 1A, member 2 |
| 359250 | 0.0674486 | carbonic anhydrase IV |
| 345034 | −1.0600859 | small inducible cytokine subfamily B (Cys-X-Cys), member |
| | | 14 (BRAK) |
| 2164744 | −0.2307289 | neural cell adhesion molecule 1 |
| 613056 | −0.3957342 | reticulocalbin 1, EF-hand calcium binding domain |
| 1410444 | −0.0368027 | amphiregulin (schwannoma-derived growth factor) |
| 593023 | −0.7516846 | dystrobrevin, beta |
| 788511 | 0.1403148 | ribosomal protein S6 kinase, 90 kD, polypeptide 1 |
| 455269 | 0.0223607 | |
| 1650927 | −0.1712163 | hypothetical protein DKFZp547E052 |
| 782427 | −0.2818886 | inhibin, beta B (activin AB beta polypeptide) |
| 71727 | −0.2796517 | T-cell acute lymphocytic leukemia 1 |
| 840942 | −0.6383764 | major histocompatibility complex, class II, DP beta 1 |
| 825470 | 0.5218731 | topoisomerase (DNA) II alpha (170 kD) |
| 595213 | 0.4824381 | hypothetical protein |
| 796694 | 0.6893307 | baculoviral IAP repeat-containing 5 (survivin) |
| 810711 | 0.9128832 | stearoyl-CoA desaturase (delta-9-desaturase) |
| 629944 | 0.5273447 | myosin VB |
| 66406 | 0.5966701 | hypothetical protein DKFZp762E1312 |
| 785840 | 0.5401209 | SEC24 (S. cerevisiae) related gene family, member D |
| 210862 | 0.4683996 | acyl-Coenzyme A oxidase 1, palmitoyl |
| 84295 | 0.3989864 | interleukin 1 receptor antagonist |
| 429182 | 0.2952867 | dolichyl-phosphate mannosyltransferase polypeptide 1, |
| | | catalytic subunit |
| 705064 | 0.4497555 | transforming, acidic coiled-coil containing protein 3 |
| 782513 | 0.4759571 | interferon, alpha-inducible protein (clone IFI-6-16) |
| 839682 | 0.5208954 | ubiquitin-conjugating enzyme E2N (homologous to yeast |
| | | UBC13) |
| 768377 | 0.3809674 | activity-dependent neuroprotective protein |
| 2309073 | 0.5226599 | frizzled (Drosophila) homolog 5 |
| 789376 | 0.4339171 | thioredoxin reductase 1 |
| 196992 | −0.1015205 | aldo-keto reductase family 1, member C1 (dihydrodiol |
| | | dehydrogenase 1; 20-alpha (3-alpha)-hydroxysteroid |
| | | dehydrogenase) |
| 785707 | 0.4708376 | protein regulator of cytokinesis 1 |
| 1505038 | 1.1904802 | hypothetical protein FLJ20171 |
| 869375 | 0.4827093 | isocitrate dehydrogenase 2 (NADP+), mitochondrial |
| 109221 | 0.3048287 | KIAA0286 protein |
| 345787 | 0.4647372 | highly expressed in cancer, rich in leucine heptad repeats |
| 46248 | 0.640944 | ADP-ribosyltransferase (NAD+; poly (ADP-ribose) |
| | | polymerase) |
| 79520 | 0.6769129 | RAB2, member RAS oncogene family |
| 469383 | 0.5558732 | chromosome 8 open reading frame 1 |
| 509588 | 0.4157059 | TATA box binding protein (TBP)-associated factor, RNA |
| | | polymerase II, J, 20 kD |
| 292936 | 0.4217115 | hypothetical protein FLJ10468 |
| 686172 | 0.6724684 | KIAA0008 gene product |
| 788655 | 0.2976191 | HTPAP protein |
| 770992 | 0.3331315 | |
| 1587847 | 0.1732392 | minichromosome maintenance deficient (mis5, S. pombe) 6 |
| 79710 | 0.4579008 | KIAA0174 gene product |
| 2056566 | 0.2341905 | integrin, alpha M (complement component receptor 3, alpha; |
| | | also known as CD11b (p170), macrophage antigen alpha |
| | | polypeptide) |
| 454339 | 0.3738929 | thiopurine S-methyltransferase |
| 594226 | 0.403676 | Homo sapiens cDNA FLJ14459 fis, clone HEMBB1002409 |
| 897813 | 0.4749348 | polyadenylate binding protein-interacting protein 1 |
| 824694 | 0.0268936 | protein tyrosine phosphatase type IVA, member 1 |
| 725454 | 0.6669973 | CDC28 protein kinase 2 |
| 79254 | 0.0732432 | MHC class I region ORF |
| 1472719 | 0.2550065 | SMT3 (suppressor of mif two 3, yeast) homolog 1 |
| 2054635 | 0.7914755 | proteasome (prosome, macropain) subunit, alpha type, 7 |
| 289978 | 0.6308861 | ubiquitin-like 4 |
| 155806 | 0.2747101 | 2′-5′-oligoadenylate synthetase 2 (69-71 kD) |
| 147834 | 0.4412795 | zinc finger protein 217 |
| 26171 | 0.3656147 | KIAA0856 protein |
| 2322367 | 0.5832711 | reticulon 4 |
| 769921 | 0.6464245 | ubiquitin carrier protein E2-C |
| 73009 | 0.4822587 | ESTs, Weakly similar to A43932 mucin 2 precursor, intestinal |
| | | [H. sapiens] |
| 2014034 | 0.6620303 | methylene tetrahydrofolate dehydrogenase (NAD+ |
| | | dependent), methenyltetrahydrofolate cyclohydrolase |
| 359887 | 0.8692353 | translocase of inner mitochondrial membrane 17 (yeast) |
| | | homolog A |
| 488964 | 0.9569176 | H2A histone family, member O |
| 1476065 | 0.289904 | stathmin 1/oncoprotein 18 |
| 1422338 | 0.5874097 | ribonucleotide reductase M2 polypeptide |
| 268946 | 0.5440003 | WD40 protein Ciao1 |
| 686552 | 0.7231319 | golgi phosphoprotein 1 |
| 149544 | 0.283642 | neuroepithelial cell transforming gene 1 |
| 770675 | 0.454729 | Homo sapiens cDNA: FLJ21323 fis, clone COL02374 |
| 346257 | 0.1515899 | minichromosome maintenance deficient (S. cerevisiae) 4 |
| 429799 | 0.6915155 | hypothetical protein FLJ21939 similar to 5-azacytidine |
| | | induced gene 2 |
| 143997 | 0.1883447 | proteasome (prosome, macropain) 26S subunit, non- |
| | | ATPase, 10 |
| 122241 | 0.7394284 | proteasome (prosome, macropain) subunit, beta type, 2 |
| 823598 | 0.9153521 | proteasome (prosome, macropain) 26S subunit, non- |
| | | ATPase, 12 |
| 814632 | 0.0960611 | splicing factor, arginine/serine-rich (transformer 2 Drosophila |
| | | homolog) 10 |
| 810316 | 0.3732635 | very long-chain acyl-CoA synthetase; lipidosin |
| 290841 | 0.8346933 | H2B histone family, member A |
| 347373 | 0.7563599 | transcription elongation factor B (SIII), polypeptide 1 (15 kD, |
| | | elongin C) |
| 700792 | 0.4949149 | cyclin-dependent kinase inhibitor 3 (CDK2-associated dual |
| | | specificity phosphatase) |
| 1554549 | 0.9764206 | hydroxyacyl glutathione hydrolase |
| 897770 | 0.4270685 | |
| 504308 | 0.5564295 | hypothetical protein FLJ10540 |
| 132828 | 0.2875519 | Down syndrome critical region gene 1-like 2 |
| 292388 | 0.112577 | |
| 1616253 | 0.567837 | breast carcinoma amplified sequence 1 |
| 796469 | 1.0269115 | HSPC150 protein similar to ubiquitin-conjugating enzyme |
| 2139152 | −0.4373776 | Homo sapiens clone 24473 mRNA sequence |
| 130276 | 0.2154295 | Homo sapiens mRNA; cDNA DKFZp586H0324 (from clone |
| | | DKFZp586H0324) |
| 34149 | 0.309569 | KIAA0227 protein |
| 150003 | 0.8251408 | hypothetical protein FLJ13187 |
| 287749 | 0.3173706 | CDC7 (cell division cycle 7, S. cerevisiae, homolog)-like 1 |
| 25380 | 0.164077 | ESTs, Moderately similar to JC5238 galactosylceramide-like |
| | | protein, GCP [H. sapiens] |
| 745083 | 0.4107735 | ubiquitin specific protease 18 |
| 810899 | 0.240336 | CD028 protein kinase 1 |
| 768059 | 0.3139886 | hypothetical protein FLJ12619 |
| 815026 | 0.4472842 | cleavage stimulation factor, 3′ pre-RNA, subunit 3, 77 kD |
| 741474 | 0.7732047 | glucose phosphate isomerase |
| 788641 | 0.3638059 | adaptor-related protein complex 1, sigma 2 subunit |
| 2043167 | 0.7167431 | BCL2-associated athanogene 3 |
| 1492780 | 0.6544659 | Homo sapiens cDNA FLJ14459 fis, clone HEMBB1002409 |
| 2306987 | 0.4402912 | secreted and transmembrane 1 |
| 754653 | 0.4405873 | cadherin, EGF LAG seven-pass G-type receptor 3, flamingo |
| | | (Drosophila) homolog |
| 743810 | 0.3667087 | hypothetical protein MGC2577 |
| 126858 | 0.2389814 | sterol O-acyltransferase (acyl-Coenzyme A: cholesterol |
| | | acyltransferase) 1 |
| 2017415 | 0.43546 | centromere protein A (17 kD) |
| 280507 | 0.2846518 | hypoxanthine phosphoribosyltransferase 1 (Lesch-Nyhan |
| | | syndrome) |
| 67237 | 0.1406869 | ESTs |
| 43833 | −0.1197546 | diacylglycerol kinase, gamma (90 kD) |
| 42076 | 0.3194462 | TRK-fused gene |
| 531319 | 0.1093932 | serine/threonine kinase 12 |
| 784129 | 0.2902598 | tyrosine 3-monooxygenase/tryptophan 5-monooxygenase |
| | | activation protein, gamma polypeptide |
| 586895 | 0.2549108 | small nuclear ribonucleoprotein polypeptide G |

### Example VIII

### Genes for Discriminating Between DCIS Grade I and Grade III

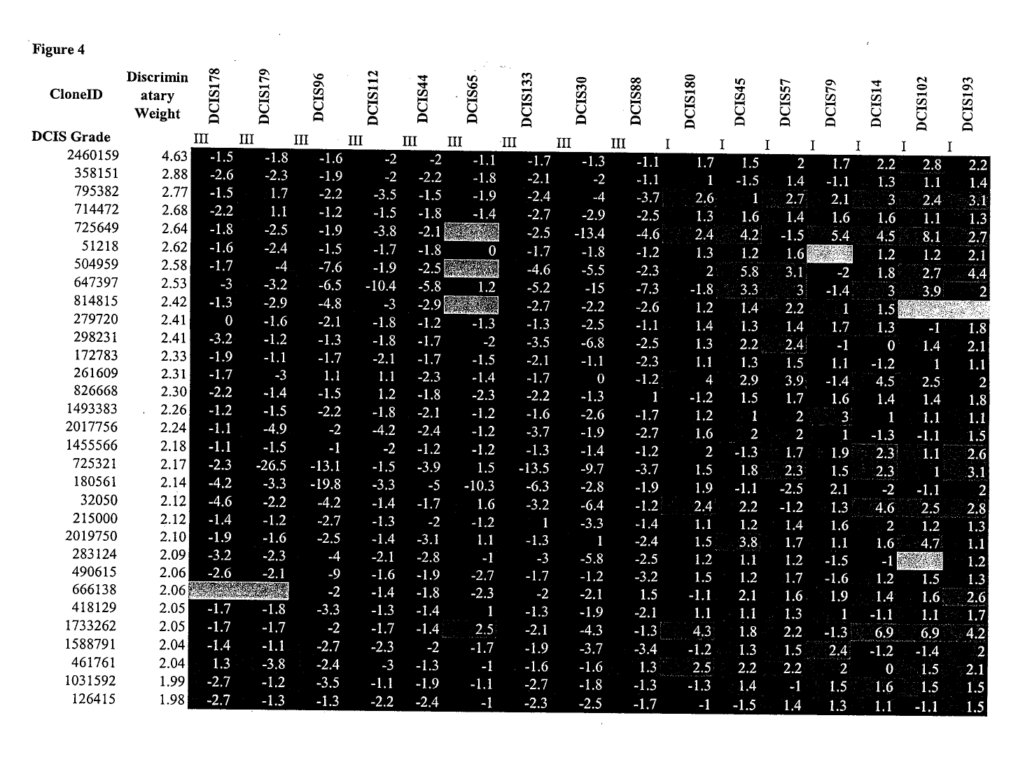

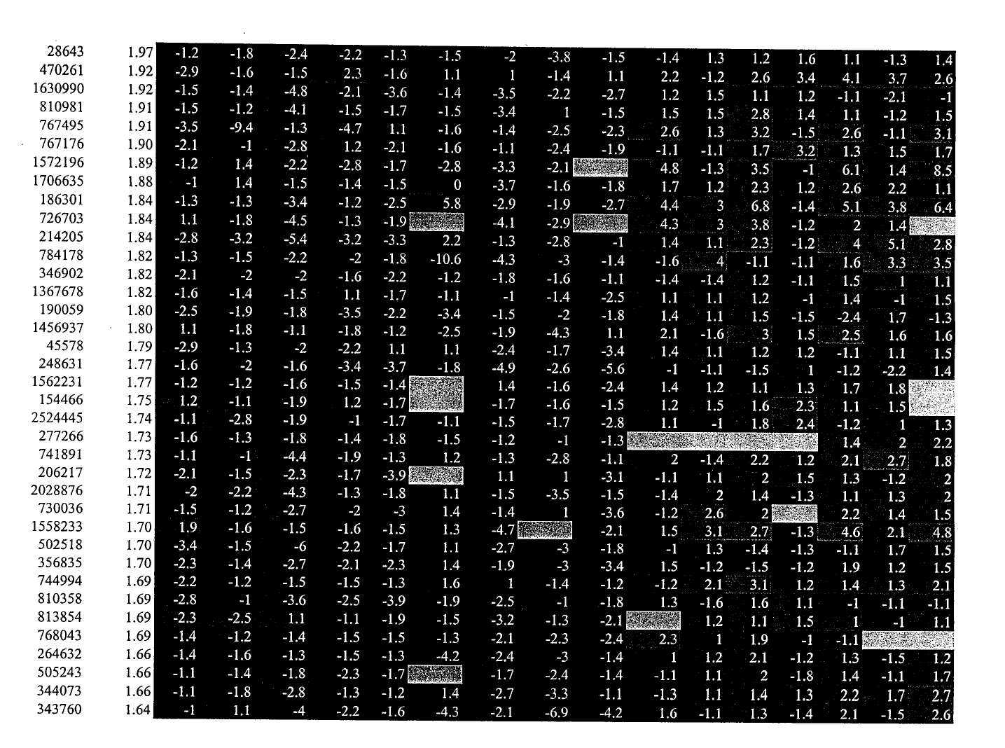

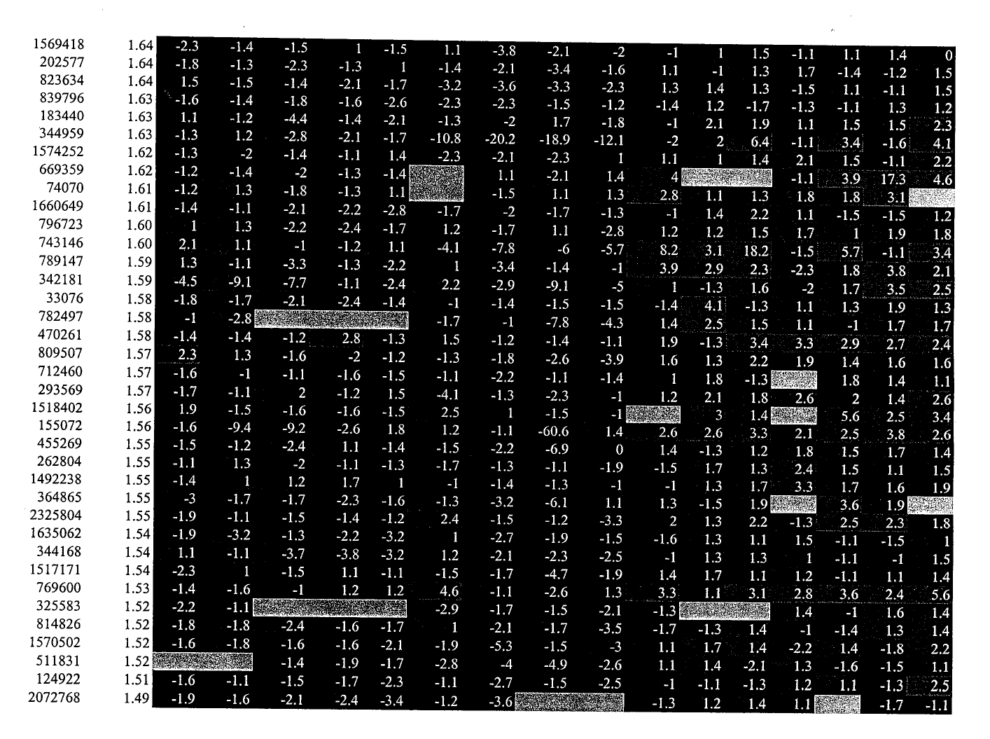

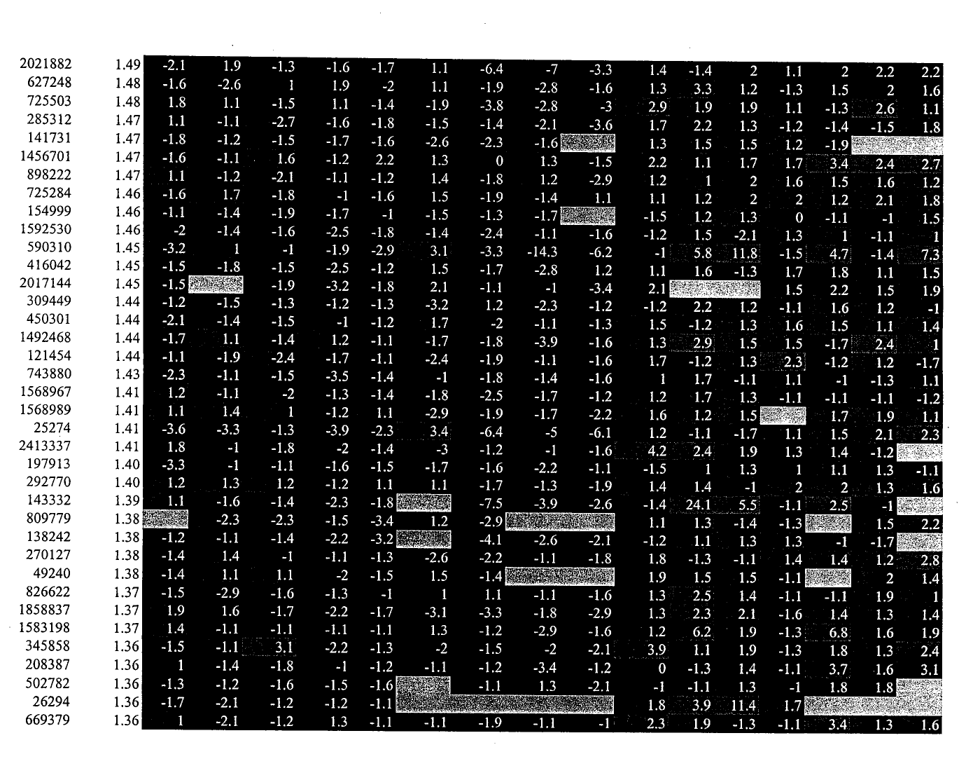

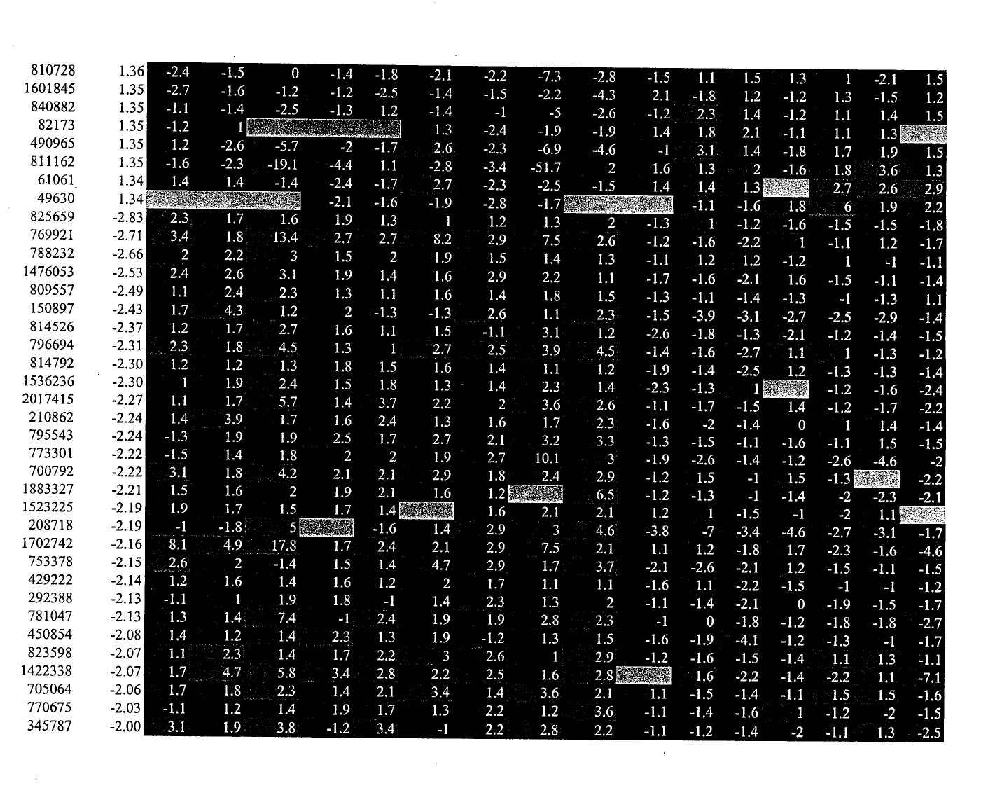

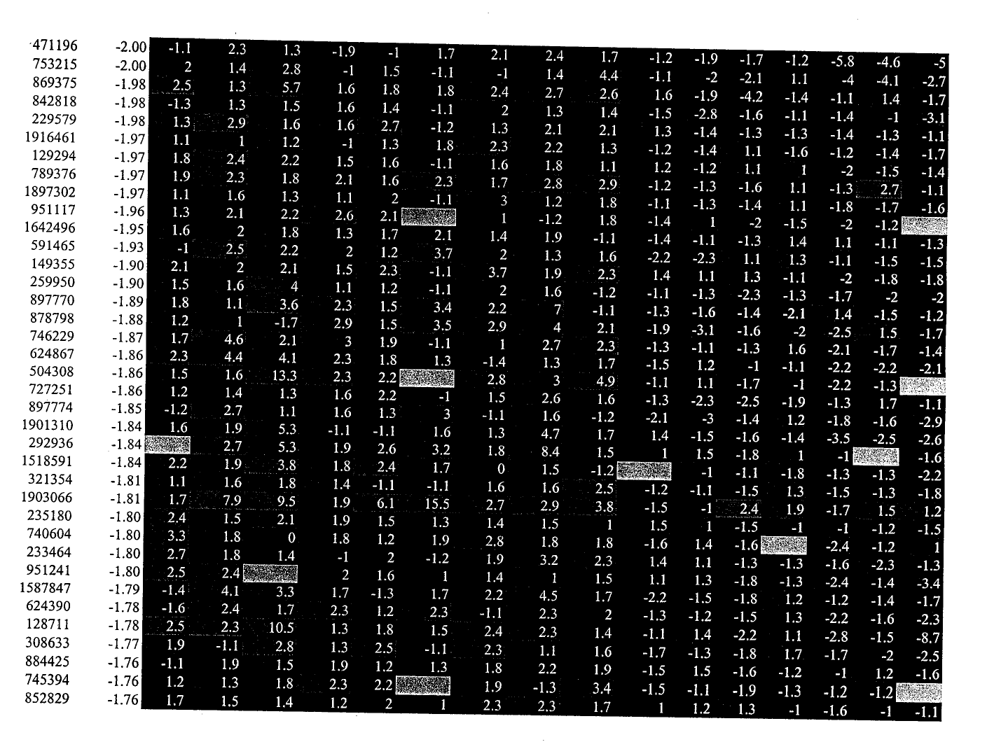

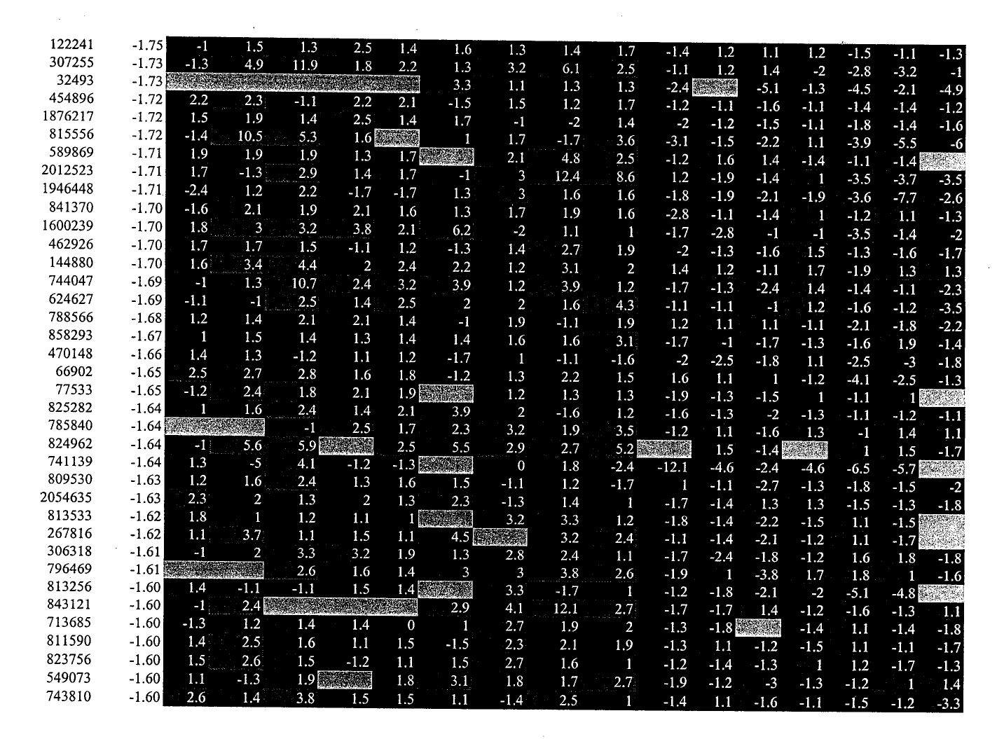

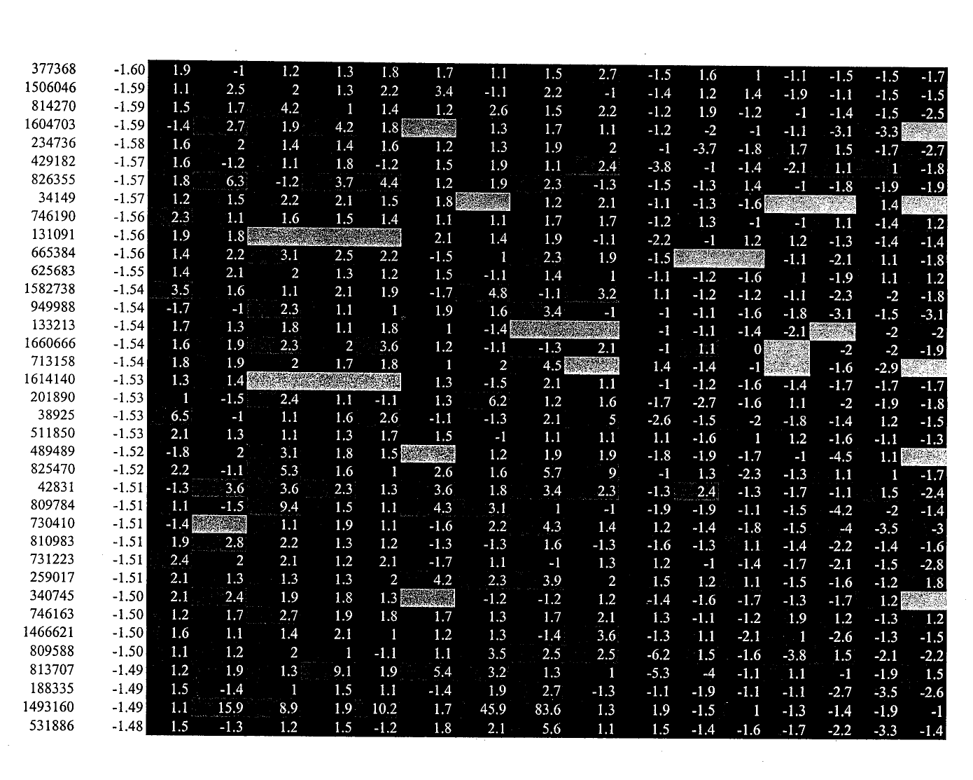

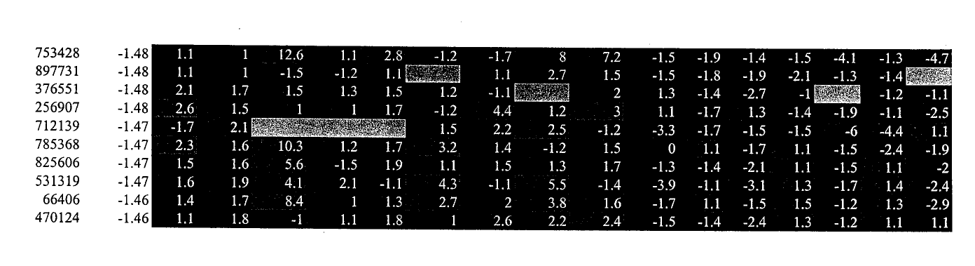

As shown in Table 7 below, 350 genes were identified as being able to discriminate between grades of DCIS. The actual data corresponding to this table is shown in FIG. 4.

TABLE 7

| CloneID | Weight | Description |
|---|---|---|
| 2460159 | 4.6268975 | tyrosine kinase, non-receptor, 1 |
| 358151 | 2.8783989 | zinc finger protein 33a (KOX 31) |
| 795382 | 2.772572 | Rap1 guanine-nucleotide-exchange factor directly activated by |
| | | cAMP |
| 714472 | 2.6829714 | KIAA0397 gene product |
| 725649 | 2.6433625 | nuclear factor of activated T-cells, cytoplasmic, calcineurin- |
| | | dependent 4 |
| 51218 | 2.6200167 | ESTs |
| 504959 | 2.5777963 | Homo sapiens mRNA; cDNA DKFZp586G0321 (from clone |
| | | DKFZp586G0321) |
| 647397 | 2.5320153 | ESTs |
| 814815 | 2.4198892 | plakophilin 4 |
| 279720 | 2.4136316 | ESTs, Moderately similar to A47582 B-cell growth factor |
| | | precursor [H. sapiens] |
| 298231 | 2.4096172 | gamma-aminobutyric acid (GABA) B receptor, 1 |
| 172783 | 2.3339699 | hypothetical protein FLJ10390 |
| 261609 | 2.311051 | ESTs, Weakly similar to S65657 alpha-1C-adrenergic receptor |
| | | splice form 2 [H. sapiens] |
| 826668 | 2.2951137 | KIAA0274 gene product |
| 1493383 | 2.2616737 | Homo sapiens mRNA; cDNA DKFZp434H2418 (from clone |
| | | DKFZp434H2418) |
| 2017756 | 2.2403622 | homolog of yeast MOG1 |
| 1455566 | 2.1756946 | adenosine A3 receptor |
| 725321 | 2.1658095 | estrogen receptor 1 |
| 180561 | 2.1393944 | glutathione S-transferase M4 |
| 32050 | 2.1165022 | Homo sapiens mRNA; cDNA DKFZp586P1124 (from clone |
| | | DKFZp586P1124) |
| 215000 | 2.1150123 | vasoactive intestinal peptide receptor 1 |
| 2019750 | 2.0957741 | SEC14 (S. cerevisiae)-like 2 |
| 283124 | 2.0921779 | ESTs, Moderately similar to LONG-CHAIN FATTY ACID |
| | | TRANSPORT PROTEIN [M. musculus] |
| 490615 | 2.0635424 | tubulin, gamma 2 |
| 666138 | 2.0625667 | hypothetical protein DKFZp761J1523 |
| 418129 | 2.0541212 | nuclear mitotic apparatus protein 1 |
| 1733262 | 2.052666 | BLu protein |
| 1588791 | 2.044069 | O-6-methylguanine-DNA methyltransferase |
| 461761 | 2.035953 | angiogenin, ribonuclease, RNase A family, 5 |
| 1031592 | 1.9930012 | kinesin protein 9 gene |
| 126415 | 1.9781057 | Homo sapiens mRNA; cDNA DKFZp566H0124 (from clone |
| | | DKFZp566H0124) |
| 28643 | 1.9733043 | hypothetical protein DKFZp564D1378 |
| 470261 | 1.9160486 | |
| 1630990 | 1.9155421 | ribosomal protein L29 |
| 810981 | 1.9132205 | hypothetical protein FLJ20699 |
| 767495 | 1.9052671 | GLI-Kruppel family member GLI3 (Greig |
| | | cephalopolysyndactyly syndrome) |
| 767176 | 1.8996792 | tumor necrosis factor (ligand) superfamily, member 13 |
| 1572196 | 1.8855996 | secreted modular calcium-binding protein 2 |
| 1706635 | 1.8754346 | bone gamma-carboxyglutamate (gla) protein (osteocalcin) |
| 186301 | 1.8424458 | Homo sapiens cDNA FLJ12924 fis, clone NT2RP2004709 |
| 726703 | 1.842399 | Homo sapiens clone 23736 mRNA sequence |
| 214205 | 1.8416657 | Homo sapiens, clone MGC: 17687 IMAGE: 3865868, mRNA, |
| | | complete cds |
| 784178 | 1.822159 | Homo sapiens mRNA; cDNA DKFZp586M0723 (from clone |
| | | DKFZp586M0723) |
| 346902 | 1.8196723 | ESTs, Weakly similar to ALU2_HUMAN ALU SUBFAMILY SB |
| | | SEQUENCE CONTAMINATION WARNING ENTRY |
| | | [H. sapiens] |
| 1367678 | 1.8184255 | KIAA0356 gene product |
| 190059 | 1.7988757 | guanine nucleotide binding protein (G protein), gamma 7 |
| 1456937 | 1.7980645 | oviductal glycoprotein 1, 120 kD (mucin 9, oviductin) |
| 45578 | 1.7935215 | mitogen-activated protein kinase kinase 6 |
| 248631 | 1.7701919 | aminomethyltransferase (glycine cleavage system protein T) |
| 1562231 | 1.7668811 | SET binding protein 1 |
| 154466 | 1.7516237 | STIP1 homology and U-Box containing protein 1 |
| 2524445 | 1.7392197 | neuronal PAS domain protein 1 |
| 277266 | 1.7307752 | Homo sapiens, clone IMAGE: 3625550, mRNA, partial cds |
| 741891 | 1.7297992 | RAB2, member RAS oncogene family-like |
| 206217 | 1.7227658 | nuclear receptor subfamily 1, group H, member 3 |
| 2028876 | 1.7128178 | splicing factor, arginine/serine-rich 5 |
| 730036 | 1.70573 | Mad4 homolog |
| 1558233 | 1.7035129 | ESTs |
| 502518 | 1.7027287 | laminin, beta 2 (laminin S) |
| 356835 | 1.7014349 | hypothetical protein MGC10500 |
| 744994 | 1.6938348 | hypothetical protein FLJ12242 |
| 810358 | 1.6924263 | acyl-Coenzyme A dehydrogenase, very long chain |
| 813854 | 1.6914133 | purine-rich element binding protein A |
| 768043 | 1.6854123 | ECSIT |
| 264632 | 1.6591646 | ESTs |
| 505243 | 1.6581518 | inositol 1,4,5-triphosphate receptor, type 2 |
| 344073 | 1.6571604 | ESTs, Weakly similar to K1CI_HUMAN KERATIN, TYPE I |
| | | CYTOSKELETAL 9 [H. sapiens] |
| 343760 | 1.6435915 | SH3 domain binding glutamic acid-rich protein like 2 |
| 1569418 | 1.6408767 | Homo sapiens cDNA FLJ11385 fis, clone HEMBA1000520 |
| 202577 | 1.6393518 | histamine N-methyltransferase |
| 823634 | 1.6366047 | ESTs |
| 839796 | 1.630887 | candidate tumor suppressor p33 ING1 homolog |
| 183440 | 1.6297915 | arylsulfatase A |
| 344959 | 1.6268043 | gene for serine/threonine protein kinase |
| 1574252 | 1.6211224 | DKFZP586D0623 protein |
| 669359 | 1.6174855 | Homo sapiens clone 24405 mRNA sequence |
| 74070 | 1.6096611 | endosulfine alpha |
| 1660649 | 1.6051582 | suppressor of white apricot homolog 2 |
| 796723 | 1.6041219 | Homo sapiens clone CDABP0014 mRNA sequence |
| 743146 | 1.597094 | hypothetical protein FLJ23403 |
| 789147 | 1.5937127 | enolase 2, (gamma, neuronal) |
| 342181 | 1.5926355 | B-cell CLL/lymphoma 2 |
| 33076 | 1.5824352 | cholinephosphotransferase 1 |
| 782497 | 1.5804781 | Homo sapiens, clone IMAGE: 3010666, mRNA, partial cds |
| 470261 | 1.5751564 | SMA5 |
| 809507 | 1.5723669 | hypothetical protein FLJ20568 |
| 712460 | 1.5721924 | natural killer-tumor recognition sequence |
| 293569 | 1.5662317 | chromosome 1 open reading frame 21 |
| 1518402 | 1.5627158 | KIAA1361 protein |
| 155072 | 1.5557559 | ESTs |
| 455269 | 1.5538593 | |
| 262804 | 1.5532348 | hypothetical protein MGC2941 |
| 1492238 | 1.5484044 | HSPC003 protein |
| 364865 | 1.5483958 | hypothetical protein FLJ21062 |
| 2325804 | 1.5461823 | 95 kDa retinoblastoma protein binding protein |
| 1635062 | 1.5449944 | DKFZP586A011 protein |
| 344168 | 1.5421405 | polymerase (DNA directed), lambda |
| 1517171 | 1.5381921 | interleukin 2 receptor, alpha |
| 769600 | 1.5335878 | uracil-DNA glycosylase 2 |
| 325583 | 1.5245817 | EST |
| 814826 | 1.5223296 | ESTs |
| 1570502 | 1.5184543 | Homo sapiens cDNA FLJ12936 fis, clone NT2RP2005018 |
| 511831 | 1.5183298 | hypothetical protein MGC12936 |
| 124922 | 1.5052004 | KRAB-zinc finger protein SZF1-1 |
| 2072768 | 1.4886791 | nuclear receptor coactivator 3 |
| 2021882 | 1.4854251 | sodium channel, nonvoltage-gated 1, delta |
| 627248 | 1.4827018 | SBBI31 protein |
| 725503 | 1.476461 | D-dopachrome tautomerase |
| 285312 | 1.4699425 | |
| 141731 | 1.469088 | |
| 1456701 | 1.4668054 | B-cell CLL/lymphoma 9 |
| 898222 | 1.4667947 | Homo sapiens clone 24418 mRNA sequence |
| 725284 | 1.4638006 | phosphorylase kinase, gamma 2 (testis) |
| 154999 | 1.4564204 | hypothetical protein FLJ21007 |
| 1592530 | 1.4558873 | mammalian inositol hexakisphosphate kinase 2 |
| 590310 | 1.4534138 | Homo sapiens mRNA; cDNA DKFZp434E2321 (from clone |
| | | DKFZp434E2321); partial cds |
| 416042 | 1.446691 | ESTs, Weakly similar to ALU1_HUMAN ALU SUBFAMILY J |
| | | SEQUENCE CONTAMINATION WARNING ENTRY |
| | | [H. sapiens] |
| 2017144 | 1.4450059 | CGI-41 protein |
| 309449 | 1.4412699 | ribosomal protein S4, Y-linked |
| 450301 | 1.4403678 | mutL (E. coli) homolog 3 |
| 1492468 | 1.4375076 | DEME-6 protein |
| 121454 | 1.4361788 | arachidonate 12-lipoxygenase |
| 743880 | 1.4294838 | KIAA0263 gene product |
| 1568967 | 1.4109448 | ESTs |
| 1568989 | 1.4094472 | ESTs |
| 25274 | 1.4078252 | g20 protein |
| 2413337 | 1.4058288 | sortilin-related receptor, L(DLR class) A repeats-containing |
| 197913 | 1.3994468 | splicing factor proline/glutamine rich (polypyrimidine tract- |
| | | binding protein-associated) |
| 292770 | 1.3952057 | Homo sapiens, clone IMAGE: 3627860, mRNA, partial cds |
| 143332 | 1.3875805 | neuropeptide Y receptor Y1 |
| 809779 | 1.3848708 | KIAA0239 protein |
| 138242 | 1.3820433 | ESTs, Moderately similar to MAS2_HUMAN MANNAN- |
| | | BINDING LECTIN SERINE PROTEASE 2 PRECURSOR |
| | | [H. sapiens] |
| 270127 | 1.3786197 | |
| 49240 | 1.3772023 | KIAA0460 protein |
| 826622 | 1.3734143 | KIAA0430 gene product |
| 1858837 | 1.3719341 | ESTs |
| 1583198 | 1.3712112 | ESTs, Weakly similar to S65824 reverse transcriptase |
| | | homolog [H. sapiens] |
| 345858 | 1.3629187 | cisplatin resistance associated |
| 208387 | 1.3597205 | KIAA1407 protein |
| 502782 | 1.3589088 | RAN binding protein 3 |
| 26294 | 1.3578289 | RNB6 |
| 669379 | 1.3559931 | Homo sapiens BAC clone RP11-505D17 from 7p22-p21 |
| 810728 | 1.355262 | hypothetical gene ZD52F10 |
| 1601845 | 1.3533864 | Ca2+-promoted Ras inactivator |
| 840882 | 1.350538 | nucleotide binding protein |
| 82173 | 1.3485351 | MYLE protein |
| 490965 | 1.3476943 | ESTs |
| 811162 | 1.3450626 | fibromodulin |
| 61061 | 1.3437368 | hypothetical protein FLJ20585 |
| 49630 | 1.3412751 | calcium channel, voltage-dependent, L type, alpha 1D subunit |
| 825659 | −2.8345933 | N-myc downstream regulated |
| 769921 | −2.712812 | ubiquitin carrier protein E2-C |
| 788232 | −2.6550962 | sestrin 2 |
| 1476053 | −2.529039 | RAD51 (S. cerevisiae) homolog (E coli RecA homolog) |
| 809557 | −2.4910488 | minichromosome maintenance deficient (S. cerevisiae) 3 |
| 150897 | −2.4302462 | UDP-GlcNAc:betaGal beta-1,3-N- |
| | | acetylglucosaminyltransferase 3 |
| 814526 | −2.37273 | seb4D |
| 796694 | −2.3104815 | baculoviral IAP repeat-containing 5 (survivin) |
| 814792 | −2.3001334 | ubiquitin specific protease 10 |
| 1536236 | −2.2971152 | hypothetical protein FLJ13154 |
| 2017415 | −2.2748559 | centromere protein A (17 kD) |
| 210862 | −2.2408114 | acyl-Coenzyme A oxidase 1, palmitoyl |
| 795543 | −2.2403389 | thioredoxin peroxidase (antioxidant enzyme) |
| 773301 | −2.2239527 | cadherin 3, type 1, P-cadherin (placental) |
| 700792 | −2.2203557 | cyclin-dependent kinase inhibitor 3 (CDK2-associated dual |
| | | specificity phosphatase) |
| 1883327 | −2.2093248 | ESTs |
| 1523225 | −2.1936598 | oncostatin M receptor |
| 208718 | −2.1882654 | annexin A1 |
| 1702742 | −2.1567809 | solute carrier family 7 (cationic amino acid transporter, y+ |
| | | system), member 5 |
| 753378 | −2.151145 | hypothetical protein FLJ22649 similar to signal peptidase |
| | | SPC22/23 |
| 429222 | −2.1351022 | CGI-107 protein |
| 292388 | −2.1343367 | |
| 781047 | −2.1262682 | budding uninhibited by benzimidazoles 1 (yeast homolog) |
| 450854 | −2.0819596 | craniofacial development protein 1 |
| 823598 | −2.0693353 | proteasome (prosome, macropain) 26S subunit, non-ATPase, |
| | | 12 |
| 1422338 | −2.0689327 | ribonucleotide reductase M2 polypeptide |
| 705064 | −2.0601901 | transforming, acidic coiled-coil containing protein 3 |
| 770675 | −2.0272207 | Homo sapiens cDNA: FLJ21323 fis, clone COL02374 |
| 345787 | −1.9992897 | highly expressed in cancer, rich in leucine heptad repeats |
| 471196 | −1.9985751 | integral membrane protein 3 |
| 753215 | −1.9954476 | guanine nucleotide binding protein (G protein), alpha inhibiting |
| | | activity polypeptide 1 |
| 869375 | −1.9803724 | isocitrate dehydrogenase 2 (NADP+), mitochondrial |
| 842818 | −1.9796507 | lysyl-tRNA synthetase |
| 229579 | −1.9782493 | Golgi apparatus protein 1 |
| 1916461 | −1.9736332 | hypothetical protein |
| 129294 | −1.9730334 | ESTs |
| 789376 | −1.9719575 | thioredoxin reductase 1 |
| 1897302 | −1.9681938 | serine/threonine kinase 17b (apoptosis-inducing) |
| 951117 | −1.9577807 | eukaryotic translation elongation factor 1 gamma |
| 1642496 | −1.9484342 | hypothetical protein MGC11266 |
| 591465 | −1.9277062 | Homo sapiens, clone MGC: 2908 IMAGE: 3029644, mRNA, |
| | | complete cds |
| 149355 | −1.8999907 | translocating chain-associating membrane protein |
| 259950 | −1.8958779 | hypothetical protein FLJ14991 |
| 897770 | −1.8887034 | |
| 878798 | −1.8774101 | beta-2-microglobulin |
| 746229 | −1.873434 | mitogen-activated protein kinase kinase kinase kinase 4 |
| 624867 | −1.8594915 | hypothetical protein FLJ20186 |
| 504308 | −1.8590049 | hypothetical protein FLJ10540 |
| 727251 | −1.8553757 | CD9 antigen (p24) |
| 897774 | −1.8532125 | adenine phosphoribosyltransferase |
| 1901310 | −1.8434048 | KIAA1209 protein |
| 292936 | −1.838542 | hypothetical protein FLJ10468 |
| 1518591 | −1.8382765 | |
| 321354 | −1.8123042 | mitochondrial ribosomal protein L15 |
| 1903066 | −1.8071117 | keratin, hair, basic, 1 |
| 235180 | −1.8004039 | ESTs, Moderately similar to ALU1_HUMAN ALU SUBFAMILY |
| | | J SEQUENCE CONTAMINATION WARNING ENTRY |
| | | [H. sapiens] |
| 740604 | −1.7995578 | interferon stimulated gene (20 kD) |
| 233464 | −1.7978669 | epithelial V-like antigen 1 |
| 951241 | −1.7961406 | clone HQ0310 PRO0310p1 |
| 1587847 | −1.7886092 | minichromosome maintenance deficient (mis5, S. pombe) 6 |
| 624390 | −1.7800152 | DC13 protein |
| 128711 | −1.7791673 | anillin (Drosophila Scraps homolog), actin binding protein |
| 308633 | −1.7667341 | hypothetical protein FLJ10339 |
| 884425 | −1.761486 | chaperonin containing TCP1, subunit 5 (epsilon) |
| 745394 | −1.7583344 | Homo sapiens cDNA: FLJ23249 fis, clone COL04196 |
| 852829 | −1.7579704 | karyopherin alpha 3 (importin alpha 4) |
| 122241 | −1.7490794 | proteasome (prosome, macropain) subunit, beta type, 2 |
| 307255 | −1.7294781 | basement membrane-induced gene |
| 32493 | −1.7273675 | integrin, alpha 6 |
| 454896 | −1.7216328 | DnaJ (Hsp40) homolog, subfamily A, member 2 |
| 1876217 | −1.7186822 | DnaJ (Hsp40) homolog, subfamily A, member 2 |
| 815556 | −1.7161709 | hypothetical protein FLJ10430 |
| 589869 | −1.7120206 | transcriptional co-activator with PDZ-binding motif (TAZ) |
| 2012523 | −1.7104314 | fatty acid binding protein 5 (psoriasis-associated) |
| 1946448 | −1.7092537 | caveolin 2 |
| 841370 | −1.7047297 | glutamic-oxaloacetic transaminase 2, mitochondrial (aspartate |
| | | aminotransferase 2) |
| 1600239 | −1.6989076 | HSPC037 protein |
| 462926 | −1.6981004 | NIMA (never in mitosis gene a)-related kinase 2 |
| 144880 | −1.6980733 | hypothetical protein from EUROIMAGE 1759349 |
| 744047 | −1.6910803 | polo (Drosophila)-like kinase |
| 624627 | −1.6888444 | ribonucleotide reductase M2 polypeptide |
| 788566 | −1.6752673 | Purkinje cell protein 4 |
| 858293 | −1.6732706 | tyrosine 3-monooxygenase/tryptophan 5-monooxygenase |
| | | activation protein, theta polypeptide |
| 470148 | −1.659943 | ESTs |
| 66902 | −1.6546363 | ESTs |
| 77533 | −1.6505418 | inositol polyphosphate-5-phosphatase, 40 kD |
| 825282 | −1.6417354 | DKFZP586L0724 protein |
| 785840 | −1.6406565 | SEC24 (S. cerevisiae) related gene family, member D |
| 824962 | −1.6399941 | karyopherin alpha 2 (RAG cohort 1, importin alpha 1) |
| 741139 | −1.6396499 | eyes absent (Drosophila) homolog 2 |
| 809530 | −1.6264151 | minichromosome maintenance deficient (S. cerevisiae) 2 |
| | | (mitotin) |
| 2054635 | −1.6254343 | proteasome (prosome, macropain) subunit, alpha type, 7 |
| 813533 | −1.6215197 | syndecan binding protein (syntenin) |
| 267816 | −1.6173276 | KIAA0551 protein |
| 306318 | −1.6083862 | origin recognition complex, subunit 6 (yeast homolog)-like |
| 796469 | −1.6065509 | HSPC150 protein similar to ubiquitin-conjugating enzyme |
| 813256 | −1.6027042 | ATP-binding cassette, sub-family B (MDR/TAP), member 1 |
| 843121 | −1.60128 | chloride intracellular channel 1 |
| 713685 | −1.6006729 | protease, serine, 2 (trypsin 2) |
| 811590 | −1.6001102 | hypothetical protein FLJ11100 |
| 823756 | −1.5995448 | mitogen inducible 2 |
| 549073 | −1.597146 | capping protein (actin filament) muscle Z-line, alpha 2 |
| 743810 | −1.5960999 | hypothetical protein MGC2577 |
| 377368 | −1.5955102 | cell death regulator aven |
| 1506046 | −1.5918448 | hypothetical protein FLJ10815 |
| 814270 | −1.5890389 | polymyositis/scleroderma autoantigen 1 (75 kD) |
| 1604703 | −1.5853167 | major histocompatibility complex, class I, F |
| 234736 | −1.5765159 | GATA-binding protein 6 |
| 429182 | −1.5699689 | dolichyl-phosphate mannosyltransferase polypeptide 1, |
| | | catalytic subunit |
| 826355 | −1.5677013 | vesicle-associated membrane protein 5 (myobrevin) |
| 34149 | −1.5659923 | KIAA0227 protein |
| 746190 | −1.5635857 | hypothetical protein DKFZp761B1514 |
| 131091 | −1.558448 | Not56 (D. melanogaster)-like protein |
| 665384 | −1.5550415 | KIAA1609 protein |
| 625683 | −1.5501423 | neighbor of COX4 |
| 1582738 | −1.544427 | uncharacterized bone marrow protein BM040 |
| 949988 | −1.5437326 | Homo sapiens mRNA; cDNA DKFZp586E1124 (from clone |
| | | DKFZp586E1124); complete cds |
| 133213 | −1.5433392 | fucosyltransferase 4 (alpha (1,3) fucosyltransferase, myeloid- |
| | | specific) |
| 1660666 | −1.5423907 | carbonic anhydrase VB, mitochondrial |
| 713158 | −1.5372729 | ESTs |
| 1614140 | −1.5341302 | Ris |
| 201890 | −1.5333626 | baculoviral IAP repeat-containing 3 |
| 38925 | −1.5304604 | ESTs, Moderately similar to A47582 B-cell growth factor |
| | | precursor [H. sapiens] |
| 511850 | −1.5294752 | proteasome (prosome, macropain) 26S subunit, ATPase, 1 |
| 489489 | −1.5169076 | lamin B receptor |
| 825470 | −1.5154306 | topoisomerase (DNA) II alpha (170 kD) |
| 42831 | −1.5136459 | N-terminal kinase-like |
| 809784 | −1.5066053 | kallikrein 6 (neurosin, zyme) |
| 730410 | −1.506168 | lymphocyte-specific protein tyrosine kinase |
| 810983 | −1.5060386 | DKFZP434H132 protein |
| 731223 | −1.5058799 | proteasome (prosome, macropain) subunit, beta type, 2 |
| 259017 | −1.5053827 | ESTs |
| 340745 | −1.5041978 | ESTs |
| 746163 | −1.5000358 | ESTs, Weakly similar to ALU1_HUMAN ALU SUBFAMILY J |
| | | SEQUENCE CONTAMINATION WARNING ENTRY |
| | | [H. sapiens] |
| 1466621 | −1.4998321 | ATPase, Ca++ transporting, type 2C, member 1 |
| 809588 | −1.49754 | gamma-glutamyl hydrolase (conjugase, |
| | | folylpolygammaglutamyl hydrolase) |
| 813707 | −1.4946872 | regulator of G-protein signalling 16 |
| 188335 | −1.4895328 | egf-like module containing, mucin-like, hormone receptor-like |
| | | sequence 2 |
| 1493160 | −1.4851884 | small inducible cytokine subfamily B (Cys-X-Cys), member 10 |
| 531886 | −1.4842053 | Homo sapiens clone FLB3344 PRO0845 mRNA, complete cds |
| 753428 | −1.4835152 | Homo sapiens, clone IMAGE: 3542597, mRNA, partial cds |
| 897731 | −1.4833877 | latrophilin |
| 376551 | −1.4811859 | ETAA16 protein |
| 256907 | −1.4755682 | glutathione S-transferase A3 |
| 712139 | −1.4735772 | ADP-ribosylation factor-like 7 |
| 785368 | −1.4680554 | PDZ-binding kinase; T-cell originated protein kinase |
| 825606 | −1.4667088 | kinesin-like 1 |
| 531319 | −1.4659871 | serine/threonine kinase 12 |
| 66406 | −1.4641179 | hypothetical protein DKFZp762E1312 |
| 470124 | −1.4635711 | RAD1 (S. pombe) homolog |

### Example IX

### Genes for Discriminating Between IDC Grade I and Grade III

As shown in Table 8 below, 300 genes were identified as being able to discriminate between two grades of IDC.

TABLE 8

| CloneID | Weight | Description |
|---|---|---|
| 1706635 | 3.279305 | bone gamma-carboxyglutamate (gla) protein (osteocalcin) |
| 666138 | 3.1366533 | hypothetical protein DKFZp761J1523 |
| 795382 | 3.0005724 | Rap1 guanine-nucleotide-exchange factor directly activated by |
| | | cAMP |
| 364865 | 2.8512705 | hypothetical protein FLJ21062 |
| 1500542 | 2.8021264 | regulator of G-protein signalling 11 |
| 270127 | 2.7455118 | |
| 588262 | 2.7127609 | Homo sapiens, Similar to RIKEN cDNA 2600001A11 gene, |
| | | clone MGC: 9907 IMAGE: 3870073, mRNA, complete cds |
| 277266 | 2.5004627 | Homo sapiens, clone IMAGE: 3625550, mRNA, partial cds |
| 2090129 | 2.4882736 | chromobox homolog 2 (Drosophila Pc class) |
| 325583 | 2.4558352 | EST |
| 358151 | 2.4309122 | zinc finger protein 33a (KOX 31) |
| 786675 | 2.3877233 | epididymis-specific, whey-acidic protein type, four-disulfide |
| | | core; putative ovarian carcinoma marker |
| 854763 | 2.378956 | Homo sapiens cDNA FLJ11341 fis, clone PLACE1010786 |
| 1592976 | 2.3417116 | microphthalmia-associated transcription factor |
| 1562231 | 2.286418 | SET binding protein 1 |
| 767176 | 2.2472282 | tumor necrosis factor (ligand) superfamily, member 13 |
| 784178 | 2.2026739 | Homo sapiens mRNA; cDNA DKFZp586M0723 (from clone |
| | | DKFZp586M0723) |
| 811162 | 2.1926501 | fibromodulin |
| 502518 | 2.1886432 | laminin, beta 2 (laminin S) |
| 754429 | 2.1363368 | ESTs, Weakly similar to T00084 hypothetical protein |
| | | KIAA0512 [H. sapiens] |
| 214205 | 2.1127961 | Homo sapiens, clone MGC: 17687 IMAGE: 3865868, mRNA, |
| | | complete cds |
| 61061 | 2.1046094 | hypothetical protein FLJ20585 |
| 344959 | 2.0756965 | gene for serine/threonine protein kinase |
| 814815 | 2.0694714 | plakophilin 4 |
| 262804 | 2.0673444 | hypothetical protein MGC2941 |
| 1455566 | 2.0489061 | adenosine A3 receptor |
| 1469149 | 1.9778151 | Homo sapiens clone 24606 mRNA sequence |
| 358217 | 1.9703763 | glypican 4 |
| 206217 | 1.9595484 | nuclear receptor subfamily 1, group H, member 3 |
| 261609 | 1.958083 | ESTs, Weakly similar to S65657 alpha-1C-adrenergic receptor |
| | | splice form 2 [H. sapiens] |
| 197525 | 1.9550731 | flavin containing monooxygenase 5 |
| 857640 | 1.9532398 | collagen, type VI, alpha 2 |
| 812143 | 1.9469226 | fibronectin leucine rich transmembrane protein 3 |
| 898222 | 1.9409805 | Homo sapiens clone 24418 mRNA sequence |
| 418129 | 1.9385868 | nuclear mitotic apparatus protein 1 |
| 2021882 | 1.9213983 | sodium channel, nonvoltage-gated 1, delta |
| 293819 | 1.919948 | oxidoreductase UCPA |
| 203003 | 1.8989675 | non-metastatic cells 4, protein expressed in |
| 705274 | 1.8986548 | diacylglycerol kinase, delta (130 kD) |
| 124922 | 1.8975916 | KRAB-zinc finger protein SZF1-1 |
| 2505310 | 1.8859615 | calcium/calmodulin-dependent protein kinase I |
| 1492238 | 1.8795818 | HSPC003 protein |
| 810358 | 1.8732889 | acyl-Coenzyme A dehydrogenase, very long chain |
| 2391494 | 1.8698169 | ephrin-A4 |
| 1592530 | 1.8669954 | mammalian inositol hexakisphosphate kinase 2 |
| 810671 | 1.8659671 | hypothetical protein FLJ22269 |
| 2108048 | 1.8648249 | DNB5 |
| 730036 | 1.8564918 | Mad4 homolog |
| 810741 | 1.831265 | GABA(A) receptor-associated protein |
| 45578 | 1.8274273 | mitogen-activated protein kinase kinase 6 |
| 647397 | 1.8263711 | ESTs |
| 767495 | 1.8149096 | GLI-Kruppel family member GLI3 (Greig |
| | | cephalopolysyndactyly syndrome) |
| 32050 | 1.8117146 | Homo sapiens mRNA; cDNA DKFZp586P1124 (from clone |
| | | DKFZp586P1124) |
| 811848 | 1.8106436 | hypothetical protein |
| 186301 | 1.8021505 | Homo sapiens cDNA FLJ12924 fis, clone NT2RP2004709 |
| 366526 | 1.7836259 | ESTs, Weakly similar to ALU5_HUMAN ALU SUBFAMILY SC |
| | | SEQUENCE CONTAMINATION WARNING ENTRY |
| | | [H. sapiens] |
| 813154 | 1.7815662 | nuclear factor I/A |
| 85195 | 1.778695 | growth arrest and DNA-damage-inducible, gamma |
| 2460159 | 1.7730834 | tyrosine kinase, non-receptor, 1 |
| 504959 | 1.7723846 | Homo sapiens mRNA; cDNA DKFZp586G0321 (from clone |
| | | DKFZp586G0321) |
| 742094 | 1.7684813 | hypothetical protein FLJ20950 |
| 256619 | 1.7658619 | hydroxysteroid (17-beta) dehydrogenase 7 |
| 726699 | 1.7594888 | ESTs, Weakly similar to AAB47496 NG5 [H. sapiens] |
| 2017144 | 1.7514699 | CGI-41 protein |
| 74070 | 1.7336683 | endosulfine alpha |
| 1762111 | 1.7236006 | natriuretic peptide receptor C/guanylate cyclase C |
| | | (atrionatriuretic peptide receptor C) |
| 795750 | 1.7116475 | Homo sapiens clone 25056 mRNA sequence |
| 2019750 | 1.7095011 | SEC14 (S. cerevisiae)-like 2 |
| 2325804 | 1.7039903 | 95 kDa retinoblastoma protein binding protein |
| 279720 | 1.693594 | ESTs, Moderately similar to A47582 B-cell growth factor |
| | | precursor [H. sapiens] |
| 132857 | 1.6875688 | Homo sapiens mRNA; cDNA DKFZp586N1323 (from clone |
| | | DKFZp586N1323) |
| 1898619 | 1.684954 | hypothetical protein MGC15737 |
| 681992 | 1.6802347 | Homo sapiens cDNA FLJ13384 fis, clone PLACE1001062, |
| | | highly similar to Homo sapiens mRNA for lysine-ketoglutarate |
| | | reductase/saccharopine dehydrogenase |
| 85450 | 1.6753438 | acyl-Coenzyme A oxidase 2, branched chain |
| 51218 | 1.6748931 | ESTs |
| 1925280 | 1.6563658 | homologous to yeast nitrogen permease (candidate tumor |
| | | suppressor) |
| 250883 | 1.6504115 | ubiquitin-activating enzyme E1-like |
| 172783 | 1.6502064 | hypothetical protein FLJ10390 |
| 208387 | 1.6498608 | KIAA1407 protein |
| 703964 | 1.6452497 | inositol polyphosphate phosphatase-like 1 |
| 298231 | 1.6428171 | gamma-aminobutyric acid (GABA) B receptor, 1 |
| 277848 | 1.6407233 | ADP-ribosylation factor 6 |
| 669359 | 1.6386236 | Homo sapiens clone 24405 mRNA sequence |
| 321455 | 1.6327309 | Homo sapiens, Similar to RIKEN cDNA 1110002C08 gene, |
| | | clone MGC: 9564 IMAGE: 3872267, mRNA, complete cds |
| 796152 | 1.6295159 | Homo sapiens cDNA FLJ11685 fis, clone HEMBA1004934 |
| 502782 | 1.614818 | RAN binding protein 3 |
| 360778 | 1.6091782 | |
| 950574 | 1.606032 | H3 histone, family 3B (H3.3B) |
| 788334 | 1.5816572 | mitochondrial ribosomal protein L23 |
| 782497 | 1.5767626 | Homo sapiens, clone IMAGE: 3010666, mRNA, partial cds |
| 1733262 | 1.5656438 | BLu protein |
| 342181 | 1.5642436 | B-cell CLL/lymphoma 2 |
| 1632248 | 1.5590794 | Homo sapiens cDNA FLJ14181 fis, clone NT2RP2004300 |
| 1526826 | 1.5576997 | homeo box B2 |
| 145132 | 1.5520317 | mannose-P-dolichol utilization defect 1 |
| 183062 | 1.5400324 | ubiquitin specific protease 21 |
| 823663 | 1.5337257 | fragile X mental retardation, autosomal homolog 2 |
| 190059 | 1.5258594 | guanine nucleotide binding protein (G protein), gamma 7 |
| 111721 | 1.5045285 | insulin induced protein 2 |
| 840882 | 1.5039692 | nucleotide binding protein |
| 1902764 | 1.5037016 | 6-phosphofructo-2-kinase/fructose-2,6-biphosphatase 3 |
| 414999 | 1.5021506 | ets variant gene 4 (E1A enhancer-binding protein, E1AF) |
| 1636523 | 1.4948923 | glutathione S-transferase subunit 13 homolog |
| 594683 | 1.4862053 | ESTs |
| 1916575 | 1.4825298 | BCL2-interacting killer (apoptosis-inducing) |
| 1456937 | 1.4730662 | oviductal glycoprotein 1, 120 kD (mucin 9, oviductin) |
| 25274 | 1.464238 | g20 protein |
| 795288 | 1.4625618 | ubiquitin specific protease 4 (proto-oncogene) |
| 1569418 | 1.4599089 | Homo sapiens cDNA FLJ11385 fis, clone HEMBA1000520 |
| 753700 | 1.4522717 | Ras-related GTP-binding protein |
| 1691237 | 1.4516493 | ESTs |
| 741891 | 1.450795 | RAB2, member RAS oncogene family-like |
| 1493383 | 1.4468163 | Homo sapiens mRNA; cDNA DKFZp434H2418 (from clone |
| | | DKFZp434H2418) |
| 1675273 | 1.445145 | RAR-related orphan receptor C |
| 293916 | 1.4265341 | FKBP-associated protein |
| 39600 | 1.4224626 | adenylate kinase 5 |
| 1573087 | 1.4200341 | KIAA0592 protein |
| 839796 | 1.4114662 | candidate tumor suppressor p33 ING1 homolog |
| 296123 | 1.4057088 | Homo sapiens PRO1851 mRNA, complete cds |
| 727263 | 1.4046408 | heterogeneous nuclear ribonucleoprotein U (scaffold |
| | | attachment factor A) |
| 265045 | 1.4041143 | Homo sapiens mRNA; cDNA DKFZp586B0918 (from clone |
| | | DKFZp586B0918) |
| 869450 | 1.4034983 | ribosomal protein L11 |
| 714472 | 1.4006155 | KIAA0397 gene product |
| 825296 | 1.3963737 | low density lipoprotein receptor defect C complementing |
| 753301 | 1.3962897 | carcinoembryonic antigen-related cell adhesion molecule 1 |
| | | (biliary glycoprotein) |
| 700527 | 1.394937 | glutaredoxin (thioltransferase) |
| 811565 | 1.3909974 | KIAA1694 protein |
| 1630990 | 1.3843882 | ribosomal protein L29 |
| 70749 | 1.3810753 | Homo sapiens cDNA: FLJ21874 fis, clone HEP02488 |
| 814826 | 1.3785178 | ESTs |
| 175103 | 1.376581 | cadherin, EGF LAG seven-pass G-type receptor 2, flamingo |
| | | (Drosophila) homolog |
| 215000 | 1.3677098 | vasoactive intestinal peptide receptor 1 |
| 810331 | 1.3656069 | quiescin Q6 |
| 1681421 | 1.3645145 | EGF-like-domain, multiple 3 |
| 502198 | 1.3619124 | protein phosphatase 1, regulatory (inhibitor) subunit 5 |
| 825365 | 1.361275 | hypothetical protein FLJ21919 |
| 1557047 | 1.3582677 | thrombospondin 3 |
| 2067500 | 1.3579293 | Z-band alternatively spliced PDZ-motif |
| 490615 | 1.3566637 | tubulin, gamma 2 |
| 743880 | 1.3528093 | KIAA0263 gene product |
| 812099 | 1.3444147 | RNA binding motif protein 5 |
| 868652 | 1.3432331 | complement component 4B |
| 120138 | 1.3384804 | J domain containing protein 1 |
| 156363 | 1.3358314 | hypothetical protein FLJ12934 |
| 813584 | 1.3327194 | p53 regulated PA26 nuclear protein |
| 726703 | 1.3247883 | Homo sapiens clone 23736 mRNA sequence |
| 809507 | 1.3225829 | hypothetical protein FLJ20568 |
| 2018423 | 1.3212314 | death-associated protein kinase 2 |
| 292806 | 1.3201283 | chromosome segregation 1 (yeast homolog)-like |
| 741977 | 1.3200635 | B-factor, properdin |
| 610326 | −4.5776738 | |
| 322494 | −3.6341126 | core-binding factor, beta subunit |
| 824962 | −3.6328144 | karyopherin alpha 2 (RAG cohort 1, importin alpha 1) |
| 825659 | −3.6247426 | N-myc downstream regulated |
| 796694 | −3.5038212 | baculoviral IAP repeat-containing 5 (survivin) |
| 149355 | −3.4959791 | translocating chain-associating membrane protein |
| 814270 | −3.3246381 | polymyositis/scleroderma autoantigen 1 (75 kD) |
| 884425 | −3.3113482 | chaperonin containing TCP1, subunit 5 (epsilon) |
| 1874367 | −3.2390912 | small inducible cytokine subfamily A (Cys—Cys), member 20 |
| 742707 | −3.2093502 | ESTs, Weakly similar to MUC2_HUMAN MUCIN 2 |
| | | PRECURSOR [H. sapiens] |
| 321354 | −3.1386611 | mitochondrial ribosomal protein L15 |
| 280375 | −3.110486 | PRO2000 protein |
| 2018154 | −3.0906181 | electron-transfer-flavoprotein, beta polypeptide |
| 814792 | −3.0561472 | ubiquitin specific protease 10 |
| 826070 | −3.0517117 | KIAA0948 protein |
| 624867 | −2.9688171 | hypothetical protein FLJ20186 |
| 202035 | −2.9361687 | uncharacterized hypothalamus protein HT010 |
| 1476053 | −2.9129535 | RAD51 (S. cerevisiae) homolog (E coli RecA homolog) |
| 194318 | −2.8913426 | hypothetical protein MGC5585 |
| 769921 | −2.8415969 | ubiquitin carrier protein E2-C |
| 1732922 | −2.8216808 | Homo sapiens mRNA; cDNA DKFZp762H106 (from clone |
| | | DKFZp762H106) |
| 1536236 | −2.7995652 | hypothetical protein FLJ13154 |
| 200402 | −2.7793137 | hypothetical protein dJ616B8.3 |
| 815556 | −2.7693 | hypothetical protein FLJ10430 |
| 1055607 | −2.7538365 | SUMO-1 activating enzyme subunit 1 |
| 1466621 | −2.7448687 | ATPase, Ca++ transporting, type 2C, member 1 |
| 770675 | −2.6929924 | Homo sapiens cDNA: FLJ21323 fis, clone COL02374 |
| 79520 | −2.6909833 | RAB2, member RAS oncogene family |
| 950355 | −2.6845343 | ESTs, Weakly similar to S13495 pregnancy zone protein |
| | | [H. sapiens] |
| 79710 | −2.6813265 | KIAA0174 gene product |
| 2011138 | −2.6569485 | KIAA1036 protein |
| 898333 | −2.6509707 | ESTs |
| 878846 | −2.6361347 | brain protein I3 |
| 842818 | −2.63282 | lysyl-tRNA synthetase |
| 1553065 | −2.627227 | hypothetical protein FLJ14993 |
| 136722 | −2.6269431 | ATPase, Na+/K+ transporting, beta 3 polypeptide |
| 308633 | −2.6261051 | hypothetical protein FLJ10339 |
| 854581 | −2.6159344 | transcription factor 4 |
| 951241 | −2.5987959 | clone HQ0310 PRO0310p1 |
| 292936 | −2.5940369 | hypothetical protein FLJ10468 |
| 781047 | −2.569753 | budding uninhibited by benzimidazoles 1 (yeast homolog) |
| 823907 | −2.5587562 | hypothetical protein FLJ10511 |
| 377368 | −2.5495557 | cell death regulator aven |
| 292388 | −2.5448438 | |
| 122241 | −2.539248 | proteasome (prosome, macropain) subunit, beta type, 2 |
| 731223 | −2.5364769 | proteasome (prosome, macropain) subunit, beta type, 2 |
| 1660666 | −2.534682 | carbonic anhydrase VB, mitochondrial |
| 825470 | −2.530053 | topoisomerase (DNA) II alpha (170 kD) |
| 1472719 | −2.526978 | SMT3 (suppressor of mif two 3, yeast) homolog 1 |
| 869375 | −2.5249164 | isocitrate dehydrogenase 2 (NADP+), mitochondrial |
| 302292 | −2.5097213 | exostoses (multiple) 2 |
| 1175007 | −2.4998247 | tumor necrosis factor receptor superfamily, member 10d, |
| | | decoy with truncated death domain |
| 1422338 | −2.488461 | ribonucleotide reductase M2 polypeptide |
| 2014034 | −2.4843808 | methylene tetrahydrofolate dehydrogenase (NAD+ |
| | | dependent), methenyltetrahydrofolate cyclohydrolase |
| 50884 | −2.4377483 | dual-specificity tyrosine-(Y)-phosphorylation regulated kinase 2 |
| 1474424 | −2.4345879 | Homo sapiens cDNA FLJ12758 fis, clone NT2RP2001328 |
| 753285 | −2.4324065 | glycogenin |
| 700792 | −2.4311609 | cyclin-dependent kinase inhibitor 3 (CDK2-associated dual |
| | | specificity phosphatase) |
| 259950 | −2.4310983 | hypothetical protein FLJ14991 |
| 610326 | −2.4214624 | |
| 284004 | −2.4185183 | ESTs |
| 756595 | −2.3963227 | S100 calcium-binding protein A10 (annexin II ligand, calpactin |
| | | I, light polypeptide (p11)) |
| 128711 | −2.3891503 | anillin (Drosophila Scraps homolog), actin binding protein |
| 1582738 | −2.3882015 | uncharacterized bone marrow protein BM040 |
| 1915867 | −2.3829702 | Homo sapiens cDNA: FLJ21086 fis, clone CAS03272 |
| 754582 | −2.3765311 | ecotropic viral integration site 2A |
| 462961 | −2.3685047 | dihydrofolate reductase |
| 705064 | −2.3682586 | transforming, acidic coiled-coil containing protein 3 |
| 1553696 | −2.3605995 | vanin 1 |
| 129294 | −2.3507956 | ESTs |
| 809588 | −2.3342142 | gamma-glutamyl hydrolase (conjugase, |
| | | folylpolygammaglutamyl hydrolase) |
| 1909526 | −2.333814 | ESTs, Weakly similar to 2004399A chromosomal protein |
| | | [H. sapiens] |
| 815771 | −2.3166739 | chromosome 7 open reading frame 2 |
| 703707 | −2.2865473 | aspartate beta-hydroxylase |
| 2017415 | −2.2832169 | centromere protein A (17 kD) |
| 1650942 | −2.2797717 | Human proteinase activated receptor-2 mRNA, 3′UTR |
| 645079 | −2.2761946 | 3′(2′), 5′-bisphosphate nucleotidase 1 |
| 133213 | −2.2753496 | fucosyltransferase 4 (alpha (1,3) fucosyltransferase, myeloid- |
| | | specific) |
| 788566 | −2.2752004 | Purkinje cell protein 4 |
| 2009574 | −2.2646007 | T brachyury (mouse) homolog |
| 1897302 | −2.2563379 | serine/threonine kinase 17b (apoptosis-inducing) |
| 78869 | −2.2539078 | cell membrane glycoprotein, 110000M(r) (surface antigen) |
| 590759 | −2.2513499 | sterol-C4-methyl oxidase-like |
| 785368 | −2.2489394 | PDZ-binding kinase; T-cell originated protein kinase |
| 1714689 | −2.2489064 | ESTs |
| 795197 | −2.2481741 | ubiquitin-conjugating enzyme E2E 2 (homologous to yeast |
| | | UBC4/5) |
| 781472 | −2.2390098 | Tax1 (human T-cell leukemia virus type I) binding protein 1 |
| 1469966 | −2.2375228 | delta (Drosophila)-like 3 |
| 1587847 | −2.2354975 | minichromosome maintenance deficient (mis5, S. pombe) 6 |
| 209066 | −2.2325695 | |
| 1035796 | −2.2308926 | ESTs |
| 233464 | −2.2280935 | epithelial V-like antigen 1 |
| 753320 | −2.2104141 | hypothetical protein FLJ20533 |
| 486626 | −2.2095389 | ESTs |
| 1505038 | −2.2060272 | hypothetical protein FLJ20171 |
| 503851 | −2.1992378 | nuclear receptor co-repressor/HDAC3 complex subunit |
| 503671 | −2.1939286 | Homo sapiens cDNA FLJ14368 fis, clone HEMBA1001122 |
| 610326 | −2.1885269 | |
| 712139 | −2.1839319 | ADP-ribosylation factor-like 7 |
| 471196 | −2.1797647 | integral membrane protein 3 |
| 753215 | −2.1781545 | guanine nucleotide binding protein (G protein), alpha inhibiting |
| | | activity polypeptide 1 |
| 183556 | −2.1734918 | gap junction protein, alpha 4, 37 kD (connexin 37) |
| 809557 | −2.1667294 | minichromosome maintenance deficient (S. cerevisiae) 3 |
| 610326 | −2.1633594 | hypothetical protein MGC12992 |
| 1600239 | −2.1602202 | HSPC037 protein |
| 1049291 | −2.1593791 | olfactory receptor, family 7, subfamily E, member 47 |
| | | pseudogene |
| 839682 | −2.1475459 | ubiquitin-conjugating enzyme E2N (homologous to yeast |
| | | UBC13) |
| 950897 | −2.1419057 | Homo sapiens mRNA; cDNA DKFZp586G1922 (from clone |
| | | DKFZp586G1922) |
| 712577 | −2.1411578 | holocytochrome c synthase (cytochrome c heme-lyase) |
| 824524 | −2.1400285 | UDP-galactose transporter related |
| 241348 | −2.1301572 | prenylcysteine lyase |
| 823930 | −2.1259223 | actin related protein 2/3 complex, subunit 1A (41 kD) |
| 150314 | −2.1206632 | lysophospholipase I |
| 795498 | −2.1190094 | putative transmembrane protein |
| 82710 | −2.1184513 | glutathione S-transferase A2 |
| 1869201 | −2.1135906 | hypothetical protein MGC2745 |
| 1694526 | −2.1126121 | hypothetical protein FLJ11029 |
| 823598 | −2.1072985 | proteasome (prosome, macropain) 26S subunit, non-ATPase, |
| | | 12 |
| 214162 | −2.1065234 | metallothionein 1H |
| 811590 | −2.1050783 | hypothetical protein FLJ11100 |
| 796904 | −2.1050163 | pleiomorphic adenoma gene-like 1 |
| 1517595 | −2.0965219 | KIAA0175 gene product |
| 282428 | −2.0954489 | ESTs, Weakly similar to A46010 X-linked retinopathy protein |
| | | [H. sapiens] |
| 1883219 | −2.0949091 | ESTs |
| 746229 | −2.0906355 | mitogen-activated protein kinase kinase kinase kinase 4 |
| 610326 | −2.0896786 | hypothetical protein MGC12992 |
| 1632225 | −2.0828168 | ESTs |
| 743810 | −2.0796998 | hypothetical protein MGC2577 |
| 1416055 | −2.0725881 | extra spindle poles, S. cerevisiae, homolog of |
| 1626297 | −2.0656173 | hypothetical protein FLJ20509 |
| 852829 | −2.0608067 | karyopherin alpha 3 (importin alpha 4) |
| 840511 | −2.0554449 | vimentin |
| 278504 | −2.0519206 | neurochondrin |
| 1534700 | −2.0506628 | KIAA0830 protein |
| 812276 | −2.0458249 | synuclein, alpha (non A4 component of amyloid precursor) |
| 306841 | −2.0449642 | T cell receptor beta locus |
| 3172883 | −2.0437672 | ESTs, Weakly similar to 1709359A dopamine D4 receptor |
| | | [H. sapiens] |
| 624627 | −2.0409259 | ribonucleotide reductase M2 polypeptide |
| 486179 | −2.0380168 | Homo sapiens cDNA FLJ10205 fis, clone HEMBA1004954 |
| 376316 | −2.0218598 | wee1+ (S. pombe) homolog |
| 37671 | −2.0190035 | hypothetical protein FLJ21610 |
| 629944 | −2.0166696 | myosin VB |
| 713158 | −2.0133834 | ESTs |
| 504308 | −2.0067977 | hypothetical protein FLJ10540 |
| 1605426 | −1.9957389 | hypothetical protein FLJ13352 |
| 810711 | −1.9920785 | stearoyl-CoA desaturase (delta-9-desaturase) |
| 263790 | −1.9904129 | isocitrate dehydrogenase 3 (NAD+) alpha |
| 549349 | −1.9893582 | hypothetical protein PRO1068 |
| 109221 | −1.9879196 | KIAA0286 protein |
| 51773 | −1.9875155 | hypothetical protein MGC3077 |

### Example X

### Crossvalidation

The following table shows the results of crossvalidation analysis (as described in Example I above) of various pair-wise comparisons. The two members of the pairwise comparison are indicated as “Class 1” and “Class 2” with the number of samples analyzed indicated under “N”. The number of genes used to predict is indicated, along with the accuracy (in percent) for each combination. “FP” and “FN” refer to “false positive” and “false negative”, respectively, as incorrectly identified for each of the two classes.

| | | | | | | Class | | Class | |
|---|---|---|---|---|---|---|---|---|---|
| Class 1 | | Class 2 | | | | 1 | | 2 | |
| Name | N | Name | N | Genes | Accuracy | FP | FN | FP | FN |
| Normal | 28 | Abnormal | 57 | 850 | 99 | 0 | 1 | 1 | 0 |
| Normal | 28 | ADH | 7 | 600 | 100 | 0 | 0 | 0 | 0 |
| Normal | 28 | DCIS | 28 | 1300 | 100 | 0 | 0 | 0 | 0 |
| ADH | 7 | DCIS | 28 | 350 | 97 | 0 | 1 | 1 | 0 |
| ADH | 7 | DCIS | 28 | 10 | 97 | 0 | 1 | 1 | 0 |
| N + | 36 | DCIS + IDC | 49 | 400 | 98 | 1 | 1 | 1 | 1 |
| ADH | | | | | | | | | |
| DCIS-I | 7 | DCIS-III | 9 | 300 | 100 | 0 | 0 | 0 | 0 |
| IDC-I | 4 | IDC-III | 8 | 300 | 100 | 0 | 0 | 0 | 0 |

### REFERENCES

DeRisi, J., et al., Use of a cDNA microarray to analyse gene expression patterns in human cancer, Nature Genetics, (1996) 14:457-460.
Hedenfalk, I., et al., Gene-Expression Profiles In Heredity Breast Cancer, The New England Journal of Medicine, (Feb. 22, 2001) 344:8:539-548.
Golub, T. R., et al., Molecular Classification of Cancer: Class Discovery and Class Prediction by Gene Expression Monitoring, Science, (Oct. 15, 1999) 286:531-537.
Perou, Charles M., et al., Molecular portraits of human breast tumours, Nature, (Aug. 17, 2000) 406:747-752.
Garber, Mitchell E., et al., Diversity of gene expression in adenocarcinoma of the lung, Proc. Natl. Acad. Sci. USA, (Nov. 20, 2001) 98:24:13784-13789.
Perou, Charles M., et al., Distinctive gene expression patterns in human mammary epithelial cells and breast cancers, Proc. Natl. Acad. Sci. USA, (August 1999) 96:9212-9217.
Sgrio, Dennis C., et al., In Vivo Gene Expression Profile Analysis of Human Breast Cancer Progression, Cancer Research, (Nov. 15, 1999) 59:5656-5661.
Sorlie, Therese, et al., Gene expression patterns of breast carcinomas distinguish tumor subclasses with clinical implications, Proc. Natl. Acad. Sci., (Sep. 11, 2001) 98:19:10869-10874.
Alizadeh, Ash A., et al., Distinct types of diffuse large B-cell lymphoma identified by gene expression profiling, Nature, (Feb. 3, 2000) 403:503-511.
Bittner, M., et al., Molecular classification of cutaneous malignant melanoma by gene expression profiling, Nature (Aug. 3, 2000) 406:536-540.
West, Mike, et al., Predicting the clinical status of human breast cancer by using gene expression profiles, Proc. Natl. Acad. Sci., (Sep. 25, 2001) 98:20:11462-11467.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

We claim:

1. A method to determine the breast cancer stage of a ductal lavage or fine needle aspiration sample from a subject comprising
assaying said sample for expression of 5 or more of the genes in any one of Tables 2-5, which genes are correlated with one or more stages of breast cancer, and
determining the breast cancer stage of said sample based on the level of expression of said genes.

2. The method of claim 1 wherein said assaying comprises preparing mRNA from said sample.

3. The method of claim 1 wherein said mRNA is amplified.

4. The method of claim 1 wherein said assaying comprises detecting expression by use of an array.

5. The method of claim 1 wherein said assaying comprises detecting expression by use of a microarray comprising polynucleotides which hybridize to said genes.

6. The method of claim 1 wherein said expression of 5 or more genes is correlated with ADH, DCIS, and/or IDC.

7. The method of claim 1 wherein said one or more genes are correlated with normal or abnormal cells.

8. The method of claim 5 wherein said array comprises polynucleotides which hybridize to more than 5 of the genes in any one of Tables 2-5.

9. The method of claim 1 wherein said subject is human.

10. The method of claim 1 wherein said subject is afflicted with, or suspected of having, breast cancer.

11. The method of claim 5 wherein said array comprises polynucleotides which hybridize to more than five of the genes in Table 2.

12. The method of claim 5 wherein said array comprises polynucleotides which hybridize to more than five of the genes in Table 3.

13. The method of claim 5 wherein said array comprises polynucleotides which hybridize to more than five of the genes in Table 4.

14. The method of claim 5 wherein said array comprises polynucleotides which hybridize to more than five of the genes in Table 5.

15. The method of claim 1 wherein said sample is a microdissected sample.

16. The method of claim 15 wherein said sample is microdissected via laser capture microdissection.

17. The method of claim 6 wherein said one or more genes are correlated with ADH or correlated with DCIS or correlated with IDC.

18. The method of claim 6 wherein said assaying comprises preparing mRNA from said sample and optionally amplifying said mRNA.

19. The method of claim 6 wherein said assaying comprises detecting expression by use of an array.

20. The method of claim 6 wherein said subject is human, optionally afflicted with, or suspected of having, breast cancer.
